(19) United States
(12) Patent Application Publication
(10) Pub. No.: US 2024/0368228 A1
(43) Pub. Date: Nov. 7, 2024

(54) CAPSID VARIANTS AND METHODS OF USING THE SAME

(71) Applicant: DYNO THERAPEUTICS, INC., Watertown, MA (US)

(72) Inventors: Sylvain Lapan, Brookline, MA (US); Hanna Levitin, Cambridge, MA (US); Lauren Wheelock, Cambridge, MA (US)

(21) Appl. No.: 18/566,272

(22) PCT Filed: Jun. 2, 2022
(86) PCT No.: PCT/US2022/032004

§ 371 Date: Dec. 1, 2023

Related U.S. Application Data
(60) Provisional application No. 63/342,455, filed on May 16, 2022.
(60) Provisional application No. 63/331,627, filed on Apr. 15, 2022.
(60) Provisional application No. 63/196,554, filed on Jun. 3, 2021.

(51) Int. Cl. C07K 14/005; A61K 48/00; C12N 15/86

(52) U.S. Cl. CPC C07K 14/005; A61K 48/0075; C12N 15/86; C12N 2750/14122; C12N 2750/14143; C12N 2750/14151

(57) ABSTRACT

The disclosure is directed in part to variant capsid polypeptides that can be used to deliver payloads.

### CROSS-REFERENCE TO RELATED APPLICATIONS

This application claims priority to U.S. Provisional Application No. 63/196,554, filed Jun. 3, 2021, U.S. Provisional Application No. 63/331,627, filed Apr. 15, 2022, and U.S. Provisional Application No. 63/342,455, filed May 16, 2022, each of which is hereby incorporated by reference in their entirety.

### BACKGROUND

Dependoparvoviruses, e.g. adeno-associated dependoparvoviruses, e.g. adeno-associated viruses (AAVs), are of interest as vectors for delivering various payloads to cells, including in human subjects.

### SUMMARY

The present disclosure provides, in part, improved variant dependoparvovirus capsid proteins (e.g. AAV2), such as VP1, methods of producing a dependoparvovirus, compositions for use in the same, as well as viral particles produced by the same. In some embodiments, the viral particles that are produced have increased ocular transduction as compared to viral particles without the mutations in the capsid proteins.

In some embodiments, the disclosure is directed, in part, to a nucleic acid comprising a sequence encoding a variant capsid protein as provided for herein. In some embodiments, the dependoparvovirus is an adeno-associated dependoparvovirus (AAV). In some embodiments, the AAV is AAV2.

In some embodiments, the disclosure is directed, in part, to a capsid polypeptide described herein.

In some embodiments, the disclosure is directed, in part, to a dependoparvovirus particle comprising a nucleic acid described herein.

In some embodiments, the disclosure is directed, in part, to a vector, e.g., a plasmid, comprising a nucleic acid described herein.

In some embodiments, the disclosure is directed, in part, to a dependoparvovirus particle comprising a nucleic acid described herein (e.g., a nucleic acid comprising a sequence encoding a capsid polypeptide, such as VP1, wherein the encoding sequence comprises a change or mutation as provided herein.

In some embodiments, the disclosure is directed, in part, to dependoparvovirus particle comprising a variant capsid polypeptide comprising a polypeptide that has at least 85, 90, 91, 92, 93, 94, 95, 96, 97, 98, 99%, or 100% identity to a VP1, VP2, or VP3 sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46.

In some embodiments, the variant capsid polypeptide comprises a mutation selected from a mutation associated with any of VAR-1 to VAR-2, and a mutation selected from a mutation associated with any of VAR-3 to VAR-16; a mutation selected from a mutation associated with any of VAR-1 to VAR-2 and a mutation selected from a mutation associated with any of VAR-17 to VAR-45; or a mutation selected from a mutation associated with any of VAR-3 to VAR-16 and a mutation selected from a mutation associated with any of VAR-17 to VAR-45.

In some embodiments, the disclosure is directed, in part, to a nucleic acid molecule comprising SEQ ID NO: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or 91, a fragment thereof, or a variant thereof having at least 85, 90, 91, 92, 93, 94, 95, 96, 97, 98, 99, or 100% sequence identity thereto.

In some embodiments, the disclosure is directed, in part, to a vector comprising a nucleic acid described herein, e.g., a nucleic acid comprising a sequence encoding a capsid polypeptide, e.g. a VP1 polypeptide, wherein the encoding sequence comprises a change or mutation as provided for herein.

In some embodiments, the disclosure is directed, in part, to a cell, cell-free system, or other translation system comprising a nucleic acid or vector described herein, e.g., comprising a sequence encoding capsid polypeptide, such as VP1, wherein the capsid polypeptide encoding sequence comprises a change or mutation as provided for herein in the encoding sequence. In some embodiments, the cell, cell-free system, or other translation system comprises a dependoparvovirus particle described herein, e.g., wherein the particle comprises a nucleic acid comprising a sequence encoding a capsid polypeptide, such as a VP1 polypeptide, wherein the encoding sequence comprises a change or mutation as provided for herein.

In some embodiments, the disclosure is directed, in part, to a cell, cell-free system, or other translation system comprising a polypeptide described herein, wherein the polypeptide encoding sequence comprises a change or mutation as provided for herein. In some embodiments, the cell, cell-free system, or other translation system comprises a dependoparvovirus particle described herein, e.g., wherein the particle comprises a nucleic acid comprising a sequence encoding a VP1 polypeptide, wherein the VP1 encoding sequence comprises a change or mutation corresponding such as provided for herein.

In some embodiments, the disclosure is directed, in part, to a method of delivering a payload to a cell comprising contacting the cell with a dependoparvovirus particle comprising a nucleic acid described herein. In some embodiments, the disclosure is directed, in part, to a method of delivering a payload to a cell comprising contacting the cell with a dependoparvovirus particle comprising a capsid polypeptide described herein.

In some embodiments, the disclosure is directed, in part, to a method of making a dependoparvovirus particle, comprising providing a cell, cell-free system, or other translation system, comprising a nucleic acid described herein (e.g., a nucleic acid comprising a sequence encoding an AAV2 capsid variant as provided for herein); and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a method of making a dependoparvovirus particle, comprising providing a cell, cell-free system, or other translation system, comprising a polypeptide described herein; and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a dependoparvovirus particle made in a cell, cell-free system, or other translation system, wherein the cell, cell-free system, or other translation system comprises a nucleic acid encoding a dependoparvovirus comprising an capsid variant as provided for herein.

In some embodiments, the disclosure is directed, in part, to a method of treating a disease or condition in a subject, comprising administering to the subject a dependoparvovirus particle described herein in an amount effective to treat the disease or condition.

The invention is further described with reference to the following numbered embodiments.

### BRIEF DESCRIPTION OF THE DRAWINGS

FIG. 1. Diagram of tissues collected in each region of the eye. In the retina (left and center figures), peripheral and central retina samples from each of the superior, nasal, inferior and temporal regions of the retina were separately collected, macula was also separately collected. In each region, neural retina and choroid/RPE layers (center figure) were separately collected. In the TM/SC region (right figure), superior, temporal, nasal and inferior samples were separately collected.

FIG. 2A-C. Multisequence alignment of representative reference capsid VP1 polypeptides. Such alignment can be used to determine the amino acid positions which correspond to positions within different reference capsid polypeptides.

FIG. 3. Data from variants included in the medium throughput study and injected via an intravitreal (IVT) route of administration. All values log 2 relative to AAV2 wild-type. Values of “−10” indicate variant was not measured.

FIG. 4. Data from variants included in the medium throughput study and injected via an intracameral (IC) route of administration. All values log 2 relative to AAV2 wild-type. Values of “−10” indicate variant was not measured.

FIG. 5A-5C. Data from bulk tissue processed from the medium throughput study for variants injected via the IVT route of administration. All values log 2 relative to AAV2 wild-type. Values of “−10” indicate variant was not measured.

FIG. 6A-6C. Data from bulk tissue processed from the medium throughput study for variants injected via the IC route of administration. All values log 2 relative to AAV2 wild-type. Values of “−10” indicate variant was not measured.

FIG. 7. Plot of bulk trabecular meshwork transduction (IC admin) vs. retina transduction (IVT admin) from Library Experiment 1. Each dot is a unique variant. Variants in dark black were selected for inclusion in the medium throughput experiment. All values log 2 relative to AAV2 wild-type.

FIG. 8. Correlation of neural retina transduction (IVT admin) for variants from Library Experiment 1 (x-axis) and medium throughput experiment (y-axis). All values log 2 relative to AAV2 wild-type.

FIG. 9. single nuclear RNA sequencing results for variants from posterior eye tissue samples from the medium throughput study, reporting number of unique transduction events for each variant. All results are normalized to the amount of each variant in the input test article. Variants labeled “VAR-05-n” correspond to “VAR-n” in Table 1, Table 2 and Table 3.

FIG. 10A-53. Relative biodistribution and transduction measurements for the indicated variants. IVT-MT-BD and IC-MT-BD plot bulk biodistribution rate measurements for the viral vector comprising the variant capsid identified in the FIGS. 10A (VAR-1), 11A (VAR-2), 12A (VAR-3), 13A (VAR-4), 14A (VAR-5), 17A (VAR-8), 18A (VAR-9), 20A (VAR-11), 21A (VAR-12), 22A (VAR-13), 23A (VAR-14), 25A (VAR-17), 27A (VAR-19), 28A (VAR-20), 29A (VAR-21), 30A (VAR-22), 31A (VAR-23), 32A (VAR-24), 34A (VAR-26), 37A (VAR-29), 38A (VAR-30), 39A (VAR-31), 41A (VAR-33), 42A (VAR-34), 43A (VAR-35), 44A (VAR-36), 45A (VAR-37), 46A (VAR-38), 47A (VAR-39), 48A (VAR-40), 49A (VAR-41), 50A (VAR-42), and 51A (VAR-43) from intravitreal injection (“IVT”) and intracameral injection (“IC”) respectively, from each of the ocular tissue samples collected in the medium-throughput experiment. IVT-MT-TD and IC-MT-TD plot bulk transduction rate measurements for the viral vector comprising the variant capsid identified in the FIGS. 10A (VAR-1), 11A (VAR-2), 12A (VAR-3), 13A (VAR-4), 14A (VAR-5), 17A (VAR-8), 18A (VAR-9), 20A (VAR-11), 21A (VAR-12), 22A (VAR-13), 23A (VAR-14), 25A (VAR-17), 27A (VAR-19), 28A (VAR-20), 29A (VAR-21), 30A (VAR-22), 31A (VAR-23), 32A (VAR-24), 34A (VAR-26), 37A (VAR-29), 38A (VAR-30), 39A (VAR-31), 41A (VAR-33), 42A (VAR-34), 43A (VAR-35), 44A (VAR-36), 45A (VAR-37), 46A (VAR-38), 47A (VAR-39), 48A (VAR-40), 49A (VAR-41), 50A (VAR-42), and 51A (VAR-43) from intravitreal injection (“IVT”) and intracameral injection (“IC”) respectively, from each of the ocular tissue samples collected in the medium-throughput experiment. All values are plotted as log(2) variant rate relative to wild-type AAV2 rate. Points represent means of barcode replicate rates (n=8). Error bars are 95% confidence intervals (+/−1.96*standard error of the mean (“SEM”)). The aggregated bulk transduction rates were computed by combining observed counts across all constituent eye regions of a tissue (choroid/RPE, neural retina, neural retina non-macula). The choroid/RPE rate was computed by combining all choroid/RPE samples collected; The neural retina rate was computed by combining all neural retina layer samples collected, including macula. Neural retina non-macula was computed by combining all neural retina layer non-macula retina samples. “Comparison” panels summarize bulk transduction and biodistribution rate measurements (as indicated in the panel) for the viral vector comprising the variant capsid identified in the FIGS. 10B (VAR-1), 11B (VAR-2), 12B (VAR-3), 13B (VAR-4), 14B (VAR-5), 15 (VAR-6), 16 (VAR-7), 17B (VAR-8), 18B (VAR-9), 19 (VAR-10), 20B (VAR-11), 21B (VAR-12), 22B (VAR-13), 23B (VAR-14), 24 (VAR-15), 25B (VAR-17), 26 (VAR-18), 27B (VAR-19), 28B (VAR-20), 29B (VAR-21), 30B (VAR-22), 31B (VAR-23), 32B (VAR-24), 33 (VAR-25), 34B (VAR-26), 35 (VAR-27), 36 (VAR-28), 37B (VAR-29), 38B (VAR-30), 39B (VAR-31), 40 (VAR-32), 41B (VAR-33), 42B (VAR-34), 43B (VAR-35), 44B (VAR-36), 45B (VAR-37), 46B (VAR-38), 47B (VAR-39), 48B (VAR-40), 49B (VAR-41), 50B (VAR-42), 51B (VAR-43), 52 (VAR-44), and 53 (VAR-45) collected in the medium-throughput (MT) experiment Library Experiment 1 and Library Experiment 2 via either IVT or IC administration, as indicated in the panel. All values plotted as log(2) variant rate relative to wild-type AAV2 rate. For Library Experiment 1, points represent log 2-transformed mean of the calculated Bayesian posterior distribution relative to the wild-type AAV2 (see Example section). Error bars represent the 95% confidence intervals using the posterior distribution (+/−1.96*standard deviation (“SD”)). For Library Experiment 2, points represent means of biological replicate rates (n=4 eyes). Error bars are 95% confidence intervals (+/−1.96*SEM). For the MT experiment, points represent means of barcode replicate rates (n=8). Error bars are 95% confidence intervals (+/−1.96*SEM). The aggregated bulk transduction rates for a tissue (e.g. choroid/RPE, neural retina, TM/SC) were computed by combining observed counts across all collected samples for regions of a tissue. Absence of a point indicates the data was not collected.

### ENUMERATED EMBODIMENTS

1. A variant capsid polypeptide comprising a polypeptide that has at least 85, 90, 91, 92, 93, 94, 95, 96, 97, 98, 99%, or 100% identity to a VP1, VP2, or VP3 sequence of SEQ ID NO: 37, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46.

2. The variant capsid polypeptide of embodiment 1, wherein the polypeptide comprises:
a mutation selected from a mutation associated with any of VAR-1 to VAR-2, and a mutation selected from a mutation associated with any of VAR-3 to VAR-16;
a mutation selected from a mutation associated with any of VAR-1 to VAR-2 and a mutation selected from a mutation associated with any of VAR-17 to VAR-45; or
a mutation selected from a mutation associated with any of VAR-3 to VAR-16 and a mutation selected from a mutation associated with any of VAR-17 to VAR-45.

3. The variant capsid polypeptide of embodiment 2, wherein:
the mutation associated with any of VAR-1 to VAR-2 comprises mutations corresponding to residues 1-36 as compared to SEQ ID NO: 1;
the mutation associated with any of VAR-3 to VAR-16 comprises mutations corresponding to residues 431-466 as compared to SEQ ID NO: 1; and
the mutation associated with any of VAR-17 to VAR45 comprises mutations corresponding to residues 552-617 as compared to SEQ ID NO: 1.

4. The variant capsid polypeptide of any of the preceding embodiments, wherein the polypeptide comprises a variant of SEQ ID NO: 1, wherein the variant capsid polypeptide comprises a mutation selected from a mutation associated with any of VAR-1 to VAR-2 and a mutation associated with any of VAR-3 to VAR-16.

5. The variant capsid polypeptide of any of the preceding embodiments, wherein the polypeptide comprises a variant of SEQ ID NO: 1, wherein the variant capsid polypeptide comprises a mutation selected from a mutation associated with any of VAR-1 to VAR-2 and a mutation associated with any of VAR-17 to VAR-45.

6. The variant capsid polypeptide of any of the preceding embodiments, wherein the polypeptide comprises a variant of SEQ ID NO: 1, wherein the variant capsid polypeptide comprises a mutation selected from a mutation associated with any of VAR-3 to VAR-16 and a mutation associated with any of VAR-17 to VAR-45.

7. The variant capsid polypeptide of any of the preceding embodiments, wherein the polypeptide comprises a variant of SEQ ID NO: 1, wherein the variant capsid polypeptide comprises a mutation that corresponds to a mutation at position 3, 6, 11, 12, 14, 15, 19, 21, 23, 24, 25, 29, 31, 33, 446, 449, 450, 451, 452, 454, 455, 456, 457, 458, 459, 460, 461, 464, 552, 554, 555, 556, 557, 558, 559, 561, 566, 575, 578, 580, 581, 585, 586, 587, 588, 589, 590, 591, 593, 594, 597, or 600, an insertion between positions 446 and 447, 447 and 448, 448 and 449, 449 and 450, 451 and 452, 453 and 454, 581 and 582, 583 and 584, 584 and 585, 585 and 586, 586 and 587, 587 and 588, 588 and 589, 589 and 590, 590 and 591, 591 and 592, or 592 and 593 according to SEQ ID NO: 1, optionally wherein the mutation comprises an insertion, a deletion or a substitution.

8. The variant capsid polypeptide of any of the preceding embodiments, wherein the polypeptide comprises a variant of SEQ ID NO: 1, wherein the variant capsid polypeptide comprises a mutation that corresponds to a mutation at position 3, 6, 11, 12, 14, 15, 19, 21, 23, 24, 25, 29, 31, 33, or any combination thereof according to SEQ ID NO: 1, and wherein the mutation comprises a deletion or a substitution.

9. The variant capsid polypeptide of any of the preceding embodiments, wherein the polypeptide comprises a variant of SEQ ID NO: 1, wherein the variant capsid polypeptide comprises a mutation that corresponds to a mutation at position 446, 448, 449, 450, 451, 452, 453, 454, 455, 456, 457, 458, 459, 460, 461, 464, or any combination thereof, an insertion between positions 446 and 447, 447 and 448, 448 and 449, 449 and 450, 451 and 452, 453 and 454, or any combination thereof according to SEQ ID NO: 1, and wherein the mutation comprises an insertion, a deletion or a substitution.

10. The variant capsid polypeptide of any of the preceding embodiments, wherein the polypeptide comprises a variant of SEQ ID NO: 1, wherein the variant capsid polypeptide comprises a mutation that corresponds to a mutation at position 552, 554, 555, 556, 557, 558, 559, 561, 566, 575, 578, 580, 581, 585, 586, 587, 588, 589, 590, 591, 593, 594, 597, 600 or any combination thereof, an insertion between positions 583 and 584, 584 and 585, 585 and 586, 586 and 587, 587 and 588, 588 and 589, 589 and 590, 590 and 591, 591 and 592, 592 and 593, or any combination thereof according to SEQ ID NO: 1, and wherein the mutation comprises an insertion, a deletion or a substitution.

11. A variant capsid polypeptide comprising a variant of SEQ ID NO: 1 comprising a mutation selected from a mutation associated with any of VAR-3 to VAR-16, wherein the mutation is:
an insertion, e.g., an insertion of 4 or more amino acids, e.g., 4-5 amino acids, e.g., 4-6 amino acids, e.g., 4-7 amino acids, e.g., 7 amino acids, that corresponds to an insertion between positions 446 and 454 as compared to SEQ ID NO: 1, and wherein the insertion comprises a polypeptide that has at least 60%, 70%, 80%, 90%, or 100% identity to SEQ ID NO: 93-122;
a substitution, e.g., a substitution of at least 2 or more residues, e.g., at least 6-10 residues, e.g., at least 7-10 residues, e.g., at least 8-10 residues, e.g., at least 9-10 residues, e.g., at least 10 residues that correspond to a substitution at positions between 445 and 465 as compared to SEQ ID NO: 1; and
any combination thereof;
   and wherein the variant comprises at least 3 mutations selected from: T455G/L/Q, T456G, Q457T, S458Q, R459G/T; and further comprises at least 3 other mutations between positions 445-465 as compared to SEQ ID NO: 1, and wherein the mutations are substitutions, insertions, or deletions.

12. A variant capsid polypeptide comprising a variant of SEQ ID NO: 1 comprising a mutation selected from a mutation associated with any of VAR-17 to VAR-45, wherein the mutation is between positions 552 and 588 as compared to SEQ ID NO:1, and wherein the mutation comprises:
an insertion, e.g., an insertion of 4 or more amino acids, e.g., 4-5 amino acids, e.g., 4-6 amino acids, that corresponds to an insertion between positions 583 and 588 as compared to SEQ ID NO: 1, and wherein the insertion comprises a polypeptide that has at least 60%, 70%, 80%, 90%, or 100% identity to SEQ ID NO: 93-122 or comprises a fragment of 4 or more amino acids of SEQ ID NO: 93-122;
a substitution, e.g., a substitution of at least 2 or more residues, e.g., at least 3-9 residues, e.g., at least 4-9 residues, e.g., at least 5-9 residues, e.g., at least 6-9 residues, e.g., at least 7-9 residues, e.g., at least 8-9 residues, e.g., at least 9 residues that correspond to a substitution at positions between 552 and 588 as compared to SEQ ID NO: 1; and
any combination thereof.

13. A variant capsid polypeptide comprising a variant of SEQ ID NO: 1 comprising a mutation selected from a mutation associated with any of VAR-17 to VAR-45, wherein the mutation is between positions 552 and 566 as compared to SEQ ID NO: 1, and wherein the mutation comprises:
(a) a substitution comprising the following consensus formula:

| (SEQ ID NO: 132) | |
|---|---|
| | A-n-L-S/A-D/R/N-L-L-L-n-S-n-n-n-n-K/A |

wherein n is wild type residue as set forth in SEQ ID NO: 1;
(b) a substitution comprising
   1, 2, 3, 4, 5, 6, or 7 of the non-naturally occurring amino acids of the consensus; or
(c) a substitution at any of positions 557, 558 or 559 as compared to SEQ ID NO: 1, and wherein the substitution introduces a leucine at any of positions 557, 558, 559, or any combination thereof.

14. A variant capsid polypeptide comprising a variant of SEQ ID NO: 1 further comprising a mutation selected from a mutation associated with any of VAR-17 to VAR-45, wherein the mutation is between positions 575 and 588 as compared to SEQ ID NO: 1, and wherein the mutation comprises:
(a) a substitution comprising the following consensus formula:

| (SEQ ID NO: 133) | |
|---|---|
| | E-n-n-A/I/D/V-n-A-A/D-n-n-n-S/del-R/Q-S |

wherein n is wild type residue as set forth in SEQ ID NO: 1 and del is a deletion;
(b) a substitution comprising
   1-5 non-naturally occurring amino acids of the consensus formula

| (SEQ ID NO: 133) | |
|---|---|
| | E-n-n-A/I/D/V-n-A-A/D-n-n-n-S/del-R/Q-S; |

or
(c) a substitution at any of positions 578, 580 or 581 as compared to SEQ ID NO: 1, and wherein the substitution at position 578 is isoleucine, substitution at position 580 is alanine, and substitution at position 581 is aspartic acid.

15. A variant capsid polypeptide comprising a variant of SEQ ID NO: 1 further comprising a mutation selected from a mutation associated with any of VAR-17 to VAR-45, wherein the mutation is between positions 552 and 588 as compared to SEQ ID NO: 1, and wherein
the mutation is a substitution between positions 552 and 566 as compared to SEQ ID NO: 1 comprising:
   (a) a substitution comprising the following consensus formula:

| (SEQ ID NO: 132) | |
|---|---|
| | A-n-L-S/A-D/R/N-L-L-L-n-S-n-n-n-n-K/A |

wherein n is wild type residue as set forth in SEQ ID NO: 1;
(b) a substitution comprising
   1, 2, 3, 4, 5, 6, or 7 of the non-naturally occurring amino acids of the consensus; or
(c) a substitution at any of positions 557, 558 or 559 as compared to SEQ ID NO: 1, and wherein the substitution introduces a leucine at any of positions 557, 558, 559, or any combination thereof; and
the mutation is a substitution between positions 575 and 588 as compared to SEQ ID NO: 1 comprising:
   (a) a substitution comprising the following consensus formula:

| (SEQ ID NO: 133) | |
|---|---|
| | E-n-n-A/I/D/V-n-A-A/D-n-n-n-S/del-R/Q-S |

wherein n is wild type residue as set forth in SEQ ID NO: 1 and del is a deletion;
(b) a substitution comprising
   1-5 non-naturally occurring amino acids of the consensus formula

| (SEQ ID NO: 133) | |
|---|---|
| | E-n-n-A/I/D/V-n-A-A/D-n-n-n-S/del-R/Q-S; |

(c) a substitution at any of positions 578, 580 or 581 as compared to SEQ ID NO: 1, and wherein the substitution at position 578 is isoleucine, substitution at position 580 is alanine, and substitution at position 581 is aspartic acid.

16. A variant capsid polypeptide comprising a variant of SEQ ID NO: 1 comprising a mutation, wherein the mutation is between positions 584 and 617 as compared to SEQ ID NO:1, and wherein the mutation comprises:
an insertion, e.g., an insertion of 1 or more amino acids, e.g., 1-5 amino acids, e.g., 5-8 amino acids, e.g., 7-10 amino acids that corresponds to an insertion between positions 584 and 593 as compared to SEQ ID NO: 1, and wherein the insertion comprises a polypeptide that has at least 60%, 70%, 80%, 90%, or 100% identity to SEQ ID NO: 93-122, or a single residue selected from: F, I, P, G, or a fragment of at least 4 amino acids of any of SEQ ID NO: 93-122;
a substitution, e.g., a substitution of at least 1 or more residues, e.g., at least 1-2 residues, e.g., at least 2-7 residues, e.g., at least 2-8 residues that correspond to a substitution at positions between 584 and 617 as compared to SEQ ID NO: 1; and
any combination thereof.

17. The variant capsid polypeptide of embodiment 17, wherein the insertion comprises 7-10 amino acids and has at least 60%, 70%, 80%, 90%, or 100% identity to LALGETTRPA (SEQ ID NO: 93) or comprises a fragment of at least 4, at least 5, at least 6, or at least 7 amino acids of LALGETTRPA (SEQ ID NO: 93), e.g., comprises LGETTRP (SEQ ID NO: 94).

18. The variant capsid polypeptide of any of the preceding embodiments, wherein the capsid polypeptide comprises a mutation that corresponds to a mutation at position 14, 15, 19, and 24 as compared to SEQ ID NO: 1.

19. The variant capsid polypeptide of any of the preceding embodiments, wherein the capsid polypeptide comprises a mutation that corresponds to a mutation at position 3, 6, 11, 12, 21, 23, 25, 29, 31, and 33 as compared to SEQ ID NO: 1.

20. The variant capsid polypeptide of any of the preceding embodiments, wherein the capsid polypeptide comprises a mutation that corresponds to a mutation at position 449, 450, 451, 455, 456, 457, 458, and 459 as compared to SEQ ID NO: 1.

21. The variant capsid polypeptide of any of the preceding embodiments, wherein the capsid polypeptide comprises a mutation that corresponds to a mutation at position 449, 450, 452, 456, 457, and 459 as compared to SEQ ID NO: 1.

22. The variant capsid polypeptide of any of the preceding embodiments, wherein the capsid polypeptide comprises a mutation that corresponds to a mutation at position 457 and 458 as compared to SEQ ID NO: 1.

23. The variant capsid polypeptide of any of the preceding embodiments, wherein the capsid polypeptide comprises a mutation that corresponds to a mutation at position 446 as compared to SEQ ID NO: 1.

24. The variant capsid polypeptide of any of the preceding embodiments, wherein the capsid polypeptide comprises a mutation that corresponds to a mutation at position 449 as compared to SEQ ID NO: 1.

25. The variant capsid polypeptide of any of the preceding embodiments, wherein the capsid polypeptide comprises a mutation that corresponds to a mutation at position 451, 455, 456, 457, 458, 459, and 461 as compared to SEQ ID NO: 1.

26. The variant capsid polypeptide of any of the preceding embodiments, wherein the capsid polypeptide comprises a mutation that corresponds to a mutation at position 448 as compared to SEQ ID NO: 1.

27. The variant capsid polypeptide of any of the preceding embodiments, wherein the capsid polypeptide comprises a mutation that corresponds to a mutation at position 453 as compared to SEQ ID NO: 1.

28. The variant capsid polypeptide of any of the preceding embodiments, wherein the capsid polypeptide comprises a mutation that corresponds to a mutation at position 456, 457, 458, 459, 460, and 461, as compared to SEQ ID NO: 1.

29. The variant capsid polypeptide of any of the preceding embodiments, wherein the capsid polypeptide comprises a mutation that corresponds to a mutation at position 449, 450, 451, 454, 455, 456, 458, 459, 461, and 464 as compared to SEQ ID NO: 1.

30. The variant capsid polypeptide of any of the preceding embodiments, wherein the capsid polypeptide comprises a mutation that corresponds to a mutation at position 446, 448, 449, 451, 453, 455, 456, 457, and 459 as compared to SEQ ID NO: 1.

31. The variant capsid polypeptide of any of the preceding embodiments, wherein the capsid polypeptide comprises a mutation that corresponds to a mutation at position 447 as compared to SEQ ID NO: 1.

32. The variant capsid polypeptide of any of the preceding embodiments, wherein the capsid polypeptide comprises a mutation that corresponds to a mutation at position 581 as compared to SEQ ID NO: 1.

33. The variant capsid polypeptide of any of the preceding embodiments, wherein the capsid polypeptide comprises a mutation that corresponds to a mutation at position 584 as compared to SEQ ID NO: 1.

34. The variant capsid polypeptide of any of the preceding embodiments, wherein the capsid polypeptide comprises a mutation that corresponds to a mutation at position 555, 561, 566, 578, 580, 581, and 585 as compared to SEQ ID NO: 1.

35. The variant capsid polypeptide of any of the preceding embodiments, wherein the capsid polypeptide comprises a mutation that corresponds to a mutation at position 557 and 578 as compared to SEQ ID NO: 1.

36. The variant capsid polypeptide of any of the preceding embodiments, wherein the capsid polypeptide comprises a mutation that corresponds to a mutation at position 556, 558, 578, and 580 as compared to SEQ ID NO: 1.

37. The variant capsid polypeptide of any of the preceding embodiments, wherein the capsid polypeptide comprises a mutation that corresponds to a mutation at position 554, 556, 559, 561, 566, 581, 585, 586, and 587 as compared to SEQ ID NO: 1.

38. The variant capsid polypeptide of any of the preceding embodiments, wherein the capsid polypeptide comprises a mutation that corresponds to a mutation at position 552, 555, 556, 559, 561, 566, 578, 581, and 586 as compared to SEQ ID NO: 1.

39. The variant capsid polypeptide of any of the preceding embodiments, wherein the capsid polypeptide comprises a mutation that corresponds to a mutation at position 575 and 578 as compared to SEQ ID NO: 1.

40. The variant capsid polypeptide of any of the preceding embodiments, wherein the capsid polypeptide comprises a mutation that corresponds to a mutation at position 583 as compared to SEQ ID NO: 1.

41. The variant capsid polypeptide of any of the preceding embodiments, wherein the capsid polypeptide comprises a mutation that corresponds to a mutation at position 585, 588, 589, 590, 591, 593, 597, and 600 as compared to SEQ ID NO: 1.

42. The variant capsid polypeptide of any of the preceding embodiments, wherein the capsid polypeptide comprises a mutation that corresponds to a mutation at position 592 as compared to SEQ ID NO: 1.

43. The variant capsid polypeptide of any of the preceding embodiments, wherein the capsid polypeptide comprises a mutation that corresponds to a mutation at position 585 as compared to SEQ ID NO: 1.

44. The variant capsid polypeptide of any of the preceding embodiments, wherein the capsid polypeptide comprises a mutation that corresponds to a mutation at position 586 as compared to SEQ ID NO: 1.

45. The variant capsid polypeptide of any of the preceding embodiments, wherein the capsid polypeptide comprises a mutation that corresponds to a mutation at position 589 as compared to SEQ ID NO: 1.

46. The variant capsid polypeptide of any of the preceding embodiments, wherein the capsid polypeptide comprises a mutation that corresponds to a mutation at position 587 as compared to SEQ ID NO: 1.

47. The variant capsid polypeptide of any of the preceding embodiments, wherein the capsid polypeptide comprises a mutation that corresponds to a mutation at position 590 as compared to SEQ ID NO: 1.

48. The variant capsid polypeptide of any of the preceding embodiments, wherein the capsid polypeptide comprises a mutation that corresponds to a mutation at position 585, 586, 587, 591, 594, 597, and 600 as compared to SEQ ID NO: 1.

49. The variant capsid polypeptide of any of the preceding embodiments, wherein the capsid polypeptide comprises a mutation that corresponds to a mutation at position 591 as compared to SEQ ID NO: 1.

50. The variant capsid polypeptide of any of the preceding embodiments, wherein the capsid polypeptide comprises a mutation that corresponds to a mutation at position 588 as compared to SEQ ID NO: 1.

51. The variant capsid polypeptide of any of the preceding embodiments, wherein the capsid polypeptide comprises an insertion, e.g., an insertion of 1 or more amino acids, e.g., 1-10 amino acids, e.g., 2-8 amino acids, e.g., 3-7 amino acids, e.g., 7 amino acids, that corresponds to an insertion between positions 446 and 447, 447 and 448, 448 and 449, 449 and 450, 451, and 452, 453 and 454, 581 and 582, 583 and 584, 584 and 585, 585 and 586, 586 and 587, 587 and 588, 588 and 589, 589 and 590, 590 and 591, 591 and 592, or 592 and 593, as compared to SEQ ID NO: 1.

52. The variant capsid polypeptide of any of the preceding embodiments, wherein the capsid polypeptide comprises a mutation that corresponds to a T14L, L15V, I19A, and K24H mutation as compared to SEQ ID NO: 1.

53. The variant capsid polypeptide of any of the preceding embodiments, wherein the capsid polypeptide comprises a mutation that corresponds to a A3V, Y6F, L11F, E12Q, Q21L, W23I, L25C, P29A, P31N, and K33R mutation as compared to SEQ ID NO: 1.

54. The variant capsid polypeptide of any of the preceding embodiments, wherein the capsid polypeptide comprises a mutation that corresponds to a N449Q, T450M, P451T, T455L, T456G, Q457T, S458Q, and R459M mutation as compared to SEQ ID NO: 1.

55. The variant capsid polypeptide of any of the preceding embodiments, wherein the capsid polypeptide comprises a mutation that corresponds to a N449Q, residue P451 deletion, S452T, T456G, Q457T, and R459G mutation as compared to SEQ ID NO: 1.

56. The variant capsid polypeptide of any of the preceding embodiments, wherein the capsid polypeptide comprises a mutation that corresponds to a Q457F and S458C mutation as compared to SEQ ID NO: 1.

57. The variant capsid polypeptide of any of the preceding embodiments, wherein the capsid polypeptide comprises a mutation that corresponds to an insertion between residues 446 and 447 as compared to SEQ ID NO: 1, wherein the insertion comprises a polypeptide of KCQEGMA (SEQ ID NO: 95) or a fragment of at least 4, at least 5, or at least 6 amino acids thereof.

58. The variant capsid polypeptide of any of the preceding embodiments, wherein the capsid polypeptide comprises a mutation that corresponds to an insertion between residues 449 and 450 as compared to SEQ ID NO: 1, wherein the insertion comprises a polypeptide of LMVDRLG (SEQ ID NO: 96) or a fragment of at least 4, at least 5, or at least 6 amino acids thereof.

59. The variant capsid polypeptide of any of the preceding embodiments, wherein the capsid polypeptide comprises a mutation that corresponds to a P451T, T455G, T456G, Q457T, S458Q, R459T, and Q461A mutation as compared to SEQ ID NO: 1.

60. The variant capsid polypeptide of any of the preceding embodiments, wherein the capsid polypeptide comprises a mutation that corresponds to an insertion between residues 448 and 449 as compared to SEQ ID NO: 1, wherein the insertion comprises a polypeptide of HCQECPI (SEQ ID NO: 97) or a fragment of at least 4, at least 5, or at least 6 amino acids thereof.

61. The variant capsid polypeptide of any of the preceding embodiments, wherein the capsid polypeptide comprises a mutation that corresponds to an insertion between residues 453 and 454 as compared to SEQ ID NO: 1, wherein the insertion comprises a polypeptide of FSGLEN (SEQ ID NO: 98) or a fragment of at least 5 amino acids thereof.

62. The variant capsid polypeptide of any of the preceding embodiments, wherein the capsid polypeptide comprises a mutation that corresponds to a residue deletion at positions T456, Q457, S458, R459, L460, and Q461 as compared to SEQ ID NO: 1.

63. The variant capsid polypeptide of any of the preceding embodiments, wherein the capsid polypeptide comprises a mutation that corresponds to a N449I, T450N, P451G, residue T454 deletion, T455Q, T456N, S458Q, R459T, Q461K, and Q464V mutation as compared to SEQ ID NO: 1.

64. The variant capsid polypeptide of any of the preceding embodiments, wherein the capsid polypeptide comprises a mutation that corresponds to a S446A, residue T448 and N449 deletion, P451Q, G453T, T455G, T456G, Q457T, and R459G mutation as compared to SEQ ID NO: 1.

65. The variant capsid polypeptide of any of the preceding embodiments, wherein the capsid polypeptide comprises a mutation that corresponds to a N449Q, T450S, S452G, T455A, Q457F, S458M, R459D, and an insertion between residues 451 and 452 as compared to SEQ ID NO: 1, wherein the insertion comprises an amino acid Y.

66. The variant capsid polypeptide of any of the preceding embodiments, wherein the capsid polypeptide comprises a mutation that corresponds to an insertion between residues 447 and 448 as compared to SEQ ID NO: 1, wherein the insertion comprises a polypeptide of HETEFNF (SEQ ID NO: 99) or a fragment of at least 4, at least 5, or at least 6 amino acids thereof.

67. The variant capsid polypeptide of any of the preceding embodiments, wherein the capsid polypeptide comprises a mutation that corresponds to a P451T, T455G, T456G, Q457T, S458Q, R459T, and Q461A mutation as compared to SEQ ID NO: 1.

68. The variant capsid polypeptide of any of the preceding embodiments, wherein the capsid polypeptide comprises a mutation that corresponds to an insertion between residues 581 and 582 as compared to SEQ ID NO: 1, wherein the insertion comprises a polypeptide of FALMEP (SEQ ID NO: 100) or a fragment of at least 4, or at least 5 amino acids thereof.

69. The variant capsid polypeptide of any of the preceding embodiments, wherein the capsid polypeptide comprises a mutation that corresponds to an insertion between residues 584 and 585 as compared to SEQ ID NO: 1, wherein the insertion comprises a polypeptide of RAYNPD (SEQ ID NO: 101) or a fragment of at least 4, or at least 5 amino acids thereof.

70. The variant capsid polypeptide of any of the preceding embodiments, wherein the capsid polypeptide comprises a mutation that corresponds to a residue R585 deletion and a E555A, D461S, R566K, S578V, S580A, and T581A mutation as compared to SEQ ID NO: 1.

71. The variant capsid polypeptide of any of the preceding embodiments, wherein the capsid polypeptide comprises a mutation that corresponds to a V557L and S578D mutation as compared to SEQ ID NO: 1.

72. The variant capsid polypeptide of any of the preceding embodiments, wherein the capsid polypeptide comprises a mutation that corresponds to a K556N, M558L, S578I, and S580A mutation as compared to SEQ ID NO: 1.

73. The variant capsid polypeptide of any of the preceding embodiments, wherein the capsid polypeptide comprises a mutation that corresponds to a I554L, K556R, I559L, D561S, R566A, T581D, R585S, G586R, and N587S mutation as compared to SEQ ID NO: 1.

74. The variant capsid polypeptide of any of the preceding embodiments, wherein the capsid polypeptide comprises a mutation that corresponds to a V552A, E555S, K556D, I559L, D561S, R566K, S578I, T581D, and G586Q mutation as compared to SEQ ID NO: 1.

75. The variant capsid polypeptide of any of the preceding embodiments, wherein the capsid polypeptide comprises a mutation that corresponds to a Q575E and S578A mutation as compared to SEQ ID NO: 1.

76. The variant capsid polypeptide of any of the preceding embodiments, wherein the capsid polypeptide comprises a mutation that corresponds to an insertion between residues 583 and 584 as compared to SEQ ID NO: 1, wherein the insertion comprises a polypeptide of LNWTAE (SEQ ID NO: 102) or a fragment of at least 4, or at least 5 amino acids thereof.

77. The variant capsid polypeptide of any of the preceding embodiments, wherein the capsid polypeptide comprises a mutation that corresponds to a R585S, R588T, Q589N, A590P, A591I, A593G, T597S, and V600A mutation as compared to SEQ ID NO: 1.

78. The variant capsid polypeptide of any of the preceding embodiments, wherein the capsid polypeptide comprises a mutation that corresponds to a R585N and G586A mutation, and an insertion between residues 584 and 585 as compared to SEQ ID NO: 1, wherein the insertion comprises a polypeptide of LAKEFTTR (SEQ ID NO: 103) or a fragment of at least 4, at least 5, at least 6, or at least 7 amino acids thereof (e.g., a fragment comprising or consisting of KEFTTR (SEQ ID NO: 104)).

79. The variant capsid polypeptide of any of the preceding embodiments, wherein the capsid polypeptide comprises a mutation that corresponds to an insertion between residues 592 and 593 as compared to SEQ ID NO: 1, wherein the insertion comprises a polypeptide of LHPLE (SEQ ID NO: 105) or a fragment of at least 4 amino acids thereof.

80. The variant capsid polypeptide of any of the preceding embodiments, wherein the capsid polypeptide comprises a mutation that corresponds to an insertion between residues 585 and 586 as compared to SEQ ID NO: 1, wherein the insertion comprises an amino acid F.

81. The variant capsid polypeptide of any of the preceding embodiments, wherein the capsid polypeptide comprises a mutation that corresponds to an insertion between residues 586 and 587 as compared to SEQ ID NO: 1, wherein the insertion comprises a polypeptide of DQDFKNR (SEQ ID NO: 106) or a fragment of at least 4, at least 5, or at least 6 amino acids thereof.

82. The variant capsid polypeptide of any of the preceding embodiments, wherein the capsid polypeptide comprises a mutation that corresponds to an insertion between residues 589 and 590 as compared to SEQ ID NO: 1, wherein the insertion comprises an amino acid I.

83. The variant capsid polypeptide of any of the preceding embodiments, wherein the capsid polypeptide comprises a mutation that corresponds to an insertion between residues 587 and 588 as compared to SEQ ID NO: 1, wherein the insertion comprises a polypeptide of LAIEQTRPA (SEQ ID NO: 107) or a fragment of at least 4, at least 5, at least 6, at least 7, or at least 8 amino acids thereof (e.g., a fragment comprising or consisting of IEQTRPA (SEQ ID NO: 108)).

84. The variant capsid polypeptide of any of the preceding embodiments, wherein the capsid polypeptide comprises a mutation that corresponds to a G586P and N587A mutation, and an insertion between residues 584 and 585 as compared to SEQ ID NO: 1, wherein the insertion comprises a polypeptide of RARLDETT (SEQ ID NO: 109) or a fragment of at least 4, at least 5, at least 6, or at least 7 amino acids thereof (e.g., a fragment comprising or consisting of RLDETT (SEQ ID NO: 110).

85. The variant capsid polypeptide of any of the preceding embodiments, wherein the capsid polypeptide comprises a mutation that corresponds to a N587A mutation, and an insertion between residues 586 and 587 as compared to SEQ ID NO: 1, wherein the insertion comprises a polypeptide of LALAEITRP (SEQ ID NO: 111) or a fragment of at least 4, at least 5, at least 6, at least 7, or at least 8 amino acids thereof (e.g., a fragment comprising or consisting of LAEITRP (SEQ ID NO: 112)).

86. The variant capsid polypeptide of any of the preceding embodiments, wherein the capsid polypeptide comprises a mutation that corresponds to a N587A mutation, and an insertion between residues 586 and 587 as compared to SEQ ID NO: 1, wherein the insertion comprises a polypeptide of LANGEQTRP (SEQ ID NO: 113) or a fragment of at least 4, at least 5, at least 6, at least 7, or at least 8 amino acids thereof (e.g., a fragment comprising or consisting of NGEQTRP (SEQ ID NO: 114)).

87. The variant capsid polypeptide of any of the preceding embodiments, wherein the capsid polypeptide comprises a mutation that corresponds to an insertion between residues 586 and 587 as compared to SEQ ID NO: 1, wherein the insertion comprises a polypeptide of ATDTKT (SEQ ID NO: 115) or a fragment of at least 4, or at least 5 amino acids thereof.

88. The variant capsid polypeptide of any of the preceding embodiments, wherein the capsid polypeptide comprises a mutation that corresponds to an insertion between residues 590 and 591 as compared to SEQ ID NO: 1, wherein the insertion comprises an amino acid P.

89. The variant capsid polypeptide of any of the preceding embodiments, wherein the capsid polypeptide comprises a mutation that corresponds to an insertion between residues 587 and 588 as compared to SEQ ID NO: 1, wherein the insertion comprises a polypeptide of APGETTRPA (SEQ ID NO: 116) or a fragment of at least 4, at least 5, at least 6, at least 7, or at least 8 amino acids thereof.

90. The variant capsid polypeptide of any of the preceding embodiments, wherein the capsid polypeptide comprises a mutation that corresponds to an insertion between residues 589 and 590 as compared to SEQ ID NO: 1, wherein the insertion comprises an amino acid P.

91. The variant capsid polypeptide of any of the preceding embodiments, wherein the capsid polypeptide comprises a mutation that corresponds to a R585S, G586S, N587A, A591E, D594R, T597A, and V600I mutation as compared to SEQ ID NO: 1.

92. The variant capsid polypeptide of any of the preceding embodiments, wherein the capsid polypeptide comprises a mutation that corresponds to an insertion between residues 586 and 587 as compared to SEQ ID NO: 1, wherein the insertion comprises a polypeptide of FHNEGKY (SEQ ID NO: 118) or a fragment of at least 4, at least 5, or at least 6 amino acids thereof.

93. The variant capsid polypeptide of any of the preceding embodiments, wherein the capsid polypeptide comprises a mutation that corresponds to an insertion between residues 591 and 592 as compared to SEQ ID NO: 1, wherein the insertion comprises an amino acid G.

94. The variant capsid polypeptide of any of the preceding embodiments, wherein the capsid polypeptide comprises a mutation that corresponds to an insertion between residues 588 and 589 as compared to SEQ ID NO: 1, wherein the insertion comprises a polypeptide of QPWEPDK (SEQ ID NO: 119) or a fragment of at least 4, at least 5, or at least 6 amino acids thereof.

95. The variant capsid polypeptide of any of the preceding embodiments, wherein the capsid polypeptide comprises a mutation that corresponds to an insertion between residues 592 and 593 as compared to SEQ ID NO: 1, wherein the insertion comprises a polypeptide of ALALSTTN (SEQ ID NO: 120) or a fragment of at least 4, at least 5, at least 6, or at least 7 amino acids thereof (e.g., a fragment comprising or consisting of ALSTTN (SEQ ID NO: 121)).

96. The variant capsid polypeptide of any of the preceding embodiments, wherein the capsid polypeptide comprises a mutation that corresponds to an insertion between residues 585 and 586 as compared to SEQ ID NO: 1, wherein the insertion comprises a polypeptide of PWGTAG (SEQ ID NO: 122) or a fragment of at least 4, or at least 5 amino acids thereof.

97. A variant capsid polypeptide, comprising (a) a polypeptide of any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46, (b) comprising the VP2 or VP3 sequence of any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46; (c) a polypeptide comprising a sequence having at least 90%, at least 95%, at least 96%, at least 97%, at least 98%, or at least 99% identity thereto, wherein said sequence comprises at least one (e.g., one, two, three or more, e.g., all) of the mutation differences associated with any of SEQ ID NO: 2 through SEQ ID NO: 46, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutation differences associated with any of SEQ ID NO: 2 through SEQ ID NO: 46, relative to SEQ ID NO: 1.

98. The variant capsid polypeptide of any of the preceding embodiments, wherein the variant capsid polypeptide is a VP1 polypeptide, a VP2 polypeptide or a VP3 polypeptide.

99. A variant capsid polypeptide comprising a mutation that corresponds to a leucine at a position corresponding to 554 of SEQ ID NO: 1 (e.g., comprising I554L as compared to SEQ ID NO: 1).

100. The variant capsid polypeptide of any of the preceding embodiments, wherein the capsid polypeptide has up to 12 additional mutations, e.g., between 8 and 12 mutations, as compared to SEQ ID NO: 1.

101. The variant capsid polypeptide of any of the preceding embodiments, wherein the capsid polypeptide has up to 16 additional mutations, e.g., between 11 and 16 mutations, as compared to VAR-22 (SEQ ID NO: 23).

102. The variant capsid polypeptide of any of the preceding embodiments, further comprising a mutation that corresponds to a serine at a position corresponding to 561 of SEQ ID NO: 1 (e.g., comprising D561S as compared to SEQ ID NO: 1).

103. The variant capsid polypeptide of any of the preceding embodiments, further comprising a mutation that corresponds to an aspartic acid at a position corresponding to 581 of SEQ ID NO: 1 (e.g., comprising T581D as compared to SEQ ID NO: 1).

104. A variant capsid polypeptide comprising a mutation that corresponds to a leucine at a position corresponding to 559 of SEQ ID NO: 1 (e.g., comprising I559L as compared to SEQ ID NO: 1).

105. The variant capsid polypeptide of any of the preceding embodiments, wherein the capsid polypeptide has up to 13 additional mutations, e.g., between 4 and 13 mutations, as compared to SEQ ID NO: 1.

106. The variant capsid polypeptide of any of the preceding embodiments, wherein the capsid polypeptide has between 8 and 19 mutations as compared to VAR-22 (SEQ ID NO: 23).

107. The variant capsid polypeptide of any of the preceding embodiments, wherein the capsid polypeptide has up to 20 additional mutations, e.g., between 5 and 20 mutations, as compared to VAR-23 (SEQ ID NO: 24).

108. The variant capsid polypeptide of any of the preceding embodiments, further comprising a mutation that corresponds to a serine at a position corresponding to 561 of SEQ ID NO: 1 (e.g., comprising D561S as compared to SEQ ID NO: 1), optionally wherein capsid polypeptide further comprises an aspartic acid at a position corresponding to 581 of SEQ ID NO: 1 (e.g., comprising a T581D as compared to SEQ ID NO: 1).

109. A variant capsid polypeptide comprising a mutation that corresponds to asparagine at a position corresponding to 556 of SEQ ID NO: 1 (e.g., comprising K556N as compared to SEQ ID NO: 1).

110. The variant capsid polypeptide of any of the preceding embodiments, wherein the capsid polypeptide has up to 12 additional mutations, e.g., between 4 and 12 mutations, as compared to SEQ ID NO: 1.

111. The variant capsid polypeptide of any of the preceding embodiments, wherein the capsid polypeptide has up to 12 additional mutations, e.g., between 9 and 12 mutations, as compared to VAR-21 (SEQ ID NO: 22).

112. A variant capsid polypeptide comprising a mutation that corresponds to a serine at a position corresponding to 561 of SEQ ID NO: 1 (e.g., comprising D561S as compared to SEQ ID NO: 1).

113. The variant capsid polypeptide of any of the preceding embodiments, wherein the capsid polypeptide has up to 12 additional mutations, e.g., between 7 and 12 mutations, as compared to SEQ ID NO: 1.

114. The variant capsid polypeptide of any of the preceding embodiments, wherein the capsid polypeptide has up to 14 additional mutations, e.g., between 11 and 14 mutations, as compared to VAR-19 (SEQ ID NO: 20).

115. The variant capsid polypeptide of any of the preceding embodiments, wherein the capsid polypeptide has up to 14 additional mutations, e.g., between 10 and 14 mutations, as compared to VAR-22 (SEQ ID NO: 23).

116. The variant capsid polypeptide of any of the preceding embodiments, wherein the capsid polypeptide has up to 14 additional mutations, e.g., between 9 and 14 mutations, as compared to VAR-23 (SEQ ID NO: 24).

117. The variant capsid polypeptide of any of the preceding embodiments, further comprising a mutation that corresponds to an aspartic acid at a position corresponding to 581 of SEQ ID NO:1 (e.g., comprising T581D as compared to SEQ ID NO: 1).

118. A capsid polypeptide comprising a mutation at a position corresponding to 587 of SEQ ID NO: 1, optionally wherein the mutation is to an alanine (e.g., wherein the mutation is N587A according to SEQ ID NO: 1), optionally wherein the capsid polypeptide further comprises an insertion (e.g., an insertion of 4 or more amino acids, optionally an insertion of 7 or more amino acids, optionally an insertion of 7-10 amino acids, optionally an insertion of 7, 8 or 9 amino acids) between two adjacent amino acids between 580 and 587 (optionally between 586 and 587) according to SEQ ID NO: 1.

119. A nucleic acid molecule comprising sequence encoding a variant capsid polypeptide of any one of embodiments 1-118.

120. The nucleic acid molecule of embodiment 119, comprising one or more regulatory elements operably linked to the sequence encoding the variant capsid polypeptide.

121. The nucleic acid molecule of any of embodiments 119-120, comprising SEQ ID NO: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, or a fragment thereof, or a variant thereof having at least 85, 90, 91, 92, 93, 94, 95, 96, 97, 98, 99, or 100% sequence identity thereto.

122. A virus particle (e.g., adeno-associated virus (“AAV”) particle) comprising the variant capsid polypeptide of any one of embodiments 1-118 or comprising a variant capsid polypeptide encoded by the nucleic acid molecule of any one of embodiments 119-121.

123. The virus particle of embodiment 122, comprising a nucleic acid comprising a heterologous transgene and one or more regulatory elements.

124. A virus particle of any of embodiments 122-123 comprising the variant capsid polypeptide of any one of embodiments 1-118, wherein said virus particle, or a virus particle comprising said variant capsid polypeptide or a virus particle comprising a variant capsid polypeptide encoded by a nucleic acid molecule of any one of embodiments 119-121 exhibits increased ocular transduction, e.g., as measured in a mouse or in NHP, e.g., as described herein, relative to wild-type AAV2 (e.g., a virus particle comprising capsid polypeptides of SEQ ID NO: 1 or encoded by SEQ ID NO: 92).

125. The nucleic acid molecule of any one of embodiments 119-121, wherein the nucleic acid molecule is double-stranded or single-stranded, optionally wherein the nucleic acid molecule is linear or circular, e.g., wherein the nucleic acid molecule is a plasmid.

126. A method of producing a virus particle comprising a variant AAV2 capsid polypeptide, said method comprising introducing a nucleic acid molecule of any one of embodiments 119-121 or 125 into a cell (e.g., a HEK293 cell), and harvesting said virus particle therefrom.

127. A method of delivering a payload (e.g., a nucleic acid) to a cell comprising contacting the cell with a dependoparvovirus particle comprising a variant capsid polypeptide of any one of embodiments 1-118 or the virus particle of any of embodiments 123-124 and a payload.

128. The method of embodiment 127, wherein the cell is an ocular cell.

129. The method of embodiment 128, wherein the ocular cell is in the retina, the macula, or the trabecular meshwork.

130. A method of delivering a payload (e.g., a nucleic acid) to a subject comprising administering to the subject a dependoparvovirus particle comprising a variant capsid polypeptide of any one of embodiments 1-118 and the payload, or administering to the subject the virus particle of any one of embodiments 122-124.

131. The method of embodiment 130, wherein the particle delivers the payload to the eye.

132. The method of embodiment 131, wherein the particle delivers the payload to the retina, the macular, or the trabecular meshwork.

133. The method of any one of embodiments 130-132, wherein the particle delivers the payload to the eye with increased transduction in one or more regions of the eye as compared to a virus particle comprising capsid polypeptides of SEQ ID NO: 1.

134. The method of embodiment 133, wherein the one or more regions of the eye is selected from the retina, the macula, the trabecular meshwork, or any combination thereof.

135. The method of embodiment 133, wherein the retina comprises non-macular retina.

136. The variant capsid polypeptide of any of embodiments 1-118, the virus particle of any of embodiments 122-124 or the method of any one of embodiments 130-135, wherein the particle (e.g., particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, or 150-times as compared to a virus particle comprising capsid polypeptides of SEQ ID NO: 1.

137. The variant capsid polypeptide of any of embodiments 1-118, the virus particle of any of embodiments 122-124 or the method of any one of embodiments 130-136, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to macular tissue.

138. The variant capsid polypeptide of any of embodiments 1-118, the virus particle of any of embodiments 122-124 or the method of any one of embodiments 130-137, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to non-macular retina tissue.

139. The variant capsid polypeptide of any of embodiments 1-118, the virus particle of any of embodiments 122-124 or the method of any one of embodiments 130-138, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to trabecular meshwork tissue.

140. The variant capsid polypeptide of any of embodiments 1-118, the virus particle of any of embodiments 122-124 or the method of any one of embodiments 130-139, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to trabecular meshwork tissue.

141. The variant capsid polypeptide of any of embodiments 1-118, the virus particle of any of embodiments 122-124 or the method of any one of embodiments 130-140, wherein the (e.g., particle comprising the variant capsid polypeptide) particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue and non-macular retina tissue relative to trabecular meshwork tissue.

142. The variant capsid polypeptide of any of embodiments 1-118, the virus particle of any of embodiments 122-124 or the method of any one of embodiments 130-141, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue and non-macular retina tissue.

143. The variant capsid polypeptide of any of embodiments 1-118, the virus particle of any of embodiments 122-124 or the method of any one of embodiments 130-142, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue.

144. The variant capsid polypeptide of any of embodiments 1-118, the virus particle of any of embodiments 122-124 or the method of any one of embodiments 130-143, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to non-macular retina tissue.

145. The variant capsid polypeptide of any of embodiments 1-118, the virus particle of any of embodiments 122-124 or the method of any one of embodiments 130-144, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue, macular tissue, and non-macular retina tissue.

146. The variant capsid polypeptide of any of embodiments 1-118, the virus particle of any of embodiments 122-124 or the method of any one of embodiments 130-145, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1 without increased biodistribution in one or more regions of the eye as compared to a virus particle comprising capsid polypeptides of SEQ ID NO: 1.

147. The method of any one of embodiments 130-146, wherein the administration to the subject is via an intravitreal injection, or an intracameral injection.

148. A method of treating a disease or condition in a subject, comprising administering to the subject a dependoparvovirus particle in an amount effective to treat the disease or condition, wherein the dependoparvovirus particle is a particle comprising a capsid polypeptide of any one of embodiments 1-118 and 136-146, or encoded by the nucleic acid of any one of embodiments 119-121 or 125, or is a virus particle of any one of embodiments 122-124.

149. A cell, cell-free system, or other translation system, comprising the capsid polypeptide, nucleic acid molecule, or virus particle of any one of embodiments 1-125 or 136-146.

150. A method of making a dependoparvovirus (e.g., an adeno-associated dependoparvovirus (AAV) particle, comprising:
providing a cell, cell-free system, or other translation system, comprising a nucleic acid of any of embodiments 119-121 or 125; and
cultivating the cell, cell-free system, or other translation system, under conditions suitable for the production of the dependoparvovirus particle,
thereby making the dependoparvovirus particle.

151. The method of embodiment 150, wherein the cell, cell-free system, or other translation system comprises a second nucleic acid molecule and said second nucleic acid molecule is packaged in the dependoparvovirus particle.

152. The method of embodiment 150, wherein the second nucleic acid comprises a payload, e.g., a heterologous nucleic acid sequence encoding a therapeutic product.

153. The method of any one of embodiments 150-152, wherein the nucleic acid of any of embodiments 119-121 or 125 mediates the production of a dependoparvovirus particle which does not include said nucleic acid of any of embodiments 119-121 or 125.

154. The method of any one of embodiments 150-153, wherein the nucleic acid of any of embodiments 119-121 or 125 mediates the production of a dependoparvovirus particle at a level at least 10%, at least 20%, at least 50%, at least 100%, at least 200% or greater than the production level mediated by the nucleic acid of SEQ ID NO: 92.

155. A composition, e.g., a pharmaceutical composition, comprising a virus particle of any one of embodiments 122-124 or a virus particle produced by the method of any one of embodiments 126 or 150-154, and a pharmaceutically acceptable carrier.

156. The variant capsid polypeptide of any of embodiments 1-118 and 136-146, the nucleic acid molecule of any of embodiments 119-121 or 125, or the virus particle of any of embodiments 122-124 and 136-146 for use in treating a disease or condition in a subject.

157. The variant capsid polypeptide of any of embodiments 1-118 and 136-146, the nucleic acid molecule of any of claims 119-121 or 125, or the virus particle of any of claims 122-124 and 136-146 for use in the manufacture of a medicament for use in treating a disease or condition in a subject.

### DETAILED DESCRIPTION

The present disclosure is directed, in part, to the variant capsid variants that can be used to generate dependoparvovirus particles. In some embodiments, the particles have increased ocular transduction that can be used to deliver a transgene or molecule of interest to an eye with higher transduction efficiency in the eye as compared to a dependoparvovirus particle without the variant capsid polypeptides. Accordingly, provided herein are variant capsid polypeptides, nucleic acid molecules encoding the same, viral particles comprising the variant capsid polypeptides, and methods of using the same.

### Definitions

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 80, 85, 90, 91, 92, 93, 94, 95, 96, 97, 98, 99, or 100% identity to the sequence in question. Correspondingly, an AAVX particle/capsid, as used herein, refers to an AAV particle/caspid comprising at least one polypeptide or polypeptide encoding nucleic acid sequence derived from a naturally occurring AAV X serotype. For example, an AAV2 particle refers to an AAV particle comprising at least one polypeptide or polypeptide encoding nucleic acid sequence derived from a naturally occurring AAV2 sequence.

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence comprising a mutant capsid polypeptide or a nucleic acid molecule encoding the same may comprise an capsid polypeptide. Use of the term exogenous in this fashion means that the polypeptide or the nucleic acid molecule encoding a polypeptide comprising the mutation in question at this position does not occur naturally, e.g., is not present in AAV2, e.g., is not present in SEQ ID NO: 1.

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide may stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Variant: As used herein, a “variant capsid polypeptide” refers to a polypeptide that differs from a reference sequence (e.g. SEQ ID NO: 1). The variant can, for example, comprise a mutation (e.g. substitution, deletion, or insertion). In some embodiments, the variant is about, or at least, 85%, 86%, 87%, 88%, 89%, 90%, 91%, 92%, 93%, 94%, 95%, 96%, 97%, 98%, or 99% identical to the reference sequence. In some embodiments, the reference sequence is a polypeptide comprising SEQ ID NO: 1.

### Capsid Polypeptides and Nucleic Acids Encoding the Same

The disclosure is directed, in part, to a nucleic acid comprising a sequence encoding an a variant capsid polypeptide comprising a mutation (insertion, deletion, or substitution) as compared to the wild-type sequence. In some embodiments, the wild-type sequence is SEQ ID NO: 1. The disclosure is directed, in part, to a variant capsid polypeptides comprising SEQ ID NO: 1 with one or more mutations as compared to SEQ ID NO: 1. The mutation can be, for example, an insertion, deletion, or substitution as compared to the wild-type sequence. In some embodiments, the wild-type sequence is SEQ ID NO: 1.

In some embodiments, the nucleic acid molecule encodes a capsid polypeptide that comprises a mutation selected from Table 2, Table 3, or Table 4. In some embodiments, the nucleic acid molecule encodes a capsid polypeptide that comprises a mutation selected from Table 2 and Table 3; Table 2 and Table 4, or Table 3 and Table 4. In some embodiments, the nucleic acid molecule encodes a capsid polypeptide that comprises a mutation selected from Table 2 and Table 3. In some embodiments, the nucleic acid molecule encodes a capsid polypeptide that comprises a mutation selected from Table 2 and Table 4. In some embodiments, the nucleic acid molecule encodes a capsid polypeptide that comprises a mutation selected from Table 3 and Table 4. In some embodiments, Table 2 comprises mutations corresponding to residues 1-36 as compared to SEQ ID NO: 1. In some embodiments, Table 3 comprises mutations corresponding to residues 431-466 as compared to SEQ ID NO: 1. In some embodiments, Table 4 comprises mutations corresponding to residues 552-617 as compared to SEQ ID NO: 1. In some embodiments, the nucleic acid molecule encodes a capsid polypeptide that comprises a mutation within the 1-36 amino acid region of SEQ ID NO: 1. In some embodiments, the nucleic acid molecule encodes a capsid polypeptide that comprises a mutation within the 431-466 amino acid region of SEQ ID NO: 1. In some embodiments, the nucleic acid molecule encodes a capsid polypeptide that comprises a mutation within the 552-617 amino acid region of SEQ ID NO: 1. In some embodiments, the nucleic acid molecule encodes a capsid polypeptide that comprises a mutation within the 1-36, 431-466, and 552-617 amino acid region of SEQ ID NO: 1. In some embodiments, the nucleic acid molecule encodes a capsid polypeptide that comprises a mutation selected from Table 3. In some embodiments, the mutation selected from Table 3 is an insertion, e.g., an insertion of 4 or more amino acids, e.g., 4-5 amino acids, e.g., 4-6 amino acids, e.g., 4-7 amino acids, e.g., 7 amino acids, that corresponds to an insertion between positions 446 and 454 as compared to SEQ ID NO: 1, and wherein the insertion comprises a polypeptide that has at least 60%, 70%, 80%, 90%, or 100% identity to SEQ ID NO: 93-122; a substitution, e.g., a substitution of at least 2 or more residues, e.g., at least 6-10 residues, e.g., at least 7-10 residues, e.g., at least 8-10 residues, e.g., at least 9-10 residues, e.g., at least 10 residues that correspond to a substitution at positions between 445 and 465 as compared to SEQ ID NO: 1; and the variant comprises at least 3 mutations selected from: T455G/L/Q, T456G, Q457T, S458Q, R459G/T; and further comprises at least 3 other mutations between positions 445-465 as compared to SEQ ID NO: 1, and wherein the mutations are substitutions, insertions, or deletions. In some embodiments, the nucleic acid molecule encodes a capsid polypeptide that comprises a mutation selected from Table 4. In some embodiments, the mutation selected from Table 4 is an insertion, e.g., an insertion of 4 or more amino acids, e.g., 4-5 amino acids, e.g., 4-6 amino acids, that corresponds to an insertion between positions 583 and 588 as compared to SEQ ID NO: 1, and wherein the insertion comprises a polypeptide that has at least 60%, 70%, 80%, 90%, or 100% identity to SEQ ID NO: 93-122; and a substitution, e.g., a substitution of at least 2 or more residues, e.g., at least 3-9 residues, e.g., at least 4-9 residues, e.g., at least 5-9 residues, e.g., at least 6-9 residues, e.g., at least 7-9 residues, e.g., at least 8-9 residues, e.g., at least 9 residues that correspond to a substitution at positions between 552 and 588 as compared to SEQ ID NO: 1. In some embodiments, the mutation selected from Table 4 is a mutation between positions 552 and 566 as compared to SEQ ID NO: 1, and the mutation is a substitution comprising the following consensus formula:

| (SEQ ID NO: 132) | |
|---|---|
| | A-n-L-S/A-D/R/N-L-L-L-n-S-n-n-n-n-K/A |

wherein n is wild type residue as set forth in SEQ ID NO: 1; or the mutation comprises a substitution comprising at least 1, 2, 3, 4, 5, 6, 7, 8, 9, or 10 of the non-wild type amino acids of the consensus formula A-n-L-S/A-D/R/N-L-L-L-n-S-n-n-n-n-K/A (SEQ ID NO: 132); or the variant comprises a substitution at any of positions 557, 558 or 559 as compared to SEQ ID NO: 1, and wherein the substitution introduces a leucine at any of positions 557, 558, 559, or any combination thereof.

In some embodiments, the mutation selected from Table 4 is a mutation between positions 575 and 588 as compared to SEQ ID NO: 1, and the mutation is a substitution comprising the following consensus formula:

| (SEQ ID NO: 133) | |
|---|---|
| | E-n-n-A/I/D/V-n-A-A/D-n-n-n-S/del-R/Q-S |

wherein n is wild type residue as set forth in SEQ ID NO: 1 (“del” is a deletion); or the mutation comprises a substitution comprising at least 1-7 of the non-wild-type amino acids of the consensus formula E-n-n-A/I/D/V-n-A-A/D-n-n-n-S/del-R/Q-S(SEQ ID NO: 133); or the variant comprises a substitution at position 578, 580 or 581 as compared to SEQ ID NO: 1, and wherein the substitution at position 578 is isoleucine, substitution at position 580 is alanine, and substitution at position 581 is aspartic acid.

In some embodiments, the mutation selected from Table 4 is a mutation between positions 552 and 588 as compared to SEQ ID NO: 1, and the mutation is a substitution between positions 552 and 566 comprising the following consensus formula:

| (SEQ ID NO: 132) | |
|---|---|
| | A-n-L-S/A-D/R/N-L-L-L-n-S-n-n-n-n-K/A |

wherein n is wild type residue as set forth in SEQ ID NO: 1; 1, 2, 3, 4, 5, 6, or 7 amino acids of the consensus; the variant further comprises a substitution at any of positions 557, 558 or 559 as compared to SEQ ID NO: 1, and wherein the substitution introduces a leucine at any of positions 557, 558, 559, or any combination thereof; and the mutation is a substitution between positions 575 and 588 comprising the following consensus formula:

| (SEQ ID NO: 133) | |
|---|---|
| | E-n-n-A/I/D/V-n-A-A/D-n-n-n-S/del-R/Q-S |

wherein n is wild type residue as set forth in SEQ ID NO: 1; 1-6 amino acids of the consensus; del is a deletion; the variant further comprises a substitution at position 578, 580 or 581 as compared to SEQ ID NO: 1, and wherein the substitution at position 578 is isoleucine, substitution at position 580 is alanine, and substitution at position 581 is aspartic acid.

In some embodiments, the mutation selected from Table 4 is a mutation between positions 584 and 617 as compared to SEQ ID NO:1, and wherein the mutation comprises:
an insertion, e.g., an insertion of 1 or more amino acids, e.g., 1-5 amino acids, e.g., 5-8 amino acids, e.g., 7-10 amino acids that corresponds to an insertion between positions 584 and 593 as compared to SEQ ID NO: 1, and wherein the insertion comprises a polypeptide that has at least 60%, 70%, 80%, 90%, or 100% identity to SEQ ID NO: 93-122, or a single residue selected from: F, I, P, G;
a substitution, e.g., a substitution of at least 1 or more residues, e.g., at least 1-2 residues, e.g., at least 2-7 residues, e.g., at least 2-8 residues that correspond to a substitution at positions between 584 and 617 as compared to SEQ ID NO: 1; and any combination thereof. In some embodiments, the insertion comprises 7-10 amino acids and has at least 60%, 70%, 80%, 90%, or 100% identity to LALGETTRPA (SEQ ID NO: 93).

In some embodiments, the nucleic acid molecule encodes a capsid polypeptide that comprises a mutation that corresponds to a mutation at position 3, 6, 11, 12, 14, 15, 19, 21, 23, 24, 25, 29, 31, 33, 446, 449, 450, 451, 452, 454, 455, 456, 457, 458, 459, 460, 461, 464, 552, 554, 555, 556, 557, 558, 559, 561, 566, 575, 578, 580, 581, 585, 586, 587, 588, 589, 590, 591, 593, 594, 597, 600, or any combination thereof, an insertion between positions 446 and 447, 447 and 448, 448 and 449, 449 and 450, 451, and 452, 453 and 454, 581 and 582, 583 and 584, 584 and 585, 585 and 586, 586 and 587, 587 and 588, 588 and 589, 589 and 590, 590 and 591, 591 and 592, 592 and 593, or any combination thereof according to SEQ ID NO: 1, optionally wherein the mutation comprises an insertion, a deletion or a substitution.

In some embodiments, the nucleic acid molecule encodes a capsid polypeptide that comprises a mutation that corresponds to a mutation at position 3 as compared to SEQ ID NO: 1. In some embodiments, the nucleic acid molecule encodes a capsid polypeptide that comprises a mutation that corresponds to a mutation at position 6 as compared to SEQ ID NO: 1. In some embodiments, the nucleic acid molecule encodes a capsid polypeptide that comprises a mutation that corresponds to a mutation at position 11 as compared to SEQ ID NO: 1. In some embodiments, the nucleic acid molecule encodes a capsid polypeptide that comprises a mutation that corresponds to a mutation at position 12 as compared to SEQ ID NO: 1. In some embodiments, the nucleic acid molecule encodes a capsid polypeptide that comprises a mutation that corresponds to a mutation at position 14 as compared to SEQ ID NO: 1. In some embodiments, the nucleic acid molecule encodes a capsid polypeptide that comprises a mutation that corresponds to a mutation at position 15 as compared to SEQ ID NO: 1. In some embodiments, the nucleic acid molecule encodes a capsid polypeptide that comprises a mutation that corresponds to a mutation at position 19 as compared to SEQ ID NO: 1. In some embodiments, the nucleic acid molecule encodes a capsid polypeptide that comprises a mutation that corresponds to a mutation at position 21 as compared to SEQ ID NO: 1. In some embodiments, the nucleic acid molecule encodes a capsid polypeptide that comprises a mutation that corresponds to a mutation at position 23 as compared to SEQ ID NO: 1. In some embodiments, the nucleic acid molecule encodes a capsid polypeptide that comprises a mutation that corresponds to a mutation at position 24 as compared to SEQ ID NO: 1. In some embodiments, the nucleic acid molecule encodes a capsid polypeptide that comprises a mutation that corresponds to a mutation at position 25 as compared to SEQ ID NO: 1. In some embodiments, the nucleic acid molecule encodes a capsid polypeptide that comprises a mutation that corresponds to a mutation at position 29 as compared to SEQ ID NO: 1. In some embodiments, the nucleic acid molecule encodes a capsid polypeptide that comprises a mutation that corresponds to a mutation at position 31 as compared to SEQ ID NO: 1. In some embodiments, the nucleic acid molecule encodes a capsid polypeptide that comprises a mutation that corresponds to a mutation at position 33 as compared to SEQ ID NO: 1. In some embodiments, the nucleic acid molecule encodes a capsid polypeptide that comprises a mutation that corresponds to a mutation at position 446 as compared to SEQ ID NO: 1. In some embodiments, the nucleic acid molecule encodes a capsid polypeptide that comprises a mutation that corresponds to a mutation at position 449 as compared to SEQ ID NO: 1. In some embodiments, the nucleic acid molecule encodes a capsid polypeptide that comprises a mutation that corresponds to a mutation at position 450 as compared to SEQ ID NO: 1. In some embodiments, the nucleic acid molecule encodes a capsid polypeptide that comprises a mutation that corresponds to a mutation at position 451 as compared to SEQ ID NO: 1. In some embodiments, the nucleic acid molecule encodes a capsid polypeptide that comprises a mutation that corresponds to a mutation at position 452 as compared to SEQ ID NO: 1. In some embodiments, the nucleic acid molecule encodes a capsid polypeptide that comprises a mutation that corresponds to a mutation at position 454 as compared to SEQ ID NO: 1. In some embodiments, the nucleic acid molecule encodes a capsid polypeptide that comprises a mutation that corresponds to a mutation at position 455 as compared to SEQ ID NO: 1. In some embodiments, the nucleic acid molecule encodes a capsid polypeptide that comprises a mutation that corresponds to a mutation at position 456 as compared to SEQ ID NO: 1. In some embodiments, the nucleic acid molecule encodes a capsid polypeptide that comprises a mutation that corresponds to a mutation at position 457 as compared to SEQ ID NO: 1. In some embodiments, the nucleic acid molecule encodes a capsid polypeptide that comprises a mutation that corresponds to a mutation at position 458 as compared to SEQ ID NO: 1. In some embodiments, the nucleic acid molecule encodes a capsid polypeptide that comprises a mutation that corresponds to a mutation at position 459 as compared to SEQ ID NO: 1. In some embodiments, the nucleic acid molecule encodes a capsid polypeptide that comprises a mutation that corresponds to a mutation at position 460 as compared to SEQ ID NO: 1. In some embodiments, the nucleic acid molecule encodes a capsid polypeptide that comprises a mutation that corresponds to a mutation at position 461 as compared to SEQ ID NO: 1. In some embodiments, the nucleic acid molecule encodes a capsid polypeptide that comprises a mutation that corresponds to a mutation at position 464 as compared to SEQ ID NO: 1. In some embodiments, the nucleic acid molecule encodes a capsid polypeptide that comprises a mutation that corresponds to a mutation at position 552 as compared to SEQ ID NO: 1. In some embodiments, the nucleic acid molecule encodes a capsid polypeptide that comprises a mutation that corresponds to a mutation at position 554 as compared to SEQ ID NO: 1. In some embodiments, the nucleic acid molecule encodes a capsid polypeptide that comprises a mutation that corresponds to a mutation at position 555 as compared to SEQ ID NO: 1. In some embodiments, the nucleic acid molecule encodes a capsid polypeptide that comprises a mutation that corresponds to a mutation at position 556 as compared to SEQ ID NO: 1. In some embodiments, the nucleic acid molecule encodes a capsid polypeptide that comprises a mutation that corresponds to a mutation at position 557 as compared to SEQ ID NO: 1. In some embodiments, the nucleic acid molecule encodes a capsid polypeptide that comprises a mutation that corresponds to a mutation at position 558 as compared to SEQ ID NO: 1. In some embodiments, the nucleic acid molecule encodes a capsid polypeptide that comprises a mutation that corresponds to a mutation at position 559 as compared to SEQ ID NO: 1. In some embodiments, the nucleic acid molecule encodes a capsid polypeptide that comprises a mutation that corresponds to a mutation at position 561 as compared to SEQ ID NO: 1. In some embodiments, the nucleic acid molecule encodes a capsid polypeptide that comprises a mutation that corresponds to a mutation at position 566 as compared to SEQ ID NO: 1. In some embodiments, the nucleic acid molecule encodes a capsid polypeptide that comprises a mutation that corresponds to a mutation at position 575 as compared to SEQ ID NO: 1. In some embodiments, the nucleic acid molecule encodes a capsid polypeptide that comprises a mutation that corresponds to a mutation at position 578 as compared to SEQ ID NO: 1. In some embodiments, the nucleic acid molecule encodes a capsid polypeptide that comprises a mutation that corresponds to a mutation at position 580 as compared to SEQ ID NO: 1. In some embodiments, the nucleic acid molecule encodes a capsid polypeptide that comprises a mutation that corresponds to a mutation at position 581 as compared to SEQ ID NO: 1. In some embodiments, the nucleic acid molecule encodes a capsid polypeptide that comprises a mutation that corresponds to a mutation at position 585 as compared to SEQ ID NO: 1. In some embodiments, the nucleic acid molecule encodes a capsid polypeptide that comprises a mutation that corresponds to a mutation at position 586 as compared to SEQ ID NO: 1. In some embodiments, the nucleic acid molecule encodes a capsid polypeptide that comprises a mutation that corresponds to a mutation at position 587 as compared to SEQ ID NO: 1. In some embodiments, the nucleic acid molecule encodes a capsid polypeptide that comprises a mutation that corresponds to a mutation at position 588 as compared to SEQ ID NO: 1. In some embodiments, the nucleic acid molecule encodes a capsid polypeptide that comprises a mutation that corresponds to a mutation at position 589 as compared to SEQ ID NO: 1. In some embodiments, the nucleic acid molecule encodes a capsid polypeptide that comprises a mutation that corresponds to a mutation at position 590 as compared to SEQ ID NO: 1. In some embodiments, the nucleic acid molecule encodes a capsid polypeptide that comprises a mutation that corresponds to a mutation at position 591 as compared to SEQ ID NO: 1. In some embodiments, the nucleic acid molecule encodes a capsid polypeptide that comprises a mutation that corresponds to a mutation at position 593 as compared to SEQ ID NO: 1. In some embodiments, the nucleic acid molecule encodes a capsid polypeptide that comprises a mutation that corresponds to a mutation at position 594 as compared to SEQ ID NO: 1. In some embodiments, the nucleic acid molecule encodes a capsid polypeptide that comprises a mutation that corresponds to a mutation at position 597 as compared to SEQ ID NO: 1. In some embodiments, the nucleic acid molecule encodes a capsid polypeptide that comprises a mutation that corresponds to a mutation at position 600 as compared to SEQ ID NO: 1.

In some embodiments, the nucleic acid molecule encodes a capsid polypeptide that comprises a mutation that corresponds to an insertion at position between positions 446 and 447 as compared to SEQ ID NO: 1. In some embodiments, the nucleic acid molecule encodes a capsid polypeptide that comprises a mutation that corresponds to an insertion at position between positions 447 and 448 as compared to SEQ ID NO: 1. In some embodiments, the nucleic acid molecule encodes a capsid polypeptide that comprises a mutation that corresponds to an insertion at position between positions 448 and 449 as compared to SEQ ID NO: 1. In some embodiments, the nucleic acid molecule encodes a capsid polypeptide that comprises a mutation that corresponds to an insertion at position between positions 449 and 450 as compared to SEQ ID NO: 1. In some embodiments, the nucleic acid molecule encodes a capsid polypeptide that comprises a mutation that corresponds to an insertion at position between positions 451 and 452 as compared to SEQ ID NO: 1. In some embodiments, the nucleic acid molecule encodes a capsid polypeptide that comprises a mutation that corresponds to an insertion at position between positions 453 and 454 as compared to SEQ ID NO: 1. In some embodiments, the nucleic acid molecule encodes a capsid polypeptide that comprises a mutation that corresponds to an insertion at position between positions 581 and 582 as compared to SEQ ID NO: 1. In some embodiments, the nucleic acid molecule encodes a capsid polypeptide that comprises a mutation that corresponds to an insertion at position between positions 583 and 584 as compared to SEQ ID NO: 1. In some embodiments, the nucleic acid molecule encodes a capsid polypeptide that comprises a mutation that corresponds to an insertion at position between positions 584 and 585 as compared to SEQ ID NO: 1. In some embodiments, the nucleic acid molecule encodes a capsid polypeptide that comprises a mutation that corresponds to an insertion at position between positions 585 and 586 as compared to SEQ ID NO: 1. In some embodiments, the nucleic acid molecule encodes a capsid polypeptide that comprises a mutation that corresponds to an insertion at position between positions 586 and 587 as compared to SEQ ID NO: 1. In some embodiments, the nucleic acid molecule encodes a capsid polypeptide that comprises a mutation that corresponds to an insertion at position between positions 587 and 588 as compared to SEQ ID NO: 1. In some embodiments, the nucleic acid molecule encodes a capsid polypeptide that comprises a mutation that corresponds to an insertion at position between positions 588 and 589 as compared to SEQ ID NO: 1. In some embodiments, the nucleic acid molecule encodes a capsid polypeptide that comprises a mutation that corresponds to an insertion at position between positions 589 and 590 as compared to SEQ ID NO: 1. In some embodiments, the nucleic acid molecule encodes a capsid polypeptide that comprises a mutation that corresponds to an insertion at position between positions 590 and 591 as compared to SEQ ID NO: 1. In some embodiments, the nucleic acid molecule encodes a capsid polypeptide that comprises a mutation that corresponds to an insertion at position between positions 591 and 592 as compared to SEQ ID NO: 1. In some embodiments, the nucleic acid molecule encodes a capsid polypeptide that comprises a mutation that corresponds to an insertion at position between positions 592 and 593 as compared to SEQ ID NO: 1.

In some embodiments, the nucleic acid molecule encodes a capsid polypeptide that comprises a mutation that corresponds to a mutation at position 3, 6, 11, 12, 14, 15, 19, 21, 23, 24, 25, 29, 31, 33, or any combination thereof according to SEQ ID NO: 1, and wherein the mutation comprises a deletion or a substitution.

In some embodiments, the nucleic acid molecule encodes a capsid polypeptide that comprises a mutation that corresponds to a mutation at position 446, 448, 449, 450, 451, 452, 453, 454, 455, 456, 457, 458, 459, 460, 461, 464, or any combination thereof, an insertion between positions 446 and 447, 447 and 448, 448 and 449, 449 and 450, 451 and 452, 453 and 454, or any combination thereof according to SEQ ID NO: 1, and wherein the mutation comprises an insertion, a deletion or a substitution.

In some embodiments, the nucleic acid molecule encodes a capsid polypeptide that comprises a mutation that corresponds to a mutation at position 552, 554, 555, 556, 557, 558, 559, 561, 566, 575, 578, 580, 581, 585, 586, 587, 588, 589, 590, 591, 593, 594, 597, 600 or any combination thereof, an insertion between positions 583 and 584, 584 and 585, 585 and 586, 586 and 587, 587 and 588, 588 and 589, 589 and 590, 590 and 591, 591 and 592, 592 and 593, or any combination thereof according to SEQ ID NO: 1, and wherein the mutation comprises an insertion, a deletion or a substitution.

In some embodiments, the nucleic acid molecule encodes a capsid polypeptide that comprises a mutation that corresponds to a mutation at position 14, 15, 19, and 24 as compared to SEQ ID NO: 1.

In some embodiments, the nucleic acid molecule encodes a capsid polypeptide that comprises a mutation that corresponds to a mutation at position 3, 6, 11, 12, 21, 23, 25, 29, 31, and 33 as compared to SEQ ID NO: 1.

In some embodiments, the nucleic acid molecule encodes a capsid polypeptide that comprises a mutation that corresponds to a mutation at position 449, 450, 451, 455, 456, 457, 458, and 459 as compared to SEQ ID NO: 1.

In some embodiments, the nucleic acid molecule encodes a capsid polypeptide that comprises a mutation that corresponds to a mutation at position 449, 450, 452, 456, 457, and 459 as compared to SEQ ID NO: 1.

In some embodiments, the nucleic acid molecule encodes a capsid polypeptide that comprises a mutation that corresponds to a mutation at position 457 and 458 as compared to SEQ ID NO: 1.

In some embodiments, the nucleic acid molecule encodes a capsid polypeptide that comprises a mutation that corresponds to a mutation at position 446 as compared to SEQ ID NO: 1.

In some embodiments, the nucleic acid molecule encodes a capsid polypeptide that comprises a mutation that corresponds to a mutation at position 449 as compared to SEQ ID NO: 1.

In some embodiments, the nucleic acid molecule encodes a capsid polypeptide that comprises a mutation that corresponds to a mutation at position 451, 455, 456, 457, 458, 459, and 461 as compared to SEQ ID NO: 1.

In some embodiments, the nucleic acid molecule encodes a capsid polypeptide that comprises a mutation that corresponds to a mutation at position 448 as compared to SEQ ID NO: 1.

In some embodiments, the nucleic acid molecule encodes a capsid polypeptide that comprises a mutation that corresponds to a mutation at position 453 as compared to SEQ ID NO: 1.

In some embodiments, the nucleic acid molecule encodes a capsid polypeptide that comprises a mutation that corresponds to a mutation at position 456, 457, 458, 459, 460, and 461, as compared to SEQ ID NO: 1.

In some embodiments, the nucleic acid molecule encodes a capsid polypeptide that comprises a mutation that corresponds to a mutation at position 449, 450, 451, 454, 455, 456, 458, 459, 461, and 464 as compared to SEQ ID NO: 1.

In some embodiments, the nucleic acid molecule encodes a capsid polypeptide that comprises a mutation that corresponds to a mutation at position 446, 448, 449, 451, 453, 455, 456, 457, and 459 as compared to SEQ ID NO: 1.

In some embodiments, the nucleic acid molecule encodes a capsid polypeptide that comprises a mutation that corresponds to a mutation at position 447 as compared to SEQ ID NO: 1.

In some embodiments, the nucleic acid molecule encodes a capsid polypeptide that comprises a mutation that corresponds to a mutation at position 581 as compared to SEQ ID NO: 1.

In some embodiments, the nucleic acid molecule encodes a capsid polypeptide that comprises a mutation that corresponds to a mutation at position 584 as compared to SEQ ID NO: 1.

In some embodiments, the nucleic acid molecule encodes a capsid polypeptide that comprises a mutation that corresponds to a mutation at position 555, 561, 566, 578, 580, 581, and 585 as compared to SEQ ID NO: 1.

In some embodiments, the nucleic acid molecule encodes a capsid polypeptide that comprises a mutation that corresponds to a mutation at position 557 and 578 as compared to SEQ ID NO: 1.

In some embodiments, the nucleic acid molecule encodes a capsid polypeptide that comprises a mutation that corresponds to a mutation at position 556, 558, 578, and 580 as compared to SEQ ID NO: 1.

In some embodiments, the nucleic acid molecule encodes a capsid polypeptide that comprises a mutation that corresponds to a mutation at position 554, 556, 559, 561, 566, 581, 585, 586, and 587 as compared to SEQ ID NO: 1.

In some embodiments, the nucleic acid molecule encodes a capsid polypeptide that comprises a mutation that corresponds to a mutation at position 552, 555, 556, 559, 561, 566, 578, 581, and 586 as compared to SEQ ID NO: 1.

In some embodiments, the nucleic acid molecule encodes a capsid polypeptide that comprises a mutation that corresponds to a mutation at position 575 and 578 as compared to SEQ ID NO: 1.

In some embodiments, the nucleic acid molecule encodes a capsid polypeptide that comprises a mutation that corresponds to a mutation at position 583 as compared to SEQ ID NO: 1.

In some embodiments, the nucleic acid molecule encodes a capsid polypeptide that comprises a mutation that corresponds to a mutation at position 585, 588, 589, 590, 591, 593, 597, and 600 as compared to SEQ ID NO: 1.

In some embodiments, the nucleic acid molecule encodes a capsid polypeptide that comprises a mutation that corresponds to a mutation at position 592 as compared to SEQ ID NO: 1.

In some embodiments, the nucleic acid molecule encodes a capsid polypeptide that comprises a mutation that corresponds to a mutation at position 585 as compared to SEQ ID NO: 1.

In some embodiments, the nucleic acid molecule encodes a capsid polypeptide that comprises a mutation that corresponds to a mutation at position 586 as compared to SEQ ID NO: 1.

In some embodiments, the nucleic acid molecule encodes a capsid polypeptide that comprises a mutation that corresponds to a mutation at position 589 as compared to SEQ ID NO: 1.

In some embodiments, the nucleic acid molecule encodes a capsid polypeptide that comprises a mutation that corresponds to a mutation at position 587 as compared to SEQ ID NO: 1.

In some embodiments, the nucleic acid molecule encodes a capsid polypeptide that comprises a mutation that corresponds to a mutation at position 590 as compared to SEQ ID NO: 1.

In some embodiments, the nucleic acid molecule encodes a capsid polypeptide that comprises a mutation that corresponds to a mutation at position 585, 586, 587, 591, 594, 597, and 600 as compared to SEQ ID NO: 1.

In some embodiments, the nucleic acid molecule encodes a capsid polypeptide that comprises a mutation that corresponds to a mutation at position 591 as compared to SEQ ID NO: 1.

In some embodiments, the nucleic acid molecule encodes a capsid polypeptide that comprises a mutation that corresponds to a mutation at position 588 as compared to SEQ ID NO: 1.

In some embodiments, the nucleic acid molecule encodes a capsid polypeptide that comprises a mutation that comprises an insertion, e.g., an insertion of 1 or more amino acids, e.g., 1-10 amino acids, e.g., 2-8 amino acids, e.g., 3-7 amino acids, e.g., 7 amino acids, that corresponds to an insertion between positions 446 and 447, 447 and 448, 448 and 449, 449 and 450, 451, and 452, 453 and 454, 581 and 582, 583 and 584, 584 and 585, 585 and 586, 586 and 587, 587 and 588, 588 and 589, 589 and 590, 590 and 591, 591 and 592, or 592 and 593, as compared to SEQ ID NO: 1.

In some embodiments, the nucleic acid molecule encodes a capsid polypeptide that comprises a mutation that comprises an insertion between positions 446 and 447 as compared to SEQ ID NO: 1.

In some embodiments, the nucleic acid molecule encodes a capsid polypeptide that comprises a mutation that comprises an insertion between positions 447 and 448 as compared to SEQ ID NO: 1.

In some embodiments, the nucleic acid molecule encodes a capsid polypeptide that comprises a mutation that comprises an insertion between positions 448 and 449 as compared to SEQ ID NO: 1.

In some embodiments, the nucleic acid molecule encodes a capsid polypeptide that comprises a mutation that comprises an insertion between positions 449 and 450 as compared to SEQ ID NO: 1.

In some embodiments, the nucleic acid molecule encodes a capsid polypeptide that comprises a mutation that comprises an insertion between positions 451, and 452 as compared to SEQ ID NO: 1.

In some embodiments, the nucleic acid molecule encodes a capsid polypeptide that comprises a mutation that comprises an insertion between positions 453 and 454 as compared to SEQ ID NO: 1.

In some embodiments, the nucleic acid molecule encodes a capsid polypeptide that comprises a mutation that comprises an insertion between positions 581 and 582 as compared to SEQ ID NO: 1.

In some embodiments, the nucleic acid molecule encodes a capsid polypeptide that comprises a mutation that comprises an insertion between positions 583 and 584 as compared to SEQ ID NO: 1.

In some embodiments, the nucleic acid molecule encodes a capsid polypeptide that comprises a mutation that comprises an insertion between positions 584 and 585 as compared to SEQ ID NO: 1.

In some embodiments, the nucleic acid molecule encodes a capsid polypeptide that comprises a mutation that comprises an insertion between positions 585 and 586 as compared to SEQ ID NO: 1.

In some embodiments, the nucleic acid molecule encodes a capsid polypeptide that comprises a mutation that comprises an insertion between positions 586 and 587 as compared to SEQ ID NO: 1.

In some embodiments, the nucleic acid molecule encodes a capsid polypeptide that comprises a mutation that comprises an insertion between positions 587 and 588 as compared to SEQ ID NO: 1.

In some embodiments, the nucleic acid molecule encodes a capsid polypeptide that comprises a mutation that comprises an insertion between positions 588 and 589 as compared to SEQ ID NO: 1.

In some embodiments, the nucleic acid molecule encodes a capsid polypeptide that comprises a mutation that comprises an insertion between positions 589 and 590 as compared to SEQ ID NO: 1.

In some embodiments, the nucleic acid molecule encodes a capsid polypeptide that comprises a mutation that comprises an insertion between positions 590 and 591 as compared to SEQ ID NO: 1.

In some embodiments, the nucleic acid molecule encodes a capsid polypeptide that comprises a mutation that comprises an insertion between positions 591 and 592 as compared to SEQ ID NO: 1.

In some embodiments, the nucleic acid molecule encodes a capsid polypeptide that comprises a mutation that comprises an insertion between positions 592 and 593 as compared to SEQ ID NO: 1.

In some embodiments, the nucleic acid molecule encodes a capsid polypeptide that comprises a mutation that corresponds to a mutation at position 449, 450, 452, 455, 457, 458, 459, and an insertion between positions 451 and 452 as compared to SEQ ID NO: 1.

In some embodiments, the nucleic acid molecule encodes a capsid polypeptide that comprises a mutation that corresponds to a mutation at position 585 and 586 and an insertion between positions 584 and 585 as compared to SEQ ID NO: 1.

In some embodiments, the nucleic acid molecule encodes a capsid polypeptide that comprises a mutation that corresponds to a mutation at position 586 and 587 and an insertion between positions 584 and 585 as compared to SEQ ID NO: 1.

In some embodiments, the nucleic acid molecule encodes a capsid polypeptide that comprises a mutation that corresponds to a mutation at position 587 and an insertion between positions 586 and 587 as compared to SEQ ID NO: 1.

In some embodiments, the nucleic acid molecule encodes a capsid polypeptide that comprises a mutation that corresponds to a T14L, L15V, I19A, and K24H mutation as compared to SEQ ID NO: 1.

In some embodiments, the nucleic acid molecule encodes a capsid polypeptide that comprises a mutation that corresponds to a A3V, Y6F, L11F, E12Q, Q21L, W23I, L25C, P29A, P31N, and K33R mutation as compared to SEQ ID NO: 1.

In some embodiments, the nucleic acid molecule encodes a capsid polypeptide that comprises a mutation that corresponds to a N449Q, T450M, P451T, T455L, T456G, Q457T, S458Q, and R459M mutation as compared to SEQ ID NO: 1.

In some embodiments, the nucleic acid molecule encodes a capsid polypeptide that comprises a mutation that corresponds to a N449Q, residue 451 deletion, S452T, T456G, Q457T, and R459G mutation as compared to SEQ ID NO: 1.

In some embodiments, the nucleic acid molecule encodes a capsid polypeptide that comprises a mutation that corresponds to a Q457F and S458C mutation as compared to SEQ ID NO: 1.

In some embodiments, the nucleic acid molecule encodes a capsid polypeptide that comprises a mutation that corresponds to an insertion between residues 446 and 447 as compared to SEQ ID NO: 1, wherein the insertion comprises, e.g., consists of, a polypeptide of KCQEGMA (SEQ ID NO: 95). In some embodiments, the insertion comprises a polypeptide that has at least 57.1%, 71.4%, 85.7%, or 100% identity to KCQEGMA (SEQ ID NO: 95). In some embodiments, the insertion comprises a polypeptide that has at least 1, 2, or 3 mutations as compared to KCQEGMA (SEQ ID NO: 95). In some embodiments, the insertion comprises a polypeptide comprising a fragment of at least 4 amino acids of KCQEGMA (SEQ ID NO: 95). In some embodiments, the insertion comprises a polypeptide comprising a fragment of at least 5 amino acids of KCQEGMA (SEQ ID NO: 95). In some embodiments, the insertion comprises a polypeptide comprising a fragment of at least 6 amino acids of KCQEGMA (SEQ ID NO: 95).

In some embodiments, the nucleic acid molecule encodes a capsid polypeptide that comprises a mutation that corresponds to an insertion between residues 449 and 450 as compared to SEQ ID NO: 1, wherein the insertion comprises, e.g., consists of, a polypeptide of LMVDRLG (SEQ ID NO: 96). In some embodiments, the insertion comprises a polypeptide that has at least 57.1%, 71.4%, 85.7%, or 100% identity to LMVDRLG (SEQ ID NO: 96). In some embodiments, the insertion comprises a polypeptide that has at least 1, 2, or 3 mutations as compared to LMVDRLG (SEQ ID NO: 96). In some embodiments, the insertion comprises a polypeptide comprising a fragment of at least 4 amino acids of LMVDRLG (SEQ ID NO: 96). In some embodiments, the insertion comprises a polypeptide comprising a fragment of at least 5 amino acids of LMVDRLG (SEQ ID NO: 96). In some embodiments, the insertion comprises a polypeptide comprising a fragment of at least 6 amino acids of LMVDRLG (SEQ ID NO: 96).

In some embodiments, the nucleic acid molecule encodes a capsid polypeptide that comprises a mutation that corresponds to a P451T, T455G, T456G, Q457T, S458Q, R459T, and Q461A mutation as compared to SEQ ID NO: 1.

In some embodiments, the nucleic acid molecule encodes a capsid polypeptide that comprises a mutation that corresponds to an insertion between residues 448 and 449 as compared to SEQ ID NO: 1, wherein the insertion comprises, e.g., consists of, a polypeptide of HCQECPI (SEQ ID NO: 97). In some embodiments, the insertion comprises a polypeptide that has at least 57.1%, 71.4%, 85.7%, or 100% identity to HCQECPI (SEQ ID NO: 97). In some embodiments, the insertion comprises a polypeptide that has at least 1, 2, or 3 mutations as compared to HCQECPI (SEQ ID NO: 97). In some embodiments, the insertion comprises a polypeptide comprising a fragment of at least 4 amino acids of HCQECPI (SEQ ID NO: 97). In some embodiments, the insertion comprises a polypeptide comprising a fragment of at least 5 amino acids of HCQECPI (SEQ ID NO: 97). In some embodiments, the insertion comprises a polypeptide comprising a fragment of at least 6 amino acids of HCQECPI (SEQ ID NO: 97).

In some embodiments, the nucleic acid molecule encodes a capsid polypeptide that comprises a mutation that corresponds to an insertion between residues 453 and 454 as compared to SEQ ID NO: 1, wherein the insertion comprises, e.g., consists of, a polypeptide of FSGLEN (SEQ ID NO: 98). In some embodiments, the insertion comprises a polypeptide that has at least 50%, 66.67%, 83.3%, or 100% identity to FSGLEN (SEQ ID NO: 98). In some embodiments, the insertion comprises a polypeptide that has at least 1, 2, or 3 mutations as compared to FSGLEN (SEQ ID NO: 98). In some embodiments, the insertion comprises a polypeptide comprising a fragment of at least 4 amino acids of FSGLEN (SEQ ID NO: 98). In some embodiments, the insertion comprises a polypeptide comprising a fragment of at least 3 amino acids of FSGLEN (SEQ ID NO: 98).

In some embodiments, the nucleic acid molecule encodes a capsid polypeptide that comprises a mutation that corresponds to a residue deletion at positions 456, 457, 458, 459, 460, and 461 as compared to SEQ ID NO: 1.

In some embodiments, the nucleic acid molecule encodes a capsid polypeptide that comprises a mutation that corresponds to a N449I, T450N, P451G, residue 454 deletion, T455Q, T456N, S458Q, R459T, Q461K, and Q464V mutation as compared to SEQ ID NO: 1.

In some embodiments, the nucleic acid molecule encodes a capsid polypeptide that comprises a mutation that corresponds to a S446A, residue 448 and 449 deletion, P451Q, G453T, T455G, T456G, Q457T, and R459G mutation as compared to SEQ ID NO: 1.

In some embodiments, the nucleic acid molecule encodes a capsid polypeptide that comprises a mutation that corresponds to a N449Q, T450S, S452G, T455A, Q457F, S458M, R459D, and an insertion between residues 451 and 452 as compared to SEQ ID NO: 1, wherein the insertion comprises an amino acid Y.

In some embodiments, the nucleic acid molecule encodes a capsid polypeptide that comprises a mutation that corresponds to an insertion between residues 447 and 448 as compared to SEQ ID NO: 1, wherein the insertion comprises, e.g., consists of, a polypeptide of HETEFNF (SEQ ID NO: 99). In some embodiments, the insertion comprises a polypeptide that has at least 57.1%, 71.4%, 85.7%, or 100% identity to HETEFNF (SEQ ID NO: 99). In some embodiments, the insertion comprises a polypeptide that has at least 1, 2, or 3 mutations as compared to HETEFNF (SEQ ID NO: 99). In some embodiments, the insertion comprises a polypeptide comprising a fragment of at least 4 amino acids of HETEFNF (SEQ ID NO: 99). In some embodiments, the insertion comprises a polypeptide comprising a fragment of at least 5 amino acids of HETEFNF (SEQ ID NO: 99). In some embodiments, the insertion comprises a polypeptide comprising a fragment of at least 6 amino acids of HETEFNF (SEQ ID NO: 99).

In some embodiments, the nucleic acid molecule encodes a capsid polypeptide that comprises a mutation that corresponds to a P451T, T455G, T456G, Q457T, S458Q, R459T, and Q461A mutation as compared to SEQ ID NO: 1.

In some embodiments, the nucleic acid molecule encodes a capsid polypeptide that comprises a mutation that corresponds to an insertion between residues 581 and 582 as compared to SEQ ID NO: 1, wherein the insertion comprises, e.g., consists of, a polypeptide of FALMEP (SEQ ID NO: 100). In some embodiments, the insertion comprises a polypeptide that has at least 50%, 66.67%, 83.3%, or 100% identity to FALMEP (SEQ ID NO: 100). In some embodiments, the insertion comprises a polypeptide that has at least 1, 2, or 3 mutations as compared to FALMEP (SEQ ID NO: 100). In some embodiments, the insertion comprises a polypeptide comprising a fragment of at least 4 amino acids of FALMEP (SEQ ID NO: 100). In some embodiments, the insertion comprises a polypeptide comprising a fragment of at least 5 amino acids of FALMEP (SEQ ID NO: 100).

In some embodiments, the nucleic acid molecule encodes a capsid polypeptide that comprises a mutation that corresponds to an insertion between residues 584 and 585 as compared to SEQ ID NO: 1, wherein the insertion comprises, e.g., consists of, a polypeptide of RAYNPD (SEQ ID NO: 101). In some embodiments, the insertion comprises a polypeptide that has at least 50%, 66.67%, 83.3%, or 100% identity to RAYNPD (SEQ ID NO: 101). In some embodiments, the insertion comprises a polypeptide that has at least 1, 2, or 3 mutations as compared to RAYNPD (SEQ ID NO: 101). In some embodiments, the insertion comprises a polypeptide comprising a fragment of at least 4 amino acids of RAYNPD (SEQ ID NO: 101). In some embodiments, the insertion comprises a polypeptide comprising a fragment of at least 5 amino acids of RAYNPD (SEQ ID NO: 101).

In some embodiments, the nucleic acid molecule encodes a capsid polypeptide that comprises a mutation that corresponds to a residue 585 deletion and a E555A, D461S, R566K, S578V, S580A, and T581A mutation as compared to SEQ ID NO: 1.

In some embodiments, the nucleic acid molecule encodes a capsid polypeptide that comprises a mutation that corresponds to a V557L and S578D mutation as compared to SEQ ID NO: 1.

In some embodiments, the nucleic acid molecule encodes a capsid polypeptide that comprises a mutation that corresponds to a K556N, M558L, S578I, and S580A mutation as compared to SEQ ID NO: 1.

In some embodiments, the nucleic acid molecule encodes a capsid polypeptide that comprises a mutation that corresponds to a I554L, K556R, I559L, D561S, R566A, T581D, R585S, G586R, and N587S mutation as compared to SEQ ID NO: 1.

In some embodiments, the nucleic acid molecule encodes a capsid polypeptide that comprises a mutation that corresponds to a V552A, E555S, K556D, I559L, D561S, R566K, S578I, T581D, and G586Q mutation as compared to SEQ ID NO: 1.

In some embodiments, the nucleic acid molecule encodes a capsid polypeptide that comprises a mutation that corresponds to a Q575E and S578A mutation as compared to SEQ ID NO: 1.

In some embodiments, the nucleic acid molecule encodes a capsid polypeptide that comprises a mutation that corresponds to an insertion between residues 583 and 584 as compared to SEQ ID NO: 1, wherein the insertion comprises, e.g., consists of, a polypeptide of LNWTAE (SEQ ID NO: 102). In some embodiments, the insertion comprises a polypeptide that has at least 50%, 66.67%, 83.3%, or 100% identity to LNWTAE (SEQ ID NO: 102). In some embodiments, the insertion comprises a polypeptide that has at least 1, 2, or 3 mutations as compared to LNWTAE (SEQ ID NO: 102). In some embodiments, the insertion comprises a polypeptide comprising a fragment of at least 4 amino acids of LNWTAE (SEQ ID NO: 102). In some embodiments, the insertion comprises a polypeptide comprising a fragment of at least 5 amino acids of LNWTAE (SEQ ID NO: 102).

In some embodiments, the nucleic acid molecule encodes a capsid polypeptide that comprises a mutation that corresponds to a R585S, R588T, Q589N, A590P, A591I, A593G, T597S, and V600A mutation as compared to SEQ ID NO: 1.

In some embodiments, the nucleic acid molecule encodes a capsid polypeptide that comprises a mutation that corresponds to a R585N and G586A mutation, and an insertion between residues 584 and 585 as compared to SEQ ID NO: 1, wherein the insertion comprises, e.g., consists of, a polypeptide of LAKEFTTR (SEQ ID NO: 103). In some embodiments, the insertion comprises a polypeptide that has at least 50%, 62.5%, 75%, 87.5%, or 100% identity to LAKEFTTR (SEQ ID NO: 103). In some embodiments, the insertion comprises a polypeptide that has at least 1, 2, 3, or 4 mutations as compared to LAKEFTTR (SEQ ID NO: 103). In some embodiments, the insertion comprises a polypeptide comprising a fragment of at least 4 amino acids of LAKEFTTR (SEQ ID NO: 103). In some embodiments, the insertion comprises a polypeptide comprising a fragment of at least 5 amino acids of LAKEFTTR (SEQ ID NO: 103). In some embodiments, the insertion comprises a polypeptide comprising a fragment of at least 6 amino acids of LAKEFTTR (SEQ ID NO: 103). In some embodiments, the insertion comprises a polypeptide comprising a fragment of at least 7 amino acids of LAKEFTTR (SEQ ID NO: 103). In some embodiments, the insertion comprises, e.g., consists of, KEFTTR (SEQ ID NO: 104).

In some embodiments, the nucleic acid molecule encodes a capsid polypeptide that comprises a mutation that corresponds to an insertion between residues 592 and 593 as compared to SEQ ID NO: 1, wherein the insertion comprises, e.g., consists of, a polypeptide of LHPLE (SEQ ID NO: 105). In some embodiments, the insertion comprises a polypeptide that has at least 60%, 80%, or 100% identity to LHPLE (SEQ ID NO: 105). In some embodiments, the insertion comprises a polypeptide that has at least 1, or 2 mutations as compared to LHPLE (SEQ ID NO: 105). In some embodiments, the insertion comprises a polypeptide comprising a fragment of at least 4 amino acids of LHPLE (SEQ ID NO: 105).

In some embodiments, the nucleic acid molecule encodes a capsid polypeptide that comprises a mutation that corresponds to an insertion between residues 585 and 586 as compared to SEQ ID NO: 1, wherein the insertion comprises an amino acid F.

In some embodiments, the nucleic acid molecule encodes a capsid polypeptide that comprises a mutation that corresponds to an insertion between residues 586 and 587 as compared to SEQ ID NO: 1, wherein the insertion comprises, e.g., consists of, a polypeptide of DQDFKNR (SEQ ID NO: 106). In some embodiments, the insertion comprises a polypeptide that has at least 57.1%, 71.4%, 85.7%, or 100% identity to DQDFKNR (SEQ ID NO: 106). In some embodiments, the insertion comprises a polypeptide that has at least 1, 2, or 3 mutations as compared to DQDFKNR (SEQ ID NO: 106). In some embodiments, the insertion comprises a polypeptide comprising a fragment of at least 4 amino acids of DQDFKNR (SEQ ID NO: 106). In some embodiments, the insertion comprises a polypeptide comprising a fragment of at least 5 amino acids of DQDFKNR (SEQ ID NO: 106). In some embodiments, the insertion comprises a polypeptide comprising a fragment of at least 6 amino acids of DQDFKNR (SEQ ID NO: 106).

In some embodiments, the nucleic acid molecule encodes a capsid polypeptide that comprises a mutation that corresponds to an insertion between residues 589 and 590 as compared to SEQ ID NO: 1, wherein the insertion comprises an amino acid I.

In some embodiments, the nucleic acid molecule encodes a capsid polypeptide that comprises a mutation that corresponds to an insertion between residues 587 and 588 as compared to SEQ ID NO: 1, wherein the insertion comprises, e.g., consists of, a polypeptide of LAIEQTRPA (SEQ ID NO: 107). In some embodiments, the insertion comprises a polypeptide that has at least 55.5%, 66.6%, 77.7%, 88.8%, or 100% identity to LAIEQTRPA (SEQ ID NO: 107). In some embodiments, the insertion comprises a polypeptide that has at least 1, 2, 3, or 4 mutations as compared to LAIEQTRPA (SEQ ID NO: 107). In some embodiments, the insertion comprises a polypeptide comprising a fragment of at least 4 amino acids of LAIEQTRPA (SEQ ID NO: 107). In some embodiments, the insertion comprises a polypeptide comprising a fragment of at least 5 amino acids of LAIEQTRPA (SEQ ID NO: 107). In some embodiments, the insertion comprises a polypeptide comprising a fragment of at least 6 amino acids of LAIEQTRPA (SEQ ID NO: 107). In some embodiments, the insertion comprises a polypeptide comprising a fragment of at least 7 amino acids of LAIEQTRPA (SEQ ID NO: 107). In some embodiments, the insertion comprises a polypeptide comprising a fragment of at least 8 amino acids of LAIEQTRPA (SEQ ID NO: 107). In some embodiments, the insertion comprises, e.g., consists of, IEQTRPA (SEQ ID NO: 108).

In some embodiments, the nucleic acid molecule encodes a capsid polypeptide that comprises a mutation that corresponds to a G586P and N587A mutation, and an insertion between residues 584 and 585 as compared to SEQ ID NO: 1, wherein the insertion comprises, e.g., consists of, a polypeptide of RARLDETT (SEQ ID NO: 109). In some embodiments, the insertion comprises a polypeptide that has at least 50%, 62.5%, 75%, 87.5%, or 100% identity to RARLDETT (SEQ ID NO: 109). In some embodiments, the insertion comprises a polypeptide that has at least 1, 2, 3, or 4 mutations as compared to RARLDETT (SEQ ID NO: 109). In some embodiments, the insertion comprises a polypeptide comprising a fragment of at least 4 amino acids of RARLDETT (SEQ ID NO: 109). In some embodiments, the insertion comprises a polypeptide comprising a fragment of at least 5 amino acids of RARLDETT (SEQ ID NO: 109). In some embodiments, the insertion comprises a polypeptide comprising a fragment of at least 6 amino acids of RARLDETT (SEQ ID NO: 109). In some embodiments, the insertion comprises a polypeptide comprising a fragment of at least 7 amino acids of RARLDETT (SEQ ID NO: 109). In some embodiments, the insertion comprises, e.g., consists of, RLDETT (SEQ ID NO: 110).

In some embodiments, the nucleic acid molecule encodes a capsid polypeptide that comprises a mutation that corresponds to a N587A mutation, and an insertion between residues 586 and 587 as compared to SEQ ID NO: 1, wherein the insertion comprises, e.g., consists of, a polypeptide of LALAEITRP (SEQ ID NO: 111). In some embodiments, the insertion comprises a polypeptide that has at least 55.5%, 66.6%, 77.7%, 88.8%, or 100% identity to LALAEITRP (SEQ ID NO: 111). In some embodiments, the insertion comprises a polypeptide that has at least 1, 2, 3, or 4 mutations as compared to LALAEITRP (SEQ ID NO: 111). In some embodiments, the insertion comprises a polypeptide comprising a fragment of at least 4 amino acids of LALAEITRP (SEQ ID NO: 111). In some embodiments, the insertion comprises a polypeptide comprising a fragment of at least 5 amino acids of LALAEITRP (SEQ ID NO: 111). In some embodiments, the insertion comprises a polypeptide comprising a fragment of at least 6 amino acids of LALAEITRP (SEQ ID NO: 111). In some embodiments, the insertion comprises a polypeptide comprising a fragment of at least 7 amino acids of LALAEITRP (SEQ ID NO: 111). In some embodiments, the insertion comprises a polypeptide comprising a fragment of at least 8 amino acids of LALAEITRP (SEQ ID NO: 111). In some embodiments, the insertion comprises, e.g., consists of, LAEITRP (SEQ ID NO: 112).

In some embodiments, the nucleic acid molecule encodes a capsid polypeptide that comprises a mutation that corresponds to a N587A mutation, and an insertion between residues 586 and 587 as compared to SEQ ID NO: 1, wherein the insertion comprises, e.g., consists of, a polypeptide of LANGEQTRP (SEQ ID NO: 113). In some embodiments, the insertion comprises a polypeptide that has at least 55.5%, 66.6%, 77.7%, 88.8%, or 100% identity to LANGEQTRP (SEQ ID NO: 113). In some embodiments, the insertion comprises a polypeptide that has at least 1, 2, 3, or 4 mutations as compared to LANGEQTRP (SEQ ID NO: 113). In some embodiments, the insertion comprises a polypeptide comprising a fragment of at least 4 amino acids of LANGEQTRP (SEQ ID NO: 113). In some embodiments, the insertion comprises a polypeptide comprising a fragment of at least 5 amino acids of LANGEQTRP (SEQ ID NO: 113). In some embodiments, the insertion comprises a polypeptide comprising a fragment of at least 6 amino acids of LANGEQTRP (SEQ ID NO: 113). In some embodiments, the insertion comprises a polypeptide comprising a fragment of at least 7 amino acids of LANGEQTRP (SEQ ID NO: 113). In some embodiments, the insertion comprises a polypeptide comprising a fragment of at least 8 amino acids of LANGEQTRP (SEQ ID NO: 113). In some embodiments, the insertion comprises, e.g., consists of, NGEQTRP (SEQ ID NO: 114).

In some embodiments, the nucleic acid molecule encodes a capsid polypeptide that comprises a mutation that corresponds to an insertion between residues 586 and 587 as compared to SEQ ID NO: 1, wherein the insertion comprises, e.g., consists of, a polypeptide of ATDTKT (SEQ ID NO: 115). In some embodiments, the insertion comprises a polypeptide that has at least 50%, 66.67%, 83.3%, or 100% identity to ATDTKT (SEQ ID NO: 115). In some embodiments, the insertion comprises a polypeptide that has at least 1, 2, or 3 mutations as compared to ATDTKT (SEQ ID NO: 115). In some embodiments, the insertion comprises a polypeptide comprising a fragment of at least 4 amino acids of ATDTKT (SEQ ID NO: 115). In some embodiments, the insertion comprises a polypeptide comprising a fragment of at least 5 amino acids of ATDTKT (SEQ ID NO: 115).

In some embodiments, the nucleic acid molecule encodes a capsid polypeptide that comprises a mutation that corresponds to an insertion between residues 590 and 591 as compared to SEQ ID NO: 1, wherein the insertion comprises an amino acid P.

In some embodiments, the nucleic acid molecule encodes a capsid polypeptide that comprises a mutation that corresponds to an insertion between residues 587 and 588 as compared to SEQ ID NO: 1, wherein the insertion comprises, e.g., consists of, a polypeptide of APGETTRPA (SEQ ID NO: 116). In some embodiments, the insertion comprises a polypeptide that has at least 55.5%, 66.6%, 77.7%, 88.8%, or 100% identity to APGETTRPA (SEQ ID NO: 116). In some embodiments, the insertion comprises a polypeptide that has at least 1, 2, 3, or 4 mutations as compared to APGETTRPA (SEQ ID NO: 116). In some embodiments, the insertion comprises a polypeptide comprising a fragment of at least 4 amino acids of APGETTRPA (SEQ ID NO: 116). In some embodiments, the insertion comprises a polypeptide comprising a fragment of at least 5 amino acids of APGETTRPA (SEQ ID NO: 116). In some embodiments, the insertion comprises a polypeptide comprising a fragment of at least 6 amino acids of APGETTRPA (SEQ ID NO: 116). In some embodiments, the insertion comprises a polypeptide comprising a fragment of at least 7 amino acids of APGETTRPA (SEQ ID NO: 116). In some embodiments, the insertion comprises a polypeptide comprising a fragment of at least 8 amino acids of APGETTRPA (SEQ ID NO: 116). In some embodiments, the insertion comprises, e.g., consists of, GETTRP (SEQ ID NO: 117).

In some embodiments, the nucleic acid molecule encodes a capsid polypeptide that comprises a mutation that corresponds to an insertion between residues 589 and 590 as compared to SEQ ID NO: 1, wherein the insertion comprises an amino acid P.

In some embodiments, the nucleic acid molecule encodes a capsid polypeptide that comprises a mutation that corresponds to a R585S, G586S, N587A, A591E, D594R, T597A, and V600I mutation as compared to SEQ ID NO: 1.

In some embodiments, the nucleic acid molecule encodes a capsid polypeptide that comprises a mutation that corresponds to an insertion between residues 586 and 587 as compared to SEQ ID NO: 1, wherein the insertion comprises, e.g., consists of, a polypeptide of FHNEGKY (SEQ ID NO: 118). In some embodiments, the insertion comprises a polypeptide that has at least 57.1%, 71.4%, 85.7%, or 100% identity to FHNEGKY (SEQ ID NO: 118). In some embodiments, the insertion comprises a polypeptide that has at least 1, 2, or 3 mutations as compared to FHNEGKY (SEQ ID NO: 118). In some embodiments, the insertion comprises a polypeptide comprising a fragment of at least 4 amino acids of FHNEGKY (SEQ ID NO: 118). In some embodiments, the insertion comprises a polypeptide comprising a fragment of at least 5 amino acids of FHNEGKY (SEQ ID NO: 118). In some embodiments, the insertion comprises a polypeptide comprising a fragment of at least 6 amino acids of FHNEGKY (SEQ ID NO: 118).

In some embodiments, the nucleic acid molecule encodes a capsid polypeptide that comprises a mutation that corresponds to an insertion between residues 591 and 592 as compared to SEQ ID NO: 1, wherein the insertion comprises an amino acid G.

In some embodiments, the nucleic acid molecule encodes a capsid polypeptide that comprises a mutation that corresponds to an insertion between residues 588 and 589 as compared to SEQ ID NO: 1, wherein the insertion comprises, e.g., consists of, a polypeptide of QPWEPDK (SEQ ID NO: 119). In some embodiments, the insertion comprises a polypeptide that has at least 57.1%, 71.4%, 85.7%, or 100% identity to QPWEPDK (SEQ ID NO: 119). In some embodiments, the insertion comprises a polypeptide that has at least 1, 2, or 3 mutations as compared to QPWEPDK (SEQ ID NO: 119). In some embodiments, the insertion comprises a polypeptide comprising a fragment of at least 4 amino acids of QPWEPDK (SEQ ID NO: 119). In some embodiments, the insertion comprises a polypeptide comprising a fragment of at least 5 amino acids of QPWEPDK (SEQ ID NO: 119). In some embodiments, the insertion comprises a polypeptide comprising a fragment of at least 6 amino acids of QPWEPDK (SEQ ID NO: 119).

In some embodiments, the nucleic acid molecule encodes a capsid polypeptide that comprises a mutation that corresponds to an insertion between residues 592 and 593 as compared to SEQ ID NO: 1, wherein the insertion comprises, e.g., consists of, a polypeptide of ALALSTTN (SEQ ID NO: 120). In some embodiments, the insertion comprises a polypeptide that has at least 50%, 62.5%, 75%, 87.5%, or 100% identity to ALALSTTN (SEQ ID NO: 120). In some embodiments, the insertion comprises a polypeptide that has at least 1, 2, 3, or 4 mutations as compared to ALALSTTN (SEQ ID NO: 120). In some embodiments, the insertion comprises a polypeptide comprising a fragment of at least 4 amino acids of ALALSTTN (SEQ ID NO: 120). In some embodiments, the insertion comprises a polypeptide comprising a fragment of at least 5 amino acids of ALALSTTN (SEQ ID NO: 120). In some embodiments, the insertion comprises a polypeptide comprising a fragment of at least 6 amino acids of ALALSTTN (SEQ ID NO: 120). In some embodiments, the insertion comprises a polypeptide comprising a fragment of at least 7 amino acids of ALALSTTN (SEQ ID NO: 120). In some embodiments, the insertion comprises, e.g., consists of, ALSTTN (SEQ ID NO: 121).

In some embodiments, the nucleic acid molecule encodes a capsid polypeptide that comprises a mutation that corresponds to an insertion between residues 585 and 586 as compared to SEQ ID NO: 1, wherein the insertion comprises, e.g., consists of, a polypeptide of PWGTAG (SEQ ID NO: 122). In some embodiments, the insertion comprises a polypeptide that has at least 50%, 66.7%, 83.3%, or 100% identity to PWGTAG (SEQ ID NO: 122). In some embodiments, the insertion comprises a polypeptide that has at least 1, 2, or 3 mutations as compared to PWGTAG (SEQ ID NO: 122). In some embodiments, the insertion comprises a polypeptide comprising a fragment of at least 4 amino acids of PWGTAG (SEQ ID NO: 122). In some embodiments, the insertion comprises a polypeptide comprising a fragment of at least 5 amino acids of PWGTAG (SEQ ID NO: 122).

In some embodiments, the nucleic acid molecule encodes a capsid polypeptide as provided herein. In some embodiments, the nucleic acid molecule encodes a capsid polypeptide that is at least 70%, 75%, 80%, 85%, 90%, 91%, 92%, 93%, 94%, 95%, 96%, 97%, 98%, 99%, or 100% identical to a capsid polypeptide as provided herein.

In some embodiments, a capsid polypeptide is provided that comprises a capsid polypeptide that is at least 70%, 75%, 80%, 85%, 90%, 91%, 92%, 93%, 94%, 95%, 96%, 97%, 98%, 99%, or 100% identical to a capsid polypeptide as provided herein.

In some embodiments, the capsid polypeptide, or the reference polypeptide for purposes of % identity, comprises a sequence of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46. In some embodiments, the capsid polypeptide, or the reference polypeptide for purposes of % identity, comprises a sequence of SEQ ID NO: 2. In some embodiments, the capsid polypeptide, or the reference polypeptide for purposes of % identity, comprises a sequence of SEQ ID NO: 3. In some embodiments, the capsid polypeptide, or the reference polypeptide for purposes of % identity, comprises a sequence of SEQ ID NO: 4. In some embodiments, the capsid polypeptide, or the reference polypeptide for purposes of % identity, comprises a sequence of SEQ ID NO: 5. In some embodiments, the capsid polypeptide, or the reference polypeptide for purposes of % identity, comprises a sequence of SEQ ID NO: 6. In some embodiments, the capsid polypeptide, or the reference polypeptide for purposes of % identity, comprises a sequence of SEQ ID NO: 7. In some embodiments, the capsid polypeptide, or the reference polypeptide for purposes of % identity, comprises a sequence of SEQ ID NO: 8. In some embodiments, the capsid polypeptide, or the reference polypeptide for purposes of % identity, comprises a sequence of SEQ ID NO: 9. In some embodiments, the capsid polypeptide, or the reference polypeptide for purposes of % identity, comprises a sequence of SEQ ID NO: 10. In some embodiments, the capsid polypeptide, or the reference polypeptide for purposes of % identity, comprises a sequence of SEQ ID NO: 11. In some embodiments, the capsid polypeptide, or the reference polypeptide for purposes of % identity, comprises a sequence of SEQ ID NO: 12. In some embodiments, the capsid polypeptide, or the reference polypeptide for purposes of % identity, comprises a sequence of SEQ ID NO: 13. In some embodiments, the capsid polypeptide, or the reference polypeptide for purposes of % identity, comprises a sequence of SEQ ID NO: 14. In some embodiments, the capsid polypeptide, or the reference polypeptide for purposes of % identity, comprises a sequence of SEQ ID NO: 15. In some embodiments, the capsid polypeptide, or the reference polypeptide for purposes of % identity, comprises a sequence of SEQ ID NO: 16. In some embodiments, the capsid polypeptide, or the reference polypeptide for purposes of % identity, comprises a sequence of SEQ ID NO: 17. In some embodiments, the capsid polypeptide, or the reference polypeptide for purposes of % identity, comprises a sequence of SEQ ID NO: 18. In some embodiments, the capsid polypeptide, or the reference polypeptide for purposes of % identity, comprises a sequence of SEQ ID NO: 19. In some embodiments, the capsid polypeptide, or the reference polypeptide for purposes of % identity, comprises a sequence of SEQ ID NO: 20. In some embodiments, the capsid polypeptide, or the reference polypeptide for purposes of % identity, comprises a sequence of SEQ ID NO: 21. In some embodiments, the capsid polypeptide, or the reference polypeptide for purposes of % identity, comprises a sequence of SEQ ID NO: 22. In some embodiments, the capsid polypeptide, or the reference polypeptide for purposes of % identity, comprises a sequence of SEQ ID NO: 23. In some embodiments, the capsid polypeptide, or the reference polypeptide for purposes of % identity, comprises a sequence of SEQ ID NO: 24. In some embodiments, the capsid polypeptide, or the reference polypeptide for purposes of % identity, comprises a sequence of SEQ ID NO: 25. In some embodiments, the capsid polypeptide, or the reference polypeptide for purposes of % identity, comprises a sequence of SEQ ID NO: 26. In some embodiments, the capsid polypeptide, or the reference polypeptide for purposes of % identity, comprises a sequence of SEQ ID NO: 27. In some embodiments, the capsid polypeptide, or the reference polypeptide for purposes of % identity, comprises a sequence of SEQ ID NO: 28. In some embodiments, the capsid polypeptide, or the reference polypeptide for purposes of % identity, comprises a sequence of SEQ ID NO: 29. In some embodiments, the capsid polypeptide, or the reference polypeptide for purposes of % identity, comprises a sequence of SEQ ID NO: 30. In some embodiments, the capsid polypeptide, or the reference polypeptide for purposes of % identity, comprises a sequence of SEQ ID NO: 31. In some embodiments, the capsid polypeptide, or the reference polypeptide for purposes of % identity, comprises a sequence of SEQ ID NO: 32. In some embodiments, the capsid polypeptide, or the reference polypeptide for purposes of % identity, comprises a sequence of SEQ ID NO: 33. In some embodiments, the capsid polypeptide, or the reference polypeptide for purposes of % identity, comprises a sequence of SEQ ID NO: 34. In some embodiments, the capsid polypeptide, or the reference polypeptide for purposes of % identity, comprises a sequence of SEQ ID NO: 35. In some embodiments, the capsid polypeptide, or the reference polypeptide for purposes of % identity, comprises a sequence of SEQ ID NO: 36. In some embodiments, the capsid polypeptide, or the reference polypeptide for purposes of % identity, comprises a sequence of SEQ ID NO: 37. In some embodiments, the capsid polypeptide, or the reference polypeptide for purposes of % identity, comprises a sequence of SEQ ID NO: 38. In some embodiments, the capsid polypeptide, or the reference polypeptide for purposes of % identity, comprises a sequence of SEQ ID NO: 39. In some embodiments, the capsid polypeptide, or the reference polypeptide for purposes of % identity, comprises a sequence of SEQ ID NO: 40. In some embodiments, the capsid polypeptide, or the reference polypeptide for purposes of % identity, comprises a sequence of SEQ ID NO: 41. In some embodiments, the capsid polypeptide, or the reference polypeptide for purposes of % identity, comprises a sequence of SEQ ID NO: 42. In some embodiments, the capsid polypeptide, or the reference polypeptide for purposes of % identity, comprises a sequence of SEQ ID NO: 43. In some embodiments, the capsid polypeptide, or the reference polypeptide for purposes of % identity, comprises a sequence of SEQ ID NO: 44. In some embodiments, the capsid polypeptide, or the reference polypeptide for purposes of % identity, comprises a sequence of SEQ ID NO: 45. In some embodiments, the capsid polypeptide, or the reference polypeptide for purposes of % identity, comprises a sequence of SEQ ID NO: 46.

In some embodiments, the capsid polypeptide, or the reference polypeptide for purposes of % identity, comprises a nucleotide sequence of SEQ ID NOs: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or 91. In some embodiments, the capsid polypeptide, or the reference polypeptide for purposes of % identity, comprises a nucleotide sequence of SEQ ID NO: 47. In some embodiments, the capsid polypeptide, or the reference polypeptide for purposes of % identity, comprises a nucleotide sequence of SEQ ID NO: 48. In some embodiments, the capsid polypeptide, or the reference polypeptide for purposes of % identity, comprises a nucleotide sequence of SEQ ID NO: 49. In some embodiments, the capsid polypeptide, or the reference polypeptide for purposes of % identity, comprises a nucleotide sequence of SEQ ID NO: 50. In some embodiments, the capsid polypeptide, or the reference polypeptide for purposes of % identity, comprises a nucleotide sequence of SEQ ID NO: 51. In some embodiments, the capsid polypeptide, or the reference polypeptide for purposes of % identity, comprises a nucleotide sequence of SEQ ID NO: 52. In some embodiments, the capsid polypeptide, or the reference polypeptide for purposes of % identity, comprises a nucleotide sequence of SEQ ID NO: 53. In some embodiments, the capsid polypeptide, or the reference polypeptide for purposes of % identity, comprises a nucleotide sequence of SEQ ID NO: 54. In some embodiments, the capsid polypeptide, or the reference polypeptide for purposes of % identity, comprises a nucleotide sequence of SEQ ID NO: 55. In some embodiments, the capsid polypeptide, or the reference polypeptide for purposes of % identity, comprises a nucleotide sequence of SEQ ID NO: 56. In some embodiments, the capsid polypeptide, or the reference polypeptide for purposes of % identity, comprises a nucleotide sequence of SEQ ID NO: 57. In some embodiments, the capsid polypeptide, or the reference polypeptide for purposes of % identity, comprises a nucleotide sequence of SEQ ID NO: 58. In some embodiments, the capsid polypeptide, or the reference polypeptide for purposes of % identity, comprises a nucleotide sequence of SEQ ID NO: 59. In some embodiments, the capsid polypeptide, or the reference polypeptide for purposes of % identity, comprises a nucleotide sequence of SEQ ID NO: 60. In some embodiments, the capsid polypeptide, or the reference polypeptide for purposes of % identity, comprises a nucleotide sequence of SEQ ID NO: 61. In some embodiments, the capsid polypeptide, or the reference polypeptide for purposes of % identity, comprises a nucleotide sequence of SEQ ID NO: 62. In some embodiments, the capsid polypeptide, or the reference polypeptide for purposes of % identity, comprises a nucleotide sequence of SEQ ID NO: 63. In some embodiments, the capsid polypeptide, or the reference polypeptide for purposes of % identity, comprises a nucleotide sequence of SEQ ID NO: 64. In some embodiments, the capsid polypeptide, or the reference polypeptide for purposes of % identity, comprises a nucleotide sequence of SEQ ID NO: 65. In some embodiments, the capsid polypeptide, or the reference polypeptide for purposes of % identity, comprises a nucleotide sequence of SEQ ID NO: 66. In some embodiments, the capsid polypeptide, or the reference polypeptide for purposes of % identity, comprises a nucleotide sequence of SEQ ID NO: 67. In some embodiments, the capsid polypeptide, or the reference polypeptide for purposes of % identity, comprises a nucleotide sequence of SEQ ID NO: 68. In some embodiments, the capsid polypeptide, or the reference polypeptide for purposes of % identity, comprises a nucleotide sequence of SEQ ID NO: 69. In some embodiments, the capsid polypeptide, or the reference polypeptide for purposes of % identity, comprises a nucleotide sequence of SEQ ID NO: 70. In some embodiments, the capsid polypeptide, or the reference polypeptide for purposes of % identity, comprises a nucleotide sequence of SEQ ID NO: 71. In some embodiments, the capsid polypeptide, or the reference polypeptide for purposes of % identity, comprises a nucleotide sequence of SEQ ID NO: 72. In some embodiments, the capsid polypeptide, or the reference polypeptide for purposes of % identity, comprises a nucleotide sequence of SEQ ID NO: 73. In some embodiments, the capsid polypeptide, or the reference polypeptide for purposes of % identity, comprises a nucleotide sequence of SEQ ID NO: 74. In some embodiments, the capsid polypeptide, or the reference polypeptide for purposes of % identity, comprises a nucleotide sequence of SEQ ID NO: 75. In some embodiments, the capsid polypeptide, or the reference polypeptide for purposes of % identity, comprises a nucleotide sequence of SEQ ID NO: 76. In some embodiments, the capsid polypeptide, or the reference polypeptide for purposes of % identity, comprises a nucleotide sequence of SEQ ID NO: 77. In some embodiments, the capsid polypeptide, or the reference polypeptide for purposes of % identity, comprises a nucleotide sequence of SEQ ID NO: 78. In some embodiments, the capsid polypeptide, or the reference polypeptide for purposes of % identity, comprises a nucleotide sequence of SEQ ID NO: 79. In some embodiments, the capsid polypeptide, or the reference polypeptide for purposes of % identity, comprises a nucleotide sequence of SEQ ID NO: 80. In some embodiments, the capsid polypeptide, or the reference polypeptide for purposes of % identity, comprises a nucleotide sequence of SEQ ID NO: 81. In some embodiments, the capsid polypeptide, or the reference polypeptide for purposes of % identity, comprises a nucleotide sequence of SEQ ID NO: 82. In some embodiments, the capsid polypeptide, or the reference polypeptide for purposes of % identity, comprises a nucleotide sequence of SEQ ID NO: 83. In some embodiments, the capsid polypeptide, or the reference polypeptide for purposes of % identity, comprises a nucleotide sequence of SEQ ID NO: 84. In some embodiments, the capsid polypeptide, or the reference polypeptide for purposes of % identity, comprises a nucleotide sequence of SEQ ID NO: 85. In some embodiments, the capsid polypeptide, or the reference polypeptide for purposes of % identity, comprises a nucleotide sequence of SEQ ID NO: 86. In some embodiments, the capsid polypeptide, or the reference polypeptide for purposes of % identity, comprises a nucleotide sequence of SEQ ID NO: 87. In some embodiments, the capsid polypeptide, or the reference polypeptide for purposes of % identity, comprises a nucleotide sequence of SEQ ID NO: 88. In some embodiments, the capsid polypeptide, or the reference polypeptide for purposes of % identity, comprises a nucleotide sequence of SEQ ID NO: 89. In some embodiments, the capsid polypeptide, or the reference polypeptide for purposes of % identity, comprises a nucleotide sequence of SEQ ID NO: 90. In some embodiments, the capsid polypeptide, or the reference polypeptide for purposes of % identity, comprises a nucleotide sequence of SEQ ID NO: 91.

In some embodiments, the capsid polypeptide, or the reference polypeptide for purposes of % identity, comprises a nucleotide sequence of SEQ ID NOs: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or 91, that encodes a sequence of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46. In some embodiments, the capsid polypeptide, or the reference polypeptide for purposes of % identity, comprises a nucleotide sequence of SEQ ID NO: 47 that encodes a sequence of SEQ ID NO: 2. In some embodiments, the capsid polypeptide, or the reference polypeptide for purposes of % identity, comprises a nucleotide sequence of SEQ ID NO: 48 that encodes a sequence of SEQ ID NO: 3. In some embodiments, the capsid polypeptide, or the reference polypeptide for purposes of % identity, comprises a nucleotide sequence of SEQ ID NO: 49 that encodes a sequence of SEQ ID NO: 4. In some embodiments, the capsid polypeptide, or the reference polypeptide for purposes of % identity, comprises a nucleotide sequence of SEQ ID NO: 50 that encodes a sequence of SEQ ID NO: 5. In some embodiments, the capsid polypeptide, or the reference polypeptide for purposes of % identity, comprises a nucleotide sequence of SEQ ID NO: 51 that encodes a sequence of SEQ ID NO: 6. In some embodiments, the capsid polypeptide, or the reference polypeptide for purposes of % identity, comprises a nucleotide sequence of SEQ ID NO: 52 that encodes a sequence of SEQ ID NO: 7. In some embodiments, the capsid polypeptide, or the reference polypeptide for purposes of % identity, comprises a nucleotide sequence of SEQ ID NO: 53 that encodes a sequence of SEQ ID NO: 8. In some embodiments, the capsid polypeptide, or the reference polypeptide for purposes of % identity, comprises a nucleotide sequence of SEQ ID NO: 54 that encodes a sequence of SEQ ID NO: 9. In some embodiments, the capsid polypeptide, or the reference polypeptide for purposes of % identity, comprises a nucleotide sequence of SEQ ID NO: 55 that encodes a sequence of SEQ ID NO: 10. In some embodiments, the capsid polypeptide, or the reference polypeptide for purposes of % identity, comprises a nucleotide sequence of SEQ ID NO: 56 that encodes a sequence of SEQ ID NO: 11. In some embodiments, the capsid polypeptide, or the reference polypeptide for purposes of % identity, comprises a nucleotide sequence of SEQ ID NO: 57 that encodes a sequence of SEQ ID NO: 12. In some embodiments, the capsid polypeptide, or the reference polypeptide for purposes of % identity, comprises a nucleotide sequence of SEQ ID NO: 58 that encodes a sequence of SEQ ID NO: 13. In some embodiments, the capsid polypeptide, or the reference polypeptide for purposes of % identity, comprises a nucleotide sequence of SEQ ID NO: 59 that encodes a sequence of SEQ ID NO: 14. In some embodiments, the capsid polypeptide, or the reference polypeptide for purposes of % identity, comprises a nucleotide sequence of SEQ ID NO: 60 that encodes a sequence of SEQ ID NO: 15. In some embodiments, the capsid polypeptide, or the reference polypeptide for purposes of % identity, comprises a nucleotide sequence of SEQ ID NO: 61 that encodes a sequence of SEQ ID NO: 16. In some embodiments, the capsid polypeptide, or the reference polypeptide for purposes of % identity, comprises a nucleotide sequence of SEQ ID NO: 62 that encodes a sequence of SEQ ID NO: 17. In some embodiments, the capsid polypeptide, or the reference polypeptide for purposes of % identity, comprises a nucleotide sequence of SEQ ID NO: 63 that encodes a sequence of SEQ ID NO: 18. In some embodiments, the capsid polypeptide, or the reference polypeptide for purposes of % identity, comprises a nucleotide sequence of SEQ ID NO: 64 that encodes a sequence of SEQ ID NO: 19. In some embodiments, the capsid polypeptide, or the reference polypeptide for purposes of % identity, comprises a nucleotide sequence of SEQ ID NO: 65 that encodes a sequence of SEQ ID NO: 20. In some embodiments, the capsid polypeptide, or the reference polypeptide for purposes of % identity, comprises a nucleotide sequence of SEQ ID NO: 66 that encodes a sequence of SEQ ID NO: 21. In some embodiments, the capsid polypeptide, or the reference polypeptide for purposes of % identity, comprises a nucleotide sequence of SEQ ID NO: 67 that encodes a sequence of SEQ ID NO: 22. In some embodiments, the capsid polypeptide, or the reference polypeptide for purposes of % identity, comprises a nucleotide sequence of SEQ ID NO: 68 that encodes a sequence of SEQ ID NO: 23. In some embodiments, the capsid polypeptide, or the reference polypeptide for purposes of % identity, comprises a nucleotide sequence of SEQ ID NO: 69 that encodes a sequence of SEQ ID NO: 24. In some embodiments, the capsid polypeptide, or the reference polypeptide for purposes of % identity, comprises a nucleotide sequence of SEQ ID NO: 70 that encodes a sequence of SEQ ID NO: 25. In some embodiments, the capsid polypeptide, or the reference polypeptide for purposes of % identity, comprises a nucleotide sequence of SEQ ID NO: 71 that encodes a sequence of SEQ ID NO: 26. In some embodiments, the capsid polypeptide, or the reference polypeptide for purposes of % identity, comprises a nucleotide sequence of SEQ ID NO: 72 that encodes a sequence of SEQ ID NO: 27. In some embodiments, the capsid polypeptide, or the reference polypeptide for purposes of % identity, comprises a nucleotide sequence of SEQ ID NO: 73 that encodes a sequence of SEQ ID NO: 28. In some embodiments, the capsid polypeptide, or the reference polypeptide for purposes of % identity, comprises a nucleotide sequence of SEQ ID NO: 74 that encodes a sequence of SEQ ID NO: 29. In some embodiments, the capsid polypeptide, or the reference polypeptide for purposes of % identity, comprises a nucleotide sequence of SEQ ID NO: 75 that encodes a sequence of SEQ ID NO: 30. In some embodiments, the capsid polypeptide, or the reference polypeptide for purposes of % identity, comprises a nucleotide sequence of SEQ ID NO: 76 that encodes a sequence of SEQ ID NO: 31. In some embodiments, the capsid polypeptide, or the reference polypeptide for purposes of % identity, comprises a nucleotide sequence of SEQ ID NO: 77 that encodes a sequence of SEQ ID NO: 32. In some embodiments, the capsid polypeptide, or the reference polypeptide for purposes of % identity, comprises a nucleotide sequence of SEQ ID NO: 78 that encodes a sequence of SEQ ID NO: 33. In some embodiments, the capsid polypeptide, or the reference polypeptide for purposes of % identity, comprises a nucleotide sequence of SEQ ID NO: 79 that encodes a sequence of SEQ ID NO: 34. In some embodiments, the capsid polypeptide, or the reference polypeptide for purposes of % identity, comprises a nucleotide sequence of SEQ ID NO: 80 that encodes a sequence of SEQ ID NO: 35. In some embodiments, the capsid polypeptide, or the reference polypeptide for purposes of % identity, comprises a nucleotide sequence of SEQ ID NO: 81 that encodes a sequence of SEQ ID NO: 36. In some embodiments, the capsid polypeptide, or the reference polypeptide for purposes of % identity, comprises a nucleotide sequence of SEQ ID NO: 82 that encodes a sequence of SEQ ID NO: 37. In some embodiments, the capsid polypeptide, or the reference polypeptide for purposes of % identity, comprises a nucleotide sequence of SEQ ID NO: 83 that encodes a sequence of SEQ ID NO: 38. In some embodiments, the capsid polypeptide, or the reference polypeptide for purposes of % identity, comprises a nucleotide sequence of SEQ ID NO: 84 that encodes a sequence of SEQ ID NO: 39. In some embodiments, the capsid polypeptide, or the reference polypeptide for purposes of % identity, comprises a nucleotide sequence of SEQ ID NO: 85 that encodes a sequence of SEQ ID NO: 40. In some embodiments, the capsid polypeptide, or the reference polypeptide for purposes of % identity, comprises a nucleotide sequence of SEQ ID NO: 86 that encodes a sequence of SEQ ID NO: 41. In some embodiments, the capsid polypeptide, or the reference polypeptide for purposes of % identity, comprises a nucleotide sequence of SEQ ID NO: 87 that encodes a sequence of SEQ ID NO: 42. In some embodiments, the capsid polypeptide, or the reference polypeptide for purposes of % identity, comprises a nucleotide sequence of SEQ ID NO: 88 that encodes a sequence of SEQ ID NO: 43. In some embodiments, the capsid polypeptide, or the reference polypeptide for purposes of % identity, comprises a nucleotide sequence of SEQ ID NO: 89 that encodes a sequence of SEQ ID NO: 44. In some embodiments, the capsid polypeptide, or the reference polypeptide for purposes of % identity, comprises a nucleotide sequence of SEQ ID NO: 90 that encodes a sequence of SEQ ID NO: 45. In some embodiments, the capsid polypeptide, or the reference polypeptide for purposes of % identity, comprises a nucleotide sequence of SEQ ID NO: 91 that encodes a sequence of SEQ ID NO: 46.

In some embodiments, the capsid polypeptide, or the reference polypeptide for purposes of % identity, comprises a sequence of SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 that is encoded by a nucleotide sequence of SEQ ID NOs: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or 91. In some embodiments, the capsid polypeptide, or the reference polypeptide for purposes of % identity, comprises a sequence of SEQ ID NO: 2 that is encoded by a nucleotide sequence of SEQ ID NO: 47. In some embodiments, the capsid polypeptide, or the reference polypeptide for purposes of % identity, comprises a sequence of SEQ ID NO: 3 that is encoded by a nucleotide sequence of SEQ ID NO: 48. In some embodiments, the capsid polypeptide, or the reference polypeptide for purposes of % identity, comprises a sequence of SEQ ID NO: 4 that is encoded by a nucleotide sequence of SEQ ID NO: 49. In some embodiments, the capsid polypeptide, or the reference polypeptide for purposes of % identity, comprises a sequence of SEQ ID NO: 5 that is encoded by a nucleotide sequence of SEQ ID NO: 50. In some embodiments, the capsid polypeptide, or the reference polypeptide for purposes of % identity, comprises a sequence of SEQ ID NO: 6 that is encoded by a nucleotide sequence of SEQ ID NO: 51. In some embodiments, the capsid polypeptide, or the reference polypeptide for purposes of % identity, comprises a sequence of SEQ ID NO: 7 that is encoded by a nucleotide sequence of SEQ ID NO: 52. In some embodiments, the capsid polypeptide, or the reference polypeptide for purposes of % identity, comprises a sequence of SEQ ID NO: 8 that is encoded by a nucleotide sequence of SEQ ID NO: 53. In some embodiments, the capsid polypeptide, or the reference polypeptide for purposes of % identity, comprises a sequence of SEQ ID NO: 9 that is encoded by a nucleotide sequence of SEQ ID NO: 54. In some embodiments, the capsid polypeptide, or the reference polypeptide for purposes of % identity, comprises a sequence of SEQ ID NO: 10 that is encoded by a nucleotide sequence of SEQ ID NO: 55. In some embodiments, the capsid polypeptide, or the reference polypeptide for purposes of % identity, comprises a sequence of SEQ ID NO: 11 that is encoded by a nucleotide sequence of SEQ ID NO: 56. In some embodiments, the capsid polypeptide, or the reference polypeptide for purposes of % identity, comprises a sequence of SEQ ID NO: 12 that is encoded by a nucleotide sequence of SEQ ID NO: 57. In some embodiments, the capsid polypeptide, or the reference polypeptide for purposes of % identity, comprises a sequence of SEQ ID NO: 13 that is encoded by a nucleotide sequence of SEQ ID NO: 58. In some embodiments, the capsid polypeptide, or the reference polypeptide for purposes of % identity, comprises a sequence of SEQ ID NO: 14 that is encoded by a nucleotide sequence of SEQ ID NO: 59. In some embodiments, the capsid polypeptide, or the reference polypeptide for purposes of % identity, comprises a sequence of SEQ ID NO: 15 that is encoded by a nucleotide sequence of SEQ ID NO: 60. In some embodiments, the capsid polypeptide, or the reference polypeptide for purposes of % identity, comprises a sequence of SEQ ID NO: 16 that is encoded by a nucleotide sequence of SEQ ID NO: 61. In some embodiments, the capsid polypeptide, or the reference polypeptide for purposes of % identity, comprises a sequence of SEQ ID NO: 17 that is encoded by a nucleotide sequence of SEQ ID NO: 62. In some embodiments, the capsid polypeptide, or the reference polypeptide for purposes of % identity, comprises a sequence of SEQ ID NO: 18 that is encoded by a nucleotide sequence of SEQ ID NO: 63. In some embodiments, the capsid polypeptide, or the reference polypeptide for purposes of % identity, comprises a sequence of SEQ ID NO: 19 that is encoded by a nucleotide sequence of SEQ ID NO: 64. In some embodiments, the capsid polypeptide, or the reference polypeptide for purposes of % identity, comprises a sequence of SEQ ID NO: 20 that is encoded by a nucleotide sequence of SEQ ID NO: 65. In some embodiments, the capsid polypeptide, or the reference polypeptide for purposes of % identity, comprises a sequence of SEQ ID NO: 21 that is encoded by a nucleotide sequence of SEQ ID NO: 66. In some embodiments, the capsid polypeptide, or the reference polypeptide for purposes of % identity, comprises a sequence of SEQ ID NO: 22 that is encoded by a nucleotide sequence of SEQ ID NO: 67. In some embodiments, the capsid polypeptide, or the reference polypeptide for purposes of % identity, comprises a sequence of SEQ ID NO: 23 that is encoded by a nucleotide sequence of SEQ ID NO: 68. In some embodiments, the capsid polypeptide, or the reference polypeptide for purposes of % identity, comprises a sequence of SEQ ID NO: 24 that is encoded by a nucleotide sequence of SEQ ID NO: 69. In some embodiments, the capsid polypeptide, or the reference polypeptide for purposes of % identity, comprises a sequence of SEQ ID NO: 25 that is encoded by a nucleotide sequence of SEQ ID NO: 70. In some embodiments, the capsid polypeptide, or the reference polypeptide for purposes of % identity, comprises a sequence of SEQ ID NO: 26 that is encoded by a nucleotide sequence of SEQ ID NO: 71. In some embodiments, the capsid polypeptide, or the reference polypeptide for purposes of % identity, comprises a sequence of SEQ ID NO: 27 that is encoded by a nucleotide sequence of SEQ ID NO: 72. In some embodiments, the capsid polypeptide, or the reference polypeptide for purposes of % identity, comprises a sequence of SEQ ID NO: 28 that is encoded by a nucleotide sequence of SEQ ID NO: 73. In some embodiments, the capsid polypeptide, or the reference polypeptide for purposes of % identity, comprises a sequence of SEQ ID NO: 29 that is encoded by a nucleotide sequence of SEQ ID NO: 74. In some embodiments, the capsid polypeptide, or the reference polypeptide for purposes of % identity, comprises a sequence of SEQ ID NO: 30 that is encoded by a nucleotide sequence of SEQ ID NO: 75. In some embodiments, the capsid polypeptide, or the reference polypeptide for purposes of % identity, comprises a sequence of SEQ ID NO: 31 that is encoded by a nucleotide sequence of SEQ ID NO: 76. In some embodiments, the capsid polypeptide, or the reference polypeptide for purposes of % identity, comprises a sequence of SEQ ID NO: 32 that is encoded by a nucleotide sequence of SEQ ID NO: 77. In some embodiments, the capsid polypeptide, or the reference polypeptide for purposes of % identity, comprises a sequence of SEQ ID NO: 33 that is encoded by a nucleotide sequence of SEQ ID NO: 78. In some embodiments, the capsid polypeptide, or the reference polypeptide for purposes of % identity, comprises a sequence of SEQ ID NO: 34 that is encoded by a nucleotide sequence of SEQ ID NO: 79.

In some embodiments, the capsid polypeptide, or the reference polypeptide for purposes of % identity, comprises a sequence of SEQ ID NO: 33 that is encoded by a nucleotide sequence of SEQ ID NO: 78. In some embodiments, the capsid polypeptide, or the reference polypeptide for purposes of % identity, comprises a sequence of SEQ ID NO: 34 that is encoded by a nucleotide sequence of SEQ ID NO: 79. In some embodiments, the capsid polypeptide, or the reference polypeptide for purposes of % identity, comprises a sequence of SEQ ID NO: 35 that is encoded by a nucleotide sequence of SEQ ID NO: 80. In some embodiments, the capsid polypeptide, or the reference polypeptide for purposes of % identity, comprises a sequence of SEQ ID NO: 36 that is encoded by a nucleotide sequence of SEQ ID NO: 81. In some embodiments, the capsid polypeptide, or the reference polypeptide for purposes of % identity, comprises a sequence of SEQ ID NO: 37 that is encoded by a nucleotide sequence of SEQ ID NO: 82. In some embodiments, the capsid polypeptide, or the reference polypeptide for purposes of % identity, comprises a sequence of SEQ ID NO: 38 that is encoded by a nucleotide sequence of SEQ ID NO: 83. In some embodiments, the capsid polypeptide, or the reference polypeptide for purposes of % identity, comprises a sequence of SEQ ID NO: 39 that is encoded by a nucleotide sequence of SEQ ID NO: 84. In some embodiments, the capsid polypeptide, or the reference polypeptide for purposes of % identity, comprises a sequence of SEQ ID NO: 40 that is encoded by a nucleotide sequence of SEQ ID NO: 85. In some embodiments, the capsid polypeptide, or the reference polypeptide for purposes of % identity, comprises a sequence of SEQ ID NO: 41 that is encoded by a nucleotide sequence of SEQ ID NO: 86. In some embodiments, the capsid polypeptide, or the reference polypeptide for purposes of % identity, comprises a sequence of SEQ ID NO: 42 that is encoded by a nucleotide sequence of SEQ ID NO: 87. In some embodiments, the capsid polypeptide, or the reference polypeptide for purposes of % identity, comprises a sequence of SEQ ID NO: 43 that is encoded by a nucleotide sequence of SEQ ID NO: 88. In some embodiments, the capsid polypeptide, or the reference polypeptide for purposes of % identity, comprises a sequence of SEQ ID NO: 44 that is encoded by a nucleotide sequence of SEQ ID NO: 89. In some embodiments, the capsid polypeptide, or the reference polypeptide for purposes of % identity, comprises a sequence of SEQ ID NO: 45 that is encoded by a nucleotide sequence of SEQ ID NO: 90. In some embodiments, the capsid polypeptide, or the reference polypeptide for purposes of % identity, comprises a sequence of SEQ ID NO: 46 that is encoded by a nucleotide sequence of SEQ ID NO: 91.

In some embodiments, the capsid polypeptide comprises a sequence that includes all of the mutation differences associated with any one of VAR-1 through VAR-45 (e.g., as indicated in Table 1, column 7), and further includes no more than 30, no more than 20, no more than 10, no more than 9, no more than 8, no more than 7, no more than 6, no more than 5, no more than 4, no more than 3, no more than 2 or no more than 1 additional mutations relative to SEQ ID NO: 1.

In some embodiments, the capsid polypeptide is a VP1 capsid polypeptide. In embodiments, the capsid polypeptide is a VP2 capsid polypeptide. In embodiments, the capsid polypeptide is a VP3 capsid polypeptide. With respect to reference sequence SEQ ID NO: 1, a VP1 capsid polypeptide comprises amino acids 1-724 of SEQ ID NO: 1. With respect to reference sequence SEQ ID NO: 1, a VP2 capsid polypeptide comprises amino acids 138-724 of SEQ ID NO: 1. With respect to reference sequence SEQ ID NO: 1, a VP3 capsid polypeptide comprises amino acids 203-724 of SEQ ID NO: 1.

Table 1 lists information regarding exemplary variant dependoparvovirus particles comprising nucleic acids comprising the variant capsid regarding the ocular transduction properties and production characteristics of said non-limiting exemplary variants. Exemplary sequences of capsid polypeptides and nucleic acid molecules encoding the same are provided in Table 2, Table 3, and Table 4.

Table 1. Transduction and virus production of exemplary variant dependoparvovirus (e.g., AVV) particles comprising variant capsid polypeptides. Macular and retinal transduction are as measured following IVT injection; trabecular transduction is as measured following IC injection. Substitutions are notated as n###N where “N” is the final amino acid, “n” is the reference amino acid and “###” is the reference amino acid position of SEQ ID NO:1; deletions are notated as n###- where “-” indicates the deletion of “n” at position “###” of the reference sequence SEQ ID NO: 1; insertions are notated as ###_Naa_###_(n)y, where “###” are the amino acid positions in the reference sequence SEQ ID NO: 1 between which the insertion occurs, “Naa” refers to the length of the insertion (having “N” amino acids) and “(n)y” providing the sequence of the insertion). Each individual Mutation Difference (e.g., within a row, each mutation in quotations (‘ ’) in column 7) and combinations of such individual mutation differences is sometimes referred to herein as a “mutation associated with VAR-X”, where VAR-X is the variant identifier listed in the “Name column.”

TABLE 1

| | | Macular | Retinal | Trabecular | Virus | |
|---|---|---|---|---|---|---|
| | SEQ | Transduction | Transduction | Transduction | Production | |
| | ID NO: | as compared | as compared | as compared | as compared | |
| | of VP1 | to wild-type | to wild-type | to wild-type | to wild-type | Mutation Differences as |
| | capsid | SEQ ID NO: 1 | SEQ ID NO: 1 | SEQ ID NO: 1 | SEQ ID NO: 1 | compared to SEQ ID NO: 1 |
| Name | polypeptide | (Log2) | (Log2) | (Log2) | (Log2) | (Collectively the “Mutation Set”) |
| VAR-1 | 2 | −0.623109184 | −3.703538304 | 1.725067698 | −0.970942107 | [‘T14L’, ‘L15V’, ‘I19A’, ‘K24H’] |
| VAR-2 | 3 | −0.048551556 | −2.614465707 | 2.659319064 | −3.169014336 | [‘A3V’, ‘Y6F’, ‘L11F’, ‘E12Q’, |
| | | | | | | ‘Q21L’, ‘W23I’, ‘L25C’, ‘P29A’, |
| | | | | | | ‘P31N’, ‘K33R’] |
| VAR-3 | 4 | 5.790478596 | 3.036979883 | 0.395414334 | −1.00780873 | [‘N449Q’, ‘T450M’, ‘P451T’, ‘T455L’, |
| | | | | | | ‘T456G’, ‘Q457T’, ‘S458Q’, ‘R459M’] |
| VAR-4 | 5 | 5.614983257 | 4.644634839 | 1.892617279 | 0.250924135 | [‘N449Q’, ‘P451-’, ‘S452T’, ‘T456G’, |
| | | | | | | ‘Q457T’, ‘R459G’] |
| VAR-5 | 6 | 3.88486946 | −2.450075546 | −1.634402766 | −1.233590205 | [‘Q457F’, ‘S458C’] |
| VAR-6 | 7 | 2.853842111 | −1.792540736 | −2.923205527 | −1.532404634 | [‘446_7aa_447_KCQEGMA’] |
| VAR-7 | 8 | 2.970149877 | −1.540722356 | −1.140865448 | −3.184814278 | [‘449_7aa_450_LMVDRLG’] |
| VAR-8 | 9 | 5.582852851 | 5.427442711 | 2.525830415 | 1.385285762 | [‘P451T’, ‘T455G’, ‘T456G’, ‘Q457T’, |
| | | | | | | ‘S458Q’, ‘R459T’, ‘Q461A’] |
| VAR-9 | 10 | 4.669316134 | 3.927282285 | −3.376530605 | 0.001142983 | [‘448_7aa_449_HCQECPI’] |
| VAR-10 | 11 | 3.292909035 | 3.163362277 | −1.978073355 | 1.240848456 | [‘453_6aa_454_FSGLEN’] |
| VAR-11 | 12 | −0.576693086 | 3.596174251 | 3.834588967 | −2.671820443 | [‘T456-’, ‘Q457-’, ‘S458-’, ‘R459-’, |
| | | | | | | ‘L460-’, ‘Q461-’] |
| VAR-12 | 13 | −0.113423367 | 1.044477801 | 3.674269913 | −3.189453528 | [‘N449I’, ‘T450N’, ‘P451G’, ‘T454-’, |
| | | | | | | ‘T455Q’, ‘T456N’, ‘S458Q’, ‘R459T’, |
| | | | | | | ‘Q461K’, ‘Q464V’] |
| VAR-13 | 14 | 3.165665208 | 3.89324002 | 3.100397565 | 0.842518983 | [‘S446A’, ‘T448-’, ‘N449-’, ‘P451Q’, |
| | | | | | | ‘G453T’, ‘T455G’, ‘T456G’, ‘Q457T’, |
| | | | | | | ‘R459G’] |
| VAR-14 | 15 | 2.075188868 | 3.289224002 | 3.023792623 | −0.475270261 | [‘N449Q’, ‘T450S’, ‘451_1aa_452_Y’, |
| | | | | | | ‘S452G’, ‘T455A’, ‘Q457F’, ‘S458M’, |
| | | | | | | ‘R459D’] |
| VAR-15 | 16 | −0.376914592 | −3.500304156 | 1.324154172 | −2.458171594 | [‘447_7aa_448_HETEFNF’] |
| VAR-16 | 17 | 5.582852851 | 5.427442711 | 2.525830415 | 1.385285762 | [‘P451T’, ‘T455G’, ‘T456G’, ‘Q457T’, |
| | | | | | | ‘S458Q’, ‘R459T’, ‘Q461A’] |
| VAR-17 | 18 | 2.655350309 | −3.112635564 | −3.049750846 | −2.601297654 | [‘581_6aa_582_FALMEP’] |
| VAR-18 | 19 | 4.005041582 | 4.890414229 | −2.277159374 | 0.742690925 | [‘584_6aa_585_RAYNPD’] |
| VAR-19 | 20 | −0.533212469 | 0.980979298 | 3.697444744 | 0.090864036 | [‘E555A’, ‘D561S’, ‘R566K’, ‘S578V’, |
| | | | | | | ‘S580A’, ‘T581A’, ‘R585-’] |
| VAR-20 | 21 | 2.556229802 | 1.402914506 | 3.663931804 | −1.767018218 | [‘V557L’, ‘S578D’] |
| VAR-21 | 22 | 0.208673258 | 3.174958358 | 3.619270294 | 0.907816878 | [‘K556N’, ‘M558L’, ‘S578I’, ‘S580A’] |
| VAR-22 | 23 | −1.163302315 | 0.807138873 | 2.946707073 | 0.724043413 | [‘I554L’, ‘K556R’, ‘I559L’, ‘D561S’, |
| | | | | | | ‘R566A’, ‘T581D’, ‘R585S’, ‘G586R’, |
| | | | | | | ‘N587S’] |
| VAR-23 | 24 | −0.603741502 | −3.96314209 | 2.195605843 | −1.54313115 | [‘V552A’, ‘E555S’, ‘K556D’, ‘I559L’, |
| | | | | | | ‘D561S’, ‘R566K’, ‘S578I’, ‘T581D’, |
| | | | | | | ‘G586Q’] |
| VAR-24 | 25 | −0.32559336 | −2.523773182 | 3.018751224 | −2.351824168 | [‘Q575E’, ‘S578A’] |
| VAR-25 | 26 | −0.764009051 | −3.347174497 | 1.221512579 | −2.743972813 | [‘583_6aa_584_LNWTAE’] |
| VAR-26 | 27 | 6.543457411 | 4.274472882 | Not measured | 2.647908003 | [‘R585S’, ‘R588T’, ‘Q589N’, ‘A590P’, |
| | | | | | | ‘A591I’, ‘A593G’, ‘T597S’, ‘V600A’] |
| VAR-27 | 28 | 2.378409668 | −2.554934829 | −3.294730082 | −1.661215309 | [‘584_8aa_585_LAKEFTTR’, |
| | | | | | | ‘R585N’, ‘G586A’] |
| VAR-28 | 29 | 3.179099645 | −1.033272432 | −3.418068966 | −0.287722121 | [‘592_5aa_593_LHPLE’] |
| VAR-29 | 30 | 0.106530846 | 5.978252156 | −1.307152207 | −1.835575632 | [‘585_1aa_586_F’] |
| VAR-30 | 31 | 2.388880748 | 5.767603532 | −0.598614376 | −1.37771365 | [‘586_7aa_587_DQDFKNR’] |
| VAR-31 | 32 | 1.231859812 | 5.703988038 | −0.109487377 | −0.461783742 | [‘589_1aa_590_I’] |
| VAR-32 | 33 | 7.17868729 | 5.676618671 | 1.847520944 | 1.47820717 | [‘587_9aa_588_LAIEQTRPA’] |
| VAR-33 | 34 | 6.718793621 | 5.633141613 | 0.915715523 | 0.089517923 | [‘584_8aa_585_RARLDETT’, |
| | | | | | | ‘G586P’, ‘N587A’] |
| VAR-34 | 35 | 7.147097174 | 5.009347977 | 2.548103321 | −0.417075 | [‘586_9aa_587_LALAEITRP’, |
| | | | | | | ‘N587A’] |
| VAR-35 | 36 | 7.144189999 | 4.822397494 | 0.295037676 | 0.656194018 | [‘586_9aa_587_LANGEQTRP’, |
| | | | | | | ‘N587A’] |
| VAR-36 | 37 | 5.69301826 | 5.642147204 | 1.165262957 | 1.961105857 | [‘586_6aa_587_ATDTKT’] |
| VAR-37 | 38 | 5.426413987 | 5.646189636 | 2.526214031 | 0.606956149 | [‘590_1aa_591_P’] |
| VAR-38 | 39 | 5.256650619 | 5.61442957 | 3.562509958 | 1.182074152 | [‘587_9aa_588_APGETTRPA’] |
| VAR-39 | 40 | 5.560027745 | 5.255320701 | 2.631680376 | 1.835598668 | [‘589_1aa_590_P’] |
| VAR-40 | 41 | 5.342177909 | 5.237000927 | −0.042394845 | 0.280289485 | [‘R585S’, ‘G586S’, ‘N587A’, ‘A591E’, |
| | | | | | | ‘D594R’, ‘T597A’, ‘V600I’] |
| VAR-41 | 42 | 6.012003904 | 2.91024213 | −2.709889212 | −1.935204488 | [‘586_7aa_587_FHNEGKY’] |
| VAR-42 | 43 | 4.249223885 | 4.499692005 | −2.506762655 | 0.70070977 | [‘591_1aa_592_G’] |
| VAR-43 | 44 | 0.482055709 | 1.250967689 | 3.121559452 | −0.741624797 | [‘588_7aa_589_QPWEPDK’] |
| VAR-44 | 45 | −0.029969745 | −1.721769787 | 1.831873906 | −2.556026398 | [‘592_8aa_593_ALALSTTN’] |
| VAR-45 | 46 | 0.8687088 | −1.294220763 | 2.096329284 | −1.272924619 | [‘585_6aa_586_PWGTAG’] |

TABLE 2

| | Amino Acid | |
|---|---|---|
| | Sequence of | |
| | VP1 capsid | |
| | polypeptide | |
| | (SEQ ID NO; | |
| | starting amino | |
| | acid of VP2 is | |
| | underlined; | |
| | starting | Exemplary Nucleic Acid |
| Capsid | amino acid of | Molecule Sequence |
| Variant | VP3 is in bold. | (SEQ ID NO) |
| VAR-1 | MAADGYLPDWLEDLVSE | ATGGCTGCCGATGGTTATCTTCCAGATTG |
| | GARQWWHLKPGPPPPKP | GCTCGAGGACCTGGTATCTGAAGGAGCGA |
| | AERHKDDSRGLVLPGYK | GACAGTGGTGGCATCTCAAACCTGGCCCA |
| | YLGPFNGLDKGEPVNEA | CCACCACCAAAGCCCGCAGAGCGGCATAA |
| | DAAALEHDKAYDRQLDS | GGACGACAGCAGGGGTCTTGTGCTTCCTG |
| | GDNPYLKYNHADAEFQE | GGTACAAGTACCTCGGACCCTTCAACGGA |
| | RLKEDTSFGGNLGRAVF | CTCGACAAGGGAGAGCCGGTCAACGAGGC |
| | QAKKRVLEPLGLVEEPV | AGACGCCGCGGCCCTCGAGCACGACAAAG |
| | KTAPGKKRPVEHSPVEP | CCTACGACCGGCAGCTCGACAGCGGAGAC |
| | DSSSGTGKAGQQPARKR | AACCCGTACCTCAAGTACAACCACGCCGA |
| | LNFGQTGDADSVPDPQP | CGCGGAGTTTCAGGAGCGCCTTAAAGAAG |
| | LGQPPAAPSGLGTNTMA | ATACGTCTTTTGGGGGCAACCTCGGACGA |
| | TGSGAPMADNNEGADGV | GCAGTCTTCCAGGCGAAAAAGAGGGTTCT |
| | GNSSGNWHCDSTWMGDR | TGAACCTCTGGGCCTGGTTGAGGAACCTG |
| | VITTSTRTWALPTYNNH | TTAAGACGGCTCCGGGAAAAAAGAGGCCG |
| | LYKQISSQSGASNDNHY | GTAGAGCACTCTCCTGTGGAGCCAGACTC |
| | FGYSTPWGYFDFNRFHC | CTCCTCGGGAACCGGAAAGGCGGGCCAGC |
| | HFSPRDWQRLINNNWGF | AGCCTGCAAGAAAAAGATTGAATTTTGGT |
| | RPKRLNFKLFNIQVKEV | CAGACTGGAGACGCAGACTCAGTACCTGA |
| | TQNDGTTTIANNLTSTV | CCCCCAGCCTCTCGGACAGCCACCAGCAG |
| | QVFTDSEYQLPYVLGSA | CCCCCTCTGGTCTGGGAACTAATACGATG |
| | HQGCLPPFPADVFMVPQ | GCTACAGGCAGTGGCGCACCAATGGCAGA |
| | YGYLTLNNGSQAVGRSS | CAATAACGAGGGCGCCGACGGAGTGGGTA |
| | FYCLEYFPSQMLRTGNN | ATTCCTCGGGAAATTGGCATTGCGATTCC |
| | FTFSYTFEDVPFHSSYA | ACATGGATGGGCGACAGAGTCATCACCAC |
| | HSQSLDRLMNPLIDQYL | CAGCACCCGAACCTGGGCCCTGCCCACCT |
| | YYLSRTNTPSGTTTQSR | ACAACAACCACCTCTACAAACAAATTTCC |
| | LQFSQAGASDIRDQSRN | AGCCAATCAGGAGCCTCGAACGACAATCA |
| | WLPGPCYRQQRVSKTSA | CTACTTTGGCTACAGCACCCCTTGGGGGT |
| | DNNNSEYSWTGATKYHL | ATTTTGACTTCAACAGATTCCACTGCCAC |
| | NGRDSLVNPGPAMASHK | TTTTCACCACGTGACTGGCAAAGACTCAT |
| | DDEEKFFPQSGVLIFGK | CAACAACAACTGGGGATTCCGACCCAAGA |
| | QGSEKTNVDIEKVMITD | GACTCAACTTCAAGCTCTTTAACATTCAA |
| | EEEIRTTNPVATEQYGS | GTCAAAGAGGTCACGCAGAATGACGGTAC |
| | VSTNLQRGNRQAATADV | GACGACGATTGCCAATAACCTTACCAGCA |
| | NTQGVLPGMVWQDRDVY | CGGTTCAGGTGTTTACTGACTCGGAGTAC |
| | LQGPIWAKIPHTDGHFH | CAGCTCCCGTACGTCCTCGGCTCGGCGCA |
| | PSPLMGGFGLKHPPPQI | TCAAGGATGCCTCCCGCCGTTCCCAGCAG |
| | LIKNTPVPANPSTTFSA | ACGTCTTCATGGTGCCACAGTATGGATAC |
| | AKFASFITQYSTGQVSV | CTCACCCTGAACAACGGGAGTCAGGCAGT |
| | EIEWELQKENSKRWNPE | AGGACGCTCTTCATTTTACTGCCTGGAGT |
| | IQYTSNYNKSVNVDFTV | ACTTTCCTTCTCAGATGCTGCGTACCGGA |
| | DTNGVYSEPRPIGTRYL | AACAACTTTACCTTCAGCTACACTTTTGA |
| | TRNL* (SEQ ID NO: | GGACGTTCCTTTCCACAGCAGCTACGCTC |
| | 2) | ACAGCCAGAGTCTGGACCGTCTCATGAAT |
| | | CCTCTCATCGACCAGTACCTGTATTACTT |
| | | GAGCAGAACAAACACTCCAAGTGGAACCA |
| | | CCACGCAGTCAAGGCTTCAGTTTTCTCAG |
| | | GCCGGAGCGAGTGACATTCGGGACCAGTC |
| | | TAGGAACTGGCTTCCTGGACCCTGTTACC |
| | | GCCAGCAGCGAGTATCAAAGACATCTGCG |
| | | GATAACAACAACAGTGAATACTCGTGGAC |
| | | TGGAGCTACCAAGTACCACCTCAATGGCA |
| | | GAGACTCTCTGGTGAATCCGGGCCCGGCC |
| | | ATGGCAAGCCACAAGGACGATGAAGAAAA |
| | | GTTTTTTCCTCAGAGCGGGGTTCTCATCT |
| | | TTGGGAAGCAAGGCTCAGAGAAAACAAAT |
| | | GTGGACATTGAAAAGGTCATGATTACAGA |
| | | CGAAGAGGAAATCAGGACAACCAATCCCG |
| | | TGGCTACGGAGCAGTATGGTTCTGTATCT |
| | | ACCAACCTCCAGAGAGGCAACAGACAAGC |
| | | AGCTACCGCAGATGTCAACACACAAGGCG |
| | | TTCTTCCAGGCATGGTCTGGCAGGACAGA |
| | | GATGTGTACCTTCAGGGGCCCATCTGGGC |
| | | AAAGATTCCACACACGGACGGACATTTTC |
| | | ACCCCTCTCCCCTCATGGGTGGATTCGGA |
| | | CTTAAACACCCTCCTCCACAGATTCTCAT |
| | | CAAGAACACCCCGGTACCTGCGAATCCTT |
| | | CGACCACCTTCAGTGCGGCAAAGTTTGCT |
| | | TCCTTCATCACACAGTACTCCACGGGACA |
| | | GGTCAGCGTGGAGATCGAGTGGGAGCTGC |
| | | AGAAGGAAAACAGCAAACGCTGGAATCCC |
| | | GAAATTCAGTACACTTCCAACTACAACAA |
| | | GTCTGTTAATGTGGACTTTACTGTGGACA |
| | | CTAATGGCGTGTATTCAGAGCCTCGCCCC |
| | | ATTGGCACCAGATACCTGACTCGTAATCT |
| | | GTAA (SEQ ID NO: 47) |
| VAR-2 | MAVDGFLPDWFQDTLSE | ATGGCTGTTGATGGTTTTCTTCCAGATTG |
| | GIRLWIKCKPGAPNPRP | GTTTCAAGACACTCTCTCTGAAGGAATAA |
| | AERHKDDSRGLVLPGYK | GATTATGGATCAAGTGCAAACCTGGCGCG |
| | YLGPFNGLDKGEPVNEA | CCAAACCCACGCCCCGCAGAGCGGCATAA |
| | DAAALEHDKAYDRQLDS | GGACGACAGCAGGGGTCTTGTGCTTCCTG |
| | GDNPYLKYNHADAEFQE | GGTACAAGTACCTCGGACCCTTCAACGGA |
| | RLKEDTSFGGNLGRAVF | CTCGACAAGGGAGAGCCGGTCAACGAGGC |
| | QAKKRVLEPLGLVEEPV | AGACGCCGCGGCCCTCGAGCACGACAAAG |
| | KTAPGKKRPVEHSPVEP | CCTACGACCGGCAGCTCGACAGCGGAGAC |
| | DSSSGTGKAGQQPARKR | AACCCGTACCTCAAGTACAACCACGCCGA |
| | LNFGQTGDADSVPDPQP | CGCGGAGTTTCAGGAGCGCCTTAAAGAAG |
| | LGQPPAAPSGLGTNTMA | ATACGTCTTTTGGGGGCAACCTCGGACGA |
| | TGSGAPMADNNEGADGV | GCAGTCTTCCAGGCGAAAAAGAGGGTTCT |
| | GNSSGNWHCDSTWMGDR | TGAACCTCTGGGCCTGGTTGAGGAACCTG |
| | VITTSTRTWALPTYNNH | TTAAGACGGCTCCGGGAAAAAAGAGGCCG |
| | LYKQISSQSGASNDNHY | GTAGAGCACTCTCCTGTGGAGCCAGACTC |
| | FGYSTPWGYFDFNRFHC | CTCCTCGGGAACCGGAAAGGCGGGCCAGC |
| | HFSPRDWQRLINNNWGF | AGCCTGCAAGAAAAAGATTGAATTTTGGT |
| | RPKRLNFKLFNIQVKEV | CAGACTGGAGACGCAGACTCAGTACCTGA |
| | TQNDGTTTIANNLTSTV | CCCCCAGCCTCTCGGACAGCCACCAGCAG |
| | QVFTDSEYQLPYVLGSA | CCCCCTCTGGTCTGGGAACTAATACGATG |
| | HQGCLPPFPADVFMVPQ | GCTACAGGCAGTGGCGCACCAATGGCAGA |
| | YGYLTLNNGSQAVGRSS | CAATAACGAGGGCGCCGACGGAGTGGGTA |
| | FYCLEYFPSQMLRTGNN | ATTCCTCGGGAAATTGGCATTGCGATTCC |
| | FTFSYTFEDVPFHSSYA | ACATGGATGGGCGACAGAGTCATCACCAC |
| | HSQSLDRLMNPLIDQYL | CAGCACCCGAACCTGGGCCCTGCCCACCT |
| | YYLSRTNTPSGTTTQSR | ACAACAACCACCTCTACAAACAAATTTCC |
| | LQFSQAGASDIRDQSRN | AGCCAATCAGGAGCCTCGAACGACAATCA |
| | WLPGPCYRQQRVSKTSA | CTACTTTGGCTACAGCACCCCTTGGGGGT |
| | DNNNSEYSWTGATKYHL | ATTTTGACTTCAACAGATTCCACTGCCAC |
| | NGRDSLVNPGPAMASHK | TTTTCACCACGTGACTGGCAAAGACTCAT |
| | DDEEKFFPQSGVLIFGK | CAACAACAACTGGGGATTCCGACCCAAGA |
| | QGSEKTNVDIEKVMITD | GACTCAACTTCAAGCTCTTTAACATTCAA |
| | EEEIRTTNPVATEQYGS | GTCAAAGAGGTCACGCAGAATGACGGTAC |
| | VSTNLQRGNRQAATADV | GACGACGATTGCCAATAACCTTACCAGCA |
| | NTQGVLPGMVWQDRDVY | CGGTTCAGGTGTTTACTGACTCGGAGTAC |
| | LQGPIWAKIPHTDGHFH | CAGCTCCCGTACGTCCTCGGCTCGGCGCA |
| | PSPLMGGFGLKHPPPQI | TCAAGGATGCCTCCCGCCGTTCCCAGCAG |
| | LIKNTPVPANPSTTFSA | ACGTCTTCATGGTGCCACAGTATGGATAC |
| | AKFASFITQYSTGQVSV | CTCACCCTGAACAACGGGAGTCAGGCAGT |
| | EIEWELQKENSKRWNPE | AGGACGCTCTTCATTTTACTGCCTGGAGT |
| | IQYTSNYNKSVNVDFTV | ACTTTCCTTCTCAGATGCTGCGTACCGGA |
| | DTNGVYSEPRPIGTRYL | AACAACTTTACCTTCAGCTACACTTTTGA |
| | TRNL* (SEQ ID NO: | GGACGTTCCTTTCCACAGCAGCTACGCTC |
| | 3) | ACAGCCAGAGTCTGGACCGTCTCATGAAT |
| | | CCTCTCATCGACCAGTACCTGTATTACTT |
| | | GAGCAGAACAAACACTCCAAGTGGAACCA |
| | | CCACGCAGTCAAGGCTTCAGTTTTCTCAG |
| | | GCCGGAGCGAGTGACATTCGGGACCAGTC |
| | | TAGGAACTGGCTTCCTGGACCCTGTTACC |
| | | GCCAGCAGCGAGTATCAAAGACATCTGCG |
| | | GATAACAACAACAGTGAATACTCGTGGAC |
| | | TGGAGCTACCAAGTACCACCTCAATGGCA |
| | | GAGACTCTCTGGTGAATCCGGGCCCGGCC |
| | | ATGGCAAGCCACAAGGACGATGAAGAAAA |
| | | GTTTTTTCCTCAGAGCGGGGTTCTCATCT |
| | | TTGGGAAGCAAGGCTCAGAGAAAACAAAT |
| | | GTGGACATTGAAAAGGTCATGATTACAGA |
| | | CGAAGAGGAAATCAGGACAACCAATCCCG |
| | | TGGCTACGGAGCAGTATGGTTCTGTATCT |
| | | ACCAACCTCCAGAGAGGCAACAGACAAGC |
| | | AGCTACCGCAGATGTCAACACACAAGGCG |
| | | TTCTTCCAGGCATGGTCTGGCAGGACAGA |
| | | GATGTGTACCTTCAGGGGCCCATCTGGGC |
| | | AAAGATTCCACACACGGACGGACATTTTC |
| | | ACCCCTCTCCCCTCATGGGTGGATTCGGA |
| | | CTTAAACACCCTCCTCCACAGATTCTCAT |
| | | CAAGAACACCCCGGTACCTGCGAATCCTT |
| | | CGACCACCTTCAGTGCGGCAAAGTTTGCT |
| | | TCCTTCATCACACAGTACTCCACGGGACA |
| | | GGTCAGCGTGGAGATCGAGTGGGAGCTGC |
| | | AGAAGGAAAACAGCAAACGCTGGAATCCC |
| | | GAAATTCAGTACACTTCCAACTACAACAA |
| | | GTCTGTTAATGTGGACTTTACTGTGGACA |
| | | CTAATGGCGTGTATTCAGAGCCTCGCCCC |
| | | ATTGGCACCAGATACCTGACTCGTAATCT |
| | | GTAA (SEQ ID NO: 48) |

TABLE 3

| | Amino Acid Sequence of VP1 | |
|---|---|---|
| | capsid polypeptide (SEQ ID | |
| | NO; starting amino acid of | |
| Capsid | VP2 is underlined; starting | Exemplary Nucleic Acid Molecule Sequence(SEQ |
| Variant | amino acid of VP3 is in bold. | ID NO) |
| VAR-3 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TQMTSGTLGTQMLQFSQA | CCGAACCTGGGCCCTGCCCACCTACAACA |
| | GASDIRDQSRNWLPGPCY | ACCACCTCTACAAACAAATTTCCAGCCAAT |
| | RQQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 4) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGGCTCATGAATCCTCTCATCGACCAGTA |
| | | CCTGTATTACTTGAGCAGAACACAGATGA |
| | | CCAGTGGAACCTTGGGTACACAGATGCTTC |
| | | AGTTTTCTCAGGCCGGAGCGAGTGACATTC |
| | | GGGACCAGTCTAGGAACTGGCTTCCTGGA |
| | | CCCTGTTACCGCCAGCAGCGAGTATCAAA |
| | | GACATCTGCGGATAACAACAACAGTGAAT |
| | | ACTCGTGGACTGGAGCTACCAAGTACCAC |
| | | CTCAATGGCAGAGACTCTCTGGTGAATCCG |
| | | GGCCCGGCCATGGCAAGCCACAAGGACGA |
| | | TGAAGAAAAGTTTTTTCCTCAGAGCGGGGT |
| | | TCTCATCTTTGGGAAGCAAGGCTCAGAGA |
| | | AAACAAATGTGGACATTGAAAAGGTCATG |
| | | ATTACAGACGAAGAGGAAATCAGGACAAC |
| | | CAATCCCGTGGCTACGGAGCAGTATGGTTC |
| | | TGTATCTACCAACCTCCAGAGAGGCAACA |
| | | GACAAGCAGCTACCGCAGATGTCAACACA |
| | | CAAGGCGTTCTTCCAGGCATGGTCTGGCAG |
| | | GACAGAGATGTGTACCTTCAGGGGCCCAT |
| | | CTGGGCAAAGATTCCACACACGGACGGAC |
| | | ATTTTCACCCCTCTCCCCTCATGGGTGGAT |
| | | TCGGACTTAAACACCCTCCTCCACAGATTC |
| | | TCATCAAGAACACCCCGGTACCTGCGAAT |
| | | CCTTCGACCACCTTCAGTGCGGCAAAGTTT |
| | | GCTTCCTTCATCACACAGTACTCCACGGGA |
| | | CAGGTCAGCGTGGAGATCGAGTGGGAGCT |
| | | GCAGAAGGAAAACAGCAAACGCTGGAATC |
| | | CCGAAATTCAGTACACTTCCAACTACAACA |
| | | AGTCTGTTAATGTGGACTTTACTGTGGACA |
| | | CTAATGGCGTGTATTCAGAGCCTCGCCCCA |
| | | TTGGCACCAGATACCTGACTCGTAATCTGT |
| | | AA (SEQ ID NO: 49) |
| VAR-4 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TQTTGTTGTSGLQFSQAG | CCGAACCTGGGCCCTGCCCACCTACAACA |
| | ASDIRDQSRNWLPGPCYR | ACCACCTCTACAAACAAATTTCCAGCCAAT |
| | QQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 5) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CAGACTCATGAATCCTCTCATCGACCAGTA |
| | | CCTGTATTACTTGAGCAGAACACAAACTAC |
| | | TGGAACCACCGGTACCTCAGGACTTCAGTT |
| | | TTCTCAGGCCGGAGCGAGTGACATTCGGG |
| | | ACCAGTCTAGGAACTGGCTTCCTGGACCCT |
| | | GTTACCGCCAGCAGCGAGTATCAAAGACA |
| | | TCTGCGGATAACAACAACAGTGAATACTC |
| | | GTGGACTGGAGCTACCAAGTACCACCTCA |
| | | ATGGCAGAGACTCTCTGGTGAATCCGGGC |
| | | CCGGCCATGGCAAGCCACAAGGACGATGA |
| | | AGAAAAGTTTTTTCCTCAGAGCGGGGTTCT |
| | | CATCTTTGGGAAGCAAGGCTCAGAGAAAA |
| | | CAAATGTGGACATTGAAAAGGTCATGATT |
| | | ACAGACGAAGAGGAAATCAGGACAACCA |
| | | ATCCCGTGGCTACGGAGCAGTATGGTTCTG |
| | | TATCTACCAACCTCCAGAGAGGCAACAGA |
| | | CAAGCAGCTACCGCAGATGTCAACACACA |
| | | AGGCGTTCTTCCAGGCATGGTCTGGCAGG |
| | | ACAGAGATGTGTACCTTCAGGGGCCCATCT |
| | | GGGCAAAGATTCCACACACGGACGGACAT |
| | | TTTCACCCCTCTCCCCTCATGGGTGGATTC |
| | | GGACTTAAACACCCTCCTCCACAGATTCTC |
| | | ATCAAGAACACCCCGGTACCTGCGAATCC |
| | | TTCGACCACCTTCAGTGCGGCAAAGTTTGC |
| | | TTCCTTCATCACACAGTACTCCACGGGACA |
| | | GGTCAGCGTGGAGATCGAGTGGGAGCTGC |
| | | AGAAGGAAAACAGCAAACGCTGGAATCCC |
| | | GAAATTCAGTACACTTCCAACTACAACAA |
| | | GTCTGTTAATGTGGACTTTACTGTGGACAC |
| | | TAATGGCGTGTATTCAGAGCCTCGCCCCAT |
| | | TGGCACCAGATACCTGACTCGTAATCTGTA |
| | | A (SEQ ID NO: 50) |
| VAR-5 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNTPSGTTTFCRLQFSQAG | CCGAACCTGGGCCCTGCCCACCTACAACA |
| | ASDIRDQSRNWLPGPCYR | ACCACCTCTACAAACAAATTTCCAGCCAAT |
| | QQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 6) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTTATGAATCCTCTCATCGACCAGTA |
| | | CCTGTATTACTTGAGCAGAACAAACACTCC |
| | | AAGTGGAACCACCACGTTTTGTAGGCTTCA |
| | | GTTTTCTCAGGCCGGAGCGAGTGACATTCG |
| | | GGACCAGTCTAGGAACTGGCTTCCTGGAC |
| | | CCTGTTACCGCCAGCAGCGAGTATCAAAG |
| | | ACATCTGCGGATAACAACAACAGTGAATA |
| | | CTCGTGGACTGGAGCTACCAAGTACCACCT |
| | | CAATGGCAGAGACTCTCTGGTGAATCCGG |
| | | GCCCGGCCATGGCAAGCCACAAGGACGAT |
| | | GAAGAAAAGTTTTTTCCTCAGAGCGGGGTT |
| | | CTCATCTTTGGGAAGCAAGGCTCAGAGAA |
| | | AACAAATGTGGACATTGAAAAGGTCATGA |
| | | TTACAGACGAAGAGGAAATCAGGACAACC |
| | | AATCCCGTGGCTACGGAGCAGTATGGTTCT |
| | | GTATCTACCAACCTCCAGAGAGGCAACAG |
| | | ACAAGCAGCTACCGCAGATGTCAACACAC |
| | | AAGGCGTTCTTCCAGGCATGGTCTGGCAG |
| | | GACAGAGATGTGTACCTTCAGGGGCCCAT |
| | | CTGGGCAAAGATTCCACACACGGACGGAC |
| | | ATTTTCACCCCTCTCCCCTCATGGGTGGAT |
| | | TCGGACTTAAACACCCTCCTCCACAGATTC |
| | | TCATCAAGAACACCCCGGTACCTGCGAAT |
| | | CCTTCGACCACCTTCAGTGCGGCAAAGTTT |
| | | GCTTCCTTCATCACACAGTACTCCACGGGA |
| | | CAGGTCAGCGTGGAGATCGAGTGGGAGCT |
| | | GCAGAAGGAAAACAGCAAACGCTGGAATC |
| | | CCGAAATTCAGTACACTTCCAACTACAACA |
| | | AGTCTGTTAATGTGGACTTTACTGTGGACA |
| | | CTAATGGCGTGTATTCAGAGCCTCGCCCCA |
| | | TTGGCACCAGATACCTGACTCGTAATCTGT |
| | | AA (SEQ ID NO: 51) |
| VAR-6 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSK | ATGGGCGACAGAGTCATCACCACCAGCAC |
| | CQEGMARTNTPSGTTTQS | CCGAACCTGGGCCCTGCCCACCTACAACA |
| | RLQFSQAGASDIRDQSRN | ACCACCTCTACAAACAAATTTCCAGCCAAT |
| | WLPGPCYRQQRVSKTSAD | CAGGAGCCTCGAACGACAATCACTACTTT |
| | NNNSEYSWTGATKYHLN | GGCTACAGCACCCCTTGGGGGTATTTTGAC |
| | GRDSLVNPGPAMASHKD | TTCAACAGATTCCACTGCCACTTTTCACCA |
| | DEEKFFPQSGVLIFGKQGS | CGTGACTGGCAAAGACTCATCAACAACAA |
| | EKTNVDIEKVMITDEEEIR | CTGGGGATTCCGACCCAAGAGACTCAACT |
| | TTNPVATEQYGSVSTNLQ | TCAAGCTCTTTAACATTCAAGTCAAAGAGG |
| | RGNRQAATADVNTQGVL | TCACGCAGAATGACGGTACGACGACGATT |
| | PGMVWQDRDVYLQGPIW | GCCAATAACCTTACCAGCACGGTTCAGGT |
| | AKIPHTDGHFHPSPLMGG | GTTTACTGACTCGGAGTACCAGCTCCCGTA |
| | FGLKHPPPQILIKNTPVPA | CGTCCTCGGCTCGGCGCATCAAGGATGCCT |
| | NPSTTFSAAKFASFITQYS | CCCGCCGTTCCCAGCAGACGTCTTCATGGT |
| | TGQVSVEIEWELQKENSK | GCCACAGTATGGATACCTCACCCTGAACA |
| | RWNPEIQYTSNYNKSVNV | ACGGGAGTCAGGCAGTAGGACGCTCTTCA |
| | DFTVDTNGVYSEPRPIGTR | TTTTACTGCCTGGAGTACTTTCCTTCTCAG |
| | YLTRNL* (SEQ ID NO: 7) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTGATGAATCCTCTCATCGACCAGTA |
| | | CCTGTATTACTTGAGCAAGTGCCAGGAAG |
| | | GAATGGCTAGAACAAACACTCCAAGTGGA |
| | | ACCACCACGCAGTCAAGGCTTCAGTTTTCT |
| | | CAGGCCGGAGCGAGTGACATTCGGGACCA |
| | | GTCTAGGAACTGGCTTCCTGGACCCTGTTA |
| | | CCGCCAGCAGCGAGTATCAAAGACATCTG |
| | | CGGATAACAACAACAGTGAATACTCGTGG |
| | | ACTGGAGCTACCAAGTACCACCTCAATGG |
| | | CAGAGACTCTCTGGTGAATCCGGGCCCGG |
| | | CCATGGCAAGCCACAAGGACGATGAAGAA |
| | | AAGTTTTTTCCTCAGAGCGGGGTTCTCATC |
| | | TTTGGGAAGCAAGGCTCAGAGAAAACAAA |
| | | TGTGGACATTGAAAAGGTCATGATTACAG |
| | | ACGAAGAGGAAATCAGGACAACCAATCCC |
| | | GTGGCTACGGAGCAGTATGGTTCTGTATCT |
| | | ACCAACCTCCAGAGAGGCAACAGACAAGC |
| | | AGCTACCGCAGATGTCAACACACAAGGCG |
| | | TTCTTCCAGGCATGGTCTGGCAGGACAGA |
| | | GATGTGTACCTTCAGGGGCCCATCTGGGCA |
| | | AAGATTCCACACACGGACGGACATTTTCA |
| | | CCCCTCTCCCCTCATGGGTGGATTCGGACT |
| | | TAAACACCCTCCTCCACAGATTCTCATCAA |
| | | GAACACCCCGGTACCTGCGAATCCTTCGAC |
| | | CACCTTCAGTGCGGCAAAGTTTGCTTCCTT |
| | | CATCACACAGTACTCCACGGGACAGGTCA |
| | | GCGTGGAGATCGAGTGGGAGCTGCAGAAG |
| | | GAAAACAGCAAACGCTGGAATCCCGAAAT |
| | | TCAGTACACTTCCAACTACAACAAGTCTGT |
| | | TAATGTGGACTTTACTGTGGACACTAATGG |
| | | CGTGTATTCAGAGCCTCGCCCCATTGGCAC |
| | | CAGATACCTGACTCGTAATCTGTAA (SEQ |
| | | ID NO: 52) |
| VAR-7 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNLMVDRLGTPSGTTTQS | CCGAACCTGGGCCCTGCCCACCTACAACA |
| | RLQFSQAGASDIRDQSRN | ACCACCTCTACAAACAAATTTCCAGCCAAT |
| | WLPGPCYRQQRVSKTSAD | CAGGAGCCTCGAACGACAATCACTACTTT |
| | NNNSEYSWTGATKYHLN | GGCTACAGCACCCCTTGGGGGTATTTTGAC |
| | GRDSLVNPGPAMASHKD | TTCAACAGATTCCACTGCCACTTTTCACCA |
| | DEEKFFPQSGVLIFGKQGS | CGTGACTGGCAAAGACTCATCAACAACAA |
| | EKTNVDIEKVMITDEEEIR | CTGGGGATTCCGACCCAAGAGACTCAACT |
| | TTNPVATEQYGSVSTNLQ | TCAAGCTCTTTAACATTCAAGTCAAAGAGG |
| | RGNRQAATADVNTQGVL | TCACGCAGAATGACGGTACGACGACGATT |
| | PGMVWQDRDVYLQGPIW | GCCAATAACCTTACCAGCACGGTTCAGGT |
| | AKIPHTDGHFHPSPLMGG | GTTTACTGACTCGGAGTACCAGCTCCCGTA |
| | FGLKHPPPQILIKNTPVPA | CGTCCTCGGCTCGGCGCATCAAGGATGCCT |
| | NPSTTFSAAKFASFITQYS | CCCGCCGTTCCCAGCAGACGTCTTCATGGT |
| | TGQVSVEIEWELQKENSK | GCCACAGTATGGATACCTCACCCTGAACA |
| | RWNPEIQYTSNYNKSVNV | ACGGGAGTCAGGCAGTAGGACGCTCTTCA |
| | DFTVDTNGVYSEPRPIGTR | TTTTACTGCCTGGAGTACTTTCCTTCTCAG |
| | YLTRNL* (SEQ ID NO: 8) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTAATGAATCCTCTCATCGACCAGTA |
| | | CCTGTATTACTTGAGCAGAACAAACCTCAT |
| | | GGTTGACCGACTTGGGACTCCAAGTGGAA |
| | | CCACCACGCAGTCAAGGCTTCAGTTTTCTC |
| | | AGGCCGGAGCGAGTGACATTCGGGACCAG |
| | | TCTAGGAACTGGCTTCCTGGACCCTGTTAC |
| | | CGCCAGCAGCGAGTATCAAAGACATCTGC |
| | | GGATAACAACAACAGTGAATACTCGTGGA |
| | | CTGGAGCTACCAAGTACCACCTCAATGGC |
| | | AGAGACTCTCTGGTGAATCCGGGCCCGGC |
| | | CATGGCAAGCCACAAGGACGATGAAGAAA |
| | | AGTTTTTTCCTCAGAGCGGGGTTCTCATCT |
| | | TTGGGAAGCAAGGCTCAGAGAAAACAAAT |
| | | GTGGACATTGAAAAGGTCATGATTACAGA |
| | | CGAAGAGGAAATCAGGACAACCAATCCCG |
| | | TGGCTACGGAGCAGTATGGTTCTGTATCTA |
| | | CCAACCTCCAGAGAGGCAACAGACAAGCA |
| | | GCTACCGCAGATGTCAACACACAAGGCGT |
| | | TCTTCCAGGCATGGTCTGGCAGGACAGAG |
| | | ATGTGTACCTTCAGGGGCCCATCTGGGCAA |
| | | AGATTCCACACACGGACGGACATTTTCACC |
| | | CCTCTCCCCTCATGGGTGGATTCGGACTTA |
| | | AACACCCTCCTCCACAGATTCTCATCAAGA |
| | | ACACCCCGGTACCTGCGAATCCTTCGACCA |
| | | CCTTCAGTGCGGCAAAGTTTGCTTCCTTCA |
| | | TCACACAGTACTCCACGGGACAGGTCAGC |
| | | GTGGAGATCGAGTGGGAGCTGCAGAAGGA |
| | | AAACAGCAAACGCTGGAATCCCGAAATTC |
| | | AGTACACTTCCAACTACAACAAGTCTGTTA |
| | | ATGTGGACTTTACTGTGGACACTAATGGCG |
| | | TGTATTCAGAGCCTCGCCCCATTGGCACCA |
| | | GATACCTGACTCGTAATCTGTAA (SEQ ID |
| | | NO: 53) |
| VAR-8 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNTTSGTGGTQTLAFSQA | CCGAACCTGGGCCCTGCCCACCTACAACA |
| | GASDIRDQSRNWLPGPCY | ACCACCTCTACAAACAAATTTCCAGCCAAT |
| | RQQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 9) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGACTCATGAATCCTCTCATCGACCAGTA |
| | | CCTGTATTACTTGAGCAGAACAAACACTAC |
| | | AAGTGGAACCGGAGGAACACAGACGCTTG |
| | | CTTTTTCTCAGGCCGGAGCGAGTGACATTC |
| | | GGGACCAGTCTAGGAACTGGCTTCCTGGA |
| | | CCCTGTTACCGCCAGCAGCGAGTATCAAA |
| | | GACATCTGCGGATAACAACAACAGTGAAT |
| | | ACTCGTGGACTGGAGCTACCAAGTACCAC |
| | | CTCAATGGCAGAGACTCTCTGGTGAATCCG |
| | | GGCCCGGCCATGGCAAGCCACAAGGACGA |
| | | TGAAGAAAAGTTTTTTCCTCAGAGCGGGGT |
| | | TCTCATCTTTGGGAAGCAAGGCTCAGAGA |
| | | AAACAAATGTGGACATTGAAAAGGTCATG |
| | | ATTACAGACGAAGAGGAAATCAGGACAAC |
| | | CAATCCCGTGGCTACGGAGCAGTATGGTTC |
| | | TGTATCTACCAACCTCCAGAGAGGCAACA |
| | | GACAAGCAGCTACCGCAGATGTCAACACA |
| | | CAAGGCGTTCTTCCAGGCATGGTCTGGCAG |
| | | GACAGAGATGTGTACCTTCAGGGGCCCAT |
| | | CTGGGCAAAGATTCCACACACGGACGGAC |
| | | ATTTTCACCCCTCTCCCCTCATGGGTGGAT |
| | | TCGGACTTAAACACCCTCCTCCACAGATTC |
| | | TCATCAAGAACACCCCGGTACCTGCGAAT |
| | | CCTTCGACCACCTTCAGTGCGGCAAAGTTT |
| | | GCTTCCTTCATCACACAGTACTCCACGGGA |
| | | CAGGTCAGCGTGGAGATCGAGTGGGAGCT |
| | | GCAGAAGGAAAACAGCAAACGCTGGAATC |
| | | CCGAAATTCAGTACACTTCCAACTACAACA |
| | | AGTCTGTTAATGTGGACTTTACTGTGGACA |
| | | CTAATGGCGTGTATTCAGAGCCTCGCCCCA |
| | | TTGGCACCAGATACCTGACTCGTAATCTGT |
| | | AA (SEQ ID NO: 54) |
| VAR-9 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | THCQECPINTPSGTTTQSR | CCGAACCTGGGCCCTGCCCACCTACAACA |
| | LQFSQAGASDIRDQSRNW | ACCACCTCTACAAACAAATTTCCAGCCAAT |
| | LPGPCYRQQRVSKTSADN | CAGGAGCCTCGAACGACAATCACTACTTT |
| | NNSEYSWTGATKYHLNG | GGCTACAGCACCCCTTGGGGGTATTTTGAC |
| | RDSLVNPGPAMASHKDDE | TTCAACAGATTCCACTGCCACTTTTCACCA |
| | EKFFPQSGVLIFGKQGSEK | CGTGACTGGCAAAGACTCATCAACAACAA |
| | TNVDIEKVMITDEEEIRTT | CTGGGGATTCCGACCCAAGAGACTCAACT |
| | NPVATEQYGSVSTNLQRG | TCAAGCTCTTTAACATTCAAGTCAAAGAGG |
| | NRQAATADVNTQGVLPG | TCACGCAGAATGACGGTACGACGACGATT |
| | MVWQDRDVYLQGPIWAK | GCCAATAACCTTACCAGCACGGTTCAGGT |
| | IPHTDGHFHPSPLMGGFGL | GTTTACTGACTCGGAGTACCAGCTCCCGTA |
| | KHPPPQILIKNTPVPANPST | CGTCCTCGGCTCGGCGCATCAAGGATGCCT |
| | TFSAAKFASFITQYSTGQV | CCCGCCGTTCCCAGCAGACGTCTTCATGGT |
| | SVEIEWELQKENSKRWNP | GCCACAGTATGGATACCTCACCCTGAACA |
| | EIQYTSNYNKSVNVDFTV | ACGGGAGTCAGGCAGTAGGACGCTCTTCA |
| | DTNGVYSEPRPIGTRYLTR | TTTTACTGCCTGGAGTACTTTCCTTCTCAG |
| | NL* (SEQ ID NO: 10) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTGATGAATCCTCTCATCGACCAGTA |
| | | CCTGTATTACTTGAGCAGAACACATTGTCA |
| | | GGAGTGTCCAATTAACACTCCAAGTGGAA |
| | | CCACCACGCAGTCAAGGCTTCAGTTTTCTC |
| | | AGGCCGGAGCGAGTGACATTCGGGACCAG |
| | | TCTAGGAACTGGCTTCCTGGACCCTGTTAC |
| | | CGCCAGCAGCGAGTATCAAAGACATCTGC |
| | | GGATAACAACAACAGTGAATACTCGTGGA |
| | | CTGGAGCTACCAAGTACCACCTCAATGGC |
| | | AGAGACTCTCTGGTGAATCCGGGCCCGGC |
| | | CATGGCAAGCCACAAGGACGATGAAGAAA |
| | | AGTTTTTTCCTCAGAGCGGGGTTCTCATCT |
| | | TTGGGAAGCAAGGCTCAGAGAAAACAAAT |
| | | GTGGACATTGAAAAGGTCATGATTACAGA |
| | | CGAAGAGGAAATCAGGACAACCAATCCCG |
| | | TGGCTACGGAGCAGTATGGTTCTGTATCTA |
| | | CCAACCTCCAGAGAGGCAACAGACAAGCA |
| | | GCTACCGCAGATGTCAACACACAAGGCGT |
| | | TCTTCCAGGCATGGTCTGGCAGGACAGAG |
| | | ATGTGTACCTTCAGGGGCCCATCTGGGCAA |
| | | AGATTCCACACACGGACGGACATTTTCACC |
| | | CCTCTCCCCTCATGGGTGGATTCGGACTTA |
| | | AACACCCTCCTCCACAGATTCTCATCAAGA |
| | | ACACCCCGGTACCTGCGAATCCTTCGACCA |
| | | CCTTCAGTGCGGCAAAGTTTGCTTCCTTCA |
| | | TCACACAGTACTCCACGGGACAGGTCAGC |
| | | GTGGAGATCGAGTGGGAGCTGCAGAAGGA |
| | | AAACAGCAAACGCTGGAATCCCGAAATTC |
| | | AGTACACTTCCAACTACAACAAGTCTGTTA |
| | | ATGTGGACTTTACTGTGGACACTAATGGCG |
| | | TGTATTCAGAGCCTCGCCCCATTGGCACCA |
| | | GATACCTGACTCGTAATCTGTAA (SEQ ID |
| | | NO: 55) |
| VAR-10 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNTPSGFSGLENTTTQSRL | CCGAACCTGGGCCCTGCCCACCTACAACA |
| | QFSQAGASDIRDQSRNWL | ACCACCTCTACAAACAAATTTCCAGCCAAT |
| | PGPCYRQQRVSKTSADNN | CAGGAGCCTCGAACGACAATCACTACTTT |
| | NSEYSWTGATKYHLNGR | GGCTACAGCACCCCTTGGGGGTATTTTGAC |
| | DSLVNPGPAMASHKDDEE | TTCAACAGATTCCACTGCCACTTTTCACCA |
| | KFFPQSGVLIFGKQGSEKT | CGTGACTGGCAAAGACTCATCAACAACAA |
| | NVDIEKVMITDEEEIRTTN | CTGGGGATTCCGACCCAAGAGACTCAACT |
| | PVATEQYGSVSTNLQRGN | TCAAGCTCTTTAACATTCAAGTCAAAGAGG |
| | RQAATADVNTQGVLPGM | TCACGCAGAATGACGGTACGACGACGATT |
| | VWQDRDVYLQGPIWAKIP | GCCAATAACCTTACCAGCACGGTTCAGGT |
| | HTDGHFHPSPLMGGFGLK | GTTTACTGACTCGGAGTACCAGCTCCCGTA |
| | HPPPQILIKNTPVPANPSTT | CGTCCTCGGCTCGGCGCATCAAGGATGCCT |
| | FSAAKFASFITQYSTGQVS | CCCGCCGTTCCCAGCAGACGTCTTCATGGT |
| | VEIEWELQKENSKRWNPE | GCCACAGTATGGATACCTCACCCTGAACA |
| | IQYTSNYNKSVNVDFTVD | ACGGGAGTCAGGCAGTAGGACGCTCTTCA |
| | TNGVYSEPRPIGTRYLTRN | TTTTACTGCCTGGAGTACTTTCCTTCTCAG |
| | L* (SEQ ID NO: 11) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTAATGAATCCTCTCATCGACCAGTA |
| | | CCTGTATTACTTGAGCAGAACAAACACTCC |
| | | AAGTGGATTCTCTGGCTTGGAGAACACCA |
| | | CCACGCAGTCAAGGCTTCAGTTTTCTCAGG |
| | | CCGGAGCGAGTGACATTCGGGACCAGTCT |
| | | AGGAACTGGCTTCCTGGACCCTGTTACCGC |
| | | CAGCAGCGAGTATCAAAGACATCTGCGGA |
| | | TAACAACAACAGTGAATACTCGTGGACTG |
| | | GAGCTACCAAGTACCACCTCAATGGCAGA |
| | | GACTCTCTGGTGAATCCGGGCCCGGCCATG |
| | | GCAAGCCACAAGGACGATGAAGAAAAGTT |
| | | TTTTCCTCAGAGCGGGGTTCTCATCTTTGG |
| | | GAAGCAAGGCTCAGAGAAAACAAATGTGG |
| | | ACATTGAAAAGGTCATGATTACAGACGAA |
| | | GAGGAAATCAGGACAACCAATCCCGTGGC |
| | | TACGGAGCAGTATGGTTCTGTATCTACCAA |
| | | CCTCCAGAGAGGCAACAGACAAGCAGCTA |
| | | CCGCAGATGTCAACACACAAGGCGTTCTTC |
| | | CAGGCATGGTCTGGCAGGACAGAGATGTG |
| | | TACCTTCAGGGGCCCATCTGGGCAAAGATT |
| | | CCACACACGGACGGACATTTTCACCCCTCT |
| | | CCCCTCATGGGTGGATTCGGACTTAAACAC |
| | | CCTCCTCCACAGATTCTCATCAAGAACACC |
| | | CCGGTACCTGCGAATCCTTCGACCACCTTC |
| | | AGTGCGGCAAAGTTTGCTTCCTTCATCACA |
| | | CAGTACTCCACGGGACAGGTCAGCGTGGA |
| | | GATCGAGTGGGAGCTGCAGAAGGAAAACA |
| | | GCAAACGCTGGAATCCCGAAATTCAGTAC |
| | | ACTTCCAACTACAACAAGTCTGTTAATGTG |
| | | GACTTTACTGTGGACACTAATGGCGTGTAT |
| | | TCAGAGCCTCGCCCCATTGGCACCAGATAC |
| | | CTGACTCGTAATCTGTAA (SEQ ID NO: 56) |
| VAR-11 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNTPSGTTFSQAGASDIRD | CCGAACCTGGGCCCTGCCCACCTACAACA |
| | QSRNWLPGPCYRQQRVSK | ACCACCTCTACAAACAAATTTCCAGCCAAT |
| | TSADNNNSEYSWTGATK | CAGGAGCCTCGAACGACAATCACTACTTT |
| | YHLNGRDSLVNPGPAMAS | GGCTACAGCACCCCTTGGGGGTATTTTGAC |
| | HKDDEEKFFPQSGVLIFGK | TTCAACAGATTCCACTGCCACTTTTCACCA |
| | QGSEKTNVDIEKVMITDE | CGTGACTGGCAAAGACTCATCAACAACAA |
| | EEIRTTNPVATEQYGSVST | CTGGGGATTCCGACCCAAGAGACTCAACT |
| | NLQRGNRQAATADVNTQ | TCAAGCTCTTTAACATTCAAGTCAAAGAGG |
| | GVLPGMVWQDRDVYLQG | TCACGCAGAATGACGGTACGACGACGATT |
| | PIWAKIPHTDGHFHPSPLM | GCCAATAACCTTACCAGCACGGTTCAGGT |
| | GGFGLKHPPPQILIKNTPV | GTTTACTGACTCGGAGTACCAGCTCCCGTA |
| | PANPSTTFSAAKFASFITQ | CGTCCTCGGCTCGGCGCATCAAGGATGCCT |
| | YSTGQVSVEIEWELQKEN | CCCGCCGTTCCCAGCAGACGTCTTCATGGT |
| | SKRWNPEIQYTSNYNKSV | GCCACAGTATGGATACCTCACCCTGAACA |
| | NVDFTVDTNGVYSEPRPI | ACGGGAGTCAGGCAGTAGGACGCTCTTCA |
| | GTRYLTRNL* (SEQ ID NO: | TTTTACTGCCTGGAGTACTTTCCTTCTCAG |
| | 12) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CAGACTCATGAATCCTCTCATCGACCAGTA |
| | | CCTGTATTACTTGAGCAGAACAAACACTCC |
| | | AAGTGGAACCACCTTTTCTCAGGCCGGAG |
| | | CGAGTGACATTCGGGACCAGTCTAGGAAC |
| | | TGGCTTCCTGGACCCTGTTACCGCCAGCAG |
| | | CGAGTATCAAAGACATCTGCGGATAACAA |
| | | CAACAGTGAATACTCGTGGACTGGAGCTA |
| | | CCAAGTACCACCTCAATGGCAGAGACTCT |
| | | CTGGTGAATCCGGGCCCGGCCATGGCAAG |
| | | CCACAAGGACGATGAAGAAAAGTTTTTTC |
| | | CTCAGAGCGGGGTTCTCATCTTTGGGAAGC |
| | | AAGGCTCAGAGAAAACAAATGTGGACATT |
| | | GAAAAGGTCATGATTACAGACGAAGAGGA |
| | | AATCAGGACAACCAATCCCGTGGCTACGG |
| | | AGCAGTATGGTTCTGTATCTACCAACCTCC |
| | | AGAGAGGCAACAGACAAGCAGCTACCGCA |
| | | GATGTCAACACACAAGGCGTTCTTCCAGG |
| | | CATGGTCTGGCAGGACAGAGATGTGTACC |
| | | TTCAGGGGCCCATCTGGGCAAAGATTCCA |
| | | CACACGGACGGACATTTTCACCCCTCTCCC |
| | | CTCATGGGTGGATTCGGACTTAAACACCCT |
| | | CCTCCACAGATTCTCATCAAGAACACCCCG |
| | | GTACCTGCGAATCCTTCGACCACCTTCAGT |
| | | GCGGCAAAGTTTGCTTCCTTCATCACACAG |
| | | TACTCCACGGGACAGGTCAGCGTGGAGAT |
| | | CGAGTGGGAGCTGCAGAAGGAAAACAGCA |
| | | AACGCTGGAATCCCGAAATTCAGTACACTT |
| | | CCAACTACAACAAGTCTGTTAATGTGGACT |
| | | TTACTGTGGACACTAATGGCGTGTATTCAG |
| | | AGCCTCGCCCCATTGGCACCAGATACCTGA |
| | | CTCGTAATCTGTAA (SEQ ID NO: 57) |
| VAR-12 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TINGSGQNQQTLKFSVAG | CCGAACCTGGGCCCTGCCCACCTACAACA |
| | ASDIRDQSRNWLPGPCYR | ACCACCTCTACAAACAAATTTCCAGCCAAT |
| | QQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 13) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTGATGAATCCTCTCATCGACCAGTA |
| | | CCTGTATTACTTGAGCAGAACAATCAACG |
| | | GGAGTGGACAGAACCAGCAAACTCTTAAG |
| | | TTTTCTGTGGCCGGAGCGAGTGACATTCGG |
| | | GACCAGTCTAGGAACTGGCTTCCTGGACCC |
| | | TGTTACCGCCAGCAGCGAGTATCAAAGAC |
| | | ATCTGCGGATAACAACAACAGTGAATACT |
| | | CGTGGACTGGAGCTACCAAGTACCACCTC |
| | | AATGGCAGAGACTCTCTGGTGAATCCGGG |
| | | CCCGGCCATGGCAAGCCACAAGGACGATG |
| | | AAGAAAAGTTTTTTCCTCAGAGCGGGGTTC |
| | | TCATCTTTGGGAAGCAAGGCTCAGAGAAA |
| | | ACAAATGTGGACATTGAAAAGGTCATGAT |
| | | TACAGACGAAGAGGAAATCAGGACAACCA |
| | | ATCCCGTGGCTACGGAGCAGTATGGTTCTG |
| | | TATCTACCAACCTCCAGAGAGGCAACAGA |
| | | CAAGCAGCTACCGCAGATGTCAACACACA |
| | | AGGCGTTCTTCCAGGCATGGTCTGGCAGG |
| | | ACAGAGATGTGTACCTTCAGGGGCCCATCT |
| | | GGGCAAAGATTCCACACACGGACGGACAT |
| | | TTTCACCCCTCTCCCCTCATGGGTGGATTC |
| | | GGACTTAAACACCCTCCTCCACAGATTCTC |
| | | ATCAAGAACACCCCGGTACCTGCGAATCC |
| | | TTCGACCACCTTCAGTGCGGCAAAGTTTGC |
| | | TTCCTTCATCACACAGTACTCCACGGGACA |
| | | GGTCAGCGTGGAGATCGAGTGGGAGCTGC |
| | | AGAAGGAAAACAGCAAACGCTGGAATCCC |
| | | GAAATTCAGTACACTTCCAACTACAACAA |
| | | GTCTGTTAATGTGGACTTTACTGTGGACAC |
| | | TAATGGCGTGTATTCAGAGCCTCGCCCCAT |
| | | TGGCACCAGATACCTGACTCGTAATCTGTA |
| | | A (SEQ ID NO: 58) |
| VAR-13 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLAR | ATGGGCGACAGAGTCATCACCACCAGCAC |
| | TQSTTGGTSGLQFSQAGA | CCGAACCTGGGCCCTGCCCACCTACAACA |
| | SDIRDQSRNWLPGPCYRQ | ACCACCTCTACAAACAAATTTCCAGCCAAT |
| | QRVSKTSADNNNSEYSWT | CAGGAGCCTCGAACGACAATCACTACTTT |
| | GATKYHLNGRDSLVNPGP | GGCTACAGCACCCCTTGGGGGTATTTTGAC |
| | AMASHKDDEEKFFPQSGV | TTCAACAGATTCCACTGCCACTTTTCACCA |
| | LIFGKQGSEKTNVDIEKV | CGTGACTGGCAAAGACTCATCAACAACAA |
| | MITDEEEIRTTNPVATEQY | CTGGGGATTCCGACCCAAGAGACTCAACT |
| | GSVSTNLQRGNRQAATAD | TCAAGCTCTTTAACATTCAAGTCAAAGAGG |
| | VNTQGVLPGMVWQDRDV | TCACGCAGAATGACGGTACGACGACGATT |
| | YLQGPIWAKIPHTDGHFH | GCCAATAACCTTACCAGCACGGTTCAGGT |
| | PSPLMGGFGLKHPPPQILI | GTTTACTGACTCGGAGTACCAGCTCCCGTA |
| | KNTPVPANPSTTFSAAKF | CGTCCTCGGCTCGGCGCATCAAGGATGCCT |
| | ASFITQYSTGQVSVEIEWE | CCCGCCGTTCCCAGCAGACGTCTTCATGGT |
| | LQKENSKRWNPEIQYTSN | GCCACAGTATGGATACCTCACCCTGAACA |
| | YNKSVNVDFTVDTNGVY | ACGGGAGTCAGGCAGTAGGACGCTCTTCA |
| | SEPRPIGTRYLTRNL* (SEQ | TTTTACTGCCTGGAGTACTTTCCTTCTCAG |
| | ID NO: 14) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTCTTATGAATCCTCTCATCGACCAGTA |
| | | CCTGTATTACTTGGCTAGAACTCAAAGTAC |
| | | AACCGGGGGTACCTCAGGGCTTCAGTTTTC |
| | | TCAGGCCGGAGCGAGTGACATTCGGGACC |
| | | AGTCTAGGAACTGGCTTCCTGGACCCTGTT |
| | | ACCGCCAGCAGCGAGTATCAAAGACATCT |
| | | GCGGATAACAACAACAGTGAATACTCGTG |
| | | GACTGGAGCTACCAAGTACCACCTCAATG |
| | | GCAGAGACTCTCTGGTGAATCCGGGCCCG |
| | | GCCATGGCAAGCCACAAGGACGATGAAGA |
| | | AAAGTTTTTTCCTCAGAGCGGGGTTCTCAT |
| | | CTTTGGGAAGCAAGGCTCAGAGAAAACAA |
| | | ATGTGGACATTGAAAAGGTCATGATTACA |
| | | GACGAAGAGGAAATCAGGACAACCAATCC |
| | | CGTGGCTACGGAGCAGTATGGTTCTGTATC |
| | | TACCAACCTCCAGAGAGGCAACAGACAAG |
| | | CAGCTACCGCAGATGTCAACACACAAGGC |
| | | GTTCTTCCAGGCATGGTCTGGCAGGACAG |
| | | AGATGTGTACCTTCAGGGGCCCATCTGGGC |
| | | AAAGATTCCACACACGGACGGACATTTTC |
| | | ACCCCTCTCCCCTCATGGGTGGATTCGGAC |
| | | TTAAACACCCTCCTCCACAGATTCTCATCA |
| | | AGAACACCCCGGTACCTGCGAATCCTTCG |
| | | ACCACCTTCAGTGCGGCAAAGTTTGCTTCC |
| | | TTCATCACACAGTACTCCACGGGACAGGTC |
| | | AGCGTGGAGATCGAGTGGGAGCTGCAGAA |
| | | GGAAAACAGCAAACGCTGGAATCCCGAAA |
| | | TTCAGTACACTTCCAACTACAACAAGTCTG |
| | | TTAATGTGGACTTTACTGTGGACACTAATG |
| | | GCGTGTATTCAGAGCCTCGCCCCATTGGCA |
| | | CCAGATACCTGACTCGTAATCTGTAA (SEQ |
| | | ID NO: 59) |
| VAR-14 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TQSPYGGTATFMDLQFSQ | CCGAACCTGGGCCCTGCCCACCTACAACA |
| | AGASDIRDQSRNWLPGPC | ACCACCTCTACAAACAAATTTCCAGCCAAT |
| | YRQQRVSKTSADNNNSEY | CAGGAGCCTCGAACGACAATCACTACTTT |
| | SWTGATKYHLNGRDSLV | GGCTACAGCACCCCTTGGGGGTATTTTGAC |
| | NPGPAMASHKDDEEKFFP | TTCAACAGATTCCACTGCCACTTTTCACCA |
| | QSGVLIFGKQGSEKTNVDI | CGTGACTGGCAAAGACTCATCAACAACAA |
| | EKVMITDEEEIRTTNPVAT | CTGGGGATTCCGACCCAAGAGACTCAACT |
| | EQYGSVSTNLQRGNRQAA | TCAAGCTCTTTAACATTCAAGTCAAAGAGG |
| | TADVNTQGVLPGMVWQD | TCACGCAGAATGACGGTACGACGACGATT |
| | RDVYLQGPIWAKIPHTDG | GCCAATAACCTTACCAGCACGGTTCAGGT |
| | HFHPSPLMGGFGLKHPPP | GTTTACTGACTCGGAGTACCAGCTCCCGTA |
| | QILIKNTPVPANPSTTFSAA | CGTCCTCGGCTCGGCGCATCAAGGATGCCT |
| | KFASFITQYSTGQVSVEIE | CCCGCCGTTCCCAGCAGACGTCTTCATGGT |
| | WELQKENSKRWNPEIQYT | GCCACAGTATGGATACCTCACCCTGAACA |
| | SNYNKSVNVDFTVDTNG | ACGGGAGTCAGGCAGTAGGACGCTCTTCA |
| | VYSEPRPIGTRYLTRNL* | TTTTACTGCCTGGAGTACTTTCCTTCTCAG |
| | (SEQ ID NO: 15) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGCCTCATGAATCCTCTCATCGACCAGTA |
| | | CCTGTATTACTTGAGCAGAACACAATCGCC |
| | | ATACGGGGGAACCGCCACGTTCATGGACC |
| | | TTCAGTTTTCTCAGGCCGGAGCGAGTGACA |
| | | TTCGGGACCAGTCTAGGAACTGGCTTCCTG |
| | | GACCCTGTTACCGCCAGCAGCGAGTATCA |
| | | AAGACATCTGCGGATAACAACAACAGTGA |
| | | ATACTCGTGGACTGGAGCTACCAAGTACC |
| | | ACCTCAATGGCAGAGACTCTCTGGTGAATC |
| | | CGGGCCCGGCCATGGCAAGCCACAAGGAC |
| | | GATGAAGAAAAGTTTTTTCCTCAGAGCGG |
| | | GGTTCTCATCTTTGGGAAGCAAGGCTCAGA |
| | | GAAAACAAATGTGGACATTGAAAAGGTCA |
| | | TGATTACAGACGAAGAGGAAATCAGGACA |
| | | ACCAATCCCGTGGCTACGGAGCAGTATGG |
| | | TTCTGTATCTACCAACCTCCAGAGAGGCAA |
| | | CAGACAAGCAGCTACCGCAGATGTCAACA |
| | | CACAAGGCGTTCTTCCAGGCATGGTCTGGC |
| | | AGGACAGAGATGTGTACCTTCAGGGGCCC |
| | | ATCTGGGCAAAGATTCCACACACGGACGG |
| | | ACATTTTCACCCCTCTCCCCTCATGGGTGG |
| | | ATTCGGACTTAAACACCCTCCTCCACAGAT |
| | | TCTCATCAAGAACACCCCGGTACCTGCGA |
| | | ATCCTTCGACCACCTTCAGTGCGGCAAAGT |
| | | TTGCTTCCTTCATCACACAGTACTCCACGG |
| | | GACAGGTCAGCGTGGAGATCGAGTGGGAG |
| | | CTGCAGAAGGAAAACAGCAAACGCTGGAA |
| | | TCCCGAAATTCAGTACACTTCCAACTACAA |
| | | CAAGTCTGTTAATGTGGACTTTACTGTGGA |
| | | CACTAATGGCGTGTATTCAGAGCCTCGCCC |
| | | CATTGGCACCAGATACCTGACTCGTAATCT |
| | | GTAA (SEQ ID NO: 60) |
| VAR-15 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | HETEFNFTNTPSGTTTQSR | CCGAACCTGGGCCCTGCCCACCTACAACA |
| | LQFSQAGASDIRDQSRNW | ACCACCTCTACAAACAAATTTCCAGCCAAT |
| | LPGPCYRQQRVSKTSADN | CAGGAGCCTCGAACGACAATCACTACTTT |
| | NNSEYSWTGATKYHLNG | GGCTACAGCACCCCTTGGGGGTATTTTGAC |
| | RDSLVNPGPAMASHKDDE | TTCAACAGATTCCACTGCCACTTTTCACCA |
| | EKFFPQSGVLIFGKQGSEK | CGTGACTGGCAAAGACTCATCAACAACAA |
| | TNVDIEKVMITDEEEIRTT | CTGGGGATTCCGACCCAAGAGACTCAACT |
| | NPVATEQYGSVSTNLQRG | TCAAGCTCTTTAACATTCAAGTCAAAGAGG |
| | NRQAATADVNTQGVLPG | TCACGCAGAATGACGGTACGACGACGATT |
| | MVWQDRDVYLQGPIWAK | GCCAATAACCTTACCAGCACGGTTCAGGT |
| | IPHTDGHFHPSPLMGGFGL | GTTTACTGACTCGGAGTACCAGCTCCCGTA |
| | KHPPPQILIKNTPVPANPST | CGTCCTCGGCTCGGCGCATCAAGGATGCCT |
| | TFSAAKFASFITQYSTGQV | CCCGCCGTTCCCAGCAGACGTCTTCATGGT |
| | SVEIEWELQKENSKRWNP | GCCACAGTATGGATACCTCACCCTGAACA |
| | EIQYTSNYNKSVNVDFTV | ACGGGAGTCAGGCAGTAGGACGCTCTTCA |
| | DTNGVYSEPRPIGTRYLTR | TTTTACTGCCTGGAGTACTTTCCTTCTCAG |
| | NL* (SEQ ID NO: 16) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGTTTGATGAATCCTCTCATCGACCAGTA |
| | | CCTGTATTACTTGAGCAGACACGAAACCG |
| | | AATTCAATTTTACAAACACTCCAAGTGGAA |
| | | CCACCACGCAGTCAAGGCTTCAGTTTTCTC |
| | | AGGCCGGAGCGAGTGACATTCGGGACCAG |
| | | TCTAGGAACTGGCTTCCTGGACCCTGTTAC |
| | | CGCCAGCAGCGAGTATCAAAGACATCTGC |
| | | GGATAACAACAACAGTGAATACTCGTGGA |
| | | CTGGAGCTACCAAGTACCACCTCAATGGC |
| | | AGAGACTCTCTGGTGAATCCGGGCCCGGC |
| | | CATGGCAAGCCACAAGGACGATGAAGAAA |
| | | AGTTTTTTCCTCAGAGCGGGGTTCTCATCT |
| | | TTGGGAAGCAAGGCTCAGAGAAAACAAAT |
| | | GTGGACATTGAAAAGGTCATGATTACAGA |
| | | CGAAGAGGAAATCAGGACAACCAATCCCG |
| | | TGGCTACGGAGCAGTATGGTTCTGTATCTA |
| | | CCAACCTCCAGAGAGGCAACAGACAAGCA |
| | | GCTACCGCAGATGTCAACACACAAGGCGT |
| | | TCTTCCAGGCATGGTCTGGCAGGACAGAG |
| | | ATGTGTACCTTCAGGGGCCCATCTGGGCAA |
| | | AGATTCCACACACGGACGGACATTTTCACC |
| | | CCTCTCCCCTCATGGGTGGATTCGGACTTA |
| | | AACACCCTCCTCCACAGATTCTCATCAAGA |
| | | ACACCCCGGTACCTGCGAATCCTTCGACCA |
| | | CCTTCAGTGCGGCAAAGTTTGCTTCCTTCA |
| | | TCACACAGTACTCCACGGGACAGGTCAGC |
| | | GTGGAGATCGAGTGGGAGCTGCAGAAGGA |
| | | AAACAGCAAACGCTGGAATCCCGAAATTC |
| | | AGTACACTTCCAACTACAACAAGTCTGTTA |
| | | ATGTGGACTTTACTGTGGACACTAATGGCG |
| | | TGTATTCAGAGCCTCGCCCCATTGGCACCA |
| | | GATACCTGACTCGTAATCTGTAA (SEQ ID |
| | | NO: 61) |
| VAR-16 | MAADGYLPDWLEDTLSE | ATGGCTGCCGATGGTTATCTTCCAGATTGG |
| | GIRQWWKLKPGPPPPKPA | CTCGAGGACACTCTCTCTGAAGGAATAAG |
| | ERHKDDSRGLVLPGYKYL | ACAGTGGTGGAAGCTCAAACCTGGCCCAC |
| | GPFNGLDKGEPVNEADAA | CACCACCAAAGCCCGCAGAGCGGCATAAG |
| | ALEHDKAYDRQLDSGDN | GACGACAGCAGGGGTCTTGTGCTTCCTGG |
| | PYLKYNHADAEFQERLKE | GTACAAGTACCTCGGACCCTTCAACGGACT |
| | DTSFGGNLGRAVFQAKKR | CGACAAGGGAGAGCCGGTCAACGAGGCAG |
| | VLEPLGLVEEPVKTAPGK | ACGCCGCGGCCCTCGAGCACGACAAAGCC |
| | KRPVEHSPVEPDSSSGTGK | TACGACCGGCAGCTCGACAGCGGAGACAA |
| | AGQQPARKRLNFGQTGD | CCCGTACCTCAAGTACAACCACGCCGACG |
| | ADSVPDPQPLGQPPAAPS | CGGAGTTTCAGGAGCGCCTTAAAGAAGAT |
| | GLGTNTMATGSGAPMAD | ACGTCTTTTGGGGGCAACCTCGGACGAGC |
| | NNEGADGVGNSSGNWHC | AGTCTTCCAGGCGAAAAAGAGGGTTCTTG |
| | DSTWMGDRVITTSTRTWA | AACCTCTGGGCCTGGTTGAGGAACCTGTTA |
| | LPTYNNHLYKQISSQSGAS | AGACGGCTCCGGGAAAAAAGAGGCCGGTA |
| | NDNHYFGYSTPWGYFDF | GAGCACTCTCCTGTGGAGCCAGACTCCTCC |
| | NRFHCHFSPRDWQRLINN | TCGGGAACCGGAAAGGCGGGCCAGCAGCC |
| | NWGFRPKRLNFKLFNIQV | TGCAAGAAAAAGATTGAATTTTGGTCAGA |
| | KEVTQNDGTTTIANNLTS | CTGGAGACGCAGACTCAGTACCTGACCCC |
| | TVQVFTDSEYQLPYVLGS | CAGCCTCTCGGACAGCCACCAGCAGCCCC |
| | AHQGCLPPFPADVFMVPQ | CTCTGGTCTGGGAACTAATACGATGGCTAC |
| | YGYLTLNNGSQAVGRSSF | AGGCAGTGGCGCACCAATGGCAGACAATA |
| | YCLEYFPSQMLRTGNNFT | ACGAGGGCGCCGACGGAGTGGGTAATTCC |
| | FSYTFEDVPFHSSYAHSQS | TCGGGAAATTGGCATTGCGATTCCACATGG |
| | LDRLMNPLIDQYLYYLSR | ATGGGCGACAGAGTCATCACCACCAGCAC |
| | TNTTSGTGGTQTLAFSQA | CCGAACCTGGGCCCTGCCCACCTACAACA |
| | GASDIRDQSRNWLPGPCY | ACCACCTCTACAAACAAATTTCCAGCCAAT |
| | RQQRVSKTSADNNNSEYS | CAGGAGCCTCGAACGACAATCACTACTTT |
| | WTGATKYHLNGRDSLVN | GGCTACAGCACCCCTTGGGGGTATTTTGAC |
| | PGPAMASHKDDEEKFFPQ | TTCAACAGATTCCACTGCCACTTTTCACCA |
| | SGVLIFGKQGSEKTNVDIE | CGTGACTGGCAAAGACTCATCAACAACAA |
| | KVMITDEEEIRTTNPVATE | CTGGGGATTCCGACCCAAGAGACTCAACT |
| | QYGSVSTNLQRGNRQAAT | TCAAGCTCTTTAACATTCAAGTCAAAGAGG |
| | ADVNTQGVLPGMVWQDR | TCACGCAGAATGACGGTACGACGACGATT |
| | DVYLQGPIWAKIPHTDGH | GCCAATAACCTTACCAGCACGGTTCAGGT |
| | FHPSPLMGGFGLKHPPPQI | GTTTACTGACTCGGAGTACCAGCTCCCGTA |
| | LIKNTPVPANPSTTFSAAK | CGTCCTCGGCTCGGCGCATCAAGGATGCCT |
| | FASFITQYSTGQVSVEIEW | CCCGCCGTTCCCAGCAGACGTCTTCATGGT |
| | ELQKENSKRWNPEIQYTS | GCCACAGTATGGATACCTCACCCTGAACA |
| | NYNKSVNVDFTVDTNGV | ACGGGAGTCAGGCAGTAGGACGCTCTTCA |
| | YSEPRPIGTRYLTRNL* | TTTTACTGCCTGGAGTACTTTCCTTCTCAG |
| | (SEQ ID NO: 17) | ATGCTGCGTACCGGAAACAACTTTACCTTC |
| | | AGCTACACTTTTGAGGACGTTCCTTTCCAC |
| | | AGCAGCTACGCTCACAGCCAGAGTCTGGA |
| | | CCGACTCATGAATCCTCTCATCGACCAGTA |
| | | CCTGTATTACTTGAGCAGAACAAACACTAC |
| | | AAGTGGAACCGGAGGAACACAGACGCTTG |
| | | CTTTTTCTCAGGCCGGAGCGAGTGACATTC |
| | | GGGACCAGTCTAGGAACTGGCTTCCTGGA |
| | | CCCTGTTACCGCCAGCAGCGAGTATCAAA |
| | | GACATCTGCGGATAACAACAACAGTGAAT |
| | | ACTCGTGGACTGGAGCTACCAAGTACCAC |
| | | CTCAATGGCAGAGACTCTCTGGTGAATCCG |
| | | GGCCCGGCCATGGCAAGCCACAAGGACGA |
| | | TGAAGAAAAGTTTTTTCCTCAGAGCGGGGT |
| | | TCTCATCTTTGGGAAGCAAGGCTCAGAGA |
| | | AAACAAATGTGGACATTGAAAAGGTCATG |
| | | ATTACAGACGAAGAGGAAATCAGGACAAC |
| | | CAATCCCGTGGCTACGGAGCAGTATGGTTC |
| | | TGTATCTACCAACCTCCAGAGAGGCAACA |
| | | GACAAGCAGCTACCGCAGATGTCAACACA |
| | | CAAGGCGTTCTTCCAGGCATGGTCTGGCAG |
| | | GACAGAGATGTGTACCTTCAGGGGCCCAT |
| | | CTGGGCAAAGATTCCACACACGGACGGAC |
| | | ATTTTCACCCCTCTCCCCTCATGGGTGGAT |
| | | TCGGACTTAAACACCCTCCTCCACAGATTC |
| | | TCATCAAGAACACCCCGGTACCTGCGAAT |
| | | CCTTCGACCACCTTCAGTGCGGCAAAGTTT |
| | | GCTTCCTTCATCACACAGTACTCCACGGGA |
| | | CAGGTCAGCGTGGAGATCGAGTGGGAGCT |
| | | GCAGAAGGAAAACAGCAAACGCTGGAATC |
| | | CCGAAATTCAGTACACTTCCAACTACAACA |
| | | AGTCTGTTAATGTGGACTTTACTGTGGACA |
| | | CTAATGGCGTGTATTCAGAGCCTCGCCCCA |
| | | TTGGCACCAGATACCTGACTCGTAATCTGT |
| | | AA (SEQ ID NO: 62) |

TABLE 4

| | Amino Acid Sequence of | |
|---|---|---|
| | VP1 capsid polypeptide | |
| | (SEQ ID NO; starting | |
| | amino acid of VP2 is | |
| Capsid | underlined; starting amino | Exemplary Nucleic Acid Molecule Sequence(SEQ ID |
| Variant | acid of VP3 is in bold. | NO) |
| VAR-17 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | FALMEPNLQRGNRQA | TCTTCATTTTACTGCCTGGAGTACTTTCCTTCT |
| | ATADVNTQGVLPGMV | CAGATGCTGCGTACCGGAAACAACTTTACCTT |
| | WQDRDVYLQGPIWAK | CAGCTACACTTTTGAGGACGTTCCTTTCCACA |
| | IPHTDGHFHPSPLMGG | GCAGCTACGCTCACAGCCAGAGTCTGGACCGT |
| | FGLKHPPPQILIKNTPV | CTCATGAATCCTCTCATCGACCAGTACCTGTA |
| | PANPSTTFSAAKFASFI | TTACTTGAGCAGAACAAACACTCCAAGTGGA |
| | TQYSTGQVSVEIEWEL | ACCACCACGCAGTCAAGGCTTCAGTTTTCTCA |
| | QKENSKRWNPEIQYTS | GGCCGGAGCGAGTGACATTCGGGACCAGTCT |
| | NYNKSVNVDFTVDTN | AGGAACTGGCTTCCTGGACCCTGTTACCGCCA |
| | GVYSEPRPIGTRYLTR | GCAGCGAGTATCAAAGACATCTGCGGATAAC |
| | NL* (SEQ ID NO: 18) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCTTTGCCCTCATGGAGCCGAACCTCCA |
| | | GAGAGGCAACAGACAAGCAGCTACCGCAGAT |
| | | GTCAACACACAAGGCGTTCTTCCAGGCATGGT |
| | | CTGGCAGGACAGAGATGTGTACCTTCAGGGG |
| | | CCCATCTGGGCAAAGATTCCACACACGGACG |
| | | GACATTTTCACCCCTCTCCCCTCATGGGTGGA |
| | | TTCGGACTTAAACACCCTCCTCCACAGATTCT |
| | | CATCAAGAACACCCCGGTACCTGCGAATCCTT |
| | | CGACCACCTTCAGTGCGGCAAAGTTTGCTTCC |
| | | TTCATCACACAGTACTCCACGGGACAGGTCAG |
| | | CGTGGAGATCGAGTGGGAGCTGCAGAAGGAA |
| | | AACAGCAAACGCTGGAATCCCGAAATTCAGT |
| | | ACACTTCCAACTACAACAAGTCTGTTAATGTG |
| | | GACTTTACTGTGGACACTAATGGCGTGTATTC |
| | | AGAGCCTCGCCCCATTGGCACCAGATACCTGA |
| | | CTCGTAATCTGTAA (SEQ ID NO: 63) |
| VAR-18 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRAYNPDRGNRQA | TCTTCATTTTACTGCCTGGAGTACTTTCCTTCT |
| | ATADVNTQGVLPGMV | CAGATGCTGCGTACCGGAAACAACTTTACCTT |
| | WQDRDVYLQGPIWAK | CAGCTACACTTTTGAGGACGTTCCTTTCCACA |
| | IPHTDGHFHPSPLMGG | GCAGCTACGCTCACAGCCAGAGTCTGGACCGT |
| | FGLKHPPPQILIKNTPV | CTCATGAATCCTCTCATCGACCAGTACCTGTA |
| | PANPSTTFSAAKFASFI | TTACTTGAGCAGAACAAACACTCCAAGTGGA |
| | TQYSTGQVSVEIEWEL | ACCACCACGCAGTCAAGGCTTCAGTTTTCTCA |
| | QKENSKRWNPEIQYTS | GGCCGGAGCGAGTGACATTCGGGACCAGTCT |
| | NYNKSVNVDFTVDTN | AGGAACTGGCTTCCTGGACCCTGTTACCGCCA |
| | GVYSEPRPIGTRYLTR | GCAGCGAGTATCAAAGACATCTGCGGATAAC |
| | NL* (SEQ ID NO: 19) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGCGTGCGTACAATCCTGA |
| | | TAGAGGCAACAGACAAGCAGCTACCGCAGAT |
| | | GTCAACACACAAGGCGTTCTTCCAGGCATGGT |
| | | CTGGCAGGACAGAGATGTGTACCTTCAGGGG |
| | | CCCATCTGGGCAAAGATTCCACACACGGACG |
| | | GACATTTTCACCCCTCTCCCCTCATGGGTGGA |
| | | TTCGGACTTAAACACCCTCCTCCACAGATTCT |
| | | CATCAAGAACACCCCGGTACCTGCGAATCCTT |
| | | CGACCACCTTCAGTGCGGCAAAGTTTGCTTCC |
| | | TTCATCACACAGTACTCCACGGGACAGGTCAG |
| | | CGTGGAGATCGAGTGGGAGCTGCAGAAGGAA |
| | | AACAGCAAACGCTGGAATCCCGAAATTCAGT |
| | | ACACTTCCAACTACAACAAGTCTGTTAATGTG |
| | | GACTTTACTGTGGACACTAATGGCGTGTATTC |
| | | AGAGCCTCGCCCCATTGGCACCAGATACCTGA |
| | | CTCGTAATCTGTAA (SEQ ID NO: 64) |
| VAR-19 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIAKVMITSEEEI | GTCTTCATGGTGCCACAGTATGGATACCTCAC |
| | KTTNPVATEQYGVVA | CCTGAACAACGGGAGTCAGGCAGTAGGACGC |
| | ANLQGNRQAATADVN | TCTTCATTTTACTGCCTGGAGTACTTTCCTTCT |
| | TQGVLPGMVWQDRD | CAGATGCTGCGTACCGGAAACAACTTTACCTT |
| | VYLQGPIWAKIPHTDG | CAGCTACACTTTTGAGGACGTTCCTTTCCACA |
| | HFHPSPLMGGFGLKHP | GCAGCTACGCTCACAGCCAGAGTCTGGACCGT |
| | PPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 20) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGCAAAGGTCATG |
| | | ATTACATCCGAAGAGGAAATCAAGACAACCA |
| | | ATCCCGTGGCTACGGAGCAGTATGGTGTGGTA |
| | | GCTGCTAACCTCCAGGGCAACAGACAAGCAG |
| | | CTACCGCAGATGTCAACACACAAGGCGTTCTT |
| | | CCAGGCATGGTCTGGCAGGACAGAGATGTGT |
| | | ACCTTCAGGGGCCCATCTGGGCAAAGATTCCA |
| | | CACACGGACGGACATTTTCACCCCTCTCCCCT |
| | | CATGGGTGGATTCGGACTTAAACACCCTCCTC |
| | | CACAGATTCTCATCAAGAACACCCCGGTACCT |
| | | GCGAATCCTTCGACCACCTTCAGTGCGGCAAA |
| | | GTTTGCTTCCTTCATCACACAGTACTCCACGG |
| | | GACAGGTCAGCGTGGAGATCGAGTGGGAGCT |
| | | GCAGAAGGAAAACAGCAAACGCTGGAATCCC |
| | | GAAATTCAGTACACTTCCAACTACAACAAGTC |
| | | TGTTAATGTGGACTTTACTGTGGACACTAATG |
| | | GCGTGTATTCAGAGCCTCGCCCCATTGGCACC |
| | | AGATACCTGACTCGTAATCTGTAA (SEQ ID |
| | | NO: 65) |
| VAR-20 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKLMITDEEEI | GTCTTCATGGTGCCACAGTATGGATACCTCAC |
| | RTTNPVATEQYGDVST | CCTGAACAACGGGAGTCAGGCAGTAGGACGC |
| | NLQRGNRQAATADVN | TCTTCATTTTACTGCCTGGAGTACTTTCCTTCT |
| | TQGVLPGMVWQDRD | CAGATGCTGCGTACCGGAAACAACTTTACCTT |
| | VYLQGPIWAKIPHTDG | CAGCTACACTTTTGAGGACGTTCCTTTCCACA |
| | HFHPSPLMGGFGLKHP | GCAGCTACGCTCACAGCCAGAGTCTGGACCGT |
| | PPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 21) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGCTCATG |
| | | ATTACAGACGAAGAGGAAATCAGGACAACCA |
| | | ATCCCGTGGCTACGGAGCAGTATGGTGATGTA |
| | | TCTACCAACCTCCAGAGAGGCAACAGACAAG |
| | | CAGCTACCGCAGATGTCAACACACAAGGCGT |
| | | TCTTCCAGGCATGGTCTGGCAGGACAGAGATG |
| | | TGTACCTTCAGGGGCCCATCTGGGCAAAGATT |
| | | CCACACACGGACGGACATTTTCACCCCTCTCC |
| | | CCTCATGGGTGGATTCGGACTTAAACACCCTC |
| | | CTCCACAGATTCTCATCAAGAACACCCCGGTA |
| | | CCTGCGAATCCTTCGACCACCTTCAGTGCGGC |
| | | AAAGTTTGCTTCCTTCATCACACAGTACTCCA |
| | | CGGGACAGGTCAGCGTGGAGATCGAGTGGGA |
| | | GCTGCAGAAGGAAAACAGCAAACGCTGGAAT |
| | | CCCGAAATTCAGTACACTTCCAACTACAACAA |
| | | GTCTGTTAATGTGGACTTTACTGTGGACACTA |
| | | ATGGCGTGTATTCAGAGCCTCGCCCCATTGGC |
| | | ACCAGATACCTGACTCGTAATCTGTAA (SEQ |
| | | ID NO: 66) |
| VAR-21 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIENVLITDEEEI | GTCTTCATGGTGCCACAGTATGGATACCTCAC |
| | RTTNPVATEQYGIVAT | CCTGAACAACGGGAGTCAGGCAGTAGGACGC |
| | NLQRGNRQAATADVN | TCTTCATTTTACTGCCTGGAGTACTTTCCTTCT |
| | TQGVLPGMVWQDRD | CAGATGCTGCGTACCGGAAACAACTTTACCTT |
| | VYLQGPIWAKIPHTDG | CAGCTACACTTTTGAGGACGTTCCTTTCCACA |
| | HFHPSPLMGGFGLKHP | GCAGCTACGCTCACAGCCAGAGTCTGGACCGT |
| | PPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 22) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAACGTCCTC |
| | | ATTACAGACGAAGAGGAAATCAGGACAACCA |
| | | ATCCCGTGGCTACGGAGCAGTATGGTATTGTA |
| | | GCTACCAACCTCCAGAGAGGCAACAGACAAG |
| | | CAGCTACCGCAGATGTCAACACACAAGGCGT |
| | | TCTTCCAGGCATGGTCTGGCAGGACAGAGATG |
| | | TGTACCTTCAGGGGCCCATCTGGGCAAAGATT |
| | | CCACACACGGACGGACATTTTCACCCCTCTCC |
| | | CCTCATGGGTGGATTCGGACTTAAACACCCTC |
| | | CTCCACAGATTCTCATCAAGAACACCCCGGTA |
| | | CCTGCGAATCCTTCGACCACCTTCAGTGCGGC |
| | | AAAGTTTGCTTCCTTCATCACACAGTACTCCA |
| | | CGGGACAGGTCAGCGTGGAGATCGAGTGGGA |
| | | GCTGCAGAAGGAAAACAGCAAACGCTGGAAT |
| | | CCCGAAATTCAGTACACTTCCAACTACAACAA |
| | | GTCTGTTAATGTGGACTTTACTGTGGACACTA |
| | | ATGGCGTGTATTCAGAGCCTCGCCCCATTGGC |
| | | ACCAGATACCTGACTCGTAATCTGTAA (SEQ |
| | | ID NO: 67) |
| VAR-22 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDLERVMLTSEEE | GTCTTCATGGTGCCACAGTATGGATACCTCAC |
| | IATTNPVATEQYGSVS | CCTGAACAACGGGAGTCAGGCAGTAGGACGC |
| | DNLQSRSRQAATADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 23) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACCTCGAACGTGTCATG |
| | | CTTACATCGGAAGAGGAAATCGCCACAACCA |
| | | ATCCCGTGGCTACGGAGCAGTATGGTTCTGTA |
| | | TCTGACAACCTCCAGTCGAGATCAAGACAAG |
| | | CAGCTACCGCAGATGTCAACACACAAGGCGT |
| | | TCTTCCAGGCATGGTCTGGCAGGACAGAGATG |
| | | TGTACCTTCAGGGGCCCATCTGGGCAAAGATT |
| | | CCACACACGGACGGACATTTTCACCCCTCTCC |
| | | CCTCATGGGTGGATTCGGACTTAAACACCCTC |
| | | CTCCACAGATTCTCATCAAGAACACCCCGGTA |
| | | CCTGCGAATCCTTCGACCACCTTCAGTGCGGC |
| | | AAAGTTTGCTTCCTTCATCACACAGTACTCCA |
| | | CGGGACAGGTCAGCGTGGAGATCGAGTGGGA |
| | | GCTGCAGAAGGAAAACAGCAAACGCTGGAAT |
| | | CCCGAAATTCAGTACACTTCCAACTACAACAA |
| | | GTCTGTTAATGTGGACTTTACTGTGGACACTA |
| | | ATGGCGTGTATTCAGAGCCTCGCCCCATTGGC |
| | | ACCAGATACCTGACTCGTAATCTGTAA (SEQ |
| | | ID NO: 68) |
| VAR-23 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNADISDVMLTSEEEI | GTCTTCATGGTGCCACAGTATGGATACCTCAC |
| | KTTNPVATEQYGIVSD | CCTGAACAACGGGAGTCAGGCAGTAGGACGC |
| | NLQRQNRQAATADVN | TCTTCATTTTACTGCCTGGAGTACTTTCCTTCT |
| | TQGVLPGMVWQDRD | CAGATGCTGCGTACCGGAAACAACTTTACCTT |
| | VYLQGPIWAKIPHTDG | CAGCTACACTTTTGAGGACGTTCCTTTCCACA |
| | HFHPSPLMGGFGLKHP | GCAGCTACGCTCACAGCCAGAGTCTGGACCGT |
| | PPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDINGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 24) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGCTGACATTTCTGACGTCATGC |
| | | TTACAAGCGAAGAGGAAATCAAAACAACCAA |
| | | TCCCGTGGCTACGGAGCAGTATGGTATTGTAT |
| | | CTGACAACCTCCAGAGACAGAACAGACAAGC |
| | | AGCTACCGCAGATGTCAACACACAAGGCGTT |
| | | CTTCCAGGCATGGTCTGGCAGGACAGAGATGT |
| | | GTACCTTCAGGGGCCCATCTGGGCAAAGATTC |
| | | CACACACGGACGGACATTTTCACCCCTCTCCC |
| | | CTCATGGGTGGATTCGGACTTAAACACCCTCC |
| | | TCCACAGATTCTCATCAAGAACACCCCGGTAC |
| | | CTGCGAATCCTTCGACCACCTTCAGTGCGGCA |
| | | AAGTTTGCTTCCTTCATCACACAGTACTCCAC |
| | | GGGACAGGTCAGCGTGGAGATCGAGTGGGAG |
| | | CTGCAGAAGGAAAACAGCAAACGCTGGAATC |
| | | CCGAAATTCAGTACACTTCCAACTACAACAAG |
| | | TCTGTTAATGTGGACTTTACTGTGGACACTAA |
| | | TGGCGTGTATTCAGAGCCTCGCCCCATTGGCA |
| | | CCAGATACCTGACTCGTAATCTGTAA (SEQ ID |
| | | NO: 69) |
| VAR-24 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEEYGAVST | CCTGAACAACGGGAGTCAGGCAGTAGGACGC |
| | NLQRGNRQAATADVN | TCTTCATTTTACTGCCTGGAGTACTTTCCTTCT |
| | TQGVLPGMVWQDRD | CAGATGCTGCGTACCGGAAACAACTTTACCTT |
| | VYLQGPIWAKIPHTDG | CAGCTACACTTTTGAGGACGTTCCTTTCCACA |
| | HFHPSPLMGGFGLKHP | GCAGCTACGCTCACAGCCAGAGTCTGGACCGT |
| | PPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 25) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGGAGTATGGTGCTGTA |
| | | TCTACCAACCTCCAGAGAGGCAACAGACAAG |
| | | CAGCTACCGCAGATGTCAACACACAAGGCGT |
| | | TCTTCCAGGCATGGTCTGGCAGGACAGAGATG |
| | | TGTACCTTCAGGGGCCCATCTGGGCAAAGATT |
| | | CCACACACGGACGGACATTTTCACCCCTCTCC |
| | | CCTCATGGGTGGATTCGGACTTAAACACCCTC |
| | | CTCCACAGATTCTCATCAAGAACACCCCGGTA |
| | | CCTGCGAATCCTTCGACCACCTTCAGTGCGGC |
| | | AAAGTTTGCTTCCTTCATCACACAGTACTCCA |
| | | CGGGACAGGTCAGCGTGGAGATCGAGTGGGA |
| | | GCTGCAGAAGGAAAACAGCAAACGCTGGAAT |
| | | CCCGAAATTCAGTACACTTCCAACTACAACAA |
| | | GTCTGTTAATGTGGACTTTACTGTGGACACTA |
| | | ATGGCGTGTATTCAGAGCCTCGCCCCATTGGC |
| | | ACCAGATACCTGACTCGTAATCTGTAA (SEQ |
| | | ID NO: 70) |
| VAR-25 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLLNWTAEQRGNRQA | TCTTCATTTTACTGCCTGGAGTACTTTCCTTCT |
| | ATADVNTQGVLPGMV | CAGATGCTGCGTACCGGAAACAACTTTACCTT |
| | WQDRDVYLQGPIWAK | CAGCTACACTTTTGAGGACGTTCCTTTCCACA |
| | IPHTDGHFHPSPLMGG | GCAGCTACGCTCACAGCCAGAGTCTGGACCGT |
| | FGLKHPPPQILIKNTPV | CTCATGAATCCTCTCATCGACCAGTACCTGTA |
| | PANPSTTFSAAKFASFI | TTACTTGAGCAGAACAAACACTCCAAGTGGA |
| | TQYSTGQVSVEIEWEL | ACCACCACGCAGTCAAGGCTTCAGTTTTCTCA |
| | QKENSKRWNPEIQYTS | GGCCGGAGCGAGTGACATTCGGGACCAGTCT |
| | NYNKSVNVDFTVDTN | AGGAACTGGCTTCCTGGACCCTGTTACCGCCA |
| | GVYSEPRPIGTRYLTR | GCAGCGAGTATCAAAGACATCTGCGGATAAC |
| | NL* (SEQ ID NO: 26) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCTCAATTGGACTGCCGAACA |
| | | GAGAGGCAACAGACAAGCAGCTACCGCAGAT |
| | | GTCAACACACAAGGCGTTCTTCCAGGCATGGT |
| | | CTGGCAGGACAGAGATGTGTACCTTCAGGGG |
| | | CCCATCTGGGCAAAGATTCCACACACGGACG |
| | | GACATTTTCACCCCTCTCCCCTCATGGGTGGA |
| | | TTCGGACTTAAACACCCTCCTCCACAGATTCT |
| | | CATCAAGAACACCCCGGTACCTGCGAATCCTT |
| | | CGACCACCTTCAGTGCGGCAAAGTTTGCTTCC |
| | | TTCATCACACAGTACTCCACGGGACAGGTCAG |
| | | CGTGGAGATCGAGTGGGAGCTGCAGAAGGAA |
| | | AACAGCAAACGCTGGAATCCCGAAATTCAGT |
| | | ACACTTCCAACTACAACAAGTCTGTTAATGTG |
| | | GACTTTACTGTGGACACTAATGGCGTGTATTC |
| | | AGAGCCTCGCCCCATTGGCACCAGATACCTGA |
| | | CTCGTAATCTGTAA (SEQ ID NO: 71) |
| VAR-26 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQSGNTNPITGDVNS | TCTTCATTTTACTGCCTGGAGTACTTTCCTTCT |
| | QGALPGMVWQDRDV | CAGATGCTGCGTACCGGAAACAACTTTACCTT |
| | YLQGPIWAKIPHTDGH | CAGCTACACTTTTGAGGACGTTCCTTTCCACA |
| | FHPSPLMGGFGLKHPP | GCAGCTACGCTCACAGCCAGAGTCTGGACCGT |
| | PQILIKNTPVPANPSTT | CTCATGAATCCTCTCATCGACCAGTACCTGTA |
| | FSAAKFASFITQYSTGQ | TTACTTGAGCAGAACAAACACTCCAAGTGGA |
| | VSVEIEWELQKENSKR | ACCACCACGCAGTCAAGGCTTCAGTTTTCTCA |
| | WNPEIQYTSNYNKSVN | GGCCGGAGCGAGTGACATTCGGGACCAGTCT |
| | VDFTVDTNGVYSEPRP | AGGAACTGGCTTCCTGGACCCTGTTACCGCCA |
| | IGTRYLTRNL* (SEQ ID | GCAGCGAGTATCAAAGACATCTGCGGATAAC |
| | NO: 27) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGTCTGGCAACACAAATCC |
| | | TATTACCGGAGATGTCAACTCGCAAGGCGCGC |
| | | TTCCAGGCATGGTCTGGCAGGACAGAGATGT |
| | | GTACCTTCAGGGGCCCATCTGGGCAAAGATTC |
| | | CACACACGGACGGACATTTTCACCCCTCTCCC |
| | | CTCATGGGTGGATTCGGACTTAAACACCCTCC |
| | | TCCACAGATTCTCATCAAGAACACCCCGGTAC |
| | | CTGCGAATCCTTCGACCACCTTCAGTGCGGCA |
| | | AAGTTTGCTTCCTTCATCACACAGTACTCCAC |
| | | GGGACAGGTCAGCGTGGAGATCGAGTGGGAG |
| | | CTGCAGAAGGAAAACAGCAAACGCTGGAATC |
| | | CCGAAATTCAGTACACTTCCAACTACAACAAG |
| | | TCTGTTAATGTGGACTTTACTGTGGACACTAA |
| | | TGGCGTGTATTCAGAGCCTCGCCCCATTGGCA |
| | | CCAGATACCTGACTCGTAATCTGTAA (SEQ ID |
| | | NO: 72) |
| VAR-27 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQLAKEFTTRNANRQ | TCTTCATTTTACTGCCTGGAGTACTTTCCTTCT |
| | AATADVNTQGVLPGM | CAGATGCTGCGTACCGGAAACAACTTTACCTT |
| | VWQDRDVYLQGPIWA | CAGCTACACTTTTGAGGACGTTCCTTTCCACA |
| | KIPHTDGHFHPSPLMG | GCAGCTACGCTCACAGCCAGAGTCTGGACCGT |
| | GFGLKHPPPQILIKNTP | CTCATGAATCCTCTCATCGACCAGTACCTGTA |
| | VPANPSTTFSAAKFAS | TTACTTGAGCAGAACAAACACTCCAAGTGGA |
| | FITQYSTGQVSVEIEWE | ACCACCACGCAGTCAAGGCTTCAGTTTTCTCA |
| | LQKENSKRWNPEIQYT | GGCCGGAGCGAGTGACATTCGGGACCAGTCT |
| | SNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 28) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGTTGGCGAAAGAATTCAC |
| | | TACGCGGAATGCGAACAGACAAGCAGCTACC |
| | | GCAGATGTCAACACACAAGGCGTTCTTCCAGG |
| | | CATGGTCTGGCAGGACAGAGATGTGTACCTTC |
| | | AGGGGCCCATCTGGGCAAAGATTCCACACAC |
| | | GGACGGACATTTTCACCCCTCTCCCCTCATGG |
| | | GTGGATTCGGACTTAAACACCCTCCTCCACAG |
| | | ATTCTCATCAAGAACACCCCGGTACCTGCGAA |
| | | TCCTTCGACCACCTTCAGTGCGGCAAAGTTTG |
| | | CTTCCTTCATCACACAGTACTCCACGGGACAG |
| | | GTCAGCGTGGAGATCGAGTGGGAGCTGCAGA |
| | | AGGAAAACAGCAAACGCTGGAATCCCGAAAT |
| | | TCAGTACACTTCCAACTACAACAAGTCTGTTA |
| | | ATGTGGACTTTACTGTGGACACTAATGGCGTG |
| | | TATTCAGAGCCTCGCCCCATTGGCACCAGATA |
| | | CCTGACTCGTAATCTGTAA (SEQ ID NO: 73) |
| VAR-28 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQAATLHPLE | TCTTCATTTTACTGCCTGGAGTACTTTCCTTCT |
| | ADVNTQGVLPGMVW | CAGATGCTGCGTACCGGAAACAACTTTACCTT |
| | QDRDVYLQGPIWAKIP | CAGCTACACTTTTGAGGACGTTCCTTTCCACA |
| | HTDGHFHPSPLMGGFG | GCAGCTACGCTCACAGCCAGAGTCTGGACCGT |
| | LKHPPPQILIKNTPVPA | CTCATGAATCCTCTCATCGACCAGTACCTGTA |
| | NPSTTFSAAKFASFITQ | TTACTTGAGCAGAACAAACACTCCAAGTGGA |
| | YSTGQVSVEIEWELQK | ACCACCACGCAGTCAAGGCTTCAGTTTTCTCA |
| | ENSKRWNPEIQYTSNY | GGCCGGAGCGAGTGACATTCGGGACCAGTCT |
| | NKSVNVDFTVDTNGV | AGGAACTGGCTTCCTGGACCCTGTTACCGCCA |
| | YSEPRPIGTRYLTRNL* | GCAGCGAGTATCAAAGACATCTGCGGATAAC |
| | (SEQ ID NO: 29) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAG |
| | | CAGCTACCTTACATCCGCTGGAGGCAGATGTC |
| | | AACACACAAGGCGTTCTTCCAGGCATGGTCTG |
| | | GCAGGACAGAGATGTGTACCTTCAGGGGCCC |
| | | ATCTGGGCAAAGATTCCACACACGGACGGAC |
| | | ATTTTCACCCCTCTCCCCTCATGGGTGGATTCG |
| | | GACTTAAACACCCTCCTCCACAGATTCTCATC |
| | | AAGAACACCCCGGTACCTGCGAATCCTTCGAC |
| | | CACCTTCAGTGCGGCAAAGTTTGCTTCCTTCA |
| | | TCACACAGTACTCCACGGGACAGGTCAGCGT |
| | | GGAGATCGAGTGGGAGCTGCAGAAGGAAAAC |
| | | AGCAAACGCTGGAATCCCGAAATTCAGTACA |
| | | CTTCCAACTACAACAAGTCTGTTAATGTGGAC |
| | | TTTACTGTGGACACTAATGGCGTGTATTCAGA |
| | | GCCTCGCCCCATTGGCACCAGATACCTGACTC |
| | | GTAATCTGTAA (SEQ ID NO: 74) |
| VAR-29 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRFGNRQAATADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 30) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGATTTGGCAACAGACA |
| | | AGCAGCTACCGCAGATGTCAACACACAAGGC |
| | | GTTCTTCCAGGCATGGTCTGGCAGGACAGAGA |
| | | TGTGTACCTTCAGGGGCCCATCTGGGCAAAGA |
| | | TTCCACACACGGACGGACATTTTCACCCCTCT |
| | | CCCCTCATGGGTGGATTCGGACTTAAACACCC |
| | | TCCTCCACAGATTCTCATCAAGAACACCCCGG |
| | | TACCTGCGAATCCTTCGACCACCTTCAGTGCG |
| | | GCAAAGTTTGCTTCCTTCATCACACAGTACTC |
| | | CACGGGACAGGTCAGCGTGGAGATCGAGTGG |
| | | GAGCTGCAGAAGGAAAACAGCAAACGCTGGA |
| | | ATCCCGAAATTCAGTACACTTCCAACTACAAC |
| | | AAGTCTGTTAATGTGGACTTTACTGTGGACAC |
| | | TAATGGCGTGTATTCAGAGCCTCGCCCCATTG |
| | | GCACCAGATACCTGACTCGTAATCTGTAA |
| | | (SEQ ID NO: 75) |
| VAR-30 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGDQDFKNRNRQ | TCTTCATTTTACTGCCTGGAGTACTTTCCTTCT |
| | AATADVNTQGVLPGM | CAGATGCTGCGTACCGGAAACAACTTTACCTT |
| | VWQDRDVYLQGPIWA | CAGCTACACTTTTGAGGACGTTCCTTTCCACA |
| | KIPHTDGHFHPSPLMG | GCAGCTACGCTCACAGCCAGAGTCTGGACCGT |
| | GFGLKHPPPQILIKNTP | CTCATGAATCCTCTCATCGACCAGTACCTGTA |
| | VPANPSTTFSAAKFAS | TTACTTGAGCAGAACAAACACTCCAAGTGGA |
| | FITQYSTGQVSVEIEWE | ACCACCACGCAGTCAAGGCTTCAGTTTTCTCA |
| | LQKENSKRWNPEIQYT | GGCCGGAGCGAGTGACATTCGGGACCAGTCT |
| | SNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 31) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCGACCAAGATTT |
| | | CAAAAACCGAAACAGACAAGCAGCTACCGCA |
| | | GATGTCAACACACAAGGCGTTCTTCCAGGCAT |
| | | GGTCTGGCAGGACAGAGATGTGTACCTTCAG |
| | | GGGCCCATCTGGGCAAAGATTCCACACACGG |
| | | ACGGACATTTTCACCCCTCTCCCCTCATGGGT |
| | | GGATTCGGACTTAAACACCCTCCTCCACAGAT |
| | | TCTCATCAAGAACACCCCGGTACCTGCGAATC |
| | | CTTCGACCACCTTCAGTGCGGCAAAGTTTGCT |
| | | TCCTTCATCACACAGTACTCCACGGGACAGGT |
| | | CAGCGTGGAGATCGAGTGGGAGCTGCAGAAG |
| | | GAAAACAGCAAACGCTGGAATCCCGAAATTC |
| | | AGTACACTTCCAACTACAACAAGTCTGTTAAT |
| | | GTGGACTTTACTGTGGACACTAATGGCGTGTA |
| | | TTCAGAGCCTCGCCCCATTGGCACCAGATACC |
| | | TGACTCGTAATCTGTAA (SEQ ID NO: 76) |
| VAR-31 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQIAATADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 32) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAA |
| | | TCGCAGCTACCGCAGATGTCAACACACAAGG |
| | | CGTTCTTCCAGGCATGGTCTGGCAGGACAGAG |
| | | ATGTGTACCTTCAGGGGCCCATCTGGGCAAAG |
| | | ATTCCACACACGGACGGACATTTTCACCCCTC |
| | | TCCCCTCATGGGTGGATTCGGACTTAAACACC |
| | | CTCCTCCACAGATTCTCATCAAGAACACCCCG |
| | | GTACCTGCGAATCCTTCGACCACCTTCAGTGC |
| | | GGCAAAGTTTGCTTCCTTCATCACACAGTACT |
| | | CCACGGGACAGGTCAGCGTGGAGATCGAGTG |
| | | GGAGCTGCAGAAGGAAAACAGCAAACGCTGG |
| | | AATCCCGAAATTCAGTACACTTCCAACTACAA |
| | | CAAGTCTGTTAATGTGGACTTTACTGTGGACA |
| | | CTAATGGCGTGTATTCAGAGCCTCGCCCCATT |
| | | GGCACCAGATACCTGACTCGTAATCTGTAA |
| | | (SEQ ID NO: 77) |
| VAR-32 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNLAIEQTRPAR | TCTTCATTTTACTGCCTGGAGTACTTTCCTTCT |
| | QAATADVNTQGVLPG | CAGATGCTGCGTACCGGAAACAACTTTACCTT |
| | MVWQDRDVYLQGPIW | CAGCTACACTTTTGAGGACGTTCCTTTCCACA |
| | AKIPHTDGHFHPSPLM | GCAGCTACGCTCACAGCCAGAGTCTGGACCGT |
| | GGFGLKHPPPQILIKNT | CTCATGAATCCTCTCATCGACCAGTACCTGTA |
| | PVPANPSTTFSAAKFA | TTACTTGAGCAGAACAAACACTCCAAGTGGA |
| | SFITQYSTGQVSVEIEW | ACCACCACGCAGTCAAGGCTTCAGTTTTCTCA |
| | ELQKENSKRWNPEIQY | GGCCGGAGCGAGTGACATTCGGGACCAGTCT |
| | TSNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 33) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACCTTGCTAT |
| | | CGAACAAACAAGACCAGCGAGACAAGCAGCT |
| | | ACCGCAGATGTCAACACACAAGGCGTTCTTCC |
| | | AGGCATGGTCTGGCAGGACAGAGATGTGTAC |
| | | CTTCAGGGGCCCATCTGGGCAAAGATTCCACA |
| | | CACGGACGGACATTTTCACCCCTCTCCCCTCA |
| | | TGGGTGGATTCGGACTTAAACACCCTCCTCCA |
| | | CAGATTCTCATCAAGAACACCCCGGTACCTGC |
| | | GAATCCTTCGACCACCTTCAGTGCGGCAAAGT |
| | | TTGCTTCCTTCATCACACAGTACTCCACGGGA |
| | | CAGGTCAGCGTGGAGATCGAGTGGGAGCTGC |
| | | AGAAGGAAAACAGCAAACGCTGGAATCCCGA |
| | | AATTCAGTACACTTCCAACTACAACAAGTCTG |
| | | TTAATGTGGACTTTACTGTGGACACTAATGGC |
| | | GTGTATTCAGAGCCTCGCCCCATTGGCACCAG |
| | | ATACCTGACTCGTAATCTGTAA (SEQ ID NO: |
| | | 78) |
| VAR-33 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRARLDETTRPARQ | TCTTCATTTTACTGCCTGGAGTACTTTCCTTCT |
| | AATADVNTQGVLPGM | CAGATGCTGCGTACCGGAAACAACTTTACCTT |
| | VWQDRDVYLQGPIWA | CAGCTACACTTTTGAGGACGTTCCTTTCCACA |
| | KIPHTDGHFHPSPLMG | GCAGCTACGCTCACAGCCAGAGTCTGGACCGT |
| | GFGLKHPPPQILIKNTP | CTCATGAATCCTCTCATCGACCAGTACCTGTA |
| | VPANPSTTFSAAKFAS | TTACTTGAGCAGAACAAACACTCCAAGTGGA |
| | FITQYSTGQVSVEIEWE | ACCACCACGCAGTCAAGGCTTCAGTTTTCTCA |
| | LQKENSKRWNPEIQYT | GGCCGGAGCGAGTGACATTCGGGACCAGTCT |
| | SNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 34) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGCTCGATTGGATGA |
| | | GACAACACGCCCTGCTAGACAAGCAGCTACC |
| | | GCAGATGTCAACACACAAGGCGTTCTTCCAGG |
| | | CATGGTCTGGCAGGACAGAGATGTGTACCTTC |
| | | AGGGGCCCATCTGGGCAAAGATTCCACACAC |
| | | GGACGGACATTTTCACCCCTCTCCCCTCATGG |
| | | GTGGATTCGGACTTAAACACCCTCCTCCACAG |
| | | ATTCTCATCAAGAACACCCCGGTACCTGCGAA |
| | | TCCTTCGACCACCTTCAGTGCGGCAAAGTTTG |
| | | CTTCCTTCATCACACAGTACTCCACGGGACAG |
| | | GTCAGCGTGGAGATCGAGTGGGAGCTGCAGA |
| | | AGGAAAACAGCAAACGCTGGAATCCCGAAAT |
| | | TCAGTACACTTCCAACTACAACAAGTCTGTTA |
| | | ATGTGGACTTTACTGTGGACACTAATGGCGTG |
| | | TATTCAGAGCCTCGCCCCATTGGCACCAGATA |
| | | CCTGACTCGTAATCTGTAA (SEQ ID NO: 79) |
| VAR-34 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGLALAEITRPAR | TCTTCATTTTACTGCCTGGAGTACTTTCCTTCT |
| | QAATADVNTQGVLPG | CAGATGCTGCGTACCGGAAACAACTTTACCTT |
| | MVWQDRDVYLQGPIW | CAGCTACACTTTTGAGGACGTTCCTTTCCACA |
| | AKIPHTDGHFHPSPLM | GCAGCTACGCTCACAGCCAGAGTCTGGACCGT |
| | GGFGLKHPPPQILIKNT | CTCATGAATCCTCTCATCGACCAGTACCTGTA |
| | PVPANPSTTFSAAKFA | TTACTTGAGCAGAACAAACACTCCAAGTGGA |
| | SFITQYSTGQVSVEIEW | ACCACCACGCAGTCAAGGCTTCAGTTTTCTCA |
| | ELQKENSKRWNPEIQY | GGCCGGAGCGAGTGACATTCGGGACCAGTCT |
| | TSNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 35) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCTTGGCTCTCGC |
| | | TGAGATCACCAGACCGGCGAGACAAGCAGCT |
| | | ACCGCAGATGTCAACACACAAGGCGTTCTTCC |
| | | AGGCATGGTCTGGCAGGACAGAGATGTGTAC |
| | | CTTCAGGGGCCCATCTGGGCAAAGATTCCACA |
| | | CACGGACGGACATTTTCACCCCTCTCCCCTCA |
| | | TGGGTGGATTCGGACTTAAACACCCTCCTCCA |
| | | CAGATTCTCATCAAGAACACCCCGGTACCTGC |
| | | GAATCCTTCGACCACCTTCAGTGCGGCAAAGT |
| | | TTGCTTCCTTCATCACACAGTACTCCACGGGA |
| | | CAGGTCAGCGTGGAGATCGAGTGGGAGCTGC |
| | | AGAAGGAAAACAGCAAACGCTGGAATCCCGA |
| | | AATTCAGTACACTTCCAACTACAACAAGTCTG |
| | | TTAATGTGGACTTTACTGTGGACACTAATGGC |
| | | GTGTATTCAGAGCCTCGCCCCATTGGCACCAG |
| | | ATACCTGACTCGTAATCTGTAA (SEQ ID NO: |
| | | 80) |
| VAR-35 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGLANGEQTRPA | TCTTCATTTTACTGCCTGGAGTACTTTCCTTCT |
| | RQAATADVNTQGVLP | CAGATGCTGCGTACCGGAAACAACTTTACCTT |
| | GMVWQDRDVYLQGPI | CAGCTACACTTTTGAGGACGTTCCTTTCCACA |
| | WAKIPHTDGHFHPSPL | GCAGCTACGCTCACAGCCAGAGTCTGGACCGT |
| | MGGFGLKHPPPQILIK | CTCATGAATCCTCTCATCGACCAGTACCTGTA |
| | NTPVPANPSTTFSAAK | TTACTTGAGCAGAACAAACACTCCAAGTGGA |
| | FASFITQYSTGQVSVEI | ACCACCACGCAGTCAAGGCTTCAGTTTTCTCA |
| | EWELQKENSKRWNPEI | GGCCGGAGCGAGTGACATTCGGGACCAGTCT |
| | QYTSNYNKSVNVDFT | AGGAACTGGCTTCCTGGACCCTGTTACCGCCA |
| | VDTNGVYSEPRPIGTR | GCAGCGAGTATCAAAGACATCTGCGGATAAC |
| | YLTRNL* (SEQ ID NO: | AACAACAGTGAATACTCGTGGACTGGAGCTA |
| | 36) | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCCTGGCGAACG |
| | | GCGAACAAACCAGGCCTGCGAGACAAGCAGC |
| | | TACCGCAGATGTCAACACACAAGGCGTTCTTC |
| | | CAGGCATGGTCTGGCAGGACAGAGATGTGTA |
| | | CCTTCAGGGGCCCATCTGGGCAAAGATTCCAC |
| | | ACACGGACGGACATTTTCACCCCTCTCCCCTC |
| | | ATGGGTGGATTCGGACTTAAACACCCTCCTCC |
| | | ACAGATTCTCATCAAGAACACCCCGGTACCTG |
| | | CGAATCCTTCGACCACCTTCAGTGCGGCAAAG |
| | | TTTGCTTCCTTCATCACACAGTACTCCACGGG |
| | | ACAGGTCAGCGTGGAGATCGAGTGGGAGCTG |
| | | CAGAAGGAAAACAGCAAACGCTGGAATCCCG |
| | | AAATTCAGTACACTTCCAACTACAACAAGTCT |
| | | GTTAATGTGGACTTTACTGTGGACACTAATGG |
| | | CGTGTATTCAGAGCCTCGCCCCATTGGCACCA |
| | | GATACCTGACTCGTAATCTGTAA (SEQ ID NO: |
| | | 81) |
| VAR-36 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGATDTKTNRQA | TCTTCATTTTACTGCCTGGAGTACTTTCCTTCT |
| | ATADVNTQGVLPGMV | CAGATGCTGCGTACCGGAAACAACTTTACCTT |
| | WQDRDVYLQGPIWAK | CAGCTACACTTTTGAGGACGTTCCTTTCCACA |
| | IPHTDGHFHPSPLMGG | GCAGCTACGCTCACAGCCAGAGTCTGGACCGT |
| | FGLKHPPPQILIKNTPV | CTCATGAATCCTCTCATCGACCAGTACCTGTA |
| | PANPSTTFSAAKFASFI | TTACTTGAGCAGAACAAACACTCCAAGTGGA |
| | TQYSTGQVSVEIEWEL | ACCACCACGCAGTCAAGGCTTCAGTTTTCTCA |
| | QKENSKRWNPEIQYTS | GGCCGGAGCGAGTGACATTCGGGACCAGTCT |
| | NYNKSVNVDFTVDTN | AGGAACTGGCTTCCTGGACCCTGTTACCGCCA |
| | GVYSEPRPIGTRYLTR | GCAGCGAGTATCAAAGACATCTGCGGATAAC |
| | NL* (SEQ ID NO: 37) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCGCTACGGATAC |
| | | TAAGACGAACAGACAAGCAGCTACCGCAGAT |
| | | GTCAACACACAAGGCGTTCTTCCAGGCATGGT |
| | | CTGGCAGGACAGAGATGTGTACCTTCAGGGG |
| | | CCCATCTGGGCAAAGATTCCACACACGGACG |
| | | GACATTTTCACCCCTCTCCCCTCATGGGTGGA |
| | | TTCGGACTTAAACACCCTCCTCCACAGATTCT |
| | | CATCAAGAACACCCCGGTACCTGCGAATCCTT |
| | | CGACCACCTTCAGTGCGGCAAAGTTTGCTTCC |
| | | TTCATCACACAGTACTCCACGGGACAGGTCAG |
| | | CGTGGAGATCGAGTGGGAGCTGCAGAAGGAA |
| | | AACAGCAAACGCTGGAATCCCGAAATTCAGT |
| | | ACACTTCCAACTACAACAAGTCTGTTAATGTG |
| | | GACTTTACTGTGGACACTAATGGCGTGTATTC |
| | | AGAGCCTCGCCCCATTGGCACCAGATACCTGA |
| | | CTCGTAATCTGTAA (SEQ ID NO: 82) |
| VAR-37 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQAPATADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 38) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAG |
| | | CACCAGCTACCGCAGATGTCAACACACAAGG |
| | | CGTTCTTCCAGGCATGGTCTGGCAGGACAGAG |
| | | ATGTGTACCTTCAGGGGCCCATCTGGGCAAAG |
| | | ATTCCACACACGGACGGACATTTTCACCCCTC |
| | | TCCCCTCATGGGTGGATTCGGACTTAAACACC |
| | | CTCCTCCACAGATTCTCATCAAGAACACCCCG |
| | | GTACCTGCGAATCCTTCGACCACCTTCAGTGC |
| | | GGCAAAGTTTGCTTCCTTCATCACACAGTACT |
| | | CCACGGGACAGGTCAGCGTGGAGATCGAGTG |
| | | GGAGCTGCAGAAGGAAAACAGCAAACGCTGG |
| | | AATCCCGAAATTCAGTACACTTCCAACTACAA |
| | | CAAGTCTGTTAATGTGGACTTTACTGTGGACA |
| | | CTAATGGCGTGTATTCAGAGCCTCGCCCCATT |
| | | GGCACCAGATACCTGACTCGTAATCTGTAA |
| | | (SEQ ID NO: 83) |
| VAR-38 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNAPGETTRPAR | TCTTCATTTTACTGCCTGGAGTACTTTCCTTCT |
| | QAATADVNTQGVLPG | CAGATGCTGCGTACCGGAAACAACTTTACCTT |
| | MVWQDRDVYLQGPIW | CAGCTACACTTTTGAGGACGTTCCTTTCCACA |
| | AKIPHTDGHFHPSPLM | GCAGCTACGCTCACAGCCAGAGTCTGGACCGT |
| | GGFGLKHPPPQILIKNT | CTCATGAATCCTCTCATCGACCAGTACCTGTA |
| | PVPANPSTTFSAAKFA | TTACTTGAGCAGAACAAACACTCCAAGTGGA |
| | SFITQYSTGQVSVEIEW | ACCACCACGCAGTCAAGGCTTCAGTTTTCTCA |
| | ELQKENSKRWNPEIQY | GGCCGGAGCGAGTGACATTCGGGACCAGTCT |
| | TSNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 39) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACGCACCCG |
| | | GAGAGACTACTAGGCCAGCAAGACAAGCAGC |
| | | TACCGCAGATGTCAACACACAAGGCGTTCTTC |
| | | CAGGCATGGTCTGGCAGGACAGAGATGTGTA |
| | | CCTTCAGGGGCCCATCTGGGCAAAGATTCCAC |
| | | ACACGGACGGACATTTTCACCCCTCTCCCCTC |
| | | ATGGGTGGATTCGGACTTAAACACCCTCCTCC |
| | | ACAGATTCTCATCAAGAACACCCCGGTACCTG |
| | | CGAATCCTTCGACCACCTTCAGTGCGGCAAAG |
| | | TTTGCTTCCTTCATCACACAGTACTCCACGGG |
| | | ACAGGTCAGCGTGGAGATCGAGTGGGAGCTG |
| | | CAGAAGGAAAACAGCAAACGCTGGAATCCCG |
| | | AAATTCAGTACACTTCCAACTACAACAAGTCT |
| | | GTTAATGTGGACTTTACTGTGGACACTAATGG |
| | | CGTGTATTCAGAGCCTCGCCCCATTGGCACCA |
| | | GATACCTGACTCGTAATCTGTAA (SEQ ID NO: |
| | | 84) |
| VAR-39 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQPAATADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 40) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAC |
| | | CGGCAGCTACCGCAGATGTCAACACACAAGG |
| | | CGTTCTTCCAGGCATGGTCTGGCAGGACAGAG |
| | | ATGTGTACCTTCAGGGGCCCATCTGGGCAAAG |
| | | ATTCCACACACGGACGGACATTTTCACCCCTC |
| | | TCCCCTCATGGGTGGATTCGGACTTAAACACC |
| | | CTCCTCCACAGATTCTCATCAAGAACACCCCG |
| | | GTACCTGCGAATCCTTCGACCACCTTCAGTGC |
| | | GGCAAAGTTTGCTTCCTTCATCACACAGTACT |
| | | CCACGGGACAGGTCAGCGTGGAGATCGAGTG |
| | | GGAGCTGCAGAAGGAAAACAGCAAACGCTGG |
| | | AATCCCGAAATTCAGTACACTTCCAACTACAA |
| | | CAAGTCTGTTAATGTGGACTTTACTGTGGACA |
| | | CTAATGGCGTGTATTCAGAGCCTCGCCCCATT |
| | | GGCACCAGATACCTGACTCGTAATCTGTAA |
| | | (SEQ ID NO: 85) |
| VAR-40 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQSSARQAETARVN | TCTTCATTTTACTGCCTGGAGTACTTTCCTTCT |
| | AQGILPGMVWQDRDV | CAGATGCTGCGTACCGGAAACAACTTTACCTT |
| | YLQGPIWAKIPHTDGH | CAGCTACACTTTTGAGGACGTTCCTTTCCACA |
| | FHPSPLMGGFGLKHPP | GCAGCTACGCTCACAGCCAGAGTCTGGACCGT |
| | PQILIKNTPVPANPSTT | CTCATGAATCCTCTCATCGACCAGTACCTGTA |
| | FSAAKFASFITQYSTGQ | TTACTTGAGCAGAACAAACACTCCAAGTGGA |
| | VSVEIEWELQKENSKR | ACCACCACGCAGTCAAGGCTTCAGTTTTCTCA |
| | WNPEIQYTSNYNKSVN | GGCCGGAGCGAGTGACATTCGGGACCAGTCT |
| | VDFTVDTNGVYSEPRP | AGGAACTGGCTTCCTGGACCCTGTTACCGCCA |
| | IGTRYLTRNL* (SEQ ID | GCAGCGAGTATCAAAGACATCTGCGGATAAC |
| | NO: 41) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGTCTTCTGCTAGACAAGC |
| | | AGAGACAGCACGGGTCAACGCTCAAGGCATT |
| | | CTTCCAGGCATGGTCTGGCAGGACAGAGATGT |
| | | GTACCTTCAGGGGCCCATCTGGGCAAAGATTC |
| | | CACACACGGACGGACATTTTCACCCCTCTCCC |
| | | CTCATGGGTGGATTCGGACTTAAACACCCTCC |
| | | TCCACAGATTCTCATCAAGAACACCCCGGTAC |
| | | CTGCGAATCCTTCGACCACCTTCAGTGCGGCA |
| | | AAGTTTGCTTCCTTCATCACACAGTACTCCAC |
| | | GGGACAGGTCAGCGTGGAGATCGAGTGGGAG |
| | | CTGCAGAAGGAAAACAGCAAACGCTGGAATC |
| | | CCGAAATTCAGTACACTTCCAACTACAACAAG |
| | | TCTGTTAATGTGGACTTTACTGTGGACACTAA |
| | | TGGCGTGTATTCAGAGCCTCGCCCCATTGGCA |
| | | CCAGATACCTGACTCGTAATCTGTAA (SEQ ID |
| | | NO: 86) |
| VAR-41 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGFHNEGKYNRQ | TCTTCATTTTACTGCCTGGAGTACTTTCCTTCT |
| | AATADVNTQGVLPGM | CAGATGCTGCGTACCGGAAACAACTTTACCTT |
| | VWQDRDVYLQGPIWA | CAGCTACACTTTTGAGGACGTTCCTTTCCACA |
| | KIPHTDGHFHPSPLMG | GCAGCTACGCTCACAGCCAGAGTCTGGACCGT |
| | GFGLKHPPPQILIKNTP | CTCATGAATCCTCTCATCGACCAGTACCTGTA |
| | VPANPSTTFSAAKFAS | TTACTTGAGCAGAACAAACACTCCAAGTGGA |
| | FITQYSTGQVSVEIEWE | ACCACCACGCAGTCAAGGCTTCAGTTTTCTCA |
| | LQKENSKRWNPEIQYT | GGCCGGAGCGAGTGACATTCGGGACCAGTCT |
| | SNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 42) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCTTCCATAATGA |
| | | AGGAAAATACAACAGACAAGCAGCTACCGCA |
| | | GATGTCAACACACAAGGCGTTCTTCCAGGCAT |
| | | GGTCTGGCAGGACAGAGATGTGTACCTTCAG |
| | | GGGCCCATCTGGGCAAAGATTCCACACACGG |
| | | ACGGACATTTTCACCCCTCTCCCCTCATGGGT |
| | | GGATTCGGACTTAAACACCCTCCTCCACAGAT |
| | | TCTCATCAAGAACACCCCGGTACCTGCGAATC |
| | | CTTCGACCACCTTCAGTGCGGCAAAGTTTGCT |
| | | TCCTTCATCACACAGTACTCCACGGGACAGGT |
| | | CAGCGTGGAGATCGAGTGGGAGCTGCAGAAG |
| | | GAAAACAGCAAACGCTGGAATCCCGAAATTC |
| | | AGTACACTTCCAACTACAACAAGTCTGTTAAT |
| | | GTGGACTTTACTGTGGACACTAATGGCGTGTA |
| | | TTCAGAGCCTCGCCCCATTGGCACCAGATACC |
| | | TGACTCGTAATCTGTAA (SEQ ID NO: 87) |
| VAR-42 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQAAGTADV | TCTTCATTTTACTGCCTGGAGTACTTTCCTTCT |
| | NTQGVLPGMVWQDR | CAGATGCTGCGTACCGGAAACAACTTTACCTT |
| | DVYLQGPIWAKIPHTD | CAGCTACACTTTTGAGGACGTTCCTTTCCACA |
| | GHFHPSPLMGGFGLKH | GCAGCTACGCTCACAGCCAGAGTCTGGACCGT |
| | PPPQILIKNTPVPANPST | CTCATGAATCCTCTCATCGACCAGTACCTGTA |
| | TFSAAKFASFITQYSTG | TTACTTGAGCAGAACAAACACTCCAAGTGGA |
| | QVSVEIEWELQKENSK | ACCACCACGCAGTCAAGGCTTCAGTTTTCTCA |
| | RWNPEIQYTSNYNKSV | GGCCGGAGCGAGTGACATTCGGGACCAGTCT |
| | NVDFTVDTNGVYSEPR | AGGAACTGGCTTCCTGGACCCTGTTACCGCCA |
| | PIGTRYLTRNL* (SEQ | GCAGCGAGTATCAAAGACATCTGCGGATAAC |
| | ID NO: 43) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAG |
| | | CAGCTGGGACCGCAGATGTCAACACACAAGG |
| | | CGTTCTTCCAGGCATGGTCTGGCAGGACAGAG |
| | | ATGTGTACCTTCAGGGGCCCATCTGGGCAAAG |
| | | ATTCCACACACGGACGGACATTTTCACCCCTC |
| | | TCCCCTCATGGGTGGATTCGGACTTAAACACC |
| | | CTCCTCCACAGATTCTCATCAAGAACACCCCG |
| | | GTACCTGCGAATCCTTCGACCACCTTCAGTGC |
| | | GGCAAAGTTTGCTTCCTTCATCACACAGTACT |
| | | CCACGGGACAGGTCAGCGTGGAGATCGAGTG |
| | | GGAGCTGCAGAAGGAAAACAGCAAACGCTGG |
| | | AATCCCGAAATTCAGTACACTTCCAACTACAA |
| | | CAAGTCTGTTAATGTGGACTTTACTGTGGACA |
| | | CTAATGGCGTGTATTCAGAGCCTCGCCCCATT |
| | | GGCACCAGATACCTGACTCGTAATCTGTAA |
| | | (SEQ ID NO: 88) |
| VAR-43 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQPWEPDKQ | TCTTCATTTTACTGCCTGGAGTACTTTCCTTCT |
| | AATADVNTQGVLPGM | CAGATGCTGCGTACCGGAAACAACTTTACCTT |
| | VWQDRDVYLQGPIWA | CAGCTACACTTTTGAGGACGTTCCTTTCCACA |
| | KIPHTDGHFHPSPLMG | GCAGCTACGCTCACAGCCAGAGTCTGGACCGT |
| | GFGLKHPPPQILIKNTP | CTCATGAATCCTCTCATCGACCAGTACCTGTA |
| | VPANPSTTFSAAKFAS | TTACTTGAGCAGAACAAACACTCCAAGTGGA |
| | FITQYSTGQVSVEIEWE | ACCACCACGCAGTCAAGGCTTCAGTTTTCTCA |
| | LQKENSKRWNPEIQYT | GGCCGGAGCGAGTGACATTCGGGACCAGTCT |
| | SNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 44) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAC |
| | | CGTGGGAACCGGATAAGCAAGCAGCTACCGC |
| | | AGATGTCAACACACAAGGCGTTCTTCCAGGCA |
| | | TGGTCTGGCAGGACAGAGATGTGTACCTTCAG |
| | | GGGCCCATCTGGGCAAAGATTCCACACACGG |
| | | ACGGACATTTTCACCCCTCTCCCCTCATGGGT |
| | | GGATTCGGACTTAAACACCCTCCTCCACAGAT |
| | | TCTCATCAAGAACACCCCGGTACCTGCGAATC |
| | | CTTCGACCACCTTCAGTGCGGCAAAGTTTGCT |
| | | TCCTTCATCACACAGTACTCCACGGGACAGGT |
| | | CAGCGTGGAGATCGAGTGGGAGCTGCAGAAG |
| | | GAAAACAGCAAACGCTGGAATCCCGAAATTC |
| | | AGTACACTTCCAACTACAACAAGTCTGTTAAT |
| | | GTGGACTTTACTGTGGACACTAATGGCGTGTA |
| | | TTCAGAGCCTCGCCCCATTGGCACCAGATACC |
| | | TGACTCGTAATCTGTAA (SEQ ID NO: 89) |
| VAR-44 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRGNRQAATALAL | TCTTCATTTTACTGCCTGGAGTACTTTCCTTCT |
| | STTNADVNTQGVLPG | CAGATGCTGCGTACCGGAAACAACTTTACCTT |
| | MVWQDRDVYLQGPIW | CAGCTACACTTTTGAGGACGTTCCTTTCCACA |
| | AKIPHTDGHFHPSPLM | GCAGCTACGCTCACAGCCAGAGTCTGGACCGT |
| | GGFGLKHPPPQILIKNT | CTCATGAATCCTCTCATCGACCAGTACCTGTA |
| | PVPANPSTTFSAAKFA | TTACTTGAGCAGAACAAACACTCCAAGTGGA |
| | SFITQYSTGQVSVEIEW | ACCACCACGCAGTCAAGGCTTCAGTTTTCTCA |
| | ELQKENSKRWNPEIQY | GGCCGGAGCGAGTGACATTCGGGACCAGTCT |
| | TSNYNKSVNVDFTVDT | AGGAACTGGCTTCCTGGACCCTGTTACCGCCA |
| | NGVYSEPRPIGTRYLT | GCAGCGAGTATCAAAGACATCTGCGGATAAC |
| | RNL* (SEQ ID NO: 45) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGAGAGGCAACAGACAAG |
| | | CAGCTACCGCACTGGCGCTTAGTACAACTAAT |
| | | GCAGATGTCAACACACAAGGCGTTCTTCCAGG |
| | | CATGGTCTGGCAGGACAGAGATGTGTACCTTC |
| | | AGGGGCCCATCTGGGCAAAGATTCCACACAC |
| | | GGACGGACATTTTCACCCCTCTCCCCTCATGG |
| | | GTGGATTCGGACTTAAACACCCTCCTCCACAG |
| | | ATTCTCATCAAGAACACCCCGGTACCTGCGAA |
| | | TCCTTCGACCACCTTCAGTGCGGCAAAGTTTG |
| | | CTTCCTTCATCACACAGTACTCCACGGGACAG |
| | | GTCAGCGTGGAGATCGAGTGGGAGCTGCAGA |
| | | AGGAAAACAGCAAACGCTGGAATCCCGAAAT |
| | | TCAGTACACTTCCAACTACAACAAGTCTGTTA |
| | | ATGTGGACTTTACTGTGGACACTAATGGCGTG |
| | | TATTCAGAGCCTCGCCCCATTGGCACCAGATA |
| | | CCTGACTCGTAATCTGTAA (SEQ ID NO: 90) |
| VAR-45 | MAADGYLPDWLEDTL | ATGGCTGCCGATGGTTATCTTCCAGATTGGCT |
| | SEGIRQWWKLKPGPPP | CGAGGACACTCTCTCTGAAGGAATAAGACAG |
| | PKPAERHKDDSRGLVL | TGGTGGAAGCTCAAACCTGGCCCACCACCACC |
| | PGYKYLGPFNGLDKGE | AAAGCCCGCAGAGCGGCATAAGGACGACAGC |
| | PVNEADAAALEHDKA | AGGGGTCTTGTGCTTCCTGGGTACAAGTACCT |
| | YDRQLDSGDNPYLKY | CGGACCCTTCAACGGACTCGACAAGGGAGAG |
| | NHADAEFQERLKEDTS | CCGGTCAACGAGGCAGACGCCGCGGCCCTCG |
| | FGGNLGRAVFQAKKR | AGCACGACAAAGCCTACGACCGGCAGCTCGA |
| | VLEPLGLVEEPVKTAP | CAGCGGAGACAACCCGTACCTCAAGTACAAC |
| | GKKRPVEHSPVEPDSS | CACGCCGACGCGGAGTTTCAGGAGCGCCTTA |
| | SGTGKAGQQPARKRL | AAGAAGATACGTCTTTTGGGGGCAACCTCGG |
| | NFGQTGDADSVPDPQP | ACGAGCAGTCTTCCAGGCGAAAAAGAGGGTT |
| | LGQPPAAPSGLGTNTM | CTTGAACCTCTGGGCCTGGTTGAGGAACCTGT |
| | ATGSGAPMADNNEGA | TAAGACGGCTCCGGGAAAAAAGAGGCCGGTA |
| | DGVGNSSGNWHCDST | GAGCACTCTCCTGTGGAGCCAGACTCCTCCTC |
| | WMGDRVITTSTRTWA | GGGAACCGGAAAGGCGGGCCAGCAGCCTGCA |
| | LPTYNNHLYKQISSQS | AGAAAAAGATTGAATTTTGGTCAGACTGGAG |
| | GASNDNHYFGYSTPW | ACGCAGACTCAGTACCTGACCCCCAGCCTCTC |
| | GYFDFNRFHCHFSPRD | GGACAGCCACCAGCAGCCCCCTCTGGTCTGGG |
| | WQRLINNNWGFRPKR | AACTAATACGATGGCTACAGGCAGTGGCGCA |
| | LNFKLFNIQVKEVTQN | CCAATGGCAGACAATAACGAGGGCGCCGACG |
| | DGTTTIANNLTSTVQV | GAGTGGGTAATTCCTCGGGAAATTGGCATTGC |
| | FTDSEYQLPYVLGSAH | GATTCCACATGGATGGGCGACAGAGTCATCA |
| | QGCLPPFPADVFMVPQ | CCACCAGCACCCGAACCTGGGCCCTGCCCACC |
| | YGYLTLNNGSQAVGR | TACAACAACCACCTCTACAAACAAATTTCCAG |
| | SSFYCLEYFPSQMLRT | CCAATCAGGAGCCTCGAACGACAATCACTACT |
| | GNNFTFSYTFEDVPFH | TTGGCTACAGCACCCCTTGGGGGTATTTTGAC |
| | SSYAHSQSLDRLMNPL | TTCAACAGATTCCACTGCCACTTTTCACCACG |
| | IDQYLYYLSRTNTPSG | TGACTGGCAAAGACTCATCAACAACAACTGG |
| | TTTQSRLQFSQAGASDI | GGATTCCGACCCAAGAGACTCAACTTCAAGCT |
| | RDQSRNWLPGPCYRQ | CTTTAACATTCAAGTCAAAGAGGTCACGCAGA |
| | QRVSKTSADNNNSEYS | ATGACGGTACGACGACGATTGCCAATAACCTT |
| | WTGATKYHLNGRDSL | ACCAGCACGGTTCAGGTGTTTACTGACTCGGA |
| | VNPGPAMASHKDDEE | GTACCAGCTCCCGTACGTCCTCGGCTCGGCGC |
| | KFFPQSGVLIFGKQGSE | ATCAAGGATGCCTCCCGCCGTTCCCAGCAGAC |
| | KTNVDIEKVMITDEEEI | GTCTTCATGGTGCCACAGTATGGATACCTCAC |
| | RTTNPVATEQYGSVST | CCTGAACAACGGGAGTCAGGCAGTAGGACGC |
| | NLQRPWGTAGGNRQA | TCTTCATTTTACTGCCTGGAGTACTTTCCTTCT |
| | ATADVNTQGVLPGMV | CAGATGCTGCGTACCGGAAACAACTTTACCTT |
| | WQDRDVYLQGPIWAK | CAGCTACACTTTTGAGGACGTTCCTTTCCACA |
| | IPHTDGHFHPSPLMGG | GCAGCTACGCTCACAGCCAGAGTCTGGACCGT |
| | FGLKHPPPQILIKNTPV | CTCATGAATCCTCTCATCGACCAGTACCTGTA |
| | PANPSTTFSAAKFASFI | TTACTTGAGCAGAACAAACACTCCAAGTGGA |
| | TQYSTGQVSVEIEWEL | ACCACCACGCAGTCAAGGCTTCAGTTTTCTCA |
| | QKENSKRWNPEIQYTS | GGCCGGAGCGAGTGACATTCGGGACCAGTCT |
| | NYNKSVNVDFTVDTN | AGGAACTGGCTTCCTGGACCCTGTTACCGCCA |
| | GVYSEPRPIGTRYLTR | GCAGCGAGTATCAAAGACATCTGCGGATAAC |
| | NL* (SEQ ID NO: 46) | AACAACAGTGAATACTCGTGGACTGGAGCTA |
| | | CCAAGTACCACCTCAATGGCAGAGACTCTCTG |
| | | GTGAATCCGGGCCCGGCCATGGCAAGCCACA |
| | | AGGACGATGAAGAAAAGTTTTTTCCTCAGAGC |
| | | GGGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGTA |
| | | TCTACCAACCTCCAGCGGCCTTGGGGCACAGC |
| | | TGGAGGCAACAGACAAGCAGCTACCGCAGAT |
| | | GTCAACACACAAGGCGTTCTTCCAGGCATGGT |
| | | CTGGCAGGACAGAGATGTGTACCTTCAGGGG |
| | | CCCATCTGGGCAAAGATTCCACACACGGACG |
| | | GACATTTTCACCCCTCTCCCCTCATGGGTGGA |
| | | TTCGGACTTAAACACCCTCCTCCACAGATTCT |
| | | CATCAAGAACACCCCGGTACCTGCGAATCCTT |
| | | CGACCACCTTCAGTGCGGCAAAGTTTGCTTCC |
| | | TTCATCACACAGTACTCCACGGGACAGGTCAG |
| | | CGTGGAGATCGAGTGGGAGCTGCAGAAGGAA |
| | | AACAGCAAACGCTGGAATCCCGAAATTCAGT |
| | | ACACTTCCAACTACAACAAGTCTGTTAATGTG |
| | | GACTTTACTGTGGACACTAATGGCGTGTATTC |
| | | AGAGCCTCGCCCCATTGGCACCAGATACCTGA |
| | | CTCGTAATCTGTAA (SEQ ID NO: 91) |

In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence as provided in Table 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence as provided in Table 3. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence as provided in Table 4. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 3. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 4. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 5. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 6. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 7. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 8. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 9. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 10. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 11. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 12. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 13. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 14. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 15. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 16. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 17. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 18. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 19. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 20. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 21. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 22. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 23. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 24. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 25. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 26. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 27. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 28. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 29. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 30. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 31. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 32. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 33. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 34. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 35. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 36. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 37. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 38. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 39. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 40. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 41. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 42. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 43. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 44. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 45. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 46.

In some embodiments, the capsid polypeptide comprises a reference capsid sequence, such as SEQ ID NO: 1, and at least, or about, 80%, 85%, 90%, or 95%, or 100% of the mutations (insertions, deletions, or substitutions) as shown in the Mutation Differences column of Table 1 of VAR-1, VAR-2, VAR-3, VAR-4, VAR-5, VAR-6, VAR-7, VAR-8, VAR-9, VAR-10, VAR-11, VAR-12, VAR-13, VAR-14, VAR-15, VAR-16, VAR-17, VAR-18, VAR-19, VAR-20, VAR-21, VAR-22, VAR-23, VAR-24, VAR-25, VAR-26, VAR-27, VAR-28, VAR-29, VAR-30, VAR-31, VAR-32, VAR-33, VAR-34, VAR-35, VAR-36, VAR-37, VAR-38, VAR-39, VAR-40, VAR-41, VAR-42, VAR-43, VAR-44, or VAR-45. In some embodiments, the reference capsid sequence comprises at least, about, or exactly, 80% of the mutations (insertions, deletions, or substitutions). In some embodiments, the reference capsid sequence comprises at least, about, or exactly, 85% of the mutations (insertions, deletions, or substitutions). In some embodiments, the reference capsid sequence comprises at least, about, or exactly, 90% of the mutations (insertions, deletions, or substitutions). In some embodiments, the reference capsid sequence comprises at least, about, or exactly, 95% of the mutations (insertions, deletions, or substitutions). In some embodiments, the reference capsid sequence comprises 100% of the mutations (insertions, deletions, or substitutions).

In some embodiments, the capsid polypeptide comprises a reference capsid sequence, such as SEQ ID NO: 1, and at least, or about, or exactly, 80%, 85%, 90%, or 95%, or 100% of one of the following groups of mutations (the terminology for these groups of mutations is provided for in the legend of Table 1 above):
[‘T14L’, ‘L15V’, ‘I19A’, ‘K24H’];
[‘A3V’, ‘Y6F’, ‘L11F’, ‘E12Q’, ‘Q21L’, ‘W23I’, ‘L25C’, ‘P29A’, ‘P31N’, ‘K33R’];
[‘N449Q’, ‘T450M’, ‘P451T’, ‘T455L’, ‘T456G’, ‘Q457T’, ‘S458Q’, ‘R459M’];
[‘N449Q’, ‘P451-’, ‘S452T’, ‘T456G’, ‘Q457T’, ‘R459G’];
[‘Q457F’, ‘S458C’];
[‘446_7aa_447_KCQEGMA’];
[‘449_7aa_450_LMVDRLG’];
[‘P451T’, ‘T455G’, ‘T456G’, ‘Q457T’, ‘S458Q’, ‘R459T’, ‘Q461A’];
[‘448_7aa_449_HCQECPI’];
[‘453_6aa_454_FSGLEN’];
[‘T456-’, ‘Q457-’, ‘S458-’, ‘R459-’, ‘L460-’, ‘Q461-’];
[‘N449I’, ‘T450N’, ‘P451G’, ‘T454-’, ‘T455Q’, ‘T456N’, ‘S458Q’, ‘R459T’, ‘Q461K’, ‘Q464V’];
[‘S446A’, ‘T448-’, ‘N449-’, ‘P451Q’, ‘G453T’, ‘T455G’, ‘T456G’, ‘Q457T’, ‘R459G’];
[‘N449Q’, ‘T450S’, ‘451_1aa_452_Y’, ‘S452G’, ‘T455A’, ‘Q457F’, ‘S458M’, ‘R459D’];
[‘447_7aa_448_HETEFNF’];
[‘P451T’, ‘T455G’, ‘T456G’, ‘Q457T’, ‘S458Q’, ‘R459T’, ‘Q461A’];
[‘581_6aa_582_FALMEP’];
[‘584_6aa_585_RAYNPD’];
[‘E555A’, ‘D561S’, ‘R566K’, ‘S578V’, ‘S580A’, ‘T581A’, ‘R585-’];
[‘V557L’, ‘S578D’];
[‘K556N’, ‘M558L’, ‘S578I’, ‘S580A’];
[‘I554L’, ‘K556R’, ‘I559L’, ‘D561S’, ‘R566A’, ‘T581D’, ‘R585S’, ‘G586R’, ‘N587S’];
[‘V552A’, ‘E555S’, ‘K556D’, ‘I559L’, ‘D561S’, ‘R566K’, ‘S578I’, ‘T581D’, ‘G586Q’];
[‘Q575E’, ‘S578A’];
[‘583_6aa_584_LNWTAE’];
[‘R585S’, ‘R588T’, ‘Q589N’, ‘A590P’, ‘A591I’, ‘A593G’, ‘T597S’, ‘V600A’];
[‘584_8aa_585_LAKEFTTR’, ‘R585N’, ‘G586A’];
[‘592_5aa_593_LHPLE’];
[‘585_1aa_586_F’];
[‘586_7aa_587_DQDFKNR’];
[‘589_1aa_590_I’];
[‘587_9aa_588_LAIEQTRPA’];
[‘584_8aa_585_RARLDETT’, ‘G586P’, ‘N587A’];
[‘586_9aa_587_LALAEITRP’, ‘N587A’];
[‘586_9aa_587_LANGEQTRP’, ‘N587A’];
[‘586_6aa_587_ATDTKT’];
[‘590_1aa_591_P’];
[‘587_9aa_588_APGETTRPA’];
[‘589_1aa_590_P’];
[‘R585S’, ‘G586S’, ‘N587A’, ‘A591E’, ‘D594R’, ‘T597A’, ‘V600I’];
[‘586_7aa_587_FHNEGKY’];
[‘591_1aa_592_G’];
[‘588_7aa_589_QPWEPDK’];
[‘592_8aa_593_ALALSTTN’]; or
[‘585_6aa_586_PWGTAG’].

In some embodiments, the capsid polypeptide comprises a reference capsid sequence, such as SEQ ID NO: 1, and at least, or about, or exactly, 80%, 85%, 90%, or 95%, or 100% of [‘A3V’, ‘Y6F’, ‘L11F’, ‘E12Q’, ‘Q21L’, ‘W23I’, ‘L25C’, ‘P29A’, ‘P31N’, ‘K33R’]. In some embodiments, the capsid polypeptide comprises at least 8, 9 or all of the mutations of [‘A3V’, ‘Y6F’, ‘L11F’, ‘E12Q’, ‘Q21L’, ‘W23I’, ‘L25C’, ‘P29A’, ‘P31N’, ‘K33R’].

In some embodiments, the capsid polypeptide comprises a reference capsid sequence, such as SEQ ID NO: 1, and at least, or about, or exactly, 80%, 85%, 90%, or 95%, or 100% of [‘N449Q’, ‘P451-’, ‘S452T’, ‘T456G’, ‘Q457T’, ‘R459G’]. In some embodiments, the capsid polypeptide comprises at least 5 or all of the mutations of [‘N449Q’, ‘P451-’, ‘S452T’, ‘T456G’, ‘Q457T’, ‘R459G’].

In some embodiments, the capsid polypeptide comprises a reference capsid sequence, such as SEQ ID NO: 1, and at least, or about, or exactly, 80%, 85%, 90%, or 95%, or 100% of [‘446_7aa_447_KCQEGMA’]. In some embodiments, the capsid polypeptide comprises at least 6 or all of the amino acid residues of the seven amino acid insertion.

In some embodiments, the capsid polypeptide comprises a reference capsid sequence, such as SEQ ID NO: 1, and at least, or about, or exactly, 80%, 85%, 90%, or 95%, or 100% of [‘449_7aa_450_LMVDRLG’]. In some embodiments, the capsid polypeptide comprises at least 6 or all of the amino acid residues of the seven amino acid insertion.

In some embodiments, the capsid polypeptide comprises a reference capsid sequence, such as SEQ ID NO: 1, and at least, or about, or exactly, 80%, 85%, 90%, or 95%, or 100% of [‘P451T’, ‘T455G’, ‘T456G’, ‘Q457T’, ‘S458Q’, ‘R459T’, ‘Q461A’]. In some embodiments, the capsid polypeptide comprises at least 6 or all of the mutations of [‘P451T’, ‘T455G’, ‘T456G’, ‘Q457T’, ‘S458Q’, ‘R459T’, ‘Q461A’].

In some embodiments, the capsid polypeptide comprises a reference capsid sequence, such as SEQ ID NO: 1, and at least, or about, or exactly, 80%, 85%, 90%, or 95%, or 100% of [‘448_7aa_449_HCQECPI’]. In some embodiments, the capsid polypeptide comprises at least 6 or all of the amino acid residues of the seven amino acid insertion.

In some embodiments, the capsid polypeptide comprises a reference capsid sequence, such as SEQ ID NO: 1, and at least, or about, or exactly, 80%, 85%, 90%, or 95%, or 100% of [‘453_6aa_454_FSGLEN’]. In some embodiments, the capsid polypeptide comprises at least 5 or all of the amino acid residues of the six amino acid insertion.

In some embodiments, the capsid polypeptide comprises a reference capsid sequence, such as SEQ ID NO: 1, and at least, or about, or exactly, 80%, 85%, 90%, or 95%, or 100% of [‘T456-’, ‘Q457-’, ‘S458-’, ‘R459-’, ‘L460-’, ‘Q461-’]. In some embodiments, the capsid polypeptide comprises at least 5 or all of the mutations of [‘T456-’, ‘Q457-’, ‘S458-’, ‘R459-’, ‘L460-’, ‘Q461-’].

In some embodiments, the capsid polypeptide comprises a reference capsid sequence, such as SEQ ID NO: 1, and at least, or about, or exactly, 80%, 85%, 90%, or 95%, or 100% of [‘N449I’, ‘T450N’, ‘P451G’, ‘T454-’, ‘T455Q’, ‘T456N’, ‘S458Q’, ‘R459T’, ‘Q461K’, ‘Q464V’]. In some embodiments, the capsid polypeptide comprises at least 8, 9, or all of the mutations of [‘N449I’, ‘T450N’, ‘P451G’, ‘T454-’, ‘T455Q’, ‘T456N’, ‘S458Q’, ‘R459T’, ‘Q461K’, ‘Q464V’].

In some embodiments, the capsid polypeptide comprises a reference capsid sequence, such as SEQ ID NO: 1, and at least, or about, or exactly, 80%, 85%, 90%, or 95%, or 100% of [‘S446A’, ‘T448-’, ‘N449-’, ‘P451Q’, ‘G453T’, ‘T455G’, ‘T456G’, ‘Q457T’, ‘R459G’]. In some embodiments, the capsid polypeptide comprises at least 8 or all of the mutations of [‘S446A’, ‘T448-’, ‘N449-’, ‘P451Q’, ‘G453T’, ‘T455G’, ‘T456G’, ‘Q457T’, ‘R459G’].

In some embodiments, the capsid polypeptide comprises a reference capsid sequence, such as SEQ ID NO: 1, and at least, or about, or exactly, 80%, 85%, 90%, or 95%, or 100% of [‘N449Q’, ‘T450S’, ‘451_1aa_452_Y’, ‘S452G’, ‘T455A’, ‘Q457F’, ‘S458M’, ‘R459D’]. In some embodiments, the capsid polypeptide comprises at least 7 or all of the mutations of [‘N449Q’, ‘T450S’, ‘451_1aa_452_Y’, ‘S452G’, ‘T455A’, ‘Q457F’, ‘S458M’, ‘R459D’].

In some embodiments, the capsid polypeptide comprises a reference capsid sequence, such as SEQ ID NO: 1, and at least, or about, or exactly, 80%, 85%, 90%, or 95%, or 100% of [‘R585S’, ‘G586S’, ‘N587A’, ‘A591E’, ‘D594R’, ‘T597A’, ‘V600I’]. In some embodiments, the capsid polypeptide comprises at least 6 or all of the mutations of [‘R585S’, ‘G586S’, ‘N587A’, ‘A591E’, ‘D594R’, ‘T597A’, ‘V600I’].

In some embodiments, the capsid polypeptide comprises a reference capsid sequence, such as SEQ ID NO: 1, and at least, or about, or exactly, 80%, 85%, 90%, or 95%, or 100% of [‘P451T’, ‘T455G’, ‘T456G’, ‘Q457T’, ‘S458Q’, ‘R459T’, ‘Q461A’]. In some embodiments, the capsid polypeptide comprises at least 6 or all of the mutations of [‘P451T’, ‘T455G’, ‘T456G’, ‘Q457T’, ‘S458Q’, ‘R459T’, ‘Q461A’].

In some embodiments, the capsid polypeptide comprises a reference capsid sequence, such as SEQ ID NO: 1, and at least, or about, or exactly, 80%, 85%, 90%, or 95%, or 100% of [‘E555A’, ‘D561S’, ‘R566K’, ‘S578V’, ‘S580A’, ‘T581A’, ‘R585-’]. In some embodiments, the capsid polypeptide comprises at least 6 or all of the mutations of [‘E555A’, ‘D561S’, ‘R566K’, ‘S578V’, ‘S580A’, ‘T581A’, ‘R585-’].

In some embodiments, the capsid polypeptide comprises a reference capsid sequence, such as SEQ ID NO: 1, and at least, or about, or exactly, 80%, 85%, 90%, or 95%, or 100% of [‘I554L’, ‘K556R’, ‘I559L’, ‘D561S’, ‘R566A’, ‘T581D’, ‘R585S’, ‘G586R’, ‘N587S’]. In some embodiments, the capsid polypeptide comprises at least 8 or all of the mutations of [‘I554L’, ‘K556R’, ‘I559L’, ‘D561S’, ‘R566A’, ‘T581D’, ‘R585S’, ‘G586R’, ‘N587S’].

In some embodiments, the capsid polypeptide comprises a reference capsid sequence, such as SEQ ID NO: 1, and at least, or about, or exactly, 80%, 85%, 90%, or 95%, or 100% of [‘V552A’, ‘E555S’, ‘K556D’, ‘I559L’, ‘D561S’, ‘R566K’, ‘S578I’, ‘T581D’, ‘G586Q’]. In some embodiments, the capsid polypeptide comprises at least 8 or all of the mutations of [‘V552A’, ‘E555S’, ‘K556D’, ‘I559L’, ‘D561S’, ‘R566K’, ‘S578I’, ‘T581D’, ‘G586Q’].

In some embodiments, the capsid polypeptide comprises a reference capsid sequence, such as SEQ ID NO: 1, and at least, or about, or exactly, 80%, 85%, 90%, or 95%, or 100% of [‘R585S’, ‘R588T’, ‘Q589N’, ‘A590P’, ‘A591I’, ‘A593G’, ‘T597S’, ‘V600A’]. In some embodiments, the capsid polypeptide comprises at least 7 or all of the mutations of [‘R585S’, ‘R588T’, ‘Q589N’, ‘A590P’, ‘A591I’, ‘A593G’, ‘T597S’, ‘V600A’].

In some embodiments, the capsid polypeptide comprises a reference capsid sequence, such as SEQ ID NO: 1, and at least, or about, or exactly, 80%, 85%, 90%, or 95%, or 100% of [‘447_7aa_448_HETEFNF’]. In some embodiments, the capsid polypeptide comprises at least 6 or all of the amino acid residues of the 7 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘581_6aa_582_FALMEP’]. In some embodiments, the capsid polypeptide comprises at least 5 or all of the amino acid residues of the 6 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘584_6aa_585_RAYNPD’]. In some embodiments, the capsid polypeptide comprises at least 5 or all of the amino acid residues of the 6 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘583_6aa_584_LNWTAE’]. In some embodiments, the capsid polypeptide comprises at least 5 or all of the amino acid residues of the 6 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘592_5aa_593_LHPLE’]. In some embodiments, the capsid polypeptide comprises at least 4 or all of the amino acid residues of the 5 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘586_7aa_587_DQDFKNR’]. In some embodiments, the capsid polypeptide comprises at least 6 or all of the amino acid residues of the 7 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘587_9aa_588_LAIEQTRPA’]. In some embodiments, the capsid polypeptide comprises at least 8 or all of the amino acid residues of the 9 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘586_6aa_587_ATDTKT’]. In some embodiments, the capsid polypeptide comprises at least 5 or all of the amino acid residues of the 6 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘587_9aa_588_APGETTRPA’]. In some embodiments, the capsid polypeptide comprises at least 8 or all of the amino acid residues of the 9 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘586_7aa_587_FHNEGKY’]. In some embodiments, the capsid polypeptide comprises at least 6 or all of the amino acid residues of the 7 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘588_7aa_589_QPWEPDK’]. In some embodiments, the capsid polypeptide comprises at least 6 or all of the amino acid residues of the 7 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘592_8aa_593_ALALSTTN’]. In some embodiments, the capsid polypeptide comprises at least 7 or all of the amino acid residues of the 8 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘585_6aa_586_PWGTAG’]. In some embodiments, the capsid polypeptide comprises at least 5 or all of the amino acid residues of the 6 amino acid insertion.

In some embodiments, the capsid polypeptide comprises a reference capsid sequence, such as SEQ ID NO: 1, and at least, or about, or exactly, 80%, 85%, 90%, or 95%, or 100% of [‘584_8aa_585_LAKEFTTR’, ‘R585N’, ‘G586A’]. In some embodiments, the capsid polypeptide comprises at least 8, 9, or all of the amino acid residues of the insertion and the point mutations recited in [‘584_8aa_585_LAKEFTTR’, ‘R585N’, ‘G586A’].

In some embodiments, the capsid polypeptide comprises a reference capsid sequence, such as SEQ ID NO: 1, and at least, or about, or exactly, 80%, 85%, 90%, or 95%, or 100% of [‘584_8aa_585_RARLDETT’, ‘G586P’, ‘N587A’]. In some embodiments, the capsid polypeptide comprises at least 8, 9, or all of the amino acid residues of the insertion and the point mutations recited in [‘584_8aa_585_RARLDETT’, ‘G586P’, ‘N587A’].

In some embodiments, the capsid polypeptide comprises a reference capsid sequence, such as SEQ ID NO: 1, and at least, or about, or exactly, 80%, 85%, 90%, or 95%, or 100% of [‘586_9aa_587_LALAEITRP’, ‘N587A’]. In some embodiments, the capsid polypeptide comprises at least 8, 9, or all of the amino acid residues of the insertion and the point mutations recited in [‘586_9aa_587_LALAEITRP’, ‘N587A’].

In some embodiments, the capsid polypeptide comprises a reference capsid sequence, such as SEQ ID NO: 1, and at least, or about, or exactly, 80%, 85%, 90%, or 95%, or 100% of [‘586_9aa_587_LANGEQTRP’, ‘N587A’]. In some embodiments, the capsid polypeptide comprises at least 8, 9, or all of the amino acid residues of the insertion and the point mutations recited in [‘586_9aa_587_LANGEQTRP’, ‘N587A’].

### Variant Capsids (Corresponding Positions)

The mutations to capsid polypeptide sequences described herein are described in relation to a position and/or amino acid at a position within a reference sequence, e.g., SEQ ID NO: 1. Thus, in some embodiments, the capsid polypeptides described herein are variant capsid polypeptides of the reference sequence, e.g., SEQ ID NO: 1, e.g., include capsid polypeptides comprising at least 80%, at least 85%, at least 90%, at least 91%, at least 92%, at least 93%, at least 94%, at least 95%, at least 96%, at least 97%, at least 98%, or at least 99% identity to the reference capsid polypeptide sequence (e.g., reference capsid polypeptide VP1, VP2 and/or VP3 sequence), e.g., SEQ ID NO: 1 (or VP2 or VP3 sequence comprised therein) and further including one or more mutations described herein.

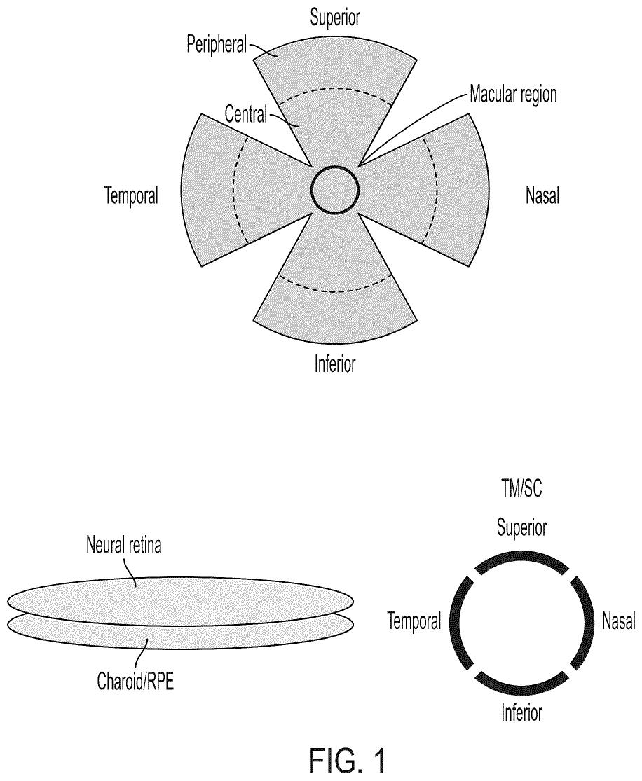

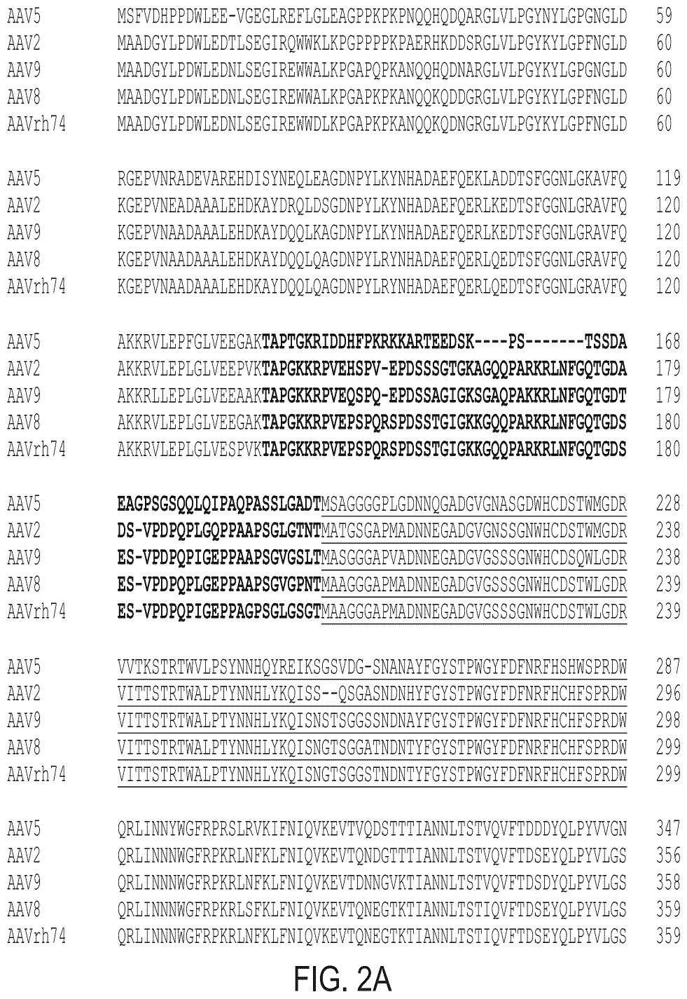

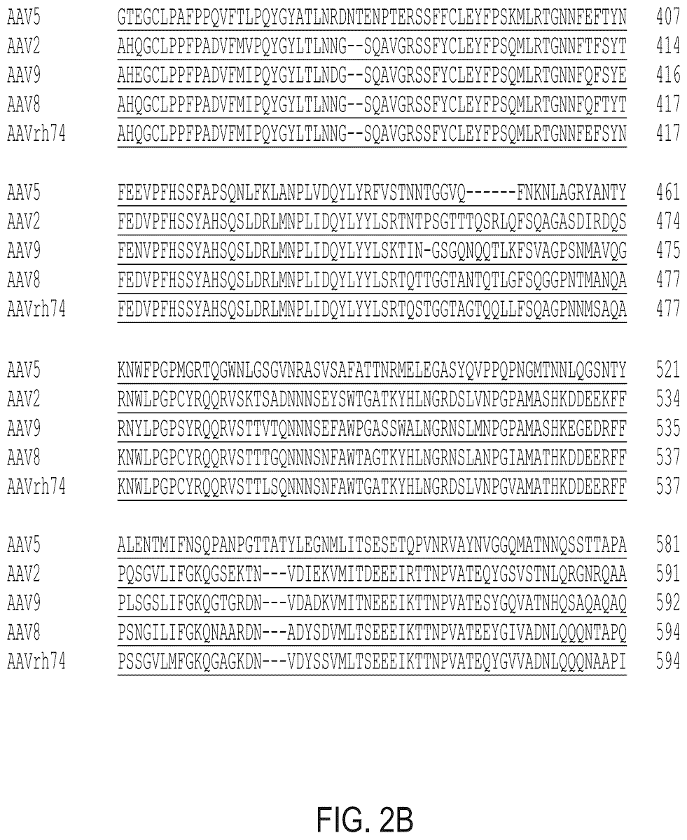

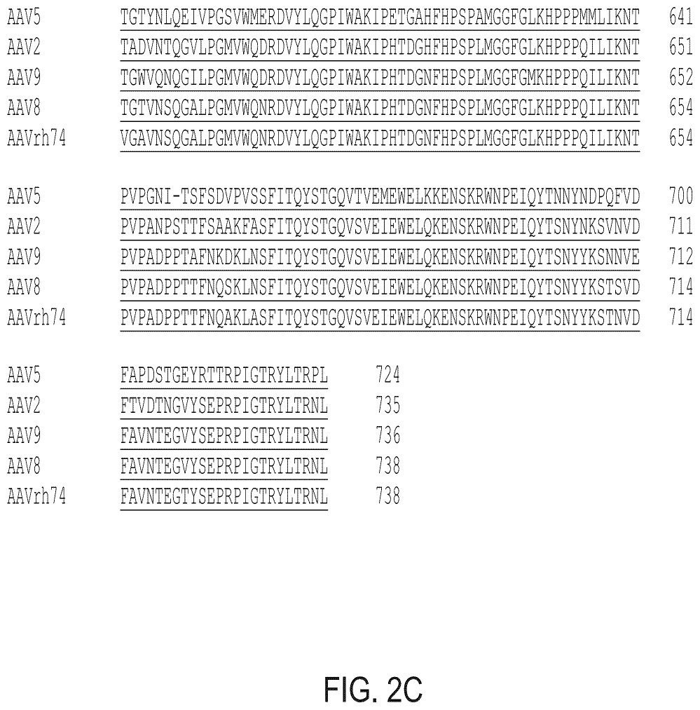

It will be understood by the skilled artisan, and without being bound by theory, that each amino acid position within a reference sequence corresponds to a position within the sequence of other capsid polypeptides such as capsid polypeptides derived from dependoparvoviruses with different serotypes. Such corresponding positions are identified using sequence alignment tools known in the art. A particularly preferred sequence alignment tool is Clustal Omega (Sievers F., et al., Mol. Syst. Biol. 7:359, 2011, DOI: 10.1038/msb.2011.75, incorporated herein by reference in its entirety). An alignment of exemplary reference capsid polypeptides is shown in FIG. 1A-1C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO: 1 (where X is the amino acid present at position nnn in SEQ ID NO: 1 and Y is the amino acid mutation at that position, e.g., described herein), the disclosure provides variant capsid polypeptides comprising at least 80%, at least 85%, at least 90%, at least 91%, at least 92%, at least 93%, at least 94%, at least 95%, at least 96%, at least 97%, at least 98%, or at least 99 identity to the reference capsid polypeptide sequence (e.g., reference capsid polypeptide VP1, VP2 and/or VP3 sequence) other than SEQ ID NO: 1 (or VP2 or VP3 sequence comprised therein) and further comprising the disclosed mutation at a position corresponding to position nnn of SEQ ID NO: 1 (e.g., comprising Y at the position in the new variant capsid polypeptide sequence that corresponds to position nnn of SEQ ID NO: 1). As described above, such corresponding position is determined using a sequence alignment tool, such as, for example, the clustal omega tool described above. Examples of corresponding amino acid positions of exemplary known AAV serotypes is provided in FIG. 2A-C. In some embodiments, the variant is a variant of the AAV2 capsid polypeptide, which can be referred to as a “variant AAV2 capsid polypeptide.”

Thus, in embodiments, the disclosure provides capsid polypeptide sequences that are variants of a reference sequence other than SEQ ID NO: 1, e.g., a reference sequence other than SEQ ID NO: 1 as described herein, which include one or more mutation corresponding to the mutations described herein. In embodiments, such variants include mutations corresponding to all of the mutations associated with any one of VAR-1 through VAR-53 as provided herein.

As used herein, the term “corresponds to” as used in reference to a position in a sequence, such as an amino acid or nucleic acid sequence, can be used in reference to an entire capsid polypeptide or polynucleotide sequence, such as the full length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full length or region) of a certain serotype of AAV, the variant polypeptide can be of the same serotype with a mutation made at such corresponding position as compared to the reference sequence (e.g., full length or region). In some embodiments, the variant capsid polypeptide is a different serotype as compared to the reference sequence.

The variant capsid polypeptides described herein are optionally variants of reference capsids serotypes known in the art. Non-limiting examples of such reference AAV serotypes include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP (3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr—B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region. In certain embodiments, a reference AAV capsid sequence comprises a VP1, VP2 and/or VP3 region, or any combination thereof. A reference VP1 sequence may be considered synonymous with a reference AAV capsid sequence.

An exemplary reference sequence of SEQ ID NO: 1 (wild-type AAV2) is as follows:

(SEQ ID NO: 1)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGITTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDINGVYSEPRPIGTRYLTRNL.

Unless otherwise noted, SEQ ID NO: 1 is the reference sequence. In the sequence above, the sequence found in VP1, VP2 and VP3 is underlined (e.g., a VP3 capsid polypeptide includes, e.g., consists of, amino acids corresponding to amino acids 203-735 of SEQ ID NO: 1), the sequence found in both VP1 and VP2 is in bold (e.g., a VP2 capsid polypeptide includes, e.g., consists of, the sequence corresponding to amino acids 138-735 of SEQ ID NO: 1) and the sequence that is not underlined or bold is found only in VP1 (e.g., a VP1 capsid polypeptide includes, e.g., consists of, amino acids corresponding to amino acids 1-735 of SEQ ID NO: 1).

An example nucleic acid sequence encoding SEQ ID NO: 1 is SEQ ID NO: 92:

(SEQ ID NO: 92)
ATGGCTGCCGATGGTTATCTTCCAGATTGGCTCGAGGACACTCTCTCTG
AAGGAATAAGACAGTGGTGGAAGCTCAAACCTGGCCCACCACCACCAAA
GCCCGCAGAGCGGCATAAGGACGACAGCAGGGGTCTTGTGCTTCCTGGG
TACAAGTACCTCGGACCCTTCAACGGACTCGACAAGGGAGAGCCGGTCA
ACGAGGCAGACGCCGCGGCCCTCGAGCACGACAAAGCCTACGACCGGCA
GCTCGACAGCGGAGACAACCCGTACCTCAAGTACAACCACGCCGACGCG
GAGTTTCAGGAGCGCCTTAAAGAAGATACGTCTTTTGGGGGCAACCTCG
GACGAGCAGTCTTCCAGGCGAAAAAGAGGGTTCTTGAACCTCTGGGCCT
GGTTGAGGAACCTGTTAAGACGGCTCCGGGAAAAAAGAGGCCGGTAGAG
CACTCTCCTGTGGAGCCAGACTCCTCCTCGGGAACCGGAAAGGCGGGCC
AGCAGCCTGCAAGAAAAAGATTGAATTTTGGTCAGACTGGAGACGCAGA
CTCAGTACCTGACCCCCAGCCTCTCGGACAGCCACCAGCAGCCCCCTCT
GGTCTGGGAACTAATACGATGGCTACAGGCAGTGGCGCACCAATGGCAG
ACAATAACGAGGGCGCCGACGGAGTGGGTAATTCCTCGGGAAATTGGCA
TTGCGATTCCACATGGATGGGCGACAGAGTCATCACCACCAGCACCCGA
ACCTGGGCCCTGCCCACCTACAACAACCACCTCTACAAACAAATTTCCA
GCCAATCAGGAGCCTCGAACGACAATCACTACTTTGGCTACAGCACCCC
TTGGGGGTATTTTGACTTCAACAGATTCCACTGCCACTTTTCACCACGT
GACTGGCAAAGACTCATCAACAACAACTGGGGATTCCGACCCAAGAGAC
TCAACTTCAAGCTCTTTAACATTCAAGTCAAAGAGGTCACGCAGAATGA
CGGTACGACGACGATTGCCAATAACCTTACCAGCACGGTTCAGGTGTTT
ACTGACTCGGAGTACCAGCTCCCGTACGTCCTCGGCTCGGCGCATCAAG
GATGCCTCCCGCCGTTCCCAGCAGACGTCTTCATGGTGCCACAGTATGG
ATACCTCACCCTGAACAACGGGAGTCAGGCAGTAGGACGCTCTTCATTT
TACTGCCTGGAGTACTTTCCTTCTCAGATGCTGCGTACCGGAAACAACT
TTACCTTCAGCTACACTTTTGAGGACGTTCCTTTCCACAGCAGCTACGC
TCACAGCCAGAGTCTGGACCGTCTCATGAATCCTCTCATCGACCAGTAC
CTGTATTACTTGAGCAGAACAAACACTCCAAGTGGAACCACCACGCAGT
CAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAG
ACATCTGCGGATAACAACAACAGTGAATACTCGTGGACTGGAGCTACCA
AGTACCACCTCAATGGCAGAGACTCTCTGGTGAATCCGGGCCCGGCCAT
GGCAAGCCACAAGGACGATGAAGAAAAGTTTTTTCCTCAGAGCGGGGTT
CTCATCTTTGGGAAGCAAGGCTCAGAGAAAACAAATGTGGACATTGAAA
AGGTCATGATTACAGACGAAGAGGAAATCAGGACAACCAATCCCGTGGC
TACGGAGCAGTATGGTTCTGTATCTACCAACCTCCAGAGAGGCAACAGA
CAAGCAGCTACCGCAGATGTCAACACACAAGGCGTTCTTCCAGGCATGG
TCTGGCAGGACAGAGATGTGTACCTTCAGGGGCCCATCTGGGCAAAGAT
TCCACACACGGACGGACATTTTCACCCCTCTCCCCTCATGGGTGGATTC
GGACTTAAACACCCTCCTCCACAGATTCTCATCAAGAACACCCCGGTAC
CTGCGAATCCTTCGACCACCTTCAGTGCGGCAAAGTTTGCTTCCTTCAT
CACACAGTACTCCACGGGACAGGTCAGCGTGGAGATCGAGTGGGAGCTG
CAGAAGGAAAACAGCAAACGCTGGAATCCCGAAATTCAGTACACTTCCA
ACTACAACAAGTCTGTTAATGTGGACTTTACTGTGGACACTAATGGCGT
GTATTCAGAGCCTCGCCCCATTGGCACCAGATACCTGACTCGTAATCTG
TAA

An exemplary reference sequence of wild type AAV5, SEQ ID NO: 123 (wild-type AAV5), is as follows:

(SEQ ID NO: 123)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTENPTERSSFFCLEYFP
SKMLRTGNNFEFTYNFEEVPFHSSFAPSQNLFKLANPLVDQYLYRFVST
NNTGGVQFNKNLAGRYANTYKNWFPGPMGRTQGWNLGSGVNRASVSAFA
TTNRMELEGASYQVPPQPNGMTNNLQGSNTYALENTMIFNSQPANPGTT
ATYLEGNMLITSESETQPVNRVAYNVGGQMAINNQSSTTAPATGTYNLQ
EIVPGSVWMERDVYLQGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL

In the sequence above, the sequence found in VP1, VP2 and VP3 is underlined (e.g., a VP3 capsid polypeptide includes, e.g., consists of, amino acids corresponding to amino acids 193-725 of SEQ ID NO: 123), the sequence found in both VP1 and VP2 is in bold (e.g., a VP2 capsid polypeptide includes, e.g., consists of, the sequence corresponding to amino acids 137-725 of SEQ ID NO: 123) and the sequence that is not underlined or bold is found only in VP1 (e.g., a VP1 capsid polypeptide includes, e.g., consists of, amino acids corresponding to amino acids 1-725 of SEQ ID NO: 123).

An example nucleic acid sequence encoding SEQ ID NO: 123 is SEQ ID NO: 124:

(SEQ ID NO: 124)
ATGTCTTTTGTTGATCACCCTCCAGATTGGTTGGAAGAAGTTGGTGAAG
GTCTTCGCGAGTTTTTGGGCCTTGAAGCGGGCCCACCGAAACCAAAACC
CAATCAGCAGCATCAAGATCAAGCCCGTGGTCTTGTGCTGCCTGGTTAT
AACTATCTCGGACCCGGAAACGGGCTCGATCGAGGAGAGCCTGTCAACA
GGGCAGACGAGGTCGCGCGAGAGCACGACATCTCGTACAACGAGCAGCT
TGAGGCGGGAGACAACCCCTACCTCAAGTACAACCACGCGGACGCCGAG
TTTCAGGAGAAGCTCGCCGACGACACATCCTTCGGGGGAAACCTCGGAA
AGGCAGTCTTTCAGGCCAAGAAAAGGGTTCTCGAACCTTTTGGCCTGGT
TGAAGAGGGTGCTAAGACGGCCCCTACCGGAAAGCGGATAGACGACCAC
TTTCCAAAAAGAAAGAAGGCTCGGACCGAAGAGGACTCCAAGCCTTCCA
CCTCGTCAGACGCCGAAGCTGGACCCAGCGGATCCCAGCAGCTGCAAAT
CCCAGCCCAACCAGCCTCAAGTTTGGGAGCTGATACAATGTCTGCGGGA
GGTGGCGGCCCATTGGGCGACAATAACCAAGGTGCCGATGGAGTGGGCA
ATGCCTCGGGAGATTGGCATTGCGATTCCACGTGGATGGGGGACAGAGT
CGTCACCAAGTCCACCCGAACCTGGGTGCTGCCCAGCTACAACAACCAC
CAGTACCGAGAGATCAAAAGCGGCTCCGTCGACGGAAGCAACGCCAACG
CCTACTTTGGATACAGCACCCCCTGGGGGTACTTTGACTTTAACCGCTT
CCACAGCCACTGGAGCCCCCGAGACTGGCAAAGACTCATCAACAACTAC
TGGGGCTTCAGACCCCGGTCCCTCAGAGTCAAAATCTTCAACATTCAAG
TCAAAGAGGTCACGGTGCAGGACTCCACCACCACCATCGCCAACAACCT
CACCTCCACCGTCCAAGTGTTTACGGACGACGACTACCAGCTGCCCTAC
GTCGTCGGCAACGGGACCGAGGGATGCCTGCCGGCCTTCCCTCCGCAGG
TCTTTACGCTGCCGCAGTACGGTTACGCGACGCTGAACCGCGACAACAC
AGAAAATCCCACCGAGAGGAGCAGCTTCTTCTGCCTAGAGTACTTTCCC
AGCAAGATGCTGAGAACGGGCAACAACTTTGAGTTTACCTACAACTTTG
AGGAGGTGCCCTTCCACTCCAGCTTCGCTCCCAGTCAGAACCTGTTCAA
GCTGGCCAACCCGCTGGTGGACCAGTACTTGTACCGCTTCGTGAGCACA
AATAACACTGGCGGAGTCCAGTTCAACAAGAACCTGGCCGGGAGATACG
CCAACACCTACAAAAACTGGTTCCCGGGGCCCATGGGCCGAACCCAGGG
CTGGAACCTGGGCTCCGGGGTCAACCGCGCCAGTGTCAGCGCCTTCGCC
ACGACCAATAGGATGGAGCTCGAGGGCGCGAGTTACCAGGTGCCCCCGC
AGCCGAACGGCATGACCAACAACCTCCAGGGCAGCAACACCTATGCCCT
GGAGAACACTATGATCTTCAACAGCCAGCCGGCGAACCCGGGCACCACC
GCCACGTACCTCGAGGGCAACATGCTCATCACCAGCGAGAGCGAGACGC
AGCCGGTGAACCGCGTGGCGTACAACGTCGGCGGGCAGATGGCCACCAA
CAACCAGAGCTCCACCACTGCCCCCGCGACCGGCACGTACAACCTCCAG
GAAATCGTGCCCGGCAGCGTGTGGATGGAGAGGGACGTGTACCTCCAAG
GACCCATCTGGGCCAAGATCCCAGAGACGGGGGCGCACTTTCACCCCTC
TCCGGCCATGGGCGGATTCGGACTCAAACACCCACCGCCCATGATGCTC
ATCAAGAACACGCCTGTGCCCGGAAATATCACCAGCTTCTCGGACGTGC
CCGTCAGCAGCTTCATCACCCAGTACAGCACCGGGCAGGTCACCGTGGA
GATGGAGTGGGAGCTCAAGAAGGAAAACTCCAAGAGGTGGAACCCAGAG
ATCCAGTACACAAACAACTACAACGACCCCCAGTTTGTGGACTTTGCCC
CGGACAGCACCGGGGAATACAGAACCACCAGACCTATCGGAACCCGATA
CCTTACCCGACCCCTTTAA

An exemplary reference sequence of wild-type AAV8, SEQ ID NO: 125 (wild-type AAV8), is as follows:

(SEQ ID NO: 125)
MAADGYLPDWLEDNLSEGIREWWALKPGAPKPKANQQKQDDGRGLVLPG
YKYLGPENGLDKGEPVNAADAAALEHDKAYDQQLQAGDNPYLRYNHADA
EFQERLQEDTSFGGNLGRAVFQAKKRVLEPLGLVEEGAKTAPGKKRPVE
PSPQRSPDSSTGIGKKGQQPARKRLNFGQTGDSESVPDPQPLGEPPAAP
SGVGPNTMAAGGGAPMADNNEGADGVGSSSGNWHCDSTWLGDRVITTST
RIWALPTYNNHLYKQISNGTSGGATNDNTYFGYSTPWGYFDFNRFHCHF
SPRDWQRLINNNWGFRPKRLSFKLENIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFQFTYTFEDVPFHSSYAHSQSLDRLMNPLI
DQYLYYLSRTQTTGGTANTQTLGFSQGGPNTMANQAKNWLPGPCYRQQR
VSTTTGQNNNSNFAWTAGTKYHLNGRNSLANPGIAMATHKDDEERFFPS
NGILIFGKQNAARDNADYSDVMLTSEEEIKTTNPVATEEYGIVADNLQQ
QNTAPQIGTVNSQGALPGMVWQNRDVYLQGPIWAKIPHTDGNFHPSPLM
GGFGLKHPPPQILIKNTPVPADPPTTFNQSKLNSFITQYSTGQVSVEIE
WELQKENSKRWNPEIQYTSNYYKSTSVDFAVNTEGVYSEPRPIGTRYLT
RNL

In the sequence above, the sequence found in VP1, VP2 and VP3 is underlined (e.g., a VP3 capsid polypeptide includes, e.g., consists of, amino acids corresponding to amino acids 204-739 of SEQ ID NO: 125), the sequence found in both VP1 and VP2 is in bold (e.g., a VP2 capsid polypeptide includes, e.g., consists of, the sequence corresponding to amino acids 138-735 of SEQ ID NO: 125) and the sequence that is not underlined or bold is found only in VP1 (e.g., a VP1 capsid polypeptide includes, e.g., consists of, amino acids corresponding to amino acids 1-739 of SEQ ID NO: 125).

An example nucleic acid sequence encoding SEQ ID NO: 125 is SEQ ID NO: 126:

(SEQ ID NO: 126)
ATGGCTGCCGATGGTTATCTTCCAGATTGGCTCGAGGACAACCTCTCTG
AGGGCATTCGCGAGTGGTGGGCGCTGAAACCTGGAGCCCCGAAGCCCAA
AGCCAACCAGCAAAAGCAGGACGACGGCCGGGGTCTGGTGCTTCCTGGC
TACAAGTACCTCGGACCCTTCAACGGACTCGACAAGGGGGAGCCCGTCA
ACGCGGCGGACGCAGCGGCCCTCGAGCACGACAAGGCCTACGACCAGCA
GCTGCAGGGGGGTGACAATCCGTACCTGCGGTATAACCACGCCGACGCC
GAGTTTCAGGAGCGTCTGCAAGAAGATACGTCTTTTGGGGGCAACCTCG
GGCGAGCAGTCTTCCAGGCCAAGAAGCGGGTTCTCGAACCTCTCGGTCT
GGTTGAGGAAGGCGCTAAGACGGCTCCTGGAAAGAAGAGACCGGTAGAG
CCATCACCCCAGCGTTCTCCAGACTCCTCTACGGGCATCGGCAAGAAAG
GCCAACAGCCCGCCAGAAAAAGACTCAATTTTGGTCAGACTGGCGACTC
AGAGTCAGTTCCAGACCCTCAACCTCTCGGAGAACCTCCAGCAGCGCCC
TCTGGTGTGGGACCTAATACAATGGCTGCAGGCGGTGGCGCACCAATGG
CAGACAATAACGAAGGCGCCGACGGAGTGGGTAGTTCCTCGGGAAATTG
GCATTGCGATTCCACATGGCTGGGCGACAGAGTCATCACCACCAGCACC
CGAACCTGGGCCCTGCCCACCTACAACAACCACCTCTACAAGCAAATCT
CCAACGGGACATCGGGAGGAGCCACCAACGACAACACCTACTTCGGCTA
CAGCACCCCCTGGGGGTATTTTGACTTTAACAGATTCCACTGCCACTTT
TCACCACGTGACTGGCAGCGACTCATCAACAACAACTGGGGATTCCGGC
CCAAGAGACTCAGCTTCAAGCTCTTCAACATCCAGGTCAAGGAGGTCAC
GCAGAATGAAGGCACCAAGACCATCGCCAATAACCTCACCAGCACCATC
CAGGTGTTTACGGACTCGGAGTACCAGCTGCCGTACGTTCTCGGCTCTG
CCCACCAGGGCTGCCTGCCTCCGTTCCCGGCGGACGTGTTCATGATTCC
CCAGTACGGCTACCTAACACTCAACAACGGTAGTCAGGCCGTGGGACGC
TCCTCCTTCTACTGCCTGGAATACTTTCCTTCGCAGATGCTGAGAACCG
GCAACAACTTCCAGTTTACTTACACCTTCGAGGACGTGCCTTTCCACAG
CAGCTACGCCCACAGCCAGAGCTTGGACCGGCTGATGAATCCTCTGATT
GACCAGTACCTGTACTACTTGTCTCGGACTCAAACAACAGGAGGCACGG
CAAATACGCAGACTCTGGGCTTCAGCCAAGGTGGGCCTAATACAATGGC
CAATCAGGCAAAGAACTGGCTGCCAGGACCCTGTTACCGCCAACAACGC
GTCTCAACGACAACCGGGCAAAACAACAATAGCAACTTTGCCTGGACTG
CTGGGACCAAATACCATCTGAATGGAAGAAATTCATTGGCTAATCCTGG
CATCGCTATGGCAACACACAAAGACGACGAGGAGCGTTTTTTTCCCAGT
AACGGGATCCTGATTTTTGGCAAACAAAATGCTGCCAGAGACAATGCGG
ATTACAGCGATGTCATGCTCACCAGCGAGGAAGAAATCAAAACCACTAA
CCCTGTGGCTACAGAGGAATACGGTATCGTGGCAGATAACTTGCAGCAG
CAAAACACGGCTCCTCAAATTGGAACTGTCAACAGCCAGGGGGCCTTAC
CCGGTATGGTCTGGCAGAACCGGGACGTGTACCTGCAGGGTCCCATCTG
GGCCAAGATTCCTCACACGGACGGCAACTTCCACCCGTCTCCGCTGATG
GGCGGCTTTGGCCTGAAACATCCTCCGCCTCAGATCCTGATCAAGAACA
CGCCTGTACCTGCGGATCCTCCGACCACCTTCAACCAGTCAAAGCTGAA
CTCTTTCATCACGCAATACAGCACCGGACAGGTCAGCGTGGAAATTGAA
TGGGAGCTGCAGAAGGAAAACAGCAAGCGCTGGAACCCCGAGATCCAGT
ACACCTCCAACTACTACAAATCTACAAGTGTGGACTTTGCTGTTAATAC
AGAAGGCGTGTACTCTGAACCCCGCCCCATTGGCACCCGTTACCTCACC
CGTAATCTGTAA

An exemplary reference sequence of wild-type AAV9, SEQ ID NO: 127 (wild-type AAV9), is as follows:

(SEQ ID NO: 127)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
AQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRN
L

In the sequence above, the sequence found in VP1, VP2 and VP3 is underlined (e.g., a VP3 capsid polypeptide includes, e.g., consists of, amino acids corresponding to amino acids 203-737 of SEQ ID NO: 127), the sequence found in both VP1 and VP2 is in bold (e.g., a VP2 capsid polypeptide includes, e.g., consists of, the sequence corresponding to amino acids 138-737 of SEQ ID NO: 127) and the sequence that is not underlined or bold is found only in VP1 (e.g., a VP1 capsid polypeptide includes, e.g., consists of, amino acids corresponding to amino acids 1-737 of SEQ ID NO: 127).

An example nucleic acid sequence encoding SEQ ID NO: 127 is SEQ ID NO: 128:

(SEQ ID NO: 128)
ATGGCTGCCGATGGTTATCTTCCAGATTGGCTCGAGGACAACCTTAGTG
AAGGTATTCGCGAGTGGTGGGCTTTGAAACCTGGAGCCCCTCAACCCAA
GGCAAATCAACAACATCAAGACAACGCTCGAGGTCTTGTGCTTCCGGGT
TACAAATACCTTGGACCCGGCAACGGACTCGACAAGGGGGAGCCGGTCA
ACGCAGCAGACGCGGCGGCCCTCGAGCACGACAAGGCCTACGACCAGCA
GCTCAAGGCCGGAGACAACCCGTACCTCAAGTACAACCACGCCGACGCC
GAGTTCCAGGAGCGGCTCAAAGAAGATACGTCTTTTGGGGGCAACCTCG
GGCGAGCAGTCTTCCAGGCCAAAAAGAGGCTTCTTGAACCTCTTGGTCT
GGTTGAGGAAGCGGCTAAGACGGCTCCTGGAAAGAAGAGGCCTGTAGAG
CAGTCTCCTCAGGAACCGGACTCCTCCGCGGGTATTGGCAAATCGGGTG
CACAGCCCGCTAAAAAGAGACTCAATTTCGGTCAGACTGGCGACACAGA
GTCAGTCCCAGACCCTCAACCAATCGGAGAACCTCCCGCAGCCCCCTCA
GGTGTGGGATCTCTTACAATGGCTTCAGGTGGTGGCGCACCAGTGGCAG
ACAATAACGAAGGTGCCGATGGAGTGGGTAGTTCCTCGGGAAATTGGCA
TTGCGATTCCCAATGGCTGGGGGACAGAGTCATCACCACCAGCACCCGA
ACCTGGGCCCTGCCCACCTACAACAATCACCTCTACAAGCAAATCTCCA
ACAGCACATCTGGAGGATCTTCAAATGACAACGCCTACTTCGGCTACAG
CACCCCCTGGGGGTATTTTGACTTCAACAGATTCCACTGCCACTTCTCA
CCACGTGACTGGCAGCGACTCATCAACAACAACTGGGGATTCCGGCCTA
AGCGACTCAACTTCAAGCTCTTCAACATTCAGGTCAAAGAGGTTACGGA
CAACAATGGAGTCAAGACCATCGCCAATAACCTTACCAGCACGGTCCAG
GTCTTCACGGACTCAGACTATCAGCTCCCGTACGTGCTCGGGTCGGCTC
ACGAGGGCTGCCTCCCGCCGTTCCCAGCGGACGTTTTCATGATTCCTCA
GTACGGGTATCTGACGCTTAATGATGGAAGCCAGGCCGTGGGTCGTTCG
TCCTTTTACTGCCTGGAATATTTCCCGTCGCAAATGCTAAGAACGGGTA
ACAACTTCCAGTTCAGCTACGAGTTTGAGAACGTACCTTTCCATAGCAG
CTACGCTCACAGCCAAAGCCTGGACCGACTAATGAATCCACTCATCGAC
CAATACTTGTACTATCTCTCAAAGACTATTAACGGTTCTGGACAGAATC
AACAAACGCTAAAATTCAGTGTGGCCGGACCCAGCAACATGGCTGTCCA
GGGAAGAAACTACATACCTGGACCCAGCTACCGACAACAACGTGTCTCA
ACCACTGTGACTCAAAACAACAACAGCGAATTTGCTTGGCCTGGAGCTT
CTTCTTGGGCTCTCAATGGACGTAATAGCTTGATGAATCCTGGACCTGC
TATGGCCAGCCACAAAGAAGGAGAGGACCGTTTCTTTCCTTTGTCTGGA
TCTTTAATTTTTGGCAAACAAGGAACTGGAAGAGACAACGTGGATGCGG
ACAAAGTCATGATAACCAACGAAGAAGAAATTAAAACTACTAACCCGGT
AGCAACGGAGTCCTATGGACAAGTGGCCACAAACCACCAGAGTGCCCAA
GCACAGGCGCAGACCGGCTGGGTTCAAAACCAAGGAATACTTCCGGGTA
TGGTTTGGCAGGACAGAGATGTGTACCTGCAAGGACCCATTTGGGCCAA
AATTCCTCACACGGACGGCAACTTTCACCCTTCTCCGCTGATGGGAGGG
TTTGGAATGAAGCACCCGCCTCCTCAGATCCTCATCAAAAACACACCTG
TACCTGCGGATCCTCCAACGGCCTTCAACAAGGACAAGCTGAACTCTTT
CATCACCCAGTATTCTACTGGCCAAGTCAGCGTGGAGATCGAGTGGGAG
CTGCAGAAGGAAAACAGCAAGCGCTGGAACCCGGAGATCCAGTACACTT
CCAACTATTACAAGTCTAATAATGTTGAATTTGCTGTTAATACTGAAGG
TGTATATAGTGAACCCCGCCCCATTGGCACCAGATACCTGACTCGTAAT
CTGTAA

An exemplary reference sequence of wild-type AAVrh74, SEQ ID NO: 129 (wild-type AAVrh74), is as follows:

(SEQ ID NO: 129)
MAADGYLPDWLEDNLSEGIREWWDLKPGAPKPKANQQKQDNGRGLVLPG
YKYLGPENGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLENIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL

An alternative exemplary reference sequence of SEQ ID NO: 130 (alternate wild-type AAVrh74) is as follows:

(SEQ ID NO: 130)
MAADGYLPDWLEDNLSEGIREWWDLKPGAPKPKANQQKQDNGRGLVLPG
YKYLGPENGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLENIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFTKAKLASFITQYSTGQVSVEIE
WELQKENSKRWNPEIQYTSNYYKSTNVDFAVNTEGTYSEPRPIGTRYLT
RNL

In the sequences above (SEQ ID NO: 129 or SEQ ID NO: 130), the sequence found in VP1, VP2 and VP3 is underlined (e.g., a VP3 capsid polypeptide includes, e.g., consists of, amino acids corresponding to amino acids 204-739 of SEQ ID NO: 129), the sequence found in both VP1 and VP2 is in bold (e.g., a VP2 capsid polypeptide includes, e.g., consists of, the sequence corresponding to amino acids 137-739 of SEQ ID NO: 129) and the sequence that is not underlined or bold is found only in VP1 (e.g., a VP1 capsid polypeptide includes, e.g., consists of, amino acids corresponding to amino acids 1-739 of SEQ ID NO: 129).

An example nucleic acid sequence encoding SEQ ID NO: 129 is SEQ ID NO: 131.

(SEQ ID NO: 131)
ATGGCTGCCGATGGTTATCTTCCAGATTGGCTCGAGGACAACCTCTCTG
AGGGCATTCGCGAGTGGTGGGACCTGAAACCTGGAGCCCCGAAACCCAA
AGCCAACCAGCAAAAGCAGGACAACGGCCGGGGTCTGGTGCTTCCTGGC
TACAAGTACCTCGGACCCTTCAACGGACTCGACAAGGGGGAGCCCGTCA
ACGCGGCGGACGCAGCGGCCCTCGAGCACGACAAGGCCTACGACCAGCA
GCTCCAAGCGGGTGACAATCCGTACCTGCGGTATAATCACGCCGACGCC
GAGTTTCAGGAGCGTCTGCAAGAAGATACGTCTTTTGGGGGCAACCTCG
GGCGCGCAGTCTTCCAGGCCAAAAAGCGGGTTCTCGAACCTCTGGGCCT
GGTTGAATCGCCGGTTAAGACGGCTCCTGGAAAGAAGAGGCCGGTAGAG
CCATCACCCCAGCGCTCTCCAGACTCCTCTACGGGCATCGGCAAGAAAG
GCCAGCAGCCCGCAAAAAAGAGACTCAATTTTGGGCAGACTGGCGACTC
AGAGTCAGTCCCCGACCCTCAACCAATCGGAGAACCACCAGCAGGCCCC
TCTGGTCTGGGATCTGGTACAATGGCTGCAGGCGGTGGCGCTCCAATGG
CAGACAATAACGAAGGCGCCGACGGAGTGGGTAGTTCCTCAGGAAATTG
GCATTGCGATTCCACATGGCTGGGCGACAGAGTCATCACCACCAGCACC
CGCACCTGGGCCCTGCCCACCTACAACAACCACCTCTACAAGCAAATCT
CCAACGGGACCTCGGGAGGAAGCACCAACGACAACACCTACTTCGGCTA
CAGCACCCCCTGGGGGTATTTTGACTTCAACAGATTCCACTGCCACTTT
TCACCACGTGACTGGCAGCGACTCATCAACAACAACTGGGGATTCCGGC
CCAAGAGGCTCAACTTCAAGCTCTTCAACATCCAAGTCAAGGAGGTCAC
GCAGAATGAAGGCACCAAGACCATCGCCAATAACCTTACCAGCACGATT
CAGGTCTTTACGGACTCGGAATACCAGCTCCCGTACGTGCTCGGCTCGG
CGCACCAGGGCTGCCTGCCTCCGTTCCCGGCGGACGTCTTCATGATTCC
TCAGTACGGGTACCTGACTCTGAACAATGGCAGTCAGGCTGTGGGCCGG
TCGTCCTTCTACTGCCTGGAGTACTTTCCTTCTCAAATGCTGAGAACGG
GCAACAACTTTGAATTCAGCTACAACTTCGAGGACGTGCCCTTCCACAG
CAGCTACGCGCACAGCCAGAGCCTGGACCGGCTGATGAACCCTCTCATC
GACCAGTACTTGTACTACCTGTCCCGGACTCAAAGCACGGGCGGTACTG
CAGGAACTCAGCAGTTGCTATTTTCTCAGGCCGGGCCTAACAACATGTC
GGCTCAGGCCAAGAACTGGCTACCCGGTCCCTGCTACCGGCAGCAACGT
GTCTCCACGACACTGTCGCAGAACAACAACAGCAACTTTGCCTGGACGG
GTGCCACCAAGTATCATCTGAATGGCAGAGACTCTCTGGTGAATCCTGG
CGTTGCCATGGCTACCCACAAGGACGACGAAGAGCGATTTTTTCCATCC
AGCGGAGTCTTAATGTTTGGGAAACAGGGAGCTGGAAAAGACAACGTGG
ACTATAGCAGCGTGATGCTAACCAGCGAGGAAGAAATAAAGACCACCAA
CCCAGTGGCCACAGAACAGTACGGCGTGGTGGCCGATAACCTGCAACAG
CAAAACGCCGCTCCTATTGTAGGGGCCGTCAATAGTCAAGGAGCCTTAC
CTGGCATGGTGTGGCAGAACCGGGACGTGTACCTGCAGGGTCCCATCTG
GGCCAAGATTCCTCATACGGACGGCAACTTTCATCCCTCGCCGCTGATG
GGAGGCTTTGGACTGAAGCATCCGCCTCCTCAGATCCTGATTAAAAACA
CACCTGTTCCCGCGGATCCTCCGACCACCTTCAATCAGGCCAAGCTGGC
TTCTTTCATCACGCAGTACAGTACCGGCCAGGTCAGCGTGGAGATCGAG
TGGGAGCTGCAGAAGGAGAACAGCAAACGCTGGAACCCAGAGATTCAGT
ACACTTCCAACTACTACAAATCTACAAATGTGGACTTTGCTGTCAATAC
TGAGGGTACTTATTCCGAGCCTCGCCCCATTGGCACCCGTTACCTCACC
CGTAATCTGTAA

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the polypeptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA-clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprise VP capsid proteins which include a Met1/AA1 amino acid (Met+/AA+) as well as corresponding VP capsid polypeptide which lack the Met1/AA1 amino acid e.g. as a result of Met/AA-clipping (Met−/AA−). Further according to the present disclosure, a reference to a specific SEQ ID NO: (whether a protein or nucleic acid) which comprises or encodes, respectively, one or more capsid polypeptides which include a Met1/AA1 amino acid (Met+/AA+) should be understood to teach the VP capsid polypeptides which lack the Met1/AA1 amino acid as upon review of the sequence, it is readily apparent that the first listed amino acid (whether or not Met1/AA1) may be absent. As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+ sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid e.g. as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, the reference AAV capsid sequence is encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above. In certain embodiments, the reference sequence is not an AAV capsid sequence and is instead a different vector (e.g., lentivirus, plasmid, etc.).

In some embodiments, a nucleic acid of the disclosure (e.g., encoding an AAV2 variant capsid protein) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and may be utilized with the compositions and methods disclosed herein.

In some embodiments, the native promoter for the transgene may be used. Without wishing to be bound by theory, the native promoter may mimic native expression of the transgene, or provide temporal, developmental, or tissue-specific expression, or expression in response to specific transcriptional stimuli. In some embodiment, the transgene may be operably linked to other native expression control elements, such as enhancer elements, polyadenylation sites or Kozak consensus sequences, e.g., to mimic the native expression.

In some embodiments, the transgene is operably linked to a tissue-specific promoter.

In some embodiments, a vector, e.g., a plasmid, carrying a transgene may also include a selectable marker or a reporter gene. Such selectable reporters or marker genes can be used to signal the presence of the vector, e.g., plasmid, in bacterial cells. Other components of the vector, e.g., plasmid, may include an origin of replication. Selection of these and other promoters and vector elements are conventional and many such sequences are available (see, e.g., Sambrook et al, and references cited therein).

In some embodiments, the capsid polypeptide present in a viral particle increases transduction in the eye as compared to a viral particle with a reference capsid polypeptide, for example, with the wild-type capsid polypeptide (SEQ ID NO: 1). In some embodiments, the capsid polypeptide present in a viral particle increases transduction in the retina as compared to a viral particle with a reference capsid polypeptide, for example, with the wild-type capsid polypeptide (SEQ ID NO: 1). In some embodiments, the capsid polypeptide present in a viral particle increases transduction in the non-macular retina as compared to a viral particle with a reference capsid polypeptide, for example, with the wild-type capsid polypeptide (SEQ ID NO: 1). In some embodiments, the capsid polypeptide present in a viral particle increases transduction in the macula as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to non-macular retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula as compared to a viral particle with a reference capsid polypeptide, for example, with the wild-type capsid polypeptide (SEQ ID NO: 1). In some embodiments, the capsid polypeptide present in a viral particle increases transduction in the macula relative to retina as compared to a viral particle with a reference capsid polypeptide, for example, with the wild-type capsid polypeptide (SEQ ID NO: 1). In some embodiments, the capsid polypeptide present in a viral particle increases transduction in the macula relative to non-macular retina as compared to a viral particle with a reference capsid polypeptide, for example, with the wild-type capsid polypeptide (SEQ ID NO: 1). In some embodiments, the capsid polypeptide present in a viral particle increases transduction in the macula relative to trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the macula relative to non-macular retina and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the macula relative to retina and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the retina relative to macula and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the non-macular retina relative to macula and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula and retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula and non-macular retina as compared to a viral particle with a reference capsid polypeptide, for example, with the wild-type capsid polypeptide (SEQ ID NO: 1).

In some embodiments, the capsid polypeptide present in a viral particle increases ocular transduction at least 1-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction at least 2-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 4-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 6-fold, e.g., as compared to a viral particle with a reference capsid polypeptide, for example, with the wild-type capsid polypeptide (SEQ ID NO: 1). In some embodiments, the capsid polypeptide present in a viral particle increases v transduction 8-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 10-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 15-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 16-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 32-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 64-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 100-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 150-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 200-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 500-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 1000-fold, e.g., as compared to a viral particle with a reference capsid polypeptide, for example, with the wild-type capsid polypeptide (SEQ ID NO: 1). In embodiments, increased ocular transduction is measured by comparing the level of mRNA in the target tissue (e.g., in a cell or population of cells of the target tissue) produced from a nucleic acid packaged in the variant viral particle with the level of mRNA in the target tissue (e.g., in a cell or population of cells of the target tissue) produced from a nucleic acid packaged in a reference viral particle (e.g., packaged in a capsid comprising capsid polypeptides of SEQ ID NO: 1).

In some embodiments, the capsid polypeptide is an isolated or purified polypeptide (e.g., isolated or purified from a cell, other biological component, or contaminant). In some embodiments, the variant polypeptide is present in a dependoparvovirus particle, e.g., described herein. In some embodiments, the variant capsid polypeptide is present in a cell, cell-free system, or translation system, e.g., described herein.

In some embodiments, the capsid polypeptide is present in a dependoparvovirus B (e.g., AAV2) particle. In some embodiments, the capsid particle has increased ocular transduction.

In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., SEQ ID NO: 2-46). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a dependoparvovirus particle or method of making the same. In some embodiments, the additional alteration improves or alters another characteristic of a dependoparvovirus particle, e.g., tropism.

### VP1 Nucleic Acids and Polypeptides

The disclosure is further directed, in part, to a nucleic acid comprising a sequence encoding a dependoparvovirus (e.g., dependoparvovirus B, e.g., an AAV2) polypeptide as provided for herein, as well as to a VP1 polypeptide encoded by the same. In some embodiments, the polypeptide comprises a sequence of SEQ ID NOs: 2, 3, 4, 5, 6, 7, 8, 9, 10, 11, 12, 13, 14, 15, 16, 17, 18, 19, 20, 21, 22, 23, 24, 25, 25, 27, 28, 29, 31, 32, 33, 34, 35, 36, 37, 38, 39, 40, 41, 42, 43, 44, 45, or 46.

### Dependoparvovirus Particles

The disclosure is also directed, in part, to a dependoparvovirus particle (e.g., a functional dependoparvovirus particle) comprising a nucleic acid or polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a capsid polypeptide provided for herein. In some embodiments, the particle comprises a polypeptide as provided for herein.

In some embodiments, the dependoparvovirus particle of the disclosure may be an AAV2 particle. In some embodiments, the AAV2 particle comprises a capsid polypeptide as provided for herein or a nucleic acid molecule encoding the same.

In some embodiments the dependoparvovirus particle comprises a capsid comprising a variant capsid polypeptide described herein. In embodiments, the dependoparvovirus particle comprises variant capsid polypeptide described herein and a nucleic acid molecule. In embodiments, the dependoparvovirus particle comprises variant capsid polypeptide described herein and a nucleic acid molecule comprising one or more inverted terminal repeat sequences (ITRs), for example, ITRs derived from an AAV2 dependoparvovirus, one or more regulatory elements (for example, a promoter), and a payload (e.g., as described herein). In embodiments, at least one of the ITRs is modified. In embodiments, the nucleic acid molecule is single-stranded. In embodiments, the nucleic acid molecule is self-complementary.

### Increased Ocular Transduction Characteristics

The disclosure is directed, in part, to nucleic acids, polypeptides, cells, cell free systems, translation systems, viral particles, and methods associated with making the same to produce virus particles that have increased ocular transduction as compared to a virus particle having capsid polypeptides of a reference sequence, e.g., with a wild-type sequence of SEQ ID NO: 1. In some embodiments, a use of a viral particle comprising the variant capsid polypeptide leads to increased ocular transduction of a transgene in the eye, and, therefore, expression of the transgene in the eye. In some embodiments, a use of a viral particle comprising the variant capsid polypeptide leads to increased ocular transduction of a transgene in the retina, and, therefore, expression of the transgene in the retina. In some embodiments, a use of a viral particle comprising the variant capsid polypeptide leads to increased ocular transduction of a transgene in the non-macular retina, and, therefore, expression of the transgene in the non-macular retina. In some embodiments, a use of a viral particle comprising the variant capsid polypeptide leads to increased ocular transduction of a transgene in the macula, and, therefore, expression of the transgene in the macula. In some embodiments, a use of a viral particle comprising the variant capsid polypeptide leads to increased ocular transduction of a transgene in the trabecular meshwork, and, therefore, expression of the transgene in the trabecular meshwork. In some embodiments, a use of a viral particle comprising the variant capsid polypeptide leads to increased ocular transduction of a transgene in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, use of a viral particle comprising the variant capsid polypeptide leads to increased ocular transduction of a transgene in the cornea, iris, ciliary body, lens, trabecular meshwork, or Schlemm's canal, or any combination thereof. In some embodiments, a use of a viral particle comprising the variant capsid polypeptide leads to increased ocular transduction of a transgene posterior to the lens, such as in the anterior hyaloid membrane and all of the optical structures behind it, such as the vitreous humor, retina, choroid or optic nerve, or any combination thereof. Accordingly, in some embodiments, use of a viral particle comprising the variant capsid polypeptide leads to increased ocular transduction of a transgene in the anterior hyaloid membrane and all of the optical structures behind it, such as the vitreous humor, retina, choroid or optic nerve, or any combination thereof. In some embodiments, a use of a viral particle comprising the variant capsid polypeptide leads to increased ocular transduction of a transgene in the front third of the eye and posterior to the lens.

In some embodiments, the increase in ocular transduction is, on a log 2 scale, about 1-5 times better (e.g., about 2-5 times better, e.g., about 3-5 times better) than a virus particle having a reference sequence capsid polypeptide, e.g., having the wild-type capsid polypeptide SEQ ID NO: 1.

In some embodiments, the capsid polypeptide present in a viral particle increases transduction without increasing the biodistribution of the variant capsid polypeptide in the eye relative to SEQ ID NO: 1. In some embodiments, the capsid polypeptide present in a viral particle increases transduction without increasing the biodistribution of the variant capsid polypeptide in the retina relative to SEQ ID NO: 1. In some embodiments, the capsid polypeptide present in a viral particle increases transduction without increasing the biodistribution of the variant capsid polypeptide in the trabecular meshwork relative to SEQ ID NO: 1.

Table 5 lists information regarding biodistribution of variant dependoparvovirus particles comprising capsid polypeptides of the indicated variant capsid in the different layers, structures, and/or parts of the eye. Biodistribution in retina is as measured following IVT injection. Biodistribution in trabecular meshwork is as measured following IC injection.

TABLE 5

| | SEQ ID NO: | Biodistribution in | Biodistribution in |
|---|---|---|---|
| | of VP1 | Retina as compared | Trabecular Meshwork as |
| | capsid | to wild-type | compared to wild-type |
| | poly- | SEQ ID NO: 1 | SEQ ID NO: 1 |
| Name | peptide | (Log2) | (Log2) |
| VAR-1 | 2 | 0.219554617 | −0.129512259 |
| VAR-2 | 3 | 0.082776163 | −0.093624787 |
| VAR-3 | 4 | 1.048865823 | −0.097505361 |
| VAR-4 | 5 | 1.985458577 | 0.752795147 |
| VAR-5 | 6 | 0.150339954 | 0.240806265 |
| VAR-6 | 7 | −0.329057077 | −0.127768486 |
| VAR-7 | 8 | −0.485573763 | 0.038632248 |
| VAR-8 | 9 | 2.592283234 | 0.639490423 |
| VAR-9 | 10 | 0.907849174 | −0.129048301 |
| VAR-10 | 11 | 0.582650953 | −0.951767221 |
| VAR-11 | 12 | 1.055569616 | 0.517659804 |
| VAR-12 | 13 | 0.38695579 | 0.536577648 |
| VAR-13 | 14 | 1.336116818 | 1.154005075 |
| VAR-14 | 15 | 0.05824263 | 0.403134632 |
| VAR-15 | 16 | −2.505454013 | −0.891040148 |
| VAR-16 | 17 | −1.08115075 | 0.639490423 |
| VAR-17 | 18 | 0.979546949 | −0.282516221 |
| VAR-18 | 19 | −0.226638005 | −0.219115256 |
| VAR-19 | 20 | 0.876781617 | 0.618096401 |
| VAR-20 | 21 | 0.837693238 | 0.750988668 |
| VAR-21 | 22 | −0.239478568 | 0.539038877 |
| VAR-22 | 23 | −1.789903254 | 0.973742845 |
| VAR-23 | 24 | −0.304881561 | 0.427104122 |
| VAR-24 | 25 | −1.041019591 | 1.040413058 |
| VAR-25 | 26 | 0.901098892 | −0.888153107 |
| VAR-26 | 27 | −1.095478038 | Not measured |
| VAR-27 | 28 | −1.157196676 | 0.001971904 |
| VAR-28 | 29 | 0.5459013 | 0.238124001 |
| VAR-29 | 30 | 1.266904701 | −0.49656868 |
| VAR-30 | 31 | 0.443890223 | 0.110293386 |
| VAR-31 | 32 | 1.695754653 | 0.781373819 |
| VAR-32 | 33 | 1.970541935 | 0.194078521 |
| VAR-33 | 34 | 1.615256454 | −0.347209059 |
| VAR-34 | 35 | 1.104478115 | 1.045899544 |
| VAR-35 | 36 | 2.189521186 | 0.671956827 |
| VAR-36 | 37 | 1.720060573 | 0.031648718 |
| VAR-37 | 38 | 1.810695682 | 1.101034688 |
| VAR-38 | 39 | 0.820878902 | 1.097873642 |
| VAR-39 | 40 | 1.282933021 | 1.250467569 |
| VAR-40 | 41 | 0.218752223 | 0.889665279 |
| VAR-41 | 42 | 0.059531262 | 0.04061276 |
| VAR-42 | 43 | −0.959157062 | 0.791762522 |
| VAR-43 | 44 | −1.779802685 | −0.20186619 |
| VAR-44 | 45 | −0.569765447 | 0.946089818 |
| VAR-45 | 46 | 0.901098892 | 0.048731959 |

According to some embodiments, disclosed herein are capsid polypeptides comprising a point mutation that corresponds to a leucine at a position corresponding to 544 of SEQ ID NO: 1 (e.g., a I554L as compared to SEQ ID NO: 1). For example, VAR-22 includes a I554L mutation as compared to SEQ ID NO: 1 (shown in Table 1). Additional variant capsid polypeptides (VAR-46, VAR-47, VAR-48, VAR-49) having a I554L mutation were evaluated, where each of these additional variant capsid polypeptides do not have a mutation at the 555 amino acid position as compared to SEQ ID NO: 1, as in VAR-22. Data for VAR-46, VAR-47, VAR-48, and VAR-49 is shown in Table 6, and like VAR-22, virus particles comprising these capsid polypeptides comprising the I554L mutation resulted in increased transduction of trabecular meshwork tissue, relative to virus particles comprising capsid polypeptides of SEQ ID NO: 1. Thus, without being bound by theory, it is surprisingly discovered that the presence of a I554L mutation contributes to increased transduction of trabecular meshwork tissue in the context of diverse capsid polypeptide sequences, relative to capsid polypeptides which do not comprise the mutation. Relative to SEQ ID NO: 1, these additional variant capsid polypeptides comprising a I554L mutation have an edit distance in the range of 8-12 (as shown in Table 6). Relative to VAR-22, these additional variant capsid polypeptides comprising a I554L mutation have an edit distance of 11-16 (as shown in Table 6). Thus, as described herein are capsid polypeptides, nucleic acid molecules that encode capsid polypeptides, virus particles, and methods of making and using the same, comprising a I554L mutation as compared to SEQ ID NO: 1. In some embodiments, the capsid polypeptide comprising a I554L mutation has greater than 95%, greater than 96%, greater than 97% or optionally greater than 98% or greater than 99% sequence identity to SEQ ID NO: 1. In some embodiments, the capsid polypeptide comprising a I554L mutation has greater than 95%, greater than 96%, greater than 97% or optionally greater than 98% or greater than 99% sequence identity to the capsid polypeptide sequence of VAR-22. In some embodiments, the capsid polypeptide comprising a I554L mutation has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. Also disclosed, in some embodiments, are nucleic acid molecules that encode a capsid polypeptide that comprises a mutation corresponding to a I554L mutation as compared to SEQ ID NO: 1. Also disclosed, in some embodiments are virus particles, for example dependoparvovirus particles, comprising a capsid polypeptide that comprises a mutation corresponding to a I554L mutation as compared to SEQ ID NO: 1.

As shown in Table 6, each of these additional variant capsid polypeptides comprising a I554L mutation have increased trabecular transduction as compared to SEQ ID NO: 1. Accordingly, in some embodiments, a capsid polypeptide, comprising a mutation that corresponds to I554L as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

TABLE 6
Library Experiment 1

| | | | Macular | Retinal | Trabecular | Virus |
|---|---|---|---|---|---|---|
| | | | Transduction | Transduction | Transduction | Production |
| | Edit | | as compared | as compared | as compared | as compared |
| | Distance | Edit | to wild-type | to wild-type | to wild-type | to wild-type |
| | to SEQ | Distance | SEQ ID NO: 1 | SEQ ID NO: 1 | SEQ ID NO: 1 | SEQ ID NO: 1 |
| Name | ID NO: 1 | to VAR-22 | (Log2) | (Log2) | (Log2) | (Log2) |
| VAR-22 | 9 | | −1.16 | 0.81 | 2.95 | 0.72 |

Library Experiment 2

| | | | Macular | Retinal | Trabecular | | |
|---|---|---|---|---|---|---|---|
| | | | Transduction | Transduction | Transduction | Virus Production as | |
| | Edit | | as compared | as compared | as compared | compared to wild-type | |
| | Distance | Edit | to wild-type | to wild-type | to wild-type | SEQ ID NO: 1 (Log2) | |
| | to SEQ | Distance | SEQ ID NO: 1 | SEQ ID NO: 1 | SEQ ID NO: 1 | Anterior | Posterior |
| Name | ID NO: 1 | to VAR-22 | (Log2) | (Log2) | (Log2) | eye library | eye library |
| VAR-22 | 9 | | −1.57 | Not Measured | −0.80 | −4.36 | 1.03 |
| VAR-46 | 8 | 11 | Not Measured | −3.23 | 2.54 | −4.83 | 0.44 |
| VAR-47 | 12 | 13 | Not Measured | −3.30 | 3.24 | −3.23 | 2.90 |
| VAR-48 | 10 | 15 | −3.92 | −3.18 | 2.10 | 2.55 | 3.04 |
| VAR-49 | 11 | 16 | Not Measured | −3.52 | 3.28 | 1.49 | −0.02 |

In some embodiments, the capsid polypeptide comprising the L at position 554 (e.g., the I554L) mutation has one or more additional mutations. In some embodiments, the capsid polypeptide comprises point mutations that correspond to a leucine at a position corresponding to 544 in SEQ ID NO: 1 and a serine at a position corresponding to 561 in SEQ ID NO:1 (e.g., comprising a I554L and D561S as compared to SEQ ID NO: 1). For example, VAR-22, VAR-46, and VAR-47 include mutations that correspond to I554L and D561S as compared to SEQ ID NO: 1. VAR-46 and VAR-47 have no mutations at the 560, 562, 563, 564, and 565 amino acid residues as compared to SEQ ID NO: 1, as in VAR-22. Thus, without being bound by theory, it is surprisingly discovered that the presence of both I554L and D561S mutations contribute to increased transduction of trabecular meshwork tissue in the context of diverse capsid polypeptide sequences, relative to capsid polypeptides which do not comprise these mutations.

In some embodiments, the capsid polypeptide comprises point mutations that correspond to I554L and T581D as compared to SEQ ID NO: 1. For example, VAR-22, VAR-46, and VAR-47 include mutations that correspond to a leucine at a position corresponding to 544 in SEQ ID NO: 1 and a aspartic acid at a position corresponding to 581 in SEQ ID NO:1 (e.g., comprising a I554L and T581D as compared to SEQ ID NO: 1. VAR-46 and VAR-47 comprise an I554L mutation and a T581D mutation, but have no mutations at the 579, 580, 582, 583, and 584 amino acid residues as compared to SEQ ID NO: 1, as in VAR-22. Thus, without being bound by theory, it is surprisingly discovered that the presence of both I554L and T581D mutations contribute to increased transduction of trabecular meshwork tissue in the context of diverse capsid polypeptide sequences, relative to capsid polypeptides which do not comprise the mutation.

In some embodiments, the capsid polypeptide comprises point mutations that correspond to a leucine at a position corresponding to 544 in SEQ ID NO: 1, a serine at a position corresponding to 561 in SEQ ID NO:1 and an aspartic acid at a position corresponding to 581 in SEQ ID NO: 1 (e.g., comprising a to I554L, D561S, and T581D as compared to SEQ ID NO: 1). For example, VAR-22, VAR-46, and VAR-47 include mutations that correspond to I554L, D561S, and T581D as compared to SEQ ID NO: 1.

Thus, as described herein are capsid polypeptides, nucleic acid molecules that encode said capsid polypeptides, virus particles comprising said capsid polypeptides, and methods of making and using the same, comprising a leucine at a position corresponding to 554 of SEQ ID NO: 1 (e.g., comprising a I554L mutation as compared to SEQ ID NO: 1) and one or both of a serine at a position corresponding to 561 and a threonine at a positions corresponding to 581 of SEQ ID NO: 1 (e.g., D561S and T581D mutations as compared to SEQ ID NO: 1). In some embodiments, the capsid polypeptide comprising a I554L mutation and one or both D561S and T581D mutations has greater than 95%, greater than 96%, greater than 97% or optionally greater than 98% or greater than 99% sequence identity to SEQ ID NO: 1. In some embodiments, the capsid polypeptide comprising a I554L mutation and one or both D561S and T581D mutations has greater than 95%, greater than 96%, greater than 97% or optionally greater than 98% or greater than 99% sequence identity to the capsid polypeptide sequence of VAR-22. In some embodiments, the capsid polypeptide comprising a I554L mutation and one or both D561S and T581D mutations has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. In some embodiments, a nucleic acid molecule encodes a capsid polypeptide that comprises a mutation corresponding to a I554L mutation as compared to SEQ ID NO: 1 and one or both D561S and T581D mutations as compared to SEQ ID NO: 1. In some embodiments, a capsid polypeptide, comprising a mutation that corresponds to I554L as compared to SEQ ID NO: 1 and one or both D561S and T581D mutations as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

According to some embodiments, disclosed herein are capsid polypeptides comprising a point mutation that corresponds to a leucine at position 559 of SEQ ID NO: 1 (e.g., a I559L as compared to SEQ ID NO: 1). For example, VAR-22 and VAR-23 include a I559L mutation as compared to SEQ ID NO: 1 (shown in Table 1). Additional variant capsid polypeptides (VAR-50, VAR-51, VAR-52) having a I559L mutation were evaluated, where each of these additional variant capsid polypeptides have no mutations at the 557, 558, and 560 amino acid residues as compared to SEQ ID NO: 1, as in VAR-22 and VAR-23. Data for VAR-50, VAR-51, and VAR-52 is shown in Table 7, and like VAR-22 and VAR-23, virus particles comprising these capsid polypeptides comprising the I559L mutation resulted in increased transduction of trabecular meshwork tissue, relative to virus particles comprising capsid polypeptides of SEQ ID NO: 1. Thus, without being bound by theory, it is surprisingly discovered that the presence of a leucine at a position corresponding to 559 of SEQ ID NO: 1 (e.g., comprising a I559L mutation) contributes to increased transduction of trabecular meshwork tissue in the context of diverse capsid polypeptide sequences, relative to capsid polypeptides which do not comprise the mutation. Relative to SEQ ID NO: 1, these additional variant capsid polypeptides comprising a I559L mutation have an edit distance in the range of 4-13 (as shown in Table 7). Relative to VAR-22, these additional variant capsid polypeptides comprising a I559L mutation have an edit distance of 8-19 (as shown in Table 7). Relative to VAR-23, these additional variant capsid polypeptides comprising a I559L mutation have an edit distance of 5-20 (as shown in Table 7). Thus, as described herein are capsid polypeptides, nucleic acid molecules that encode said capsid polypeptides, virus particles comprising said capsid polypeptides, and methods of making and using the same, comprising a leucine at a position corresponding to 559 of SEQ ID NO: 1 (e.g., a I559L mutation as compared to SEQ ID NO: 1). In some embodiments, the capsid polypeptide comprising a I559L mutation has greater than 95%, greater than 96%, greater than 97% or optionally greater than 98% or greater than 99% sequence identity to SEQ ID NO: 1. In some embodiments, the capsid polypeptide comprising a I559L mutation has greater than 95%, greater than 96%, greater than 97% or optionally greater than 98% or greater than 99% sequence identity to the capsid polypeptide sequence of VAR-22. In some embodiments, the capsid polypeptide comprising a I559L mutation has greater than 95%, greater than 96%, greater than 97% or optionally greater than 98% or greater than 99% sequence identity to the capsid polypeptide sequence of VAR-23. In some embodiments, the capsid polypeptide comprising a I559L mutation has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. In some embodiments, disclosed herein are nucleic acid molecules encoding a capsid polypeptide that comprises a leucine at a position corresponding to 559 of SEQ ID NO: 1 (e.g., a mutation corresponding to a I559L mutation as compared to SEQ ID NO: 1). Also disclosed herein are virus particles, e.g., dependoparvovirus particles, comprising a capsid polypeptide that comprises a leucine at a position corresponding to 559 of SEQ ID NO: 1 (e.g., a mutation corresponding to a I559L mutation as compared to SEQ ID NO: 1).

As shown in Table 7, each of these additional variant capsid polypeptides comprising a I559L mutation have increased trabecular transduction as compared to SEQ ID NO: 1. Accordingly, in some embodiments, a capsid polypeptide, comprising a mutation that corresponds to I559L as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

TABLE 7
Library Experiment 1

| | | | | Macular | Retinal | Trabecular | |
|---|---|---|---|---|---|---|---|
| | | | | Transduction | Transduction | Transduction | |
| | Edit | | | as compared | as compared | as compared | |
| | Distance | Edit | Edit | to wild-type | to wild-type | to wild-type | Virus Production as |
| | to SEQ | Distance | Distance | SEQ ID NO: 1 | SEQ ID NO: 1 | SEQ ID NO: 1 | compared to wild-type |
| Name | ID NO: 1 | to VAR-22 | to VAR-23 | (Log2) | (Log2) | (Log2) | SEQ ID NO: 1 (Log2) |
| VAR-22 | 9 | | | −1.16 | 0.81 | 2.95 | 0.72 |
| VAR-23 | 9 | | | −0.60 | −3.96 | 2.20 | −1.54 |
| VAR-51 | 4 | 8 | 5 | −0.48 | 2.49 | 2.85 | 1.54 |

Library Expirement 2

| | | | | Macular | Retinal | Trabecular | | |
|---|---|---|---|---|---|---|---|---|
| | | | | Transduction | Transduction | Transduction | Virus Production as | |
| | Edit | | | as compared | as compared | as compared | compared to wild-type | |
| | Distance | Edit | Edit | to wild-type | to wild-type | to wild-type | SEQ ID NO: 1 (Log2) | |
| | to SEQ | Distance | Distance | SEQ ID NO: 1 | SEQ ID NO: 1 | SEQ ID NO: 1 | Anterior | Posterior |
| Name | ID NO: 1 | to VAR-22 | to VAR-23 | (Log2) | (Log2) | (Log2) | eye library | eye library |
| VAR-22 | 9 | | | −1.57 | Not Measured | −0.80 | −4.36 | 1.03 |
| VAR-50 | 9 | 10 | 9 | Not Measured | Not Measured | 2.68 | −2.96 | 5.60 |
| VAR-51 | 4 | 8 | 5 | Not Measured | Not Measured | 1.51 | Not Measured | −0.25 |
| VAR-52 | 13 | 19 | 20 | 0.95 | −0.04 | 4.47 | 1.54 | −3.34 |

In some embodiments, the capsid polypeptide comprising the leucine at a position corresponding to 559 of SEQ ID NO: 1 (e.g., I559L mutation) has one or more additional mutations. In some embodiments, the capsid polypeptide comprises point mutations that correspond to leucine at a position corresponding to 559 of SEQ ID NO: 1 and a serine at a position corresponding to 561 of SEQ ID NO: 1 (e.g., comprising I559L and D561S as compared to SEQ ID NO: 1). For example, VAR-22, VAR-23, and VAR-50 include mutations that correspond to I559L and D561S as compared to SEQ ID NO: 1. VAR-50 has no mutations at the 562, 563, 564, and 565 amino acid residues as compared to SEQ ID NO: 1, as in VAR-22 and VAR-23. Thus, without being bound by theory, it is surprisingly discovered that the presence of both I559L and D561S mutations contribute to increased transduction of trabecular meshwork tissue in the context of diverse capsid polypeptide sequences, relative to capsid polypeptides which do not comprise these mutations.

In some embodiments, the capsid polypeptide comprises point mutations that correspond to a leucine at a position corresponding to 559 of SEQ ID NO: 1 and aspartic acid at a position corresponding to 581 of SEQ ID NO: 1 (e.g., comprising I559L and T581D as compared to SEQ ID NO: 1). For example, VAR-22, VAR-23, VAR-50, and VAR-51 include mutations that correspond to I559L and T581D as compared to SEQ ID NO: 1. VAR-50 and VAR-51 have no mutations at the 579, 582, 583, and 584 amino acid residues as compared to SEQ ID NO: 1, as in VAR-22 and VAR-23. Thus, without being bound by theory, it is surprisingly discovered that the presence of both I559L and T581D mutations contribute to increased transduction of trabecular meshwork tissue in the context of diverse capsid polypeptide sequences, relative to capsid polypeptides which do not comprise the mutation.

In some embodiments, the capsid polypeptide comprises point mutations that correspond to leucine at a position corresponding to 559 of SEQ ID NO: 1 and a serine at a position corresponding to 561 of SEQ ID NO: 1 and an aspartic acid at a position corresponding to 581 of SEQ ID NO: 1 (e.g., comprising I5549L, D561S, and T581D as compared to SEQ ID NO: 1). For example, as discussed above, VAR-22, VAR-23, and VAR-50 include mutations that correspond to I554L, D561S, and T581D as compared to SEQ ID NO: 1. Thus, as described herein are capsid polypeptides, nucleic acid molecules that encode capsid polypeptides, virus particles, and methods of making and using the same, comprising a I559L mutation as compared to SEQ ID NO: 1 and one or both D561S and T581D mutations as compared to SEQ ID NO: 1. In some embodiments, the capsid polypeptide comprising a I559L mutation and one or both D561S and T581D mutations has greater than 95%, greater than 96%, greater than 97% or optionally greater than 98% or greater than 99% sequence identity to SEQ ID NO: 1. In some embodiments, the capsid polypeptide comprising a I559L mutation and one or both D561S and T581D mutations has greater than 95%, greater than 96%, greater than 97% or optionally greater than 98% or greater than 99% sequence identity to the capsid polypeptide sequence of VAR-22. In some embodiments, the capsid polypeptide comprising a I559L mutation and one or both D561S and T581D mutations has greater than 95%, greater than 96%, greater than 97% or optionally greater than 98% or greater than 99% sequence identity to the capsid polypeptide sequence of VAR-23. In some embodiments, the capsid polypeptide comprising a I559L mutation and one or both D561S and T581D mutations has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. In some embodiments, a nucleic acid molecule encodes a capsid polypeptide that comprises a mutation corresponding to a I559L mutation as compared to SEQ ID NO: 1 and one or both D561S and T581D mutations as compared to SEQ ID NO: 1. In some embodiments, a capsid polypeptide, comprising a mutation that corresponds to I559L as compared to SEQ ID NO: 1 and one or both D561S and T581D mutations as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

In some embodiments, disclosed herein are capsid polypeptides comprising a I5549L, D561S, and/or T581D mutation has additionally comprising one or more additional mutations. In some embodiments, the capsid polypeptide comprises point mutations that correspond to I5549L, D561S, T581D, and a mutation at a position corresponding to 556 of SEQ ID NO: 1 to R, D, or N. For example, VAR-22 includes a mutation that corresponds to K556R, VAR-23 includes a mutation that corresponds to K556D, and VAR-50 includes a mutation that corresponds to K556N as compared to SEQ ID NO: 1. Thus, without being bound by theory, it is surprisingly discovered that the presence of I5549L, D561S, and/or T581D mutations, a mutation at a position corresponding to position 556 of SEQ ID NO: 1 to R, D, or N contributes to increased transduction of trabecular meshwork tissue in the context of diverse capsid polypeptide sequences, relative to capsid polypeptides which do not comprise these mutations.

In some embodiments, disclosed herein are capsid polypeptides comprising point mutations that correspond to I5549L, D561S, and/or T581D, and a mutation at position corresponding to 586 of SEQ ID NO: 1 to either R or Q. For example, VAR-22 includes a mutation that corresponds to G586R, VAR-23 includes a mutation that corresponds to G586Q, and VAR-50 includes a mutation that corresponds to G586Q as compared to SEQ ID NO: 1. Thus, without being bound by theory, it is surprisingly discovered that the presence of I5549L, D561S, and/or T581D mutations, a mutation at a position corresponding to 586 of SEQ ID NO: 1 to either R or Q contributes to increased transduction of trabecular meshwork tissue in the context of diverse capsid polypeptide sequences, relative to capsid polypeptides which do not comprise these mutations.

In some embodiments, the capsid polypeptide comprises point mutations that correspond to I5549L, D561S, and/or T581D, and a mutation at a position corresponding to 566 of SEQ ID NO: 1 to either A or K. For example, VAR-22 includes a mutation that corresponds to R566A, VAR-23 includes a mutation that corresponds to R566K, and VAR-51 includes a mutation that corresponds to R566K as compared to SEQ ID NO: 1. Thus, without being bound by theory, it is surprisingly discovered that the presence of I5549L, D561S, and/or T581D mutations, a mutation at a position corresponding to 566 in SEQ ID NO: 1 to either A or K contributes to increased transduction of trabecular meshwork tissue in the context of diverse capsid polypeptide sequences, relative to capsid polypeptides which do not comprise these mutations.

According to some embodiments, the capsid polypeptide comprises a point mutation that corresponds to an asparagine at a position corresponding to 556 of SEQ ID NO: 1 (e.g., comprising K556N as compared to SEQ ID NO: 1). For example, VAR-21 includes a K556N mutation as compared to SEQ ID NO: 1 (shown in Table 1). Additional variant capsid polypeptides (VAR-47, VAR-49, VAR-50) having a K556N mutation were evaluated, where each of these additional variant capsid polypeptides have no mutations at the 555 and 557 amino acid residues as compared to SEQ ID NO: 1, as in VAR-21. Data for VAR-47, VAR-49, and VAR-50 is shown in Table 8, and like VAR-21, virus particles comprising these capsid polypeptides comprising the asparagine at a position corresponding to 556 of SEQ ID NO: 1 (e.g., K556N mutation) resulted in increased transduction of trabecular meshwork tissue, relative to virus particles comprising capsid polypeptides of SEQ ID NO: 1. Thus, without being bound by theory, it is surprisingly discovered that the presence of an asparagine at a position corresponding to 556 of SEQ ID NO: 1 (e.g., the presence of a K556N mutation) contributes to increased transduction of trabecular meshwork tissue in the context of diverse capsid polypeptide sequences, relative to capsid polypeptides which do not comprise the mutation. Relative to SEQ ID NO: 1, these additional variant capsid polypeptides comprising a K556N mutation have an edit distance in the range of 4-12 (as shown in Table 8). Relative to VAR-21, these additional variant capsid polypeptides comprising a K556N mutation have an edit distance of 9-12 (as shown in Table 8). Thus, as described herein are capsid polypeptides, nucleic acid molecules that encode said capsid polypeptides, virus particles comprising said capsid polypeptides, and methods of making and using the same, comprising an asparagine at a position corresponding to 556 of SEQ ID NO: 1 (e.g., comprising a K556N mutation as compared to SEQ ID NO: 1). In some embodiments, the capsid polypeptide comprising a K556N mutation has greater than 95%, greater than 96%, greater than 97% or optionally greater than 98% or greater than 99% sequence identity to SEQ ID NO: 1. In some embodiments, the capsid polypeptide comprising a K556N mutation has greater than 95%, greater than 96%, greater than 97% or optionally greater than 98% or greater than 99% sequence identity to the capsid polypeptide sequence of VAR-21. In some embodiments, the capsid polypeptide comprising a K556N mutation has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1.

In some embodiments, disclosed herein are nucleic acid molecules encoding a capsid polypeptide that comprises a mutation corresponding to an asparagine at a position corresponding to 556 of SEQ ID NO: 1 (e.g., comprising a K556N mutation as compared to SEQ ID NO: 1). As shown in Table 8, each of these additional variant capsid polypeptides comprising a K556N mutation have increased trabecular transduction as compared to SEQ ID NO: 1. Accordingly, in some embodiments, a capsid polypeptide, comprising a mutation that corresponds to K556N as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

TABLE 8
Library Experiment 1

| | | | Macular | Retinal | Trabecular | |
|---|---|---|---|---|---|---|
| | | | Transduction | Transduction | Transduction | |
| | Edit | | as compared | as compared | as compared | |
| | Distance | Edit | to wild-type | to wild-type | to wild-type | Virus Production as |
| | to SEQ | Distance | SEQ ID NO: 1 | SEQ ID NO: 1 | SEQ ID NO: 1 | compared to wild-type |
| Name | ID NO: 1 | to VAR-21 | (Log2) | (Log2) | (Log2) | SEQ ID NO: 1 (Log2) |
| VAR-21 | 4 | | 0.21 | 3.17 | 3.62 | 0.91 |

Library Experiment 2

| | | | Macular | Retinal | Trabecular | | |
|---|---|---|---|---|---|---|---|
| | | | Transduction | Transduction | Transduction | Virus Production as | |
| | Edit | | as compared | as compared | as compared | compared to wild-type | |
| | Distance | Edit | to wild-type | to wild-type | to wild-type | SEQ ID NO: 1 (Log2) | |
| | to SEQ | Distance | SEQ ID NO: 1 | SEQ ID NO: 1 | SEQ ID NO: 1 | Anterior | Posterior |
| Name | ID NO: 1 | to VAR-21 | (Log2) | (Log2) | (Log2) | eye library | eye library |
| VAR-21 | 4 | | Not Measured | −4.08 | −1.08 | −2.66 | 0.67 |
| VAR-47 | 12 | 11 | Not Measured | −3.30 | 3.24 | −3.23 | 2.90 |
| VAR-49 | 11 | 12 | Not Measured | −3.52 | 3.28 | 1.49 | −0.02 |
| VAR-50 | 9 | 9 | Not Measured | Not Measured | 2.68 | −2.96 | 5.60 |

In some embodiments, the capsid polypeptide comprising the asparagine at a position corresponding to 556 of SEQ ID NO: 1 (e.g., comprising a K556N mutation) has one or more additional mutations. In some embodiments, the capsid polypeptide comprises the mutation that corresponds to K556N, and a mutation at a position corresponding to 578 of SEQ ID NO: 1 to I, V, or T. For example, VAR-21 includes a mutation that corresponds to S578I, VAR-46 includes a mutation that corresponds to S578V, and VAR-47 includes a mutation that corresponds to S578T as compared to SEQ ID NO: 1. Thus, without being bound by theory, it is surprisingly discovered that the presence of K556N mutations and a mutation at a position corresponding to 578 of SEQ ID NO: 1 to I, V, or T contribute to increased transduction of trabecular meshwork tissue in the context of diverse capsid polypeptide sequences, relative to capsid polypeptides which do not comprise these mutations.

According to some embodiments, the capsid polypeptide comprises a point mutation that corresponds to a serine at a position corresponding to 561 of SEQ ID NO: 1 (e.g., comprises D561S as compared to SEQ ID NO: 1). For example, VAR-19, VAR-22, and VAR-23 include a D561S mutation as compared to SEQ ID NO: 1 (shown in Table 1). Additional variant capsid polypeptides (VAR-46, VAR-47, VAR-50, VAR-53) having a D561S mutation were evaluated, where each of these additional variant capsid polypeptides have no mutations at the 560, 562, 563, 564, and 565 amino acid residues as compared to SEQ ID NO: 1, as in VAR-19, VAR-22, and VAR-23. Data for VAR-46, VAR-47, VAR-50, and VAR-53 is shown in Table 9, and like VAR-19, VAR-22, and VAR-23, virus particles comprising these capsid polypeptides comprising the D561S mutation resulted in increased transduction of trabecular meshwork tissue, relative to virus particles comprising capsid polypeptides of SEQ ID NO: 1. Thus, without being bound by theory, it is surprisingly discovered that the presence of a serine at a position corresponding to 561 of SEQ ID NO: 1 (e.g., a D561S mutation) contributes to increased transduction of trabecular meshwork tissue in the context of diverse capsid polypeptide sequences, relative to capsid polypeptides which do not comprise the mutation. Relative to SEQ ID NO: 1, these additional variant capsid polypeptides comprising a D561S mutation have an edit distance in the range of 7-12 (as shown in Table 9). Relative to VAR-19, these additional variant capsid polypeptides comprising a D561S mutation have an edit distance of 11-14 (as shown in Table 9). Relative to VAR-22, these additional variant capsid polypeptides comprising a D561S mutation have an edit distance of 10-14 (as shown in Table 9). Relative to VAR-23, these additional variant capsid polypeptides comprising a D561S mutation have an edit distance of 9-14 (as shown in Table 9). Thus, as described herein are capsid polypeptides, nucleic acid molecules that encode said capsid polypeptides, virus particles comprising said capsid polypeptides, and methods of making and using the same, comprising a serine at a position corresponding to 561 of SEQ ID NO: 1 (e.g., comprising a D561S mutation as compared to SEQ ID NO: 1). In some embodiments, the capsid polypeptide comprising a D561S mutation has greater than 95%, greater than 96%, greater than 97% or optionally greater than 98% or greater than 99% sequence identity to SEQ ID NO: 1. In some embodiments, the capsid polypeptide comprising a D561S mutation has greater than 95%, greater than 96%, greater than 97% or optionally greater than 98% or greater than 99% sequence identity to the capsid polypeptide sequence of VAR-19. In some embodiments, the capsid polypeptide comprising a D561S mutation has greater than 95%, greater than 96%, greater than 97% or optionally greater than 98% or greater than 99% sequence identity to the capsid polypeptide sequence of VAR-22. In some embodiments, the capsid polypeptide comprising a D561S mutation has greater than 95%, greater than 96%, greater than 97% or optionally greater than 98% or greater than 99% sequence identity to the capsid polypeptide sequence of VAR-23. In some embodiments, the capsid polypeptide comprising a D561S mutation has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. In some embodiments, disclosed herein are nucleic acid molecules encoding a capsid polypeptide that comprises a mutation corresponding to a serine at a position corresponding to 561 of SEQ ID NO: 1 (e.g., comprising a D561S mutation as compared to SEQ ID NO: 1). As shown in Table 9, each of these additional variant capsid polypeptides comprising a D561S mutation have increased trabecular transduction as compared to SEQ ID NO: 1. Accordingly, in some embodiments, a capsid polypeptide, comprising a mutation that corresponds to D561S as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

TABLE 9

| | Library Experiment 1 | | | | | | | | |
|---|---|---|---|---|---|---|---|---|---|
| | | | | | Macular | Retinal | Trabecular | | |
| | Edit | | | | Transduction | Transduction | Transduction | | |
| | Distance | | | | as compared | as compared | as compared | | |
| | (ED) to | | | | to wild-type | to wild-type | to wild-type | Virus Production as | |
| | SEQ ID | ED to | ED to | ED to | SEQ ID NO: 1 | SEQ ID NO: 1 | SEQ ID NO: 1 | compared to wild-type | |
| Name | NO: 1 | VAR-19 | VAR-22 | VAR-23 | (Log2) | (Log2) | (Log2) | SEQ ID NO: 1 (Log2) | |
| VAR-19 | 7 | | | | −0.53 | 0.98 | 3.70 | 0.09 | |
| VAR-22 | 9 | | | | −1.16 | 0.81 | 2.95 | 0.72 | |
| VAR-23 | 9 | | | | −0.60 | −3.96 | 2.20 | −1.54 | |
| | Library Experiment 2 | | | | | | | | |
| | | | | | Macular | Retinal | Trabecular | | |
| | Edit | | | | Transduction | Transduction | Transduction | Virus Production as | |
| | Distance | | | | as compared | as compared | as compared | compared to wild-type | |
| | (ED) to | | | | to wild-type | to wild-type | to wild-type | SEQ ID NO: 1 (Log2) | |
| | SEQ ID | ED to | ED to | ED to | SEQ ID NO: 1 | SEQ ID NO: 1 | SEQ ID NO: 1 | Anterior | Posterior |
| Name | NO: 1 | VAR-19 | VAR-22 | VAR-23 | (Log2) | (Log2) | (Log2) | eye library | eye library |
| VAR-22 | 9 | | | | −1.57 | Not Measured | −0.80 | −4.36 | 1.03 |
| VAR-46 | 8 | 12 | 11 | 13 | Not Measured | −3.23 | 2.54 | −4.83 | 0.44 |
| VAR-47 | 12 | 14 | 13 | 14 | Not Measured | −3.30 | 3.24 | −3.23 | 2.90 |
| VAR-50 | 9 | 11 | 10 | 9 | Not Measured | Not Measured | 2.68 | −2.96 | 5.60 |
| VAR-53 | 9 | 12 | 14 | 13 | Not Measured | Not Measured | 4.47 | 0.14 | 1.82 |

In some embodiments, the capsid polypeptide comprising the serine at a position corresponding to 561 of SEQ ID NO: 1 (e.g., comprising a D561S mutation) has one or more additional mutations. In some embodiments, the capsid polypeptide comprises point mutations that correspond to a serine at a position corresponding to 561 of SEQ ID NO: 1 and an aspartic acid at a position corresponding to 581 of SEQ ID NO: 1 (e.g., comprising a D561S and T581D mutation as compared to SEQ ID NO: 1). For example, VAR-22, VAR-23, VAR-46, VAR-47, VAR-50, and VAR-53 include mutations that correspond to D561S and T581D as compared to SEQ ID NO: 1. Thus, without being bound by theory, it is surprisingly discovered that the presence of both D561S and T581D mutations contribute to increased transduction of trabecular meshwork tissue in the context of diverse capsid polypeptide sequences, relative to capsid polypeptides which do not comprise these mutations. Thus, as described herein are capsid polypeptides, nucleic acid molecules that encode said capsid polypeptides, virus particles comprising said capsid polypeptides, and methods of making and using the same, comprising a serine at a position corresponding to 561 of SEQ ID NO:1 and an aspartic acid at a position corresponding to 581 of SEQ ID NO: 1 (e.g., comprising D561S and T581D mutations as compared to SEQ ID NO: 1). In some embodiments, the capsid polypeptide comprising D561S and T581D mutations has greater than 95%, greater than 96%, greater than 97% or optionally greater than 98% or greater than 99% sequence identity to SEQ ID NO: 1. In some embodiments, the capsid polypeptide comprising D561S and T581D mutations has greater than 95%, greater than 96%, greater than 97% or optionally greater than 98% or greater than 99% sequence identity to the capsid polypeptide sequence of VAR-22. In some embodiments, the capsid polypeptide comprising D561S and T581D mutations has greater than 95%, greater than 96%, greater than 97% or optionally greater than 98% or greater than 99% sequence identity to the capsid polypeptide sequence of VAR-23. In some embodiments, the capsid polypeptide comprising D561S and T581D mutations has 1-20, e.g., 1, 2, 3, 4, 5, 6, 7, 8, 9, 10, 11, 12, 13, 14, 15, 16, 17, 18, 19 or 20 additional mutations relative to SEQ ID NO: 1. In some embodiments, disclosed herein are nucleic acid molecules encoding a capsid polypeptide that comprises a mutation corresponding to D561S and T581D mutations as compared to SEQ ID NO: 1. In some embodiments, a capsid polypeptide, comprising D561S and T581D mutations as compared to SEQ ID NO: 1, present in a viral particle increases transduction in the trabecular meshwork as compared to a viral particle with a reference capsid polypeptide, for example, with the wild-type capsid polypeptide (SEQ ID NO: 1).

Disclosed herein are capsid polypeptides comprising a mutation at a position corresponding to 587 of SEQ ID NO: 1. In embodiments, the mutation is to an alanine. In embodiments, the capsid polypeptide comprises a N587A mutation according to SEQ ID NO: 1. Also disclosed herein are capsid polypeptides comprising the mutation, e.g., mutation to alanine, at a position corresponding to 587 of SEQ ID NO: 1 (e.g., comprising N587A according to SEQ ID NO: 1) further comprising an insertion of one or more amino acids, e.g., 4 or more amino acids, e.g., 7, 8, 9 or more amino acids, e.g., between 7-10 amino acids, between two adjacent amino acids N-terminal to the mutation at the position corresponding to 587 of SEQ ID NO: 1. In embodiments, the insertion is 7 amino acids. In embodiments, the insertion is 8 amino acids. In embodiments, the insertion is 9 amino acids. In embodiments, the insertion is 10 amino acids. In embodiments, the insertion occurs after an amino acid no more than 3 amino acids N-terminal to the mutation at the position corresponding to 587 of SEQ ID NO: 1. In embodiments, the insertion occurs between two adjacent amino acids selected from the amino acids corresponding to 580 to 587 of SEQ ID NO: 1. In embodiments, the insertion is between amino acids corresponding to 584 and 585 of SEQ ID NO: 1. In embodiments, the insertion is between amino acids corresponding to 586 and 587 of SEQ ID NO: 1. In embodiments, disclosed herein are capsid polypeptides comprising a sequence at least 85%, 90%, 95%, 96%, 97%, 98%, or 99% identical to SEQ ID NO: 1 and comprising an alanine at a position corresponding to N587 of SEQ ID NO: 1 and comprising an insertion between amino acids corresponding to positions 586 and 587 of SEQ ID NO: 1. Disclosed herein are nucleic acid molecules comprising sequence encoding said capsid polypeptides. Also disclosed are virus particles, e.g., dependoparvovirus particles, comprising said capsid polypeptides. In embodiments, such virus particles, e.g., dependoparvovirus particles, have increased transduction of the macula and/or non-macula retina tissue of the eye, for example, as measured by quantification of viral RNA from the target tissue, relative to virus particles comprising the capsid polypeptides of SEQ ID NO: 1. In embodiments, such virus particles, e.g., dependoparvovirus particles, have increased transduction of the anterior eye tissue (e.g., trabecular meshwork and/or Schlemm's canal tissue), for example, as measured by quantification of viral RNA from the target tissue, relative to virus particles comprising the capsid polypeptides of SEQ ID NO: 1.

As described herein in Example 3, the subcombinations of mutations present in each of the variants described herein were determined by computationally defining all possible 1-mutation, 2-mutation, 3-mutation, 4-mutation, etc., up to the total number of mutations in each variant (with contiguous strings of inserted amino acids counted as 1 mutation for purposes of this analysis only). The unique variants tested in library experiment 1 and Library Experiment 2 comprising at least those subcombination of mutations and at most 3 additional mutations not specified in the subcombination were pooled, and a median whole retina transduction calculated for all variants in each pool. The analysis identifies several minimal structural elements (sets of mutations) which, when present in a variant capsid polypeptide, result in better than wtAAV2 median whole retina transduction variants tested in Library Experiment 1 and Library Experiment 2. These mutation sets as well as the additional data associated with each pool are shown in Table 12 and Table 13, below.

TABLE 12
Minimum structural elements comprised in the variants described herein and associated with increased median
retina transduction relative to wild-type AAV2 identified from Library Experiment 1. In the second from
left column, all amino acid position numbering is according to SEQ ID NO: 1. Amino acids are represented by
their one-letter code; deletions are represented by a “−”; insertions are represented by the notation [last
wild-type amino acid position before the insertion]−[length of insertion]_aa-[wild-type amino acid
position after the insertion]_[identity of the insertion]. A “+” indicates a motif
with one or more additional positions which can be any amino acid.

| | | | | | |
|---|---|---|---|---|---|
| | | Number | | | |
| | | of viral | | | |
| | | particles | Average | | Number of |
| | | with | number of | | mutations from |
| | | unique VP1 | mutations from | | wild type |
| | | capsid | wild type | | AAV2 in the |
| | | polypeptide | AAV2 of all | | Common |
| | | variants | unique VP1 | | Mutation Set |
| | Common Mutation | comprising | capsid | | (for purposes |
| | Set (Numbering | the Common | polypeptides | Median whole | of this table, |
| | according to SEQ ID | Mutation Set | in the Set, | retina | contiguous |
| | NO: 1) representing a | and up to 3 | excluding the | transduction | amino acids |
| | subcombination of the | additional | mutations | measurement | inserted at |
| | mutations found in the | mutations | comprised in | from Library | the same |
| | Variant(s) listed in | from wt AAV2 | the Common | Experiment 1 | position are |
| Variant Name | the left-most column | (the “Set”) | Mutation Set | across Set | counted as 1) |

VAR-11 mutation sets with median whole retina transduction
(log2 relative to wtAAV2) greater than 1.0

| | | | | | |
|---|---|---|---|---|---|
| VAR-11 | R459−, T456− | 3 | 2.67 | 3.56 | 2 |
| VAR-11 | R459−, Q457− | 6 | 2.33 | 3.38 | 2 |
| VAR-11 | S458−, T456−, L460− | 3 | 2.67 | 3.31 | 3 |
| VAR-11 | R459−, Q457−, T456− | 6 | 2.33 | 3.19 | 3 |
| VAR-11 | Q457−, L460− | 3 | 2.67 | 3.19 | 2 |
| VAR-11 | R459−, Q457−, L460− | 5 | 2.20 | 3.19 | 3 |
| VAR-11 | R459−, S458−, Q457− | 9 | 1.89 | 3.19 | 3 |
| VAR-11 | S458−, Q457−, T456− | 9 | 2.11 | 3.08 | 3 |
| VAR-11 | L460−, S458−, R459−, Q457−, | 4 | 1.00 | 2.92 | 5 |
| | T456− | | | | |
| VAR-11 | R459−, S458−, Q457−, L460− | 6 | 1.50 | 2.86 | 4 |
| VAR-11 | R459−, S458−, Q457−, T456− | 8 | 1.75 | 2.80 | 4 |
| VAR-11 | S458−, L460− | 4 | 2.25 | 2.51 | 2 |
| VAR-11 | R459−, S458−, L460− | 6 | 1.83 | 2.51 | 3 |
| VAR-11 | R459−, S458− | 8 | 1.88 | 2.51 | 2 |
| VAR-11 | S458−, Q457− | 10 | 2.30 | 2.47 | 2 |
| VAR-11 | S458−, T456− | 6 | 2.50 | 2.38 | 2 |
| VAR-11 | R459− | 6 | 1.83 | 1.86 | 1 |
| VAR-11 | L460− | 3 | 2.00 | 1.82 | 1 |
| VAR-11 | Q461−, L460−, S458−, R459−, | 2 | 0.50 | 1.63 | 5 |
| | Q457− | | | | |
| VAR-11 | S458− | 13 | 2.00 | 1.36 | 1 |

VAR-11 mutation sets with median whole retina transduction
(log2 relative to wtAAV2) greater than 0 and less than 1.0

| | | | | | |
|---|---|---|---|---|---|
| VAR-11 | Q461−, L460− | 2 | 1.50 | 0.68 | 2 |
| VAR-11 | Q457− | 22 | 2.36 | 0.48 | 1 |
| VAR-11 | Q457−, T456− | 13 | 2.15 | 0.37 | 2 |

VAR-12 mutation sets with median whole retina transduction
(log2 relative to wtAAV2) greater than 1.0

| | | | | | |
|---|---|---|---|---|---|
| VAR-12 | S458Q, T455Q, P451G | 2 | 2.50 | 2.99 | 3 |
| VAR-12 | T456N, R459T | 5 | 2.40 | 2.57 | 2 |
| VAR-12 | N4491, T456N, R459T, T455Q | 2 | 1.00 | 2.33 | 4 |
| VAR-12 | R459T, P451G | 4 | 2.25 | 2.29 | 2 |
| VAR-12 | S458Q, T454−, T450N | 2 | 3.00 | 2.24 | 3 |
| VAR-12 | R459T, T454− | 2 | 1.00 | 2.06 | 2 |
| VAR-12 | S458Q, Q461K, R459T, Q464V | 3 | 3.00 | 2.05 | 4 |
| VAR-12 | T454−, T450N, P451G, Q464V, | 3 | 2.67 | 1.92 | 6 |
| | R459T, S458Q | | | | |
| VAR-12 | Q461K, T450N, P451G, Q464V, | 4 | 2.75 | 1.90 | 7 |
| | T456N, R459T, T455Q | | | | |
| VAR-12 | Q461K, N449I, T450N, P451G, | 5 | 2.00 | 1.83 | 8 |
| | Q464V, T456N, R459T, T455Q | | | | |
| VAR-12 | Q461K, N449I, T450N, P451G, | 4 | 2.50 | 1.79 | 6 |
| | Q464V, R459T | | | | |
| VAR-12 | Q461K, N449I, T450N, P451G, | 4 | 2.50 | 1.77 | 8 |
| | T456N, R459T, S458Q, T455Q | | | | |
| VAR-12 | S458Q, T455Q | 6 | 2.50 | 1.75 | 2 |
| VAR-12 | N4491, T456N, R459T, Q464V | 2 | 2.00 | 1.72 | 4 |
| VAR-12 | Q461K, T450N, P451G, Q464V, | 3 | 2.33 | 1.72 | 8 |
| | T456N, R459T, S458Q, T455Q | | | | |
| VAR-12 | Q461K, N449I, P451G, Q464V, | 5 | 2.80 | 1.72 | 7 |
| | T456N, R459T, T455Q | | | | |
| VAR-12 | N449I, T450N, P451G, Q464V, | 3 | 2.33 | 1.65 | 6 |
| | R459T, S458Q | | | | |
| VAR-12 | Q461K, T450N, P451G, Q464V, | 5 | 2.40 | 1.65 | 6 |
| | R459T, S458Q | | | | |
| VAR-12 | Q461K, N449I, T450N, P451G, | 5 | 2.00 | 1.65 | 7 |
| | Q464V, R459T, S458Q | | | | |
| VAR-12 | Q461K, Q464V, T456N, R459T, | 2 | 2.50 | 1.65 | 5 |
| | S458Q | | | | |
| VAR-12 | T456N, T450N | 2 | 2.50 | 1.60 | 2 |
| VAR-12 | Q461K, T454−, T450N, P451G, | 5 | 2.00 | 1.53 | 6 |
| | R459T, S458Q | | | | |
| VAR-12 | S458Q, R459T, P451G | 3 | 2.00 | 1.53 | 3 |
| VAR-12 | Q461K, T450N, P451G, Q464V, | 2 | 2.50 | 1.52 | 6 |
| | T456N, R459T | | | | |
| VAR-12 | T454−, N4491, T450N, P451G, | 2 | 2.50 | 1.42 | 6 |
| | Q464V, S458Q | | | | |
| VAR-12 | Q461K, T454−, P451G, Q464V, | 4 | 2.50 | 1.42 | 6 |
| | R459T, S458Q | | | | |
| VAR-12 | Q461K, T454−, T450N, P451G, | 4 | 2.00 | 1.42 | 7 |
| | Q464V, R459T, S458Q | | | | |
| VAR-12 | S458Q, P451G | 17 | 2.18 | 1.28 | 2 |
| VAR-12 | Q461K, N449I, T450N, P451G, | 2 | 3.00 | 1.28 | 5 |
| | Q464V | | | | |
| VAR-12 | Q461K, N449I, Q464V, R459T, | 4 | 2.75 | 1.28 | 5 |
| | S458Q | | | | |
| VAR-12 | Q461K, N449I, P451G, Q464V, | 4 | 2.25 | 1.28 | 6 |
| | R459T, S458Q | | | | |
| VAR-12 | Q461K, N449I, T450N, P451G, | 4 | 2.25 | 1.28 | 6 |
| | R459T, S458Q | | | | |
| VAR-12 | T456N, S458Q | 2 | 2.50 | 1.11 | 2 |
| VAR-12 | Q461K, T450N, P451G | 2 | 2.50 | 1.11 | 3 |
| VAR-12 | N4491, T456N, T455Q | 3 | 2.33 | 1.10 | 3 |
| VAR-12 | T456N, T455Q | 3 | 2.00 | 1.10 | 2 |
| VAR-12 | R459T, T455Q | 3 | 2.67 | 1.10 | 2 |
| VAR-12 | N449I, T455Q | 3 | 3.00 | 1.10 | 2 |
| VAR-12 | Q461K, T450N, P451G, T456N, | 2 | 3.00 | 1.09 | 7 |
| | R459T, S458Q, T455Q | | | | |
| VAR-12 | Q461K, N449I, T450N, P451G, | 4 | 1.75 | 1.09 | 9 |
| | Q464V, T456N, R459T, S458Q, | | | | |
| | T455Q | | | | |
| VAR-12 | S458Q, Q461K, R459T | 2 | 1.50 | 1.01 | 3 |

VAR-12 mutation sets with median whole retina transduction
(log2 relative to wtAAV2) greater than 0 and less than 1.0

| | | | | | |
|---|---|---|---|---|---|
| VAR-12 | Q461K, N449I, T450N, P451G, | 2 | 2.50 | 0.97 | 6 |
| | R459T, T455Q | | | | |
| VAR-12 | Q461K, T450N, P451G, Q464V, | 3 | 2.33 | 0.96 | 7 |
| | T456N, R459T, S458Q | | | | |
| VAR-12 | T450N, P451G, Q464V, R459T, | 3 | 3.00 | 0.96 | 5 |
| | S458Q | | | | |
| VAR-12 | Q461K, T450N, P451G, R459T, | 5 | 2.60 | 0.96 | 5 |
| | S458Q | | | | |
| VAR-12 | Q461K, P451G, Q464V, R459T, | 4 | 3.00 | 0.94 | 5 |
| | S458Q | | | | |
| VAR-12 | T454−, N449I, P451G, Q464V, | 3 | 2.33 | 0.91 | 6 |
| | R459T, S458Q | | | | |
| VAR-12 | N449I, P451G, Q464V, R459T, | 3 | 3.00 | 0.91 | 5 |
| | S458Q | | | | |
| VAR-12 | T454−, N449I, T450N, P451G, | 3 | 2.33 | 0.91 | 6 |
| | R459T, S458Q | | | | |
| VAR-12 | Q461K, N449I, T450N, R459T, | 3 | 3.00 | 0.91 | 5 |
| | S458Q | | | | |
| VAR-12 | T454−, T450N, P451G, R459T, | 3 | 2.33 | 0.91 | 5 |
| | S458Q | | | | |
| VAR-12 | Q461K, T454−, N449I, T450N, | 3 | 1.00 | 0.91 | 8 |
| | P451G, Q464V, R459T, S458Q | | | | |
| VAR-12 | T454−, N449I, Q464V, R459T, | 2 | 3.00 | 0.90 | 5 |
| | S458Q | | | | |
| VAR-12 | Q461K, T454−, N449I, P451G, | 4 | 2.25 | 0.90 | 6 |
| | R459T, S458Q | | | | |
| VAR-12 | Q461K, N449I, P451G, R459T, | 4 | 3.00 | 0.90 | 5 |
| | S458Q | | | | |
| VAR-12 | Q461K, T454−, P451G, R459T, | 4 | 2.50 | 0.90 | 5 |
| | S458Q | | | | |
| VAR-12 | Q461K, T454−, N449I, R459T, | 3 | 3.00 | 0.89 | 5 |
| | S458Q | | | | |
| VAR-12 | Q461K, T454−, N449I, P451G, | 5 | 2.00 | 0.89 | 7 |
| | Q464V, R459T, S458Q | | | | |
| VAR-12 | Q461K, N449I, Q464V, T456N, | 3 | 3.00 | 0.89 | 7 |
| | R459T, S458Q, T455Q | | | | |
| VAR-12 | Q461K, N449I, P451G, Q464V, | 5 | 2.40 | 0.89 | 8 |
| | T456N, R459T, S458Q, T455Q | | | | |
| VAR-12 | R459T, T454−, Q461K, P451G | 2 | 2.50 | 0.88 | 4 |
| VAR-12 | N4491, T456N, T455Q, T450N | 2 | 1.50 | 0.81 | 4 |
| VAR-12 | Q461K, T454−, N449I, T450N, | 4 | 1.75 | 0.69 | 7 |
| | P451G, R459T, S458Q | | | | |
| VAR-12 | Q461K, T454−, N449I, P451G, | 2 | 1.00 | 0.68 | 9 |
| | Q464V, T456N, R459T, S458Q, | | | | |
| | T455Q | | | | |
| VAR-12 | Q461K, T454−, N449I, P451G, | 4 | 2.75 | 0.56 | 5 |
| | R459T | | | | |
| VAR-12 | P451G | 45 | 2.18 | 0.48 | 1 |
| VAR-12 | T454− | 44 | 2.00 | 0.33 | 1 |
| VAR-12 | N449I | 13 | 1.54 | 0.32 | 1 |
| VAR-12 | T456N | 17 | 1.76 | 0.10 | 1 |
| VAR-12 | Q461K | 12 | 2.00 | 0.09 | 1 |

VAR-13 mutation sets with median whole retina transduction
(log2 relative to wtAAV2) greater than 1.0

| | | | | | |
|---|---|---|---|---|---|
| VAR-13 | S446A, P451Q, T455G, N449−, | 3 | 2.67 | 3.17 | 7 |
| | T448−, G453T, Q457T | | | | |
| VAR-13 | S446A, P451Q, T456G, T455G, | 2 | 1.50 | 2.17 | 8 |
| | N449−, T448−, G453T, Q457T | | | | |
| VAR-13 | S446A, Q457T | 2 | 3.00 | 1.79 | 2 |
| VAR-13 | S446A, T456G | 2 | 1.50 | 1.53 | 2 |

VAR-13 mutation sets with median whole retina transduction
(log2 relative to wtAAV2) greater than 0 and less than 1.0

| | | | | | |
|---|---|---|---|---|---|
| VAR-13 | S446A, P451Q, T455G, N449−, | 2 | 2.50 | 0.95 | 6 |
| | T448−, G453T | | | | |
| VAR-13 | S446A | 16 | 1.63 | 0.94 | 1 |
| VAR-13 | G453T | 7 | 1.43 | 0.71 | 1 |
| VAR-13 | T448− | 9 | 1.89 | 0.59 | 1 |
| VAR-13 | P451Q | 16 | 2.00 | 0.51 | 1 |
| VAR-13 | N449−, T448− | 9 | 1.89 | 0.42 | 2 |
| VAR-13 | N449− | 13 | 2.31 | 0.42 | 1 |

VAR-14 mutation sets with median whole retina transduction
(log2 relative to wtAAV2) greater than 1.0

| | | | | | |
|---|---|---|---|---|---|
| VAR-14 | T455A, T450S, R459D | 2 | 3.00 | 3.71 | 3 |
| VAR-14 | N449Q, T455A, T450S, R459D | 3 | 2.33 | 3.63 | 4 |
| VAR-14 | N449Q, T450S, R459D | 3 | 3.00 | 3.63 | 3 |
| VAR-14 | S452G, T455A, T450S, N449Q, | 3 | 3.00 | 2.61 | 5 |
| | R459D | | | | |
| VAR-14 | N449Q, S452G, T455A, R459D | 2 | 3.00 | 2.35 | 4 |
| VAR-14 | N449Q, T455A, R459D | 8 | 2.50 | 2.12 | 3 |
| VAR-14 | N449Q, S452G, R459D | 3 | 3.00 | 2.05 | 3 |
| VAR-14 | N449Q, R459D | 6 | 1.67 | 2.02 | 2 |
| VAR-14 | N449Q, S452G, T455A, T450S | 18 | 2.83 | 1.99 | 4 |
| VAR-14 | R459D | 19 | 1.74 | 1.67 | 1 |
| VAR-14 | S458M | 11 | 1.27 | 1.64 | 1 |
| VAR-14 | T455A, R459D | 4 | 1.75 | 1.60 | 2 |
| VAR-14 | N449Q, S452G, T455A | 26 | 2.65 | 1.13 | 3 |

VAR-14 mutation sets with median whole retina transduction
(log2 relative to wtAA V2) greater than 0 and less than 1.0

| | | | | | |
|---|---|---|---|---|---|
| VAR-14 | N449Q, T455A | 32 | 2.47 | 1.12 | 2 |
| VAR-14 | N449Q, T455A, T450S | 23 | 2.70 | 0.80 | 3 |
| VAR-14 | T455A | 66 | 2.30 | 0.52 | 1 |
| VAR-14 | S452G, T455A | 18 | 2.44 | 0.35 | 2 |
| VAR-14 | N449Q, T450S | 42 | 2.48 | 0.22 | 2 |
| VAR-14 | S452G | 50 | 2.34 | 0.22 | 1 |
| VAR-14 | N449Q, S452G, T450S | 24 | 2.92 | 0.06 | 3 |
| VAR-14 | N449Q, S452G | 36 | 2.61 | 0.05 | 2 |
| VAR-14 | T450S | 80 | 2.15 | 0.04 | 1 |
| VAR-14 | S452G, T450S | 12 | 2.58 | 0.03 | 2 |

VAR-19 mutation sets with median whole retina transduction
(log2 relative to wtAAV2) greater than 0 and less than 1.0

| | | | | | |
|---|---|---|---|---|---|
| VAR-19 | T581A | 2 | 1.00 | 10.56 | 1 |
| VAR-19 | R585− | 7 | 2.00 | 0.53 | 1 |
| VAR-19 | S578V | 57 | 2.28 | 0.32 | 1 |
| VAR-19 | D561S, S578V, S580A | 13 | 2.54 | 0.31 | 3 |
| VAR-19 | S578V, S580A | 24 | 2.63 | 0.28 | 2 |
| VAR-19 | S580A | 75 | 2.33 | 0.23 | 1 |
| VAR-19 | R566K, S578V, S580A | 15 | 2.40 | 0.20 | 3 |
| VAR-19 | D561S | 59 | 2.17 | 0.01 | 1 |

VAR-20 mutation sets with median whole retina transduction
(log2 relative to wtAAV2) greater than 1.0

| | | | | | |
|---|---|---|---|---|---|
| VAR-20 | S578D | 3 | 0.67 | 1.98 | 1 |

VAR-21 mutation sets with median whole retina transduction
(log2 relative to wtAAV2) greater than 1.0

| | | | | | |
|---|---|---|---|---|---|
| VAR-21 | S578I, M558L | 2 | 1.50 | 1.67 | 2 |
| VAR-21 | S578I, S580A | 2 | 2.50 | 1.16 | 2 |
| VAR-21 | M558L, S580A | 4 | 1.75 | 1.03 | 2 |
| VAR-21 | K556N, S578I | 3 | 2.00 | 1.03 | 2 |

VAR-21 mutation sets with median whole retina transduction
(log2 relative to wtAAV2) greater than 0 and less than 1.0

| | | | | | |
|---|---|---|---|---|---|
| VAR-21 | K556N, M558L, S580A | 2 | 1.00 | 0.6 | 3 |
| VAR-21 | K556N, M558L | 10 | 2.00 | 0.45 | 2 |
| VAR-21 | K556N | 152 | 2.21 | 0.06 | 1 |
| VAR-21 | M558L | 36 | 2.17 | 0.06 | 1 |

VAR-21 and VAR-23 common mutation set with median whole retina
transduction (log2 relative to wtAAV2) greater than 0 and less than 1.0

| | | | | | |
|---|---|---|---|---|---|
| VAR-21, | S578I | 10 | 1.80 | 0.94 | 1 |
| VAR-23 | | | | | |

VAR-22 mutation sets with median whole retina transduction
(log2 relative to wtAAV2) greater than 0 and less than 1.0

| | | | | | |
|---|---|---|---|---|---|
| VAR-22 | R585S, D561S, 1554L, G586R | 2 | 3.00 | 0.98 | 4 |
| VAR-22 | N587S | 30 | 2.20 | 0.47 | 1 |
| VAR-22 | R585S, N587S | 8 | 1.63 | 0.38 | 2 |
| VAR-22 | R566A | 8 | 1.88 | 0.22 | 1 |
| VAR-22 | T581D, R585S, I554L | 6 | 3.00 | 0.03 | 3 |
| VAR-22 | I554L | 118 | 2.23 | 0.02 | 1 |

VAR-22 and VAR-23 common mutation set with median whole retina
transduction (log2 relative to wtAAV2) greater than 1.0

| | | | | | |
|---|---|---|---|---|---|
| VAR-22, | I559L, T581D | 17 | 2.53 | 1.01 | 2 |
| VAR-23 | | | | | |

VAR-22 and VAR-23 common mutation set with median whole retina
transduction (log2 relative to wtAAV2) greater than 0 and less than 1.0

| | | | | | |
|---|---|---|---|---|---|
| VAR-22, | T581D | 54 | 2.22 | 0.65 | 1 |
| VAR-23 | | | | | |
| VAR-22, | T581D, D561S | 21 | 2.14 | 0.53 | 2 |
| VAR-23 | | | | | |
| VAR-22, | I559L, D561S, T581D | 15 | 2.73 | 0.44 | 3 |
| VAR-23 | | | | | |

VAR-22, VAR-26 and VAR-240 common mutation set with median whole retina
transduction (log2 relative to wtAAV2) greater than 0 and less than 1.0

| | | | | | |
|---|---|---|---|---|---|
| VAR-22, | R585S | 107 | 2.18 | 0.23 | 1 |
| VAR-26, | | | | | |
| VAR-40 | | | | | |

VAR-23 mutation sets with median whole retina transduction
(log2 relative to wtAAV2) greater than 1.0

| | | | | | |
|---|---|---|---|---|---|
| VAR-23 | R566K, 1559L, G586Q, T581D, | 5 | 2.60 | 1.16 | 5 |
| | E555S | | | | |
| VAR-23 | G586Q, D561S, I559L, E555S | 5 | 2.20 | 1.14 | 4 |
| VAR-23 | R566K, I559L, D561S, G586Q, | 5 | 2.40 | 1.14 | 5 |
| | E555S | | | | |
| VAR-23 | I559L, D561S, G586Q, T581D, | 7 | 2.00 | 1.14 | 5 |
| | E555S | | | | |
| VAR-23 | T581D, D561S, E555S | 13 | 2.46 | 1.14 | 3 |
| VAR-23 | R566K, D561S, I559L, T581D, | 6 | 2.50 | 1.13 | 5 |
| | E555S | | | | |
| VAR-23 | G586Q, I559L, T581D, E555S | 7 | 1.86 | 1.11 | 4 |
| VAR-23 | I559L, D561S, T581D, E555S | 10 | 2.00 | 1.11 | 4 |
| VAR-23 | I559L, T581D, E555S | 15 | 2.33 | 1.07 | 3 |
| VAR-23 | G586Q, T581D | 12 | 2.17 | 1.02 | 2 |

VAR-23 mutation sets with median whole retina transduction
(log2 relative to wtAAV2) greater than 0 and less than 1.0

| | | | | | |
|---|---|---|---|---|---|
| VAR-23 | T581D, E555S | 21 | 2.57 | 0.90 | 2 |
| VAR-23 | R566K, G586Q, D561S, I559L, | 6 | 2.50 | 0.83 | 5 |
| | T581D | | | | |
| VAR-23 | R566K, D561S, G586Q, T581D, | 8 | 2.50 | 0.83 | 5 |
| | E555S | | | | |
| VAR-23 | R566K, T581D, D561S, E555S | 7 | 2.57 | 0.80 | 4 |
| VAR-23 | I559L, V552A, T581D | 2 | 2.00 | 0.79 | 3 |
| VAR-23 | R566K, T581D | 15 | 2.47 | 0.78 | 2 |
| VAR-23 | I559L, D561S, E555S | 10 | 2.60 | 0.77 | 3 |
| VAR-23 | R566K, I559L, D561S, G586Q | 4 | 2.75 | 0.75 | 1 |
| VAR-23 | R566K, I559L, T581D, E555S | 5 | 2.20 | 0.75 | 4 |
| VAR-23 | R566K, T581D, E555S | 7 | 2.43 | 0.74 | 3 |
| VAR-23 | G586Q, T581D, E555S | 11 | 2.55 | 0.74 | 3 |
| VAR-23 | S5781, D561S, T581D, E555S | 2 | 2.50 | 0.74 | 4 |
| VAR-23 | R566K, G586Q, D561S, S578I, | 3 | 2.00 | 0.63 | 5 |
| | E555S | | | | |
| VAR-23 | G586Q, D561S, T581D, E555S | 9 | 2.44 | 0.63 | 4 |
| VAR-23 | T581D, V552A | 2 | 2.00 | 0.58 | 2 |
| VAR-23 | G586Q, D561S, T581D | 11 | 2.45 | 0.57 | 3 |
| VAR-23 | G586Q, I559L, T581D | 13 | 2.69 | 0.54 | 3 |
| VAR-23 | G586Q, I559L, E555S | 8 | 2.25 | 0.52 | 3 |
| VAR-23 | R566K, 1559L, G586Q, E555S | 3 | 2.67 | 0.52 | 4 |
| VAR-23 | G586Q, D561S, I559L, T581D | 8 | 2.13 | 0.43 | 4 |
| VAR-23 | R566K, I559L, G586Q, T581D | 8 | 2.63 | 0.43 | 4 |
| VAR-23 | R566K, I559L, G586Q | 5 | 2.60 | 0.43 | 3 |
| VAR-23 | R566K, I559L, T581D | 12 | 2.58 | 0.43 | 3 |
| VAR-23 | G586Q, D561S, I559L | 8 | 2.63 | 0.42 | 3 |
| VAR-23 | G586Q, D561S, E555S | 10 | 2.20 | 0.41 | 3 |
| VAR-23 | R566K, T581D, G586Q, E555S | 6 | 2.17 | 0.41 | 4 |
| VAR-23 | G586Q | 63 | 2.30 | 0.38 | 1 |
| VAR-23 | R566K, I559L, D561S, T581D | 8 | 2.50 | 0.31 | 4 |
| VAR-23 | R566K, I559L, E555S | 6 | 2.50 | 0.26 | 3 |
| VAR-23 | R566K, I559L, D561S, G586Q, | 9 | 2.44 | 0.26 | 6 |
| | T581D, E555S | | | | |
| VAR-23 | R566K, 1559L, D561S, E555S | 6 | 2.67 | 0.22 | 4 |
| VAR-23 | R566K, G586Q, E555S | 5 | 2.40 | 0.19 | 3 |
| VAR-23 | R566K, T581D, G586Q | 9 | 2.67 | 0.19 | 3 |
| VAR-23 | R566K, G586Q | 9 | 2.44 | 0.19 | 2 |
| VAR-23 | R566K, T581D, D561S, G586Q | 8 | 2.63 | 0.14 | 4 |
| VAR-23 | R566K, G586Q, D561S, E555S | 9 | 2.67 | 0.12 | 4 |
| VAR-23 | G586Q, I559L | 12 | 2.08 | 0.10 | 2 |
| VAR-23 | V552A | 72 | 2.19 | 0.08 | 1 |
| VAR-23 | G586Q, D561S | 14 | 2.36 | 0.01 | 2 |

VAR-24 mutation set with median whole retina transduction
(log2 relative to wtAAV2) greater than 1.0

| | | | | | |
|---|---|---|---|---|---|
| VAR-24 | Q575E | 18 | 2.06 | 1.10 | 1 |

VAR-26 mutation set with median whole retina transduction
(log2 relative to wtAAV2) greater than 1.0

| | | | | | |
|---|---|---|---|---|---|
| VAR-26 | R588T, R585S, T597S, A591I, | 2 | 3.00 | 3.59 | 5 |
| | V600A | | | | |
| VAR-26 | R588T, R585S, Q589N, A590P, | 2 | 2.50 | 3.25 | 6 |
| | T597S, A591I | | | | |
| VAR-26 | A593G, R585S, T597S, A591I, | 2 | 3.00 | 2.88 | 5 |
| | V600A | | | | |
| VAR-26 | R588T, R585S, A590P, T597S, | 2 | 2.50 | 2.50 | 6 |
| | A591I, V600A | | | | |
| VAR-26 | R588T, A593G, R585S, Q589N, | 2 | 2.00 | 2.48 | 6 |
| | A590P, V600A | | | | |
| VAR-26 | R588T, A593G, Q589N, A590P, | 2 | 2.50 | 2.41 | 5 |
| | A591I | | | | |
| VAR-26 | R588T, R585S, Q589N, A590P, | 2 | 3.00 | 2.11 | 5 |
| | T597S | | | | |
| VAR-26 | R588T, R585S, Q589N, A590P, | 2 | 2.50 | 2.03 | 6 |
| | T597S, V600A | | | | |
| VAR-26 | T597S, A591I | 8 | 2.38 | 1.65 | 2 |
| VAR-26 | R585S, T597S | 5 | 2.60 | 1.58 | 2 |
| VAR-26 | R585S, T597S, V600A | 3 | 2.33 | 1.54 | 3 |
| VAR-26 | T597S, V600A | 16 | 2.38 | 1.46 | 2 |
| VAR-26 | A590P, T597S, V600A | 16 | 2.81 | 1.23 | 3 |
| VAR-26 | T597S, A591I, V600A | 10 | 2.50 | 1.17 | 3 |
| VAR-26 | A590P, T597S, A591I | 9 | 2.67 | 1.02 | 3 |
| VAR-26 | A593G, T597S, A591I | 10 | 2.40 | 1.02 | 3 |
| VAR-26 | R588T, Q589N | 2 | 3.00 | 1.01 | 2 |
| VAR-26 | R588T, A590P, T597S, A591I, | 4 | 2.75 | 1.01 | 5 |
| | V600A | | | | |

VAR-26 mutation sets with median whole retina transduction
(log2 relative to wtAAV2) greater than 0 and less than 1.0

| | | | | | |
|---|---|---|---|---|---|
| VAR-26 | A591I, V600A | 13 | 2.31 | 0.95 | 2 |
| VAR-26 | V600A | 93 | 2.28 | 0.91 | 1 |
| VAR-26 | R585S, V600A | 14 | 2.29 | 0.91 | 2 |
| VAR-26 | A593G, T597S, A591I, V600A | 5 | 2.40 | 0.90 | 4 |
| VAR-26 | R588T, T597S | 9 | 2.33 | 0.88 | 2 |
| VAR-26 | A593G, T597S, V600A | 8 | 2.25 | 0.87 | 3 |
| VAR-26 | A593G, A591I, V600A | 6 | 2.67 | 0.84 | 3 |
| VAR-26 | R588T, T597S, V600A | 8 | 2.75 | 0.79 | 3 |
| VAR-26 | A593G, A590P, T597S | 12 | 2.58 | 0.77 | 3 |
| VAR-26 | A590P, V600A | 35 | 2.40 | 0.76 | 2 |
| VAR-26 | A590P, T597S, A591I, V600A | 6 | 2.17 | 0.74 | 4 |
| VAR-26 | A590P, T597S | 23 | 2.22 | 0.73 | 2 |
| VAR-26 | R588T, A593G, A591I, V600A | 5 | 2.00 | 0.72 | 4 |
| VAR-26 | R588T, A591I, V600A | 7 | 2.29 | 0.72 | 3 |
| VAR-26 | A591I | 36 | 2.17 | 0.68 | 1 |
| VAR-26 | R588T, V600A | 36 | 2.33 | 0.65 | 2 |
| VAR-26 | T597S | 50 | 2.22 | 0.63 | 1 |
| VAR-26 | R588T, A593G, R585S, Q589N, | 3 | 3.00 | 0.60 | 5 |
| | A590P | | | | |
| VAR-26 | Q589N | 5 | 1.80 | 0.59 | 1 |
| VAR-26 | R588T, A590P, T597S | 17 | 2.65 | 0.57 | 3 |
| VAR-26 | A590P, A591I, V600A | 10 | 2.60 | 0.51 | 3 |
| VAR-26 | A593G, A590P, A591I, V600A | 6 | 2.50 | 0.50 | 4 |
| VAR-26 | R588T, T597S, A591I | 5 | 3.00 | 0.49 | 3 |
| VAR-26 | A593G, T597S | 20 | 2.00 | 0.49 | 2 |
| VAR-26 | R588T, A593G, T597S, A591I, | 5 | 2.40 | 0.44 | 5 |
| | V600A | | | | |
| VAR-26 | A593G, A591I | 11 | 2.18 | 0.40 | 2 |
| VAR-26 | A590P | 129 | 2.25 | 0.38 | 1 |
| VAR-26 | A593G, V600A | 33 | 2.30 | 0.38 | 2 |
| VAR-26 | A593G, A590P, T597S, A591I, | 8 | 2.63 | 0.38 | 5 |
| | V600A | | | | |
| VAR-26 | R588T | 117 | 2.19 | 0.37 | 1 |
| VAR-26 | A590P, A591I | 16 | 2.19 | 0.33 | 2 |
| VAR-26 | R588T, A591I | 10 | 2.30 | 0.33 | 2 |
| VAR-26 | R588T, A593G, A591I | 11 | 2.45 | 0.32 | 3 |
| VAR-26 | A593G | 82 | 2.22 | 0.29 | 1 |
| VAR-26 | A590P, R585S | 21 | 2.43 | 0.28 | 2 |
| VAR-26 | R588T, A593G, T597S | 19 | 2.79 | 0.28 | 3 |
| VAR-26 | R588T, A593G, T597S, A591I | 9 | 2.67 | 0.27 | 4 |
| VAR-26 | A590P, T597S, R585S | 9 | 2.67 | 0.26 | 3 |
| VAR-26 | R588T, A590P, A591I, T597S | 6 | 3.00 | 0.24 | 4 |
| VAR-26 | A593G, T597S, A591I, A590P | 10 | 2.70 | 0.23 | 4 |
| VAR-26 | R588T, A593G, V600A | 32 | 2.63 | 0.23 | 3 |
| VAR-26 | R588T, A590P | 48 | 2.42 | 0.21 | 2 |
| VAR-26 | R588T, A593G, R585S, A590P, | 5 | 2.40 | 0.21 | 7 |
| | T597S, A591I, V600A | | | | |
| VAR-26 | R588T, A593G, R585S, A590P, | 5 | 2.20 | 0.21 | 6 |
| | T597S, A591I | | | | |
| VAR-26 | R588T, A593G, R585S, T597S, | 5 | 2.20 | 0.21 | 5 |
| | A591I | | | | |
| VAR-26 | R588T, A593G, A590P, T597S, | 11 | 2.64 | 0.21 | 5 |
| | A591I | | | | |
| VAR-26 | A593G, R585S, V600A | 15 | 2.53 | 0.20 | 3 |
| VAR-26 | R588T, R585S, V600A | 16 | 2.56 | 0.20 | 3 |
| VAR-26 | A593G, A590P, T597S, V600A | 13 | 2.92 | 0.20 | 4 |
| VAR-26 | A590P, R585S, V600A | 13 | 2.77 | 0.19 | 3 |
| VAR-26 | R588T, A593G, A590P, T597S, | 6 | 2.33 | 0.19 | 6 |
| | A591I, V600A | | | | |
| VAR-26 | R588T, A593G, A590P, A591I | 12 | 2.50 | 0.19 | 4 |
| VAR-26 | A593G, A590P | 34 | 2.35 | 0.16 | 2 |
| VAR-26 | A590P, T597S, R585S, V600A | 14 | 2.86 | 0.15 | 4 |
| VAR-26 | A593G, A590P, V600A | 28 | 2.64 | 0.15 | 3 |
| VAR-26 | R588T, A590P, A591I | 8 | 2.50 | 0.14 | 3 |
| VAR-26 | A593G, R585S | 17 | 2.35 | 0.13 | 2 |
| VAR-26 | R588T, A590P, V600A | 37 | 2.68 | 0.11 | 3 |
| VAR-26 | R588T, A590P, R585S | 38 | 2.82 | 0.11 | 3 |
| VAR-26 | A593G, A590P, R585S, V600A | 26 | 2.69 | 0.11 | 4 |
| VAR-26 | R588T, A590P, R585S, V600A | 40 | 2.78 | 0.11 | 4 |
| VAR-26 | R588T, A593G, T597S, V600A | 17 | 2.71 | 0.08 | 4 |
| VAR-26 | R588T, A593G, R585S, V600A | 33 | 2.76 | 0.08 | 4 |
| VAR-26 | R588T, R585S | 23 | 2.39 | 0.08 | 2 |
| VAR-26 | R588T, A593G, A590P, R585S | 74 | 2.55 | 0.08 | 4 |
| VAR-26 | R588T, A593G | 44 | 2.52 | 0.06 | 2 |
| VAR-26 | R588T, A590P, T597S, V600A | 17 | 2.82 | 0.03 | 4 |
| VAR-26 | A593G, A590P, R585S | 32 | 2.75 | 0.02 | 3 |

VAR-27 mutation sets with median whole retina transduction
(log2 relative to wtAAV2) greater than 0 and less than 1.0

| | | | | | |
|---|---|---|---|---|---|
| VAR-27 | G586A, R585N | 4 | 2.00 | 0.68 | 2 |
| VAR-27 | G586A | 62 | 2.29 | 0.29 | 1 |

VAR-28 mutation set with median whole retina transduction
(log2 relative to wtAAV2) greater than 1.0

| | | | | | |
|---|---|---|---|---|---|
| VAR-28 | 592_2 + aa_593_L+ | 2 | 1.00 | 2.82 | 1 |

VAR-3 mutation set with median whole retina transduction
(log2 relative to wtAAV2) greater than 1.0

| | | | | | |
|---|---|---|---|---|---|
| VAR-3 | P451T, T456G, T455L, N449Q, | 2 | 2.50 | 4.16 | 6 |
| | S458Q, Q457T | | | | |
| VAR-3 | P451T, T456G, T450M, N449Q, | 2 | 2.50 | 4.07 | 6 |
| | S458Q, Q457T | | | | |
| VAR-3 | N449Q, T455L, T456G, S458Q | 2 | 3.00 | 2.58 | 4 |
| VAR-3 | P451T, T455L, N449Q, S458Q, | 2 | 3.00 | 2.46 | 5 |
| | Q457T | | | | |
| VAR-3 | P451T, T456G, T455L, N449Q, | 2 | 2.50 | 2.42 | 5 |
| | Q457T | | | | |
| VAR-3 | P451T, T456G, R459M, N449Q, | 2 | 3.00 | 2.09 | 5 |
| | Q457T | | | | |
| VAR-3 | P451T, T456G, R459M, N449Q, | 3 | 2.67 | 1.87 | 5 |
| | S458Q | | | | |
| VAR-3 | N449Q, P451T, Q457T, T450M | 2 | 3.00 | 1.87 | 4 |
| VAR-3 | N449Q, S458Q, T456G, Q457T | 20 | 2.65 | 1.65 | 4 |
| VAR-3 | R459M | 4 | 0.75 | 1.51 | 1 |
| VAR-3 | N449Q, P451T, R459M, Q457T | 2 | 2.00 | 1.49 | 4 |
| VAR-3 | N449Q, P451T, T456G | 22 | 2.73 | 1.15 | 3 |
| VAR-3 | N449Q, P451T, T455L, Q457T | 3 | 3.00 | 1.15 | 4 |

VAR-3 mutation sets with median whole retina transduction
(log2 relative to wtAAV2) greater than 0 and less than 1.0

| | | | | | |
|---|---|---|---|---|---|
| VAR-3 | N449Q, P451T, T456G, Q457T | 35 | 2.66 | 0.99 | 4 |
| VAR-3 | N449Q, S458Q, Q457T | 29 | 2.72 | 0.95 | 3 |
| VAR-3 | P451T, T456G, N449Q, S458Q, | 23 | 2.65 | 0.93 | 5 |
| | Q457T | | | | |
| VAR-3 | N449Q, P451T, Q457T | 46 | 2.74 | 0.77 | 3 |
| VAR-3 | N449Q, P451T, T456G, R459M | 2 | 3.00 | 0.67 | 4 |
| VAR-3 | T450M | 8 | 1.25 | 0.50 | 1 |
| VAR-3 | N449Q, P451T, Q457T, S458Q | 31 | 2.68 | 0.44 | 4 |
| VAR-3 | N449Q, P451T, T456G, S458Q | 23 | 2.65 | 0.43 | 4 |
| VAR-3 | N449Q, P451T, S458Q | 25 | 2.80 | 0.33 | 3 |
| VAR-3 | N449Q, S458Q, T456G | 21 | 2.52 | 0.22 | 3 |
| VAR-3 | N449Q, P451T | 36 | 2.58 | 0.15 | 2 |
| VAR-3 | N449Q, S458Q | 35 | 2.54 | 0.09 | 2 |
| VAR-3 | N449Q, T455L | 15 | 2.20 | 0.06 | 2 |

VAR-3 and VAR-4 common mutation sets with median whole retina
transduction (log2 relative to wtAAV2) greater than 1.0

| | | | | | |
|---|---|---|---|---|---|
| VAR-3, VAR-4 | N449Q, T456G | 40 | 2.60 | 1.51 | 2 |
| VAR-3, VAR-4 | N449Q, T456G, Q457T | 28 | 2.68 | 1.37 | 3 |
| VAR-3, VAR-4 | N449Q, Q457T | 47 | 2.62 | 1.03 | 2 |

VAR-3, VAR-4 and VAR-14 common mutation set with median whole retina
transduction (log2 relative to wtAAV2) greater than 0.0 and less than 1.0

| | | | | | |
|---|---|---|---|---|---|
| VAR-3, VAR-4, | N449Q | 122 | 2.36 | 0.32 | 1 |
| VAR-14 | | | | | |

VAR-3, VAR-4 and VAR-13 common mutation sets with median whole
retina transduction (log2 relative to wtAAV2) greater than 1.0

| | | | | | |
|---|---|---|---|---|---|
| VAR-3, VAR-4, | T456G | 74 | 2.22 | 1.27 | 1 |
| VAR-8, VAR-13 | | | | | |
| VAR-3, VAR-4, | T456G, Q457T | 35 | 2.60 | 1.18 | 2 |
| VAR-8, VAR-13 | | | | | |

VAR-3, VAR-4 and VAR-13 common mutation set with median whole retina
transduction (log2 relative to wtAAV2) greater than 0.0 and less than 1.0

| | | | | | |
|---|---|---|---|---|---|
| VAR-3, VAR-4, | Q457T | 77 | 2.38 | 0.72 | 1 |
| VAR-8, VAR-13 | | | | | |

VAR-3 and VAR-8 common mutation sets with median whole retina
transduction (log2 relative to wtAAV2) greater than 1.0

| | | | | | |
|---|---|---|---|---|---|
| VAR-3, VAR-8 | P451T, S458Q, T456G, Q457T | 20 | 2.75 | 1.94 | 4 |
| VAR-3, VAR-8 | S458Q, T456G, Q457T | 20 | 2.55 | 1.68 | 3 |
| VAR-3, VAR-8 | S458Q, T456G | 30 | 2.60 | 1.63 | 2 |
| VAR-3, VAR-8 | P451T, S458Q, T456G | 14 | 2.71 | 1.23 | 3 |
| VAR-3, VAR-8 | P451T, T456G | 14 | 2.29 | 1.23 | 2 |
| VAR-3, VAR-8 | P451T, T456G, Q457T | 19 | 2.68 | 1.03 | 3 |

VAR-3 and VAR-8 common mutation sets with median whole retina
transduction (log2 relative to wtAAV2) greater than 0.0 and less than 1.0

| | | | | | |
|---|---|---|---|---|---|
| VAR-3, VAR-8 | S458Q, Q457T | 32 | 2.44 | 0.91 | 2 |
| VAR-3, VAR-8 | P451T, Q457T | 19 | 2.63 | 0.77 | 2 |
| VAR-3, VAR-8 | P451T, S458Q | 16 | 2.63 | 0.35 | 2 |
| VAR-3, VAR-8 | S458Q | 72 | 2.13 | 0.29 | 1 |
| VAR-3, VAR-8 | P451T, S458Q, Q457T | 18 | 2.89 | 0.14 | 3 |

VAR-32 mutation set with median whole retina
transduction (log2 relative to wtAAV2) greater than 1.0

| | | | | | |
|---|---|---|---|---|---|
| VAR-32 | 587_7 + aa_588_+ EQTRPA | 2 | 3.00 | 3.43 | 1 |

VAR-4 mutation sets with median whole retina
transduction (log2 relative to wtAAV2) greater than 1.0

| | | | | | |
|---|---|---|---|---|---|
| VAR-4 | N449Q, T456G, S452T | 2 | 3.00 | 4.34 | 3 |
| VAR-4 | S452T, T456G, Q457T | 2 | 3.00 | 3.89 | 3 |
| VAR-4 | T456G, P451−, Q457T, N449Q, | 2 | 1.50 | 3.74 | 5 |
| | R459G | | | | |
| VAR-4 | S452T, P451−, Q457T | 4 | 3.00 | 3.61 | 3 |
| VAR-4 | N449Q, P451−, T456G, S452T | 2 | 2.50 | 3.38 | 4 |
| VAR-4 | S452T, P451− | 5 | 2.60 | 3.38 | 2 |
| VAR-4 | N449Q, Q457T, S452T | 3 | 3.00 | 2.75 | 3 |
| VAR-4 | N449Q, Q457T, R459G | 5 | 3.00 | 2.66 | 3 |
| VAR-4 | N449Q, P451−, T456G, Q457T | 8 | 2.88 | 2.26 | 4 |
| VAR-4 | P451−, R459G | 2 | 2.00 | 2.23 | 2 |
| VAR-4 | T456G, P451−, S452T, N449Q, | 3 | 2.00 | 1.87 | 5 |
| | Q457T | | | | |
| VAR-4 | N449Q, P451−, S452T | 7 | 2.86 | 1.83 | 3 |
| VAR-4 | N449Q, T456G, R459G, Q457T | 6 | 2.67 | 1.81 | 4 |
| VAR-4 | S452T, P451−, T456G, Q457T | 3 | 2.67 | 1.63 | 4 |
| VAR-4 | N449Q, P451−, Q457T, S452T | 5 | 2.40 | 1.63 | 4 |
| VAR-4 | N449Q, P451−, Q457T | 8 | 2.75 | 1.30 | 3 |
| VAR-4 | P451−, T456G | 7 | 2.14 | 1.27 | 2 |
| VAR-4 | S452T | 6 | 1.83 | 1.24 | 1 |
| VAR-4 | P451−, T456G, Q457T | 4 | 2.00 | 1.06 | 3 |

VAR-4 mutation sets with median whole retina transduction
(log2 relative to wtAAV2) greater than 0.0 and less than 1.0

| | | | | | |
|---|---|---|---|---|---|
| VAR-4 | P451− | 46 | 2.20 | 0.75 | 1 |
| VAR-4 | P451−, Q457T | 11 | 2.27 | 0.60 | 2 |
| VAR-4 | N449Q, P451− | 11 | 2.45 | 0.53 | 2 |
| VAR-4 | N449Q, P451−, T456G | 5 | 2.40 | 0.09 | 3 |

VAR-4 and VAR-13 common mutation sets with median whole retina
transduction (log2 relative to wtAAV2) greater than 1.0

| | | | | | |
|---|---|---|---|---|---|
| VAR-4, VAR-13 | T456G, R459G, Q457T | 2 | 3.00 | 2.93 | 3 |
| VAR-4, VAR-13 | R459G | 14 | 2.00 | 2.42 | 1 |

VAR-40 mutation sets with median whole retina
transduction (log2 relative to wtAAV2) greater than 1.0

| | | | | | |
|---|---|---|---|---|---|
| VAR-40 | T597A, R585S, G586S, A591E, | 2 | 2.00 | 3.67 | 6 |
| | D594R, V600I | | | | |
| VAR-40 | R585S, G586S, A591E, D594R, | 2 | 2.50 | 3.39 | 5 |
| | V600I | | | | |
| VAR-40 | A591E, T597A, R585S, D594R | 3 | 2.00 | 2.93 | 4 |
| VAR-40 | T597A, R585S, N587A, G586S, | 2 | 2.00 | 2.68 | 6 |
| | D594R, V600I | | | | |
| VAR-40 | T597A, R585S, N587A, A591E, | 2 | 1.50 | 2.54 | 5 |
| | D594R | | | | |
| VAR-40 | T597A, R585S, N587A, G586S, | 2 | 2.00 | 2.54 | 5 |
| | D594R | | | | |
| VAR-40 | A591E, T597A, R585S, G586S | 2 | 2.00 | 2.49 | 4 |
| VAR-40 | R585S, N587A, G586S, A591E, | 2 | 2.50 | 2.10 | 5 |
| | V600I | | | | |
| VAR-40 | A591E, T597A, D594R | 2 | 2.50 | 1.36 | 3 |
| VAR-40 | A591E, R585S | 2 | 3.00 | 1.32 | 2 |

VAR-40 mutation sets with median whole retina transduction
(log2 relative to wtAAV2) greater than 0.0 and less than 1.0

| | | | | | |
|---|---|---|---|---|---|
| VAR-40 | G586S, V600I | 2 | 3.00 | 0.85 | 2 |
| VAR-40 | G586S | 90 | 2.22 | 0.33 | 1 |
| VAR-40 | T597A, R585S | 9 | 2.11 | 0.31 | 2 |
| VAR-40 | A591E | 8 | 1.50 | 0.28 | 1 |
| VAR-40 | V600I | 17 | 1.88 | 0.25 | 1 |
| VAR-40 | T597A | 23 | 2.17 | 0.13 | 1 |
| VAR-40 | R585S, V600I | 5 | 2.60 | 0.13 | 2 |
| VAR-40 | R585S, G586S | 25 | 2.28 | 0.07 | 2 |

VAR-5 mutation set with median whole retina transduction
(log2 relative to wtAAV2) greater than 0.0 and less than 1.0

| | | | | | |
|---|---|---|---|---|---|
| VAR-5 | S458C | 6 | 1.17 | 0.31 | 1 |

VAR-5 and VAR-14 common mutation set with median whole retina
transduction (log2 relative to wtAAV2) greater than 0.0 and less than 1.0

| | | | | | |
|---|---|---|---|---|---|
| VAR-5, VAR-14 | Q457F | 5 | 0.80 | 0.26 | 1 |

VAR-8 mutation sets with median whole retina transduction
(log2 relative to wtAAV2) greater than 1.0

| | | | | | |
|---|---|---|---|---|---|
| VAR-8 | P451T, T456G, T455G, R459T, | 2 | 1.00 | 5.34 | 6 |
| | S458Q, Q457T | | | | |
| VAR-8 | T456G, T455G, R459T, S458Q, | 3 | 2.00 | 5.17 | 5 |
| | Q457T | | | | |
| VAR-8 | P451T, T456G, T455G, R459T, | 3 | 2.33 | 5.17 | 5 |
| | Q457T | | | | |
| VAR-8 | P451T, S458Q, Q457T, T455G | 3 | 3.00 | 5.17 | 4 |
| VAR-8 | Q461A, R459T, T456G, T455G | 3 | 2.67 | 5.05 | 4 |
| VAR-8 | S458Q, R459T, T456G, T455G | 5 | 2.80 | 5.05 | 4 |
| VAR-8 | P451T, T456G, R459T, S458Q, | 4 | 2.25 | 4.67 | 5 |
| | Q457T | | | | |
| VAR-8 | P451T, S458Q, Q457T, R459T | 4 | 2.75 | 4.67 | 4 |
| VAR-8 | P451T, S458Q, T456G, R459T | 4 | 2.75 | 4.67 | 4 |
| VAR-8 | T456G, T455G, Q461A, S458Q, | 2 | 2.00 | 4.54 | 5 |
| | Q457T | | | | |
| VAR-8 | P451T, T456G, T455G, Q461A, | 2 | 1.50 | 4.47 | 6 |
| | R459T, Q457T | | | | |
| VAR-8 | P451T, Q461A, R459T, S458Q, | 2 | 2.50 | 4.42 | 5 |
| | Q457T | | | | |
| VAR-8 | S458Q, T456G, T455G, Q457T | 4 | 3.00 | 4.38 | 4 |
| VAR-8 | P451T, T456G, Q461A, R459T, | 2 | 2.50 | 4.36 | 5 |
| | S458Q | | | | |
| VAR-8 | Q461A, R459T, T455G | 4 | 2.75 | 4.31 | 3 |
| VAR-8 | T456G, T455G, Q461A, R459T, | 4 | 2.00 | 4.31 | 5 |
| | S458Q | | | | |
| VAR-8 | Q461A, P451T, T455G | 2 | 2.50 | 4.18 | 3 |
| VAR-8 | P451T, R459T, T456G, Q457T | 5 | 2.80 | 4.17 | 4 |
| VAR-8 | S458Q, R459T, T456G, Q457T | 5 | 3.00 | 4.17 | 4 |
| VAR-8 | R459T, T456G, T455G | 2 | 2.50 | 4.12 | 3 |
| VAR-8 | S458Q, T456G, R459T | 4 | 2.50 | 4.03 | 3 |
| VAR-8 | Q461A, R459T, S458Q, T455G | 5 | 2.80 | 4.01 | 4 |
| VAR-8 | T456G, T455G, Q461A, R459T, | 3 | 1.67 | 4.01 | 6 |
| | S458Q, Q457T | | | | |
| VAR-8 | T456G, Q461A, R459T, S458Q, | 4 | 2.50 | 3.67 | 5 |
| | Q457T | | | | |
| VAR-8 | R459T, T456G | 7 | 2.29 | 3.65 | 2 |
| VAR-8 | P451T, S458Q, R459T, Q461A | 2 | 2.50 | 3.61 | 4 |
| VAR-8 | T455G, Q461A, R459T, S458Q, | 3 | 2.33 | 3.58 | 5 |
| | Q457T | | | | |
| VAR-8 | T456G, T455G, Q461A, R459T, | 3 | 2.33 | 3.58 | 5 |
| | Q457T | | | | |
| VAR-8 | Q461A, T456G, T455G, Q457T | 3 | 2.67 | 3.58 | 4 |
| VAR-8 | Q461A, R459T, T456G, Q457T | 3 | 3.00 | 3.58 | 4 |
| VAR-8 | Q461A, S458Q, R459T | 2 | 3.00 | 3.38 | 3 |
| VAR-8 | S458Q, R459T, Q457T | 5 | 2.40 | 3.25 | 3 |
| VAR-8 | R459T, Q457T | 9 | 2.00 | 3.22 | 2 |
| VAR-8 | R459T, T455G | 3 | 3.00 | 3.20 | 2 |
| VAR-8 | Q461A, R459T | 2 | 2.00 | 2.82 | 2 |
| VAR-8 | Q461A, P451T, T456G, Q457T | 2 | 2.50 | 2.79 | 4 |
| VAR-8 | P451T, T456G, R459T | 3 | 3.00 | 2.36 | 3 |
| VAR-8 | P451T, S458Q, R459T | 3 | 3.00 | 2.36 | 3 |
| VAR-8 | Q461A, T455G | 3 | 3.00 | 2.27 | 2 |
| VAR-8 | Q461A, T456G, T455G | 2 | 3.00 | 1.94 | 3 |
| VAR-8 | P451T, R459T, Q457T | 2 | 3.00 | 1.79 | 3 |
| VAR-8 | R459T, T456G, Q457T | 2 | 3.00 | 1.50 | 3 |
| VAR-8 | Q461A | 13 | 1.46 | 1.32 | 1 |

VAR-8 mutation set with median whole retina transduction
(log2 relative to wtAAV2) greater than 0.0 and less than 1.0

| | | | | | |
|---|---|---|---|---|---|
| VAR-8 | P451T, T456G, T455G | 2 | 2.00 | 0.90 | 3 |

VAR-8 and VAR-12 common mutation sets with median whole
retina transduction (log2 relative to wtAAV2) greater than 1.0

| | | | | | |
|---|---|---|---|---|---|
| VAR-8, VAR-12 | S458Q, R459T | 13 | 2.00 | 3.25 | 2 |
| VAR-8, VAR-12 | R459T | 37 | 1.95 | 2.21 | 1 |

VAR-8 and VAR-13 common mutation sets with median whole
retina transduction (log2 relative to wtAAV2) greater than 1.0

| | | | | | |
|---|---|---|---|---|---|
| VAR-8, VAR-13 | T456G, T455G | 2 | 2.50 | 1.41 | 2 |
| VAR-8, VAR-13 | Q457T, T455G | 6 | 2.50 | 1.36 | 2 |

TABLE 13
Minimum structural elements comprised in the variants described herein and associated with increased median
retina transduction relative to wild-type AAV2 identified from Library Experiment 2. In the second from
left column, all amino acid position numbering is according to SEQ ID NO: 1. Amino acids are represented by their
one-letter code; deletions are represented by a “—”; insertions are represented by the notation [last wild-
type amino acid position before the insertion]-[length of insertion]_aa-[wild-type amino acid position
after the insertion]_[identity of the insertion]. A “+” indicates a motif with one
or more additional positions which can be any amino acid.

| | | | | | |
|---|---|---|---|---|---|
| | | Number of viral | Average number | | |
| | | particles with | of mutaitons | | Number of |
| | | unique VP1 | from wild type | | mutations from |
| | | capsid | AAV2 of all | | wild type AAV2 |
| | | polypeptide | unique VP1 | | in the Common |
| | Common Mutaiton Set | variants | capsid | | Mutation Set |
| | (Numbering according | comprising the | polypeptides in | Median whole | (for purposes of |
| | to SEQ ID NO: 1) | Common | the Set, | retina | this table, |
| | representing a | Mutation Set and | mutations | transduction | ontiguous amino |
| | subcombination of the | up to 3 additional | excluding the | measurement | acids inserted at |
| | mutations found in the | mutations from | comprised in the | from Library | the same |
| Variant | Variant(s) listed in | wt AAV2 (the | Common | Experiment 1 | position are |
| Name | the left-most column | “Set”) | Mutation Set | across Set | counted as 1) |

VAR-11 mutation set with median whole retina transduction
(log2 relative to wtAAV2) greater than 0.0

| | | | | | |
|---|---|---|---|---|---|
| VAR-11 | S458− | 8 | 2.625 | 0.258 | 1 |

VAR-13 mutation sets with median whole retina transduction
(log2 relative to wtAAV2) greater than 0.0

| | | | | | |
|---|---|---|---|---|---|
| VAR-13 | T456G, Q457T, R459G, T455G | 2 | 3.000 | 0.301 | 4 |
| VAR-13 | P451Q, T455G, N449−, | 4 | 2.750 | 0.257 | 5 |
| | G453T, Q457T | | | | |
| VAR-13 | P451Q, N449−, Q457T | 2 | 3.000 | 0.206 | 3 |
| VAR-13 | P451Q, T455G, N449−, | 6 | 2.833 | 0.199 | 5 |
| | T448−, G453T | | | | |
| VAR-13 | T455G, N449−, T448−, | 6 | 2.833 | 0.199 | 5 |
| | G453T, R459G | | | | |
| VAR-13 | T456G, P451Q, T455G, | 4 | 2.750 | 0.167 | 6 |
| | N449−, T448−, G453T | | | | |
| VAR-13 | T456G, P451Q, T455G, N449−, | 3 | 1.667 | 0.083 | 7 |
| | T448−, G453T, R459G | | | | |
| VAR-13 | P451Q, T455G, N449−, | 11 | 2.455 | 0.083 | 6 |
| | T448−, G453T, R459G | | | | |
| VAR-13 | N449−, Q457T, T448− | 3 | 3.000 | 0.076 | 3 |
| VAR-13 | S446A, P451Q, T455G, | 2 | 3.000 | 0.057 | 5 |
| | N449−, G453T | | | | |
| VAR-13 | P451Q, Q457T, T448−, | 6 | 2.833 | 0.035 | 5 |
| | G453T, R459G | | | | |
| VAR-13 | P451Q, T456G, N449−, Q457T | 4 | 3.000 | 0.030 | 4 |
| VAR-13 | G453T, R459G, Q457T | 2 | 3.000 | 0.015 | 3 |
| VAR-13 | T456G, T455G, N449−, | 2 | 3.000 | 0.015 | 5 |
| | G453T, R459G | | | | |
| VAR-13 | P451Q, T456G, Q457T, | 8 | 2.500 | 0.000 | 5 |
| | N449−, R459G | | | | |
| VAR-13 | P451Q, T456G, N449−, | 18 | 2.278 | 10.000 | 5 |
| | T448−, R459G | | | | |

VAR-18 mutation set with median whole retina transduction
(log2 relative to wtAAV2) greater than 0.0

| | | | | | |
|---|---|---|---|---|---|
| VAR-18 | 584_5 + aa_585_RAYN+ | 2 | 3.000 | 0.358 | 1 |

VAR-19 and VAR-23 common mutation set with median whole retina transduction
(log2 relative to wtAAV2) greater than 0.0

| | | | | | |
|---|---|---|---|---|---|
| VAR-19, | R566K | 3 | 2.333 | 0.048 | 1 |
| VAR-23 | | | | | |

VAR-26 mutation set with median whole retina transduction
(log2 relative to wtAAV2) greater than 0.0

| | | | | | |
|---|---|---|---|---|---|
| VAR-26 | A590P, T597S, V600A | 4 | 2.500 | 0.093 | 3 |

VAR-3 mutation set with median whole retina transduction
(log2 relative to wtAAV2) greater than 0.0

| | | | | | |
|---|---|---|---|---|---|
| VAR-3 | N449Q, P451T | 2 | 1.500 | 0.680 | 2 |

VAR-3 and VAR-8 common mutation set with median whole retina transduction
(log2 relative to wtAAV2) greater than 0.0

| | | | | | |
|---|---|---|---|---|---|
| VAR-3, | P451T, S458Q, T456G, Q457T | 3 | 3.000 | 0.045 | 4 |
| VAR-8 | | | | | |

VAR-30 mutation set with median whole retina transduction
(log2 relative to wtAAV2) greater than 0.0

| | | | | | |
|---|---|---|---|---|---|
| VAR-30 | 586_6 + aa_587_DQDFK+ | 4 | 2.500 | 0.113 | 1 |

VAR-32 mutation sets with median whole retina transduction
(log2 relative to wtAAV2) greater than 0.0

| | | | | | |
|---|---|---|---|---|---|
| VAR-32 | 587_8 + aa_588_LAIEQTR+ | 2 | 2.500 | 0.175 | 1 |
| VAR-32 | 587_7 + aa_588_+EQTRPA | 9 | 2.778 | 0.126 | 1 |
| VAR-32 | 587_6 + aa_588_+QTRPA | 9 | 3.000 | 0.036 | 1 |

VAR-33 mutation sets with median whole retina transduction
(log2 relative to wtAAV2) greater than 0.0

| | | | | | |
|---|---|---|---|---|---|
| VAR-33 | 584_8 + aa_585_RARLDET+, | 200 | 2.980 | 0.505 | 2 |
| | G586P | | | | |
| VAR-33 | 584_8 + aa_585_RARLDET+, | 203 | 2.980 | 0.491 | 2 |
| | N587A | | | | |
| VAR-33 | 584_8 + aa_585_RARLDET+, | 268 | 2.239 | 0.488 | 3 |
| | G586P, N587A | | | | |
| VAR-33 | 584_7 + aa_585_RARLDE+, | 234 | 2.970 | 0.446 | 3 |
| | G586P, N587A | | | | |
| VAR-33 | 584_7 + aa_585_RARLDE+, | 7 | 3.000 | 0.368 | 2 |
| | N587A | | | | |
| VAR-33 | G586P, N587A, | 8 | 3.000 | 0.233 | 3 |
| | 584_6 + aa_585_RARLD+ | | | | |
| VAR-33 | 584_8 + aa_585_RARLDET+ | 4 | 3.000 | 0.073 | 1 |

VAR-35 mutation sets with median whole retina transduction
(log2 relative to wtAAV2) greater than 0.0

| | | | | | |
|---|---|---|---|---|---|
| VAR-35 | 586_7 + aa_587_+GEQTRP | 4 | 3.000 | 0.736 | 1 |
| VAR-35 | 586_6 + aa_587_+EQTRP, | 16 | 3.000 | 0.736 | 2 |
| | N587A | | | | |
| VAR-35 | N587A, | 7 | 2.429 | 0.494 | 2 |
| | 586_7 + aa_587_+GEQTRP | | | | |

VAR-36 mutation sets with median whole retina transduction
(log2 relative to wtAAV2) greater than 0.0

| | | | | | |
|---|---|---|---|---|---|
| VAR-36 | 586_5 + aa_587_ATDT+ | 3 | 2.333 | 0.514 | 1 |
| VAR-36 | 586_6 + aa_587_ATDTK+ | 19 | 2.842 | 0.143 | 1 |
| VAR-36 | 586_6aa_587_ATDTKT | 20 | 1.900 | 0.111 | 1 |
| VAR-36 | 586_6 + aa_587_+TDTKT | 20 | 2.750 | 0.111 | 1 |

VAR-4 mutation set with median whole retina transduction
(log2 relative to wtAAV2) greater than 0.0

| | | | | | |
|---|---|---|---|---|---|
| VAR-4 | S452T, T456G | 3 | 2.667 | 0.474 | 2 |

VAR-8 mutation sets with median whole retina transduction
(log2 relative to wtAAV2) greater than 0.0

| | | | | | |
|---|---|---|---|---|---|
| VAR-8 | P451T, T456G, Q461A, | 2 | 2.000 | 0.252 | 5 |
| | S458Q, Q457T | | | | |
| VAR-8 | S458Q, R459T, T455G | 9 | 2.778 | 0.118 | 3 |
| VAR-8 | Q461A, S458Q, T456G, Q457T | 3 | 3.000 | 0.045 | 4 |
| VAR-8 | P451T, T456G, T455G, R459T, | 2 | 1.000 | 0.043 | 6 |
| | S458Q, Q457T | | | | |
| VAR-8 | P451T, S458Q, Q457T, R459T | 3 | 3.000 | 0.041 | 4 |
| VAR-8 | P451T, S458Q, T456G, T455G | 3 | 2.667 | 0.041 | 4 |
| VAR-8 | R459T, T456G, T455G, Q457T | 3 | 3.000 | 0.041 | 4 |
| VAR-8 | T455G, Q461A, R459T, | 4 | 2.750 | 0.011 | 5 |
| | S458Q, Q457T | | | | |

Accordingly, provided herein is a capsid polypeptide that comprises a sequence at least 90%, at least 91%, at least 92%, at least 93%, at least 94%, at least 95%, at least 96%, at least 97%, at least 98%, or at least 99% identical to a VP1, VP2, or VP3 of SEQ ID NO: 1, and comprising a Common Mutation Set listed in Table 12 or Table 13. In embodiments, a virus particle comprising said capsid polypeptide exhibits increased retina transduction, relative to a virus particle comprising wild-type AAV2, for example, as determined by NGS sequencing of viral cDNA from bulk retina tissue as described herein. In embodiments, the virus particle has at least 2-fold increased retina transduction relative to wild-type AAV2. for example, as determined by NGS sequencing of viral cDNA from bulk retina tissue as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-11. In embodiments, the Common Mutation Set comprises or consists of: (1) R459-, and T456-, (2) R459-, and Q457-, (3) S458-, T456-, and L460-, (4) R459-, Q457-, and T456-, (5) Q457-, and L460-, (6) R459-, Q457-, and L460-, (7) R459-, S458-, and Q457-, (8) S458-, Q457-, and T456-, (9) L460-, S458-, R459-, Q457-, and T456-, (10) R459-, S458-, Q457-, and L460-, (11) R459-, S458-, Q457-, and T456-, (12) S458-, and L460-, (13) R459-, S458-, L460-, (14) R459-, and S458-, (15) S458-, and Q457-, or (16) S458-, and T456-. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold or at least 4-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-12. In embodiments, the Common Mutation Set comprises or consists of: (1) S458Q, T455Q, and P451G, (2) T456N, and R459T, (3) N449I, T456N, R459T, and T455Q, (4) R459T, and P451G, (5) S458Q, T454-, and T450N, (6) R459T, and T454-, or (7) S458Q, Q461K, R459T, and Q464V. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 4-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-13. In embodiments, the Common Mutation Set comprises or consists of: (1) S446A, P451Q, T455G, N449-, T448-, G453T, and Q457T, (2) S446A, P451Q, T456G, T455G, N449-, T448-, G453T, and Q457T, (3) S446A, and Q457T, or (4) S446A, and T456G. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2 fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-14. In embodiments, the Common Mutation Set comprises or consists of: (1) T455A, T450S, and R459D, (2) N449Q, T455A, T450S, and R459D, (3) N449Q, T450S, and R459D, (4) S452G, T455A, T450S, N449Q, and R459D, (5) N449Q, S452G, T455A, and R459D, (6) N449Q, T455A, and R459D, (7) N449Q, S452G, and R459D, or (8) N449Q, and R459D. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90% identical to a VP1, VP2 or VP3 of SEQ ID NO: 1, and comprising a S578D mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-21. In embodiments, the Common Mutation Set comprises or consists of: (1) S578I, and M558L, (2) S578I, and S580A, (3) M558L, and S580A, (4) K556N, and S578I. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90% identical to a VP1, VP2 or VP3 of SEQ ID NO: 1, and comprising a S578I mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue.

Provided herein is a capsid polypeptide, for example, a capsid polypeptide comprising a sequence that is at least 90% identical to a VP1, VP2 or VP3 of SEQ ID NO: 1, and comprising a I559L, and T581D mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-26. In embodiments, the Common Mutation Set comprises or consists of: (1) R588T, R585S, T597S, A591I, and V600A, (2) R588T, R585S, Q589N, A590P, T597S, and A591I, (3) A593G, R585S, T597S, A591I, and V600A, (4) R588T, R585S, A590P, T597S, A591I, and V600A, (5) R588T, A593G, R585S, Q589N, A590P, and V600A, (6) R588T, A593G, Q589N, A590P, and A591I, (7) R588T, R585S, Q589N, A590P, and T597S, or (8) R588T, R585S, Q589N, A590P, T597S, and V600A. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90% identical to a VP1, VP2 or VP3 of SEQ ID NO: 1, and comprising an insertion at any position between amino acid 580 and amino acid 600 of SEQ ID NO:1, preferably after position 592 of SEQ ID NO:1, wherein the insertion comprises LX1X2, wherein X1 and X2 are any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-3. In embodiments, the Common Mutation Set comprises or consists of: (1) P451T, T456G, T455L, N449Q, S458Q, and Q457T, (2) P451T, T456G, T450M, N449Q, S458Q, and Q457T, (3) N449Q, T455L, T456G, and S458Q, (4) P451T, T455L, N449Q, S458Q, and Q457T, (5) P451T, T456G, T455L, N449Q, and Q457T, (6) P451T, T456G, R459M, N449Q, and Q457T. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-3, VAR-4 VAR-8, VAR-13 and/or VAR-14. In embodiments, the Common Mutation Set comprises or consists of: (1) N449Q, (2) N449Q, and T456G, (3) N449Q, T456G, and Q457T, (4) T456G, (5) T456G, and Q457T, (6) Q457T or (7) N449Q, and Q457T. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 6 amino acids following any of amino acid positions 580-600 of SEQ ID NO: 1, for example, following amino acid 587 according to SEQ ID NO: 1, wherein the insertion comprises or consists of X1X2EQTRPA (SEQ ID NO: 134), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-4. In embodiments, the Common Mutation Set comprises or consists of: (1) N449Q, T456G, and S452T, (2) S452T, T456G, and Q457T, (3) T456G, P451-, Q457T, N449Q, and R459G, (4) S452T, P451-, and Q457T, (5) N449Q, P451-, T456G, and S452T, (6) S452T, and P451-, (7) N449Q, Q457T, and S452T, (8) N449Q, Q457T, and R459G, (9) N449Q, P451-, T456G, and Q457T, or (10) P451-, and R459G. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-4 and VAR-13. In embodiments, the Common Mutation Set comprises or consists of: (1) T456G, R459G, and Q457T, (2) R459G. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-40. In embodiments, the Common Mutation Set comprises or consists of: (1) T597A, R585S, G586S, A591E, D594R, and V600I, (2) R585S, G586S, A591E, D594R, and V600I, (3) A591E, T597A, R585S, and D594R, (4) T597A, R585S, N587A, G586S, D594R, and V600I, (5) T597A, R585S, N587A, A591E, and D594R, (6) T597A, R585S, N587A, G586S, and D594R, (7) A591E, T597A, R585S, and G586S, or (8) R585S, N587A, G586S, A591E, and V600I. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-8. In embodiments, the Common Mutation Set comprises or consists of: (1) P451T, T456G, T455G, R459T, S458Q, and Q457T, (2) T456G, T455G, R459T, S458Q, and Q457T, (3) P451T, T456G, T455G, R459T, and Q457T, (4) P451T, S458Q, Q457T, and T455G, (5) Q461A, R459T, T456G, and T455G, (6) S458Q, R459T, T456G, and T455G, (7) P451T, T456G, R459T, S458Q, and Q457T, (8) P451T, S458Q, Q457T, and R459T, (9) P451T, S458Q, T456G, and R459T, (10) T456G, T455G, Q461A, S458Q, and Q457T, (11) P451T, T456G, T455G, Q461A, R459T, and Q457T, (12) P451T, Q461A, R459T, S458Q, and Q457T, (13) S458Q, T456G, T455G, and Q457T, (14) P451T, T456G, Q461A, R459T, and S458Q, (15) Q461A, R459T, and T455G, (16) T456G, T455G, Q461A, R459T, and S458Q, (17) Q461A, P451T, and T455G, (18) P451T, R459T, T456G, and Q457T, (19) S458Q, R459T, T456G, and Q457T, (20) R459T, T456G, and T455G, (21) S458Q, T456G, and R459T, (22) Q461A, R459T, S458Q, and T455G, (23) T456G, T455G, Q461A, R459T, S458Q, and Q457T, (24) T456G, Q461A, R459T, S458Q, and Q457T, (25) R459T, and T456G, (26) P451T, S458Q, R459T, and Q461A, (27) T455G, Q461A, R459T, S458Q, and Q457T, (28) T456G, T455G, Q461A, R459T, and Q457T, (29) Q461A, T456G, T455G, and Q457T, (30) Q461A, R459T, T456G, and Q457T, (31) Q461A, S458Q, and R459T (32) S458Q, R459T, and Q457T, (33) R459T, and Q457T, (34) R459T, and T455G, (35) Q461A, and R459T, (36) Q461A, P451T, T456G, and Q457T, (37) P451T, T456G, and R459T, (38) P451T, S458Q, and R459T, or (39) Q461A, and T455G. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 32-fold, at least 16-fold, at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising a Common Mutation Set associated with VAR-8 and VAR-12 or VAR-13. In embodiments, the Common Mutation Set comprises or consists of: (1) S458Q, and R459T, (2) R459T, (3) T456G, and T455G, or (4) Q457T, and T455G. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 32-fold, at least 16-fold, at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 4 amino acids following any of amino acid positions 580-600 of SEQ ID NO: 1, for example, following amino acid 584 according to SEQ ID NO: 1, wherein the insertion comprises or consists of RAYNX1X2 (SEQ ID NO: 135), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90% identical to a VP1, VP2 or VP3 of SEQ ID NO: 1, and comprising a R566K mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 4 amino acids following any of amino acid positions 580-600 of SEQ ID NO: 1, for example, following amino acid 586 according to SEQ ID NO: 1, wherein the insertion comprises or consists of DQDFKX1X2 (SEQ ID NO: 136), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 587 according to SEQ ID NO: 1, wherein the insertion comprises or consists of IEQTRX1X2 (SEQ ID NO: 137) or LAIEQTRX1X2 (SEQ ID NO: 138), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 6 amino acids following any of amino acid positions 580-600 of SEQ ID NO: 1, for example, following amino acid 587 according to SEQ ID NO: 1, wherein the insertion comprises or consists of X1X2EQTRPA (SEQ ID NO: 132), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 587 according to SEQ ID NO: 1, wherein the insertion comprises or consists of X1X2QTRPA (SEQ ID NO: 139), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 584 according to SEQ ID NO: 1, wherein the insertion comprises or consists of RARLDX1X2 (SEQ ID NO: 140), RARLDEX1X2 (SEQ ID NO: 141) or RARLDETX1X2 (SEQ ID NO: 142), wherein X1 and X2 are independently selected from any amino acid or not present. In embodiments the capsid polypeptide further comprises a G586P mutation. In embodiments, the capsid polypeptide further comprises a N587A mutation. In embodiments, the capsid polypeptide further comprises a G586P and N587A mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 7 amino acids following any of amino acid positions 580-600 of SEQ ID NO: 1, for example, following amino acid 584 according to SEQ ID NO: 1, wherein the insertion comprises or consists of RARLDETX1X2 (SEQ ID NO: 142), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 586 according to SEQ ID NO: 1, wherein the insertion comprises or consists of X1X2GEQTRP (SEQ ID NO: 143) or X1X2EQTRP (SEQ ID NO: 144), wherein X1 and X2 are independently selected from any amino acid or not present. In embodiments, the capsid polypeptide further comprises a N587A mutation. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 4 amino acids following any of amino acid positions 580-600 of SEQ ID NO: 1, for example, following amino acid 586 according to SEQ ID NO: 1, wherein the insertion comprises or consists of ATDTX1X2 (SEQ ID NO: 145), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 586 according to SEQ ID NO: 1, wherein the insertion comprises or consists of ATDTKX1X2 (SEQ ID NO: 146), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 6 amino acids following any of amino acid positions 580-600 of SEQ ID NO: 1, for example, following amino acid 586 according to SEQ ID NO: 1, wherein the insertion comprises or consists of ATDTKT (SEQ ID NO: 115), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

Provided herein is a capsid polypeptide, for example, a capsid polypeptide comprising a sequence that is at least 90%, at least 91%, at least 92%, at least 93%, at least 94%, at least 95%, at least 96%, at least 97%, at least 98%, or at least 99% identical to a VP1, VP2 or VP3 of SEQ ID NO: 1, and comprising an insertion of at least 5 amino acids following any of amino acid positions 580-600 of SEQ ID NO: 1, for example, following amino acid 586 according to SEQ ID NO: 1, wherein the insertion comprises or consists of X1X2TDTKT (SEQ ID NO: 147), wherein X1 and X2 are independently selected from any amino acid or not present. In embodiments, the capsid polypeptide comprises fewer than 10, fewer than 9, fewer than 8, fewer than 7, fewer than 6, fewer than 5, fewer than 4, fewer than 3, fewer than 2 or no additional mutations relative to the VP1, VP2 or VP3 sequence of SEQ ID NO: 1. In embodiments, a virus particle comprising said capsid polypeptide exhibits increased retina transduction, relative to a virus particle comprising wild-type AAV2, for example, as determined by NGS sequencing of viral cDNA from bulk retina tissue. In embodiments, the virus particle exhibits at least 8-fold, at least 4-fold or at least 2-fold increased retina transduction, relative to wild-type AAV2, for example as determined as described herein.

In embodiments, provided herein is a nucleic acid molecule encoding a capsid polypeptide, for example a VP1, VP2 or VP3 capsid polypeptide, described above. In embodiments, provided herein is a virus particle comprising a capsid polypeptide, for example, a VP1, VP2 or VP3 capsid polypeptide, described above.

### Methods of Making Compositions Described Herein

The disclosure is directed, in part, to a method of making a capsid polypeptide described herein or a dependoparvovirus particle, e.g., a dependoparvovirus particle described herein. In some embodiments, a method of making dependoparvovirus particle comprises providing a cell, cell-free system, or other translation system, comprising a nucleic acid described herein encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, providing a cell comprising a nucleic acid described herein comprises introducing the nucleic acid to the cell, e.g., transfecting or transforming the cell with the nucleic acid. The nucleic acids of the disclosure may be situated as a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon. Such a vector may be delivered by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus variant capsid polypeptide as provided for herein or a fragment thereof. In some embodiments, vectors of the disclosure comprises sequences encoding a dependoparvovirus rep protein or a fragment thereof. In some embodiments, such vectors may contain sequence encoding both dependoparvovirus cap (e.g., a variant capsid polypeptide described herein) and rep proteins. In vectors in which both AAV rep and cap are provided, the dependoparvovirus rep and dependoparvovirus cap sequences may both be of the same dependoparvovirus species or serotype origin, such as AAV2. Alternatively, the present disclosure also provides vectors in which the rep sequences are from a dependoparvovirus species or serotype which differs from that from which the cap sequences are derived. In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector. In some embodiments, the vectors of the invention further contain a payload, e.g., a minigene comprising a selected transgene (e.g., a payload as described herein), e.g., flanked by dependoparvovirus 5′ ITR and dependoparvovirus 3′ ITR.

The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In some embodiments, the disclosure provides a method of making a dependoparvovirus particle (e.g., a dependoparvovirus B particle, e.g., an AAV2 particle or particle comprising a variant capsid polypeptide as described herein), or a portion thereof. In some embodiments, the method comprises culturing a host cell which contains a nucleic acid sequence encoding a dependoparvovirus variant capsid polypeptide as provided for herein, or fragment thereof; a functional rep gene; a payload (e.g., as described herein), e.g., a minigene comprising dependoparvovirus inverted terminal repeats (ITRs) and a transgene, optionally under the control of a regulatory element such as a promoter; and sufficient helper functions to promote packaging of the payload, e.g., minigene, into the dependoparvovirus capsid. The components necessary to be cultured in the host cell to package a payload, e.g., minigene, in a dependoparvovirus capsid may be provided to the host cell in trans. In some embodiments, any one or more of the required components (e.g., payload (e.g., minigene), rep sequences, cap sequences, and/or helper functions) may be provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required component may be under the control of a constitutive promoter. Examples of suitable inducible and constitutive promoters are provided herein and further examples are known to those of skill in the art. In some embodiments, a selected host cell which has been engineered to stably comprise one or more components may comprise a component under the control of a constitutive promoter and another component under the control of one or more inducible promoters. For example, a host cell which has been engineered to stably comprise the required components may be generated from 293 cells (e.g., which comprise helper functions under the control of a constitutive promoter), which comprises the rep and/or cap proteins under the control of one or more inducible promoters.

The payload (e.g., minigene), rep sequences, cap sequences, and helper functions required for producing a dependoparvovirus particle of the disclosure may be delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, may be readily selected from among any dependoparvovirus species and serotypes, e.g., AAV1, AAV2, AAV3, AAV4, AAV5, AAV6, AAV7, AAV9. ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

The dependoparvovirus particles (e.g., including a variant capsid polypeptide and, for example, a payload) of the disclosure may be produced using any invertebrate cell type which allows for production of dependoparvovirus or biologic products and which can be maintained in culture. In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38.

In some embodiments, the methods of the disclosure can be carried out with any mammalian cell type which allows for replication of dependoparvovirus or production of biologic products, and which can be maintained in culture. In some embodiments, the mammalian cells used can be HEK293, HEK293T, HeLa, CHO, NS0, SP2/0, PER.C6, Vero, RD, BHK, HT 1080, A549, Cos-7, ARPE-19 or MRC-5 cells. In some embodiments the culture is an adherent cell culture. In some embodiments, the culture is a suspension cell culture.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid.

In some embodiments, a nucleic acid sequence encoding an dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating cells, cell-free systems, and other translation systems are known to those of skill in the art. In some embodiments, cultivating a cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid (e.g., a capsid comprising a variant capsid polypeptide provided for herein), expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, a nucleic acid or polypeptide described herein is produced by a method known to one of skill in the art. The nucleic acids, polypeptides, and fragments thereof of the disclosure may be produced by any suitable means, including recombinant production, chemical synthesis, or other synthetic means. Such production methods are within the knowledge of those of skill in the art and are not a limitation of the present invention.

### Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises a sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises a sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

A cell of the disclosure may be generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

In some embodiments, the virus particle comprising the variant capsid is produced at a level at least 10%, at least 20%, at least 50%, at least 100%, at least 200% or greater than the production level of wt AAV2 from the same producer cell type, e.g., from HEK293 cells, e.g., from adherent culture of HEK293 cells.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell.

### Virus Particles and Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) comprising the payload. In some embodiments, the dependoparvovirus particle is a dependoparvovirus particle described herein and comprises a payload described herein. In some embodiments, the cell is an ocular cell. In some embodiments, the ocular cell is in the retina, macula, or trabecular meshwork. In some embodiments, the ocular cell is in the retina. In some embodiments, the ocular cell is in the macula. In some embodiments, the ocular cell is in the trabecular meshwork.

In some embodiments, the ocular cell is in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, the cell is in the cornea, iris, ciliary body, lens, trabecular meshwork, or Schlemm's canal, or any combination thereof.

In some embodiments, the ocular cell is posterior to the lens, such as in the anterior hyaloid membrane and all of the optical structures behind it, such as the vitreous humor, retina, choroid or optic nerve, or any combination thereof. Accordingly, in some embodiments, the cell is in the anterior hyaloid membrane and all of the optical structures behind it, such as the vitreous humor, retina, choroid or optic nerve, or any combination thereof.

The disclosure is further directed in part to a virus particle comprising a capsid polypeptide described herein. In embodiments, the virus particle comprises a capsid polypeptide described herein and a nucleic acid expression construct. In embodiments the nucleic acid expression construct of the virus particle comprises a payload.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. The nucleic acid of the transgene may be operatively linked to a regulatory component in a manner sufficient to promote transgene transcription, translation, and/or expression in a host cell.

A transgene may be any polypeptide or RNA encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., β-lactamase, β-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene may be used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle may comprise or deliver multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) may be used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein may be encoded by the same transgene, e.g., a single transgene encoding each of the subunits with the DNA for each subunit separated by an internal ribozyme entry site (IRES) or enzymatically cleavable sequence (e.g., a furin cleavage site). In some embodiments, the DNA may be separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther 8(10):811-817 (May 2001).

In some embodiments, virus particles comprising a genome are provided, wherein the genome includes a nucleic acid expression construct. The nucleic acid expression construct can include a payload, for example a payload comprising a heterologous transgene and one or more regulatory elements.

In some embodiments, the particle delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, or 150-times as compared to a virus particle comprising capsid polypeptides of SEQ ID NO: 1. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to macular tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue and non-macular retina tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue and non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue, macular tissue, and non-macular retina tissue. In some embodiments, the particle delivers the payload to the eye with increased transduction specificity in one or more regions of the eye as compared to a virus particle comprising capsid polypeptides of SEQ ID NO: 1 without increased biodistribution in one or more regions of the eye as compared to a virus particle comprising capsid polypeptides of SEQ ID NO: 1. In any of the aforementioned embodiments, increased transduction or biodistribution is as measured as described herein in Example 1 (for example, with respect to transduction, as measured by quantification of viral cDNA isolated from the bulk tissue, e.g., NHP tissue, of interest normalized to prevalence of that virus particle in the test article, and with respect to biodistribution, as measured by quantification of viral DNA isolated from bulk tissue, e.g., NHP tissue, of interest normalized to prevalence of that virus particle in the test article).

In some embodiments, the regulatory elements include a promotor. In some embodiments, the promoter is a ubiquitous or constitutive promoter active in a mammalian cell, for example a human cell, for example, in a human cell type of interest. In some embodiments, the cell type is an ocular cell such as, for example, a neural retinal cell, a photoreceptive retinal ganglion cell, a bipolar cell, a horizontal cell, a amacrine cell, a photoreceptor (e.g., a rod or a cone cell), an endothelial cell (e.g., a retinal pigmented epithelial cell), and endothelial-like cell, and the like. Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and optionally the splice acceptor of the rabbit beta globin gene), chicken-beta actin promoter, CBA promoter, CMV promoter, human PGK promoter, ubiquitin promoter, human EF1-alpha promoter and fragments thereof. In some embodiments, the promoter is a tissue-specific promoter, for example, a promoter specific in ocular tissue or cells of the eye. Examples of ocular tissue-specific promoters include but are not limited to TBG promoters, hAAT promoters, CK8 promoters and SPc5-12 promoters, rho promoters, which are active in rods, or opsin promoters, which are active in cones. In some embodiments, the regulatory element includes a photoreceptor cell-specific regulatory element (e.g., promoter) such as, e.g., a rhodopsin promoter; a rhodopsin kinase promoter; a beta phosphodiesterase gene promoter; a retinitis pigmentosa gene promoter; an interphotoreceptor retinoid-binding protein (IRBP) gene enhancer; an IRBP gene promoter, an opsin gene promoter, a retinoschisin gene promoter, a CRX homeodomain protein gene promoter, a guanine nucleotide binding protein alpha transducing activity polypeptide 1 (GNAT1) gene promoter, a neural retina-specific leucine zipper protein (NRL) gene promoter, human cone arrestin (hCAR) promoter, and the PR2.1, PR1.7, PR1.5, and PR1.1 promoters. In some embodiments, the regulatory element includes, a retinal pigment epithelia (RPE) cell-specific regulatory element (e.g., a RPE-specific promoter), e.g., a regulatory element that confers selective expression of the operably linked gene in a RPE cell, such as, e.g., an RPE65 gene promoter, a cellular retinaldehyde-binding protein (CRALBP) gene promoter, a pigment epithelium-derived factor (PEDF aka serpin F1) gene promoter, and a vitelliform macular dystrophy (VMD2) promoter. In some embodiments, the regulatory element includes a promoter specific to a glial cell, e.g., a regulatory element that confers selective expression of the operably linked payload in a retinal glial cell, such as, e.g., a glial fibrillary acidic protein (GFAP) promoter. In some instances, the regulatory element includes a promoter that is specific to a bipolar cell (e.g., a bipolar-specific promoter), e.g., a regulatory element that confers selective expression of the operably linked payload in a bipolar cell, such as, e.g., a GRM6 promoter. In embodiments, the promoter sequence is between 100 and 1000 nucleotides in length. In embodiments, the promoter sequence is about 100, about 200, about 300, about 400, about 500, about 600, about 700, about 800, about 900 or about 1000 nucleotides in length. As used in the preceding sentence, “about” refers to a value within 50 nucleotides of the recited length. Suitable regulatory elements, e.g., promoters, may be readily selected by persons of skill in the art, such as those, but not limited to, those described herein.

In some embodiments, the nucleic acid expression construct comprises an intron. The intron may be disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b-globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfereing nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. Examples of diseases (and heterologous transgenes or molecules encoded by said heterologous transgenes) according to the present disclosure include: MPSI (alpha-L-iduronidase (IDUA)); MPS II—Hunter syndrome (iduronate-2-sulfatase (IDS)); Ceroid lipofuscinosis-Batten disease (CLN1, CLN2, CLN10, CLN13, CLN5, CLN11, CLN4, CNL14, CLN3, CLN6, CLN7, CLN8, CLN12); MPS Illa—Sanfilippo Type A syndrome (heparin sulfate sulfatase (also called N-sulfoglucosamine sulfohydrolase (SGSH)); MPS IIIB—Sanfilippo Type b syndrome (N-acetyl-alpha-D-glucosaminidase (NAGLU)); MPS VI—Maroteaux-Lamy syndrome (arylsulfatase B); MPS IV A—Morquio syndrome type A (GALNS); MPS IV B—Morquio syndrome type B (GLB1); Osteogenesis Imperfecgta Type I, II, III or IV (COL1A1 and/or COL1A2); hereditary angioedema (SERPING1, C1NH); Osteogenesis Imperfecta Type V (IFITM5); Osteogenesis Imperfecta Type VI (SERPINF1); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and III (Glucocerebrosidase; GBA1); Parkinson's Disease (Glucocerebrosidase; GBA1 and/or dopamine decarboxylase); Pompe (acid maltase; GAA; hGAA); Metachromatic leukodystrophy (Aryl sulfatase A); MPS VII—Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anit-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase); Fabry Disease (alpha-galactosidase); Fucosidosis (fucosidase (FUCA1)); Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); Parkinson's disease (glutamic acid decarboxylase; FGF-2; BDGF); Spinal Muscular Atrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease la (Glucose-6-phosphatase); XLMTM (MTMl); Crigler Najjar (UGTlAl); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C—Gamma-sarcoglycanopathy (gamma-sarcoglycan); Angelman syndrome (UBE3A); Retinitis Pigmentosa (hMERTK); Age-Related Macular Degeneration (sFLT01); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy—MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphaIAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMI); Crigler-Najjar Syndrome (UGTlAl); Retinitis Pigmentosa (hPDE6B); Mucopolysaccharidosis Type 3B (hNAGLU); Duchenne Muscular Dystrophy (GALGT2); Alzheimer's Disease (NGF; ApoE4; ApoE2; ApoE3; Anti-ApoE RNAi); Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Leber's Hereditary Optical Neuropathy (PlND4v2); Aromatic Amino Acid Decarboxylase Deficiency (hAADC); Retinitis Pigmentosa (hMERKTK); and Retinitis Pigmentosa (RLBP1).

In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to: and an anti-Ab antibody (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651), anti-ALKI (e.g., ascrinvacumab), anti-C5 (e.g., tesidolumab, ravulizumab, and eculizumab), anti-CD105 (e.g., carotuximab), anti-CClQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD 19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In embodiments, the payload comprises a nucleic acid encoding a gene product linked to a disorder of the eye, or a fragment thereof. Exemplary gene products linked to a disorder of the eye include, for example, ADP-ribosylation factor-like 6 (ARL6); BBSome interacting protein 1 (BBIP1); BBSome protein 1 (BBS1); BBSome protein 2 (BBS2); BBSome protein 4 (BBS4); BBSome protein 5 (BBS5); BBSome protein 7 (BBS7); BBSome protein 9 (BBS9); BBSome protein 10 (BBS10); BBSome protein 12 (BBS12); centrosomal protein 290 kDa (CEP290); intraflagellar transport protein 172 (IFT172); intraflagellar transport protein 27 (IFT27); inositol polyphosphate-5-phosphatase E (INPP5E); inwardly-rectifying potassium channel subfamily J member 13 (KCNJ13); leucine zipper transcription factor like-1 (LZTFL1); McKusick-Kaufman syndrome protein (MKKS); Meckel syndrome type 1 protein (MKS1); nephronophthisis 3 protein (NPHP1); serologically-defined colon cancer antigen 8 (SDCCAG8); tripartite motif-containing protein 32 (TRIM32); tetratricopeptide repeat domain 8 (TTC8); Batten disease protein (CLN3); cytochrome P450 4V2 (CYP4V2); Rab escort protein 1 (CHM); PR (positive regulatory) domain-containing 13 protein (PRDM13); RPE-retinal G protein-coupled receptor (RGR); TEA domain family member 1 (TEAD1); arylhydrocarbon-interacting receptor protein-like 1 (AIPL1); cone-rod otx-like photoreceptor homeobox transcription factor (CRX); guanylate cyclase activating protein 1A (GUCA1A); retinal-specific guanylate cyclase (GUCY2D); phosphatidylinositol transfer membrane-associated family member 3 (PITPNM3); prominin 1 (PROM1); peripherin (PRPH); peripherin 2 (PRPH2); regulating synaptic membrane exocytosis protein 1 (RIMS1); semaphorin 4A (SEMA4A); human homolog of C. elegans unc119 protein (UNC119); ATP-binding cassette transporter-retinal (ABCA4); ADAM metallopeptidase domain 9 (ADAM9); activating transcription factor 6 (ATF6); chromosome 21 open reading frame 2 (C21orf2); chromosome 8 open reading frame 37 (C8orf37); calcium channel; voltage-dependent; alpha 2/delta subunit 4 (CACNA2D4); cadherin-related family member 1 (protocadherin 21) (CDHR1); ceramide kinase-like protein (CERKL); cone photoreceptor cGMP-gated cation channel alpha subunit (CNGA3); cone cyclic nucleotide-gated cation channel beta 3 subunit (CNGB3); cyclin M4 (CNNM4); guanine nucleotide binding protein (G protein); alpha transducing activity polypeptide 2 (GNAT2); potassium channel subfamily V member 2 (KCNV2); Phosphodiesterase 6C (PDE6C); Phosphodiesterase 6H (PDE6H); proteome of centriole 1 centriolar protein B (POC1B); RAB28 member of RAS oncogene family (RAB28); retina and anterior neural fold homeobox 2 transcription factor (RAX2); 11-cis retinol dehydrogenase 5 (RDH5); RP GTPase regulator-interacting protein 1 (RPGRIP1); tubulin tyrosine ligase-like family member 5 (TTLL5); L-type voltage-gated calcium channel alpha-1 subunit (CACNA1F); retinitis pigmentosa GTPase regulator (RPGR); rod transducin alpha subunit (GNAT1); rod cGMP phosphodiesterase beta subunit (PDE6B); rhodopsin (RHO); calcium binding protein 4 (CABP4); G protein-coupled receptor 179 (GPR179); rhodopsin kinase (GRK1); metabotropic glutamate receptor 6 (GRM6); leucine-rich repeat immunoglobulin-like transmembrane domains protein 3 (LRIT3); arrestin (s-antigen) (SAG); solute carrier family 24 (SLC24A1); transient receptor potential cation channel, subfamily M, member 1 (TRPM1); nyctalopin (NYX); green cone opsin (OPN1LW); red cone opsin (OPN1MW); blue cone opsin (OPN1SW); frataxin (FXN); inosine monophosphate dehydrogenase 1 (IMPDH1); orthodenticle homeobox 2 protein (OTX2); crumbs homolog 1 (CRB1); death domain containing protein 1 (DTHD1); growth differentiation factor 6 (GDF6); intraflagellar transport 140 Chlamydomonas homolog protein (IFT140); IQ motif containing B protein (IQCB1); lebercilin (LCA5); lecithin retinol acyltransferase (LRAT); nicotinamide nucleotide adenylyltransferase 1 (NMNAT1); RD3 protein (RD3); retinol dehydrogenase 12 (RDH12); retinal pigment epithelium-specific 65 kD protein (RPE65); spermatogenesis associated protein 7 (SPATA7); tubby-like protein 1 (TULP1); mitochondrial genes (KSS, LHON, MT-ATP6, MT-TH, MT-TL1, MT-TP, MT-TS2, mitochondrially encoded NADH dehydrogenases [MT-ND]); bestrophin 1 (BEST1); C1q and tumor necrosis-related protein 5 collagen (C1QTNF5); EGF-containing fibrillin-like extracellular matrix protein 1 (EFEMP1); elongation of very long fatty acids protein (ELOVL4); retinal fascin homolog 2, actin bundling protein (FSCN2); guanylate cyclase activating protein 1B (GUCAB); hemicentin 1 (HMCN1); interphotoreceptor matrix proteoglycan 1 (IMPG1); retinitis pigmentosa 1-like protein 1 (RP1L1); tissue inhibitor of metalloproteinases-3 (TIMP3); complement factor H (CFH); complement factor D (CFD); complement component 2 (C2); complement component 3 (C3); complement factor B (CFB); DNA-damage regulated autophagy modulator 2 (DRAM2); chondroitin sulfate proteoglycan 2 (VCAN); mitofusin 2 (MFN2); nuclear receptor subfamily 2 group F member 1 (NR2F1); optic atrophy 1 (OPA1); transmembrane protein 126A (TMEM126A); inner mitochondrial membrane translocase 8 homolog A (TIMM8A); carbonic anhydrase IV (CA4); hexokinase 1 (HK1); kelch-like 7 protein (KLHL7); nuclear receptor subfamily 2 group E3 (NR2E3); neural retina lucine zipper (NRL); olfactory receptor family 2 subfamily W member 3 (OR2W3); pre-mRNA processing factor 3 (PRPF3); pre-mRNA processing factor 4 (PRPF4); pre-mRNA processing factor 6 (PRPF6); pre-mRNA processing factor 8 (PRPF8); pre-mRNA processing factor 31 (PRPF31); retinal outer segment membrane protein 1 (ROM1); retinitis pigmentosa protein 1 (RP1); PIM-kinase associated protein 1 (RP9); small nuclear ribonucleoprotein 200 kDa (SNRNP200); secreted phosphoprotein 2 (SPP2); topoisomerase I binding arginine/serine rich protein (TOPORS); ADP-ribosylation factor-like 2 binding protein (ARL2BP); chromosome 2 open reading frame 71 (C2orf71); clarin-1 (CLRN1); rod cGMP-gated channel alpha subunit (CNGA1); rod cGMP-gated channel beta subunit (CNGB1); cytochrome P450 4V2 (CYP4V2); dehydrodolichyl diphosphate synthetase (DHDDS); DEAH box polypeptide 38 (DHX38); ER membrane protein complex subunit 1 (EMC1); eyes shut/spacemaker homolog (EYS); family with sequence similarity 161 member A (FAM161A); G protein-coupled receptor 125 (GPR125); heparan-alpha-glucosaminide N-acetyltransferase (HGSNAT); NAD(+)-specific isocitrate dehydrogenase 3 beta (IDH3B); interphotoreceptor matrix proteoglycan 2 (IMPG2); KIAA1549 protein (KIAA1549); kizuna centrosomal protein (KIZ); male germ-cell associated kinase (MAK); c-mer protooncogene receptor tyrosine kinase (MERTK); mevalonate kinase (MVK); NIMA (never in mitosis gene A)-related kinase 2 (NEK2); neuronal differentiation protein 1 (NEUROD1); cGMP phosphodiesterase alpha subunit (PDE6A); phosphodiesterase 6G cGMP-specific rod gamma (PDE6G); progressive rod-cone degeneration protein (PRCD); retinol binding protein 3 (RBP3); retinaldehyde-binding protein 1 (RLBP1); solute carrier family 7 member 14 (SLC7A14); usherin (USH2A); zinc finger protein 408 (ZNF408); zinc finger protein 513 (ZNF513); oral-facial-digital syndrome 1 protein (OFD1); retinitis pigmentosa 2 (RP2); retinoschisin (RS1); abhydrolase domain containing protein 12 (ABHD12); cadherin-like gene 23 (CDH23); centrosomal protein 250 kDa (CEP250); calcium and integrin binding family member 2 (CIB2); whirlin (DFNB31); monogenic audiogenic seizure susceptibility 1 homolog (GPR98); histidyl-tRNA synthetase (HARS); myosin VIIA (MYO7A); protocadherin 15 (PCDH15); harmonin (USH1C); human homolog of mouse scaffold protein containing ankyrin repeats and SAM domain (USH1G); dystrophin (DMD); norrin (NDP); phosphoglycerate kinase (PGK1); calpain 5 (CAPN5); frizzled-4 Wnt receptor homolog (FZD4); integral membrane protein 2B (ITM2B); low density lipoprotein receptor-related protein 5 (LRP5); micro RNA 204 (MIR204); retinoblastoma protein 1 (RB1); tetraspanin 12 (TSPAN12); chromosome 12 open reading frame 65 (C12orf65); cadherin 3 (CDH3); membrane-type frizzled-related protein (MFRP); ornithine aminotransferase (OAT); phospholipase A2 group V (PLA2G5); retinol-binding protein 4 (RBP4); regulator of G-protein signaling 9 (RGS9); regulator of G-protein signaling 9-binding protein (RGS9BP); ARMS2; excision repair cross-complementing rodent repair deficiency complementation group 6 protein (ERCC6); fibulin 5 (FBLN5); HtrA serine peptidase 1 (HTRA1); toll-like receptor 3 (TLR3); and toll-like receptor 4 (TLR4), opsin; rhodopsin; channel rhodopsin; halo rhodopsin, and the like.

In some embodiments, the virus particle comprises a heterologous transgene encoding a genome editing system. Examples include a CRISPR genome editing system (e.g., one or more components of a CRISPR genome editing system such as, for example, a guide RNA molecule and/or a RNA-guided nuclease such as a Cas enzyme such as Cas9, Cpf1 and the like), a zinc finger nuclease genome editing system, a TALEN genome editing system or a meganuclease genome editing system. In embodiments, the genome editing system targets a mammalian, e.g., human, genomic target sequence. In embodiments, the virus particle includes a heterologous transgene encoding a targetable transcription regulator. Examples include a CRISPR-based trascription regulator (for example, one or more components of a CRISPR-based transcription regulator, for example, a guide RNA molecule and/or a enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

The transgene may also encode any biologically active product or other product, e.g., a product desirable for study. Suitable transgenes may be readily selected by persons of skill in the art, such as those, but not limited to, those described herein.

Other examples of proteins encoded for by the transgene include, but are not limited to, colony stimulating factors (CSF); blood factors, such as β-globin, hemoglobin, tissue plasminogen activator, and coagulation factors; interleukins; soluble receptors, such as soluble TNF-α. receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines; enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

Accordingly, provided herein is a virus particle comprising a capsid polypeptide comprising (a) a VP1, VP2 or VP3 sequence of SEQ ID NO: 2, (b) a VP1, VP2 or VP3 sequence comprising the mutation set of VAR-1 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-1 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 2. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 3, (b) a VP1, VP2 or VP3 sequence comprising the mutation set of VAR-2 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-2 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 3. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 4, (b) a VP1, VP2 or VP3 sequence comprising the mutation set of VAR-3 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-3 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 4. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 5, (b) a VP1, VP2 or VP3 sequence comprising the mutation set of VAR-4 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-4 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 5. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 6, (b) a VP1, VP2 or VP3 sequence comprising the mutation set of VAR-5 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-5 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 6. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 7, (b) a VP1, VP2 or VP3 sequence comprising the mutation set of VAR-6 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-6 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 7. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 8, (b) a VP1, VP2 or VP3 sequence comprising the mutation set of VAR-7 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-7 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 8. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 9, (b) a VP1, VP2 or VP3 sequence comprising the mutation set of VAR-8 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-8 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 9. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 10, (b) a VP1, VP2 or VP3 sequence comprising the mutation set of VAR-9 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-9 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 10. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 11, (b) a VP1, VP2 or VP3 sequence comprising the mutation set of VAR-10 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-10 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 11. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 12, (b) a VP1, VP2 or VP3 sequence comprising the mutation set of VAR-11 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-11 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 12. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 13, (b) a VP1, VP2 or VP3 sequence comprising the mutation set of VAR-12 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-12 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 13. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 14, (b) a VP1, VP2 or VP3 sequence comprising the mutation set of VAR-13 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-13 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 14. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 15, (b) a VP1, VP2 or VP3 sequence comprising the mutation set of VAR-14 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-14 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 15. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 16, (b) a VP1, VP2 or VP3 sequence comprising the mutation set of VAR-15 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-15 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 14. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 17, (b) a VP1, VP2 or VP3 sequence comprising the mutation set of VAR-16 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-16 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 17. In embodiments, the capsid polypeptide comprises VP1, VP2 and VP3 sequences of SEQ ID NO: 5. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 18, (b) a VP1, VP2 or VP3 sequence comprising the mutation set of VAR-17 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-17 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 18. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 19, (b) a VP1, VP2 or VP3 sequence comprising the mutation set of VAR-18 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-18 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 19. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 20, (b) a VP1, VP2 or VP3 sequence comprising the mutation set of VAR-19 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-19 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 20. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 21, (b) a VP1, VP2 or VP3 sequence comprising the mutation set of VAR-20 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-20 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 21. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 22, (b) a VP1, VP2 or VP3 sequence comprising the mutation set of VAR-21 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-21 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 22. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 23, (b) a VP1, VP2 or VP3 sequence comprising the mutation set of VAR-22 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-22 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 23. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 24, (b) a VP1, VP2 or VP3 sequence comprising the mutation set of VAR-23 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-23 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 24. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 25, (b) a VP1, VP2 or VP3 sequence comprising the mutation set of VAR-24 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-24 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 25. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 26, (b) a VP1, VP2 or VP3 sequence comprising the mutation set of VAR-25 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-25 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 26. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 27, (b) a VP1, VP2 or VP3 sequence comprising the mutation set of VAR-26 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-26 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 27. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 28, (b) a VP1, VP2 or VP3 sequence comprising the mutation set of VAR-27 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-27 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 28. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 29, (b) a VP1, VP2 or VP3 sequence comprising the mutation set of VAR-28 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-28 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 29. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 30, (b) a VP1, VP2 or VP3 sequence comprising the mutation set of VAR-29 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-29 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 31. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 31, (b) a VP1, VP2 or VP3 sequence comprising the mutation set of VAR-30 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-30 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 31. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 32, (b) a VP1, VP2 or VP3 sequence comprising the mutation set of VAR-31 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-31 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 32. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 33, (b) a VP1, VP2 or VP3 sequence comprising the mutation set of VAR-32 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-32 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 33. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 34, (b) a VP1, VP2 or VP3 sequence comprising the mutation set of VAR-33 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-33 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 34. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 35, (b) a VP1, VP2 or VP3 sequence comprising the mutation set of VAR-34 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-34 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 35. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 36, (b) a VP1, VP2 or VP3 sequence comprising the mutation set of VAR-35 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-35 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 36. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 37, (b) a VP1, VP2 or VP3 sequence comprising the mutation set of VAR-36 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-36 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 37. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 38, (b) a VP1, VP2 or VP3 sequence comprising the mutation set of VAR-37 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-37 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 38. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 39, (b) a VP1, VP2 or VP3 sequence comprising the mutation set of VAR-38 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-38 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 39. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 40, (b) a VP1, VP2 or VP3 sequence comprising the mutation set of VAR-39 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-39 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 40. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 41, (b) a VP1, VP2 or VP3 sequence comprising the mutation set of VAR-40 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-40 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 41. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 42, (b) a VP1, VP2 or VP3 sequence comprising the mutation set of VAR-41 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-41 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 42. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 43, (b) a VP1, VP2 or VP3 sequence comprising the mutation set of VAR-42 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-42 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 43. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 44, (b) a VP1, VP2 or VP3 sequence comprising the mutation set of VAR-43 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-43 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 44. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 45, (b) a VP1, VP2 or VP3 sequence comprising the mutation set of VAR-44 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-44 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 45. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 46, (b) a VP1, VP2 or VP3 sequence comprising the mutation set of VAR-45 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-45 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 46. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

The disclosure is further directed, in part, to a method of delivering a payload to a subject, e.g., an animal or human subject. In some embodiments, a method of delivering a payload to a subject comprises administering to the subject a dependoparvovirus particle comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the eye. In some embodiments, the delivery to the eye is increased as compared to a particle without the variant capsid polypeptide or as compared to a wild-type capsid polypeptide.

### Methods of Treatment

The disclosure is directed, in part, to a method of treating a disease or condition in a subject, e.g., an animal or human subject. As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing. In some embodiments, a method of treating a disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the disease or condition. In some embodiments, the payload is a therapeutic product. In some embodiments, the payload is a nucleic acid, e.g., encoding an exogenous polypeptide.

The dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelieal carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma.

In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in the “Methods of delivering a payload” section.

The dependoparvovirus particles comprising a variant polypeptide described herein or produced by the methods described herein can be used to express one or more therapeutic proteins to treat various diseases or disorders. In some embodiments, the disease or disorder is a disease or disorder of the eye, for example, retinitis pigmentosa; macular degeneration (e.g.; wet age-related macular degeneration), optic neuritis; Leber's congenital amaurosis; Leber's hereditary optic neuropathy; achromatopsia; X-linked retinoschisis; optic neuritis; choroideremia; optic atrophy; retinal cone dystrophy; retinopathy; retinoblastoma; glaucoma; Bardet-Biedl syndrome; Usher syndrome; aniridia; Friedreich's ataxia; vitelliform macular dystrophy; retinoblastoma; Stargardt disease; Charcot-Marie-Tooth disease; Fuch's dystrophy; propionic acidemia; or color blindness; corneal dystrophy; keratoconus; night blindness; dry eye; Bardet-Biedl syndrome; Batten's Disease; Bietti's Crystalline Dystrophy; chorioretinal atrophy; chorioretinal degeneration; cone or cone-rod dystrophies (autosomal dominant, autosomal recessive, and X-linked), congenital stationary night blindness (autosomal dominant, autosomal recessive, and X-linked); disorders of color vision, including achromatopsia (including ACHM2, ACHM3, ACHM4, and ACHM5), protanopia, deuteranopia, and tritanopia; Friedreich's ataxia; Leber's congenital amaurosis (autosomal dominant and autosomal recessive), including, but not limited to, LCA1, LCA2, LCA3, LCA4, LCA6, LCA7, LCA8, LCA12, and LCA15; Leber's Hereditary Optic Neuropathy; macular dystrophy (autosomal dominant and autosomal recessive), including, but not limited to, acute macular degeneration, Best vitelliform macular dystrophy, pattern dystrophy, North Carolina Macular Dystrophy, inherited drusen, Sorsby's fundus dystrophy, malattia levantanese, and genetically-determined retinopathy of prematurity; ocular-retinal developmental disease; ocular albinism; optic atrophies (autosomal dominant, autosomal recessive, and X-linked); retinitis pigmentosa (autosomal dominant, autosomal recessive, X-linked, and mitochondrially-inherited traits), examples of which include RP1, RP2, RP3, RP10, RP20, RP38, RP40, and RP43; X-linked retinoschisis; Stargardt disease; and Usher syndrome, including, but not limited to, USH1B, USH1C, USH1D, USH1F, USH1G, USH2A, USH2C, USH2D, AND USH3. Examples of complex genetic diseases include, but are not limited to, glaucoma (open angle, angle-closure, low-tension, normal-tension, congenital, neovascular, pigmentary, pseudoexfoliation); age-related and other forms of macular degeneration, both exudative and non-exudative forms (autosomal dominant and autosomal recessive), such as acute macular degeneration, vitelliform macular degeneration; retinopathy of prematurity; and Vogt Koyanagi-Harada (VKH) syndrome. Examples of acquired diseases include, but are not limited to, acute macular neuroretinopathy; anterior ischemic optic neuropathy and posterior ischemic optic neuropathy; Behcet's disease; branch retinal vein occlusion; choroidal neovascularization; diabetic retinopathy, including proliferative diabetic retinopathy and associated complications; diabetic uveitis; edema, such as macular edema, cystoid macular edema and diabetic macular edema; epiretinal membrane disorders; macular telangiectasia; multifocal choroiditis; non-retinopathy diabetic retinal dysfunction; ocular tumors; optic atrophies; retinal detachment; retinal disorders, such as central retinal vein occlusion, proliferative vitreoretinopathy (PVR), retinal arterial and venous occlusive disease, vascular occlusion, uveitic retinal disease; uveal effusion; retinal infective and infiltrative disease; optic nerve diseases such as acquired optic atrophy. Examples of traumatic injuries include, but are not limited to, histoplasmosis; optic nerve trauma; ocular trauma which affects a posterior ocular site or location; retinal trauma; viral infection of the eye; viral infection of the optic nerve; a posterior ocular condition caused by or influenced by an ocular laser treatment; posterior ocular conditions caused by or influenced by a photodynamic therapy; photocoagulation, radiation retinopathy; and sympathetic ophthalmia.

In some embodiments, administration of a dependoparvovirus particle comprising a variant polypeptide and comprising a payload (e.g., a transgene) to a subject induces expression of the payload (e.g., transgene) in a subject. In some embodiments, the expression is induced in the eye. In some embodiments, the production is increased in the eye as compared to a similar particle with the wild-type capsid protein. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of less than about 5 g/ml. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 g/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 g/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

In some embodiments, the a dependoparvovirus particle comprising a variant polypeptide and comprising a payload (e.g., a transgene) is administered to a subject via an injection. In some embodiments, the injection is a systemic injection, for example, intravenous, intraarterial, intramuscular, or subcutaneous injection. In some embodiments, the injection is an injection to the eye. In some embodiments, the injection is an intravitreal injection, intraorbital injection, retro-orbital injection, suprachoroidal injection, subretinal injection, subconjunctivital injection, or intracameral injection. In some embodiments, the injection is an intravitreal injection. In some embodiments, the injection is an intraorbital injection. In some embodiments, the injection is a retro-orbital injection. In some embodiments, the injection is a suprachoroidal injection. In some embodiments, the injection is a subretinal injection. In some embodiments, the injection is a subconjunctivital injection. In some embodiments, the injection is an intracameral injection.

Sequences disclosed herein may be described in terms of percent identity. A person of skill will understand that such characteristics involve alignment of two or more sequences. Alignments may be performed using any of a variety of publicly or commercially available Multiple Sequence Alignment Programs, such as “Clustal W”, accessible via the Internet. As another example, nucleic acid sequences may be compared using FASTA, a program in GCG Version 6.1. FASTA provides alignments and percent sequence identity of the regions of the best overlap between the query and search sequences. For instance, percent identity between nucleic acid sequences may be determined using FASTA with its default parameters as provided in GCG Version 6.1, herein incorporated by reference. Similar programs are available for amino acid sequences, e.g., the “Clustal X” program. Additional sequence alignment tools that may be used are provided by (protein sequence alignment; (http://www.ebi.ac.uk/Tools/psa/emboss_needle/)) and (nucleic acid alignment; http://www.ebi.ac.uk/Tools/psa/emboss_needle/nucleotide.html)). Generally, any of these programs may be used at default settings, although one of skill in the art can alter these settings as needed. Alternatively, one of skill in the art can utilize another algorithm or computer program which provides at least the level of identity or alignment as that provided by the referenced algorithms and programs. Sequences disclosed herein may further be described in terms of edit distance. The minimum number of sequence edits (i.e., additions, substitutions, or deletions of a single base or nucleotide) which change one sequence into another sequence is the edit distance between the two sequences. In some embodiments, the distance between two sequences is calculated as the Levenshtein distance.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

The invention is further illustrated by the following examples. The examples are provided for illustrative purposes only and are not to be construed as limiting the scope or content of the invention in any way.

### Examples

### Example 1

### Library Creation

A library of 2.5E5 capsid variants of wild-type AAV2 were designed and cloned into plasmids to create a library of plasmids encoding the capsid variants (library for Library Experiment 1). Experimental results from Library Experiment 1 were assessed and machine learning models trained on this and other data, and used to design two separate libraries with 1E8 capsid variants of wild-type AAV2 each (libraries for Library Experiment 2). These libraries are significantly more diverse than the library tested in Library Experiment 1. Variants in one library were designed to maximize posterior eye transduction (including for example retina, macula, non-macular retina, neural retina and choroid/RPE) (posterior eye library) and variants in the other library were designed to maximize anterior eye transduction (including for example tissues of the trabecular meshwork and Schlemm's canal) (anterior eye library). Both libraries were designed to include variants with which would produce virus particles. A library of AAV variant genomes encoding each variant's capsid and a unique capsid variant barcode identifier was cloned into three ITR plasmid backbones as described previously (Ogden et al. 2019), Each plasmid backbone contained a unique genomic identifier enabling analysis of biodistribution and transduction efficiencies via different routes of administration. The libraries were produced via transient triple transfection of adherent HEK293T followed by iodixanol gradient purification.

### In Vitro Evaluation of Library

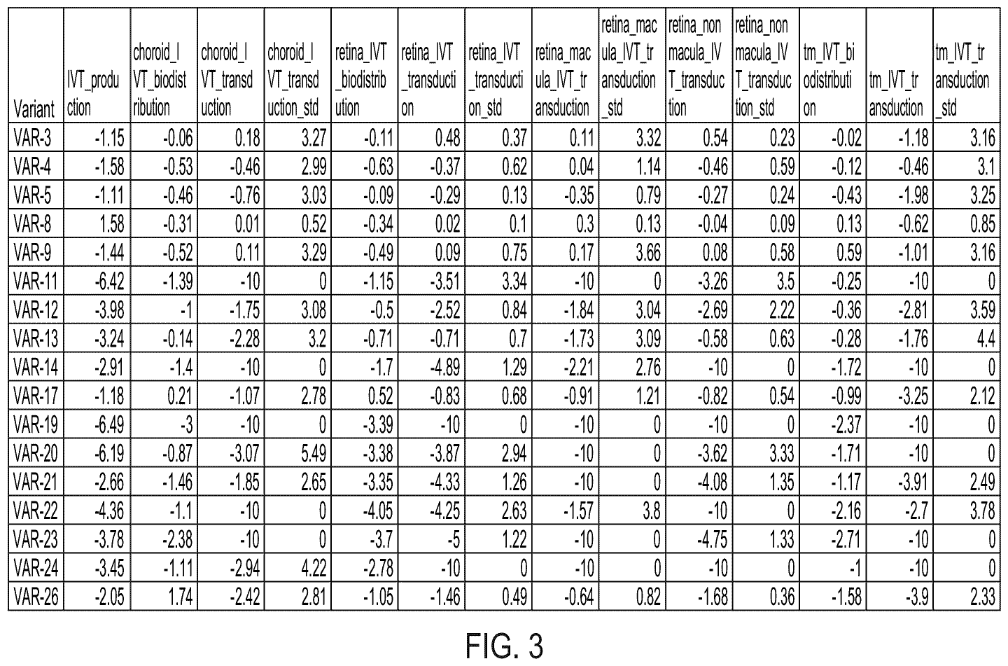

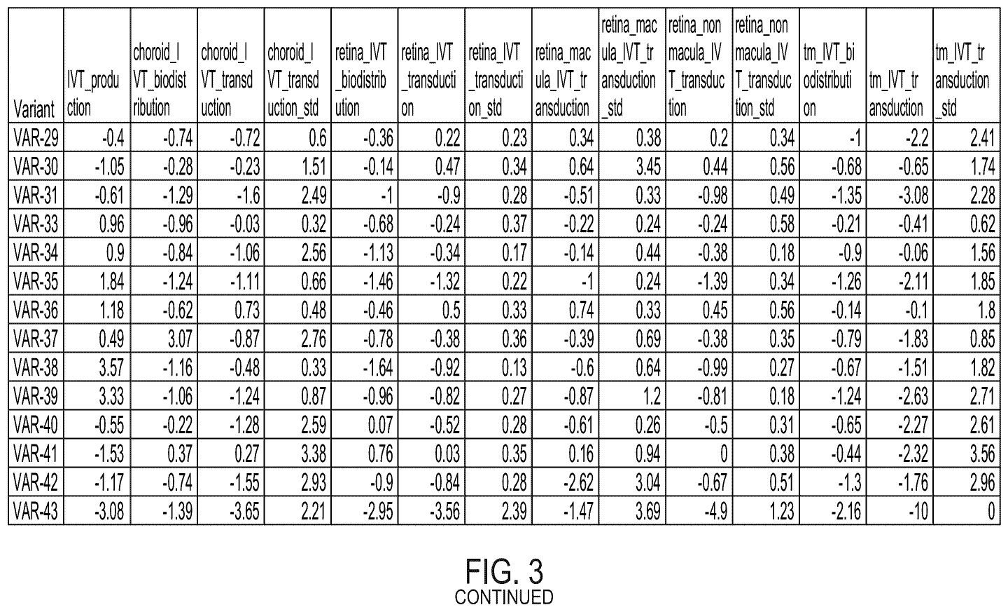

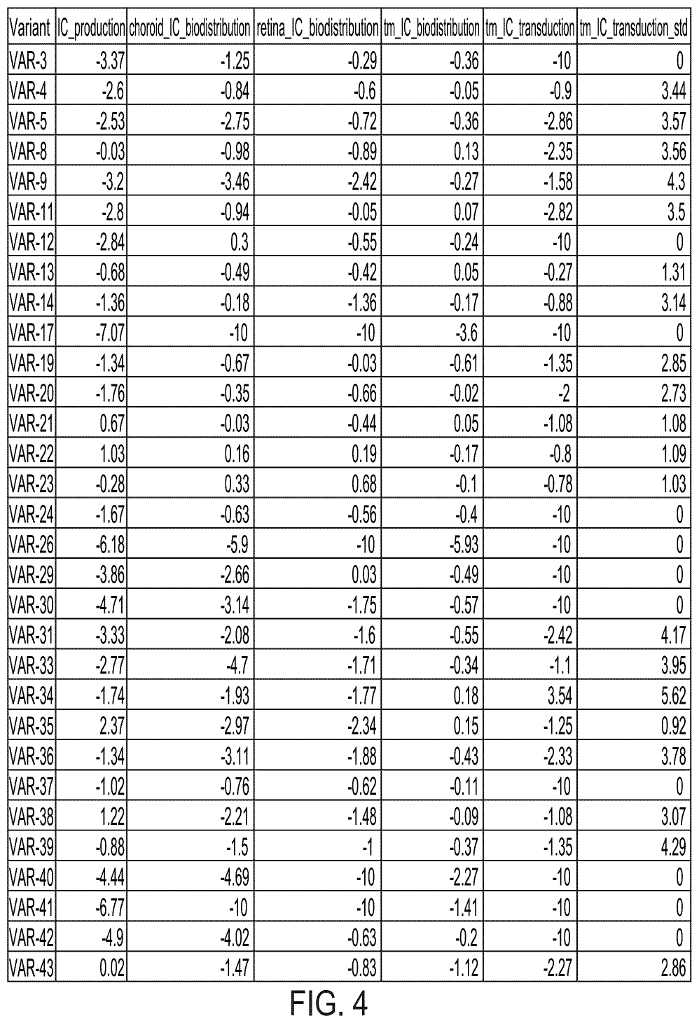

Data was prepared as described below. To measure each variant's packaging efficiency (or “production”), barcodes from vector genomes in the plasmid and produced AAV library were prepared for illumina sequencing using two rounds of PCR, Production efficiency, normalized for presence in the input plasmid library, for each variant is expressed by comparing barcode sequencing levels for each variant in the produced vector pool to the barcode sequence levels for each variant in the input plasmid library used to create the vector pool. The measurements of variant frequency in the vector library also enable downstream normalization of biodistribution and transduction measurements by variant frequency in the input vector library. Production efficiency for variants in Library Experiment 1 is reported in Table 1, and each reported value is reported as the log 2 production relative to the production of wild-type AAV2, Production efficiency for variants in the posterior eye library and anterior eye library for Library Experiment 2 are reported in Tables 6-9 and FIG. 3 and FIG. 4, and each reported value is reported as the log 2 production relative to the production of wild-type AAV2.

### In Vivo Evaluation of Library in Non-Human Primate

### Library Experiment 1

All NHP experiments were conducted in accordance with institutional policies and NIH guidelines. One young adult male and one young adult female cynomolgus macaque (Macaca fascicularis) weighing 2.4-2.9 kg seronegative for anti-AAV2 neutralizing antibodies (serum NAb titers <1:20 based on in vitro NAb assay) were selected for the study. Prior to test article administrations samples of blood, aqueous humor (50 μL) and vitreous humor (up to 50 μL) were collected. The animals were anesthetized with ketamine and dexmedetomidine and received intravitreal (IVT; 4.8E11 vg/eye in 50 μL), intracameral (IC; 8.5E11 vg/eye in 50 μL) and intravenous (IV; 1.8-2.5E13 vg/kg) injections of the vector libraries. During the in-life period the animals were monitored for signs of ocular inflammation via indirect ophthalmoscopy and slit-lamp biomicroscopy and treated with weekly IM injections of steroids (methylprednisolone, 40-80 mg) and topical steroids (Durezol), and atropine as needed according to the animal facility's SOPs and recommendations from the veterinarian. Serum samples were collected at 1 h, 4 h and 24 h, and weekly after the injections. The animals were sacrificed 4 weeks after the injections and tissues were collected for biodistribution and transduction analyses.

### Library Experiment 2

The evaluations of anterior eye library variants and posterior eye library variants in Library Experiment 2 were carried out in Cynomolgus macaques. Both eyes of two non-naive animals with low serum anti-AAV2 NAb titers (<1:10) received intravitreal injections of the posterior eye library and intracameral injections of the anterior eye library at total doses of 7.3E11 vg/eye (3.9E11 vg for IC, 3.4E11 vg for IVT), with both libraries injected via both routes of administration in each eye. Ophthalmic examinations were performed weekly during the 4 week in-life period to monitor levels of ocular inflammation.

Tissue Processing and Data Analysis for Library Experiment 1 and Library Experiment 2 Retinas and trabecular meshwork were dissected as shown in FIG. 1. A list of other tissue samples collected is shown in Table 10. All samples were collected into RNAlater® (Sigma-Aldrich) and incubated overnight at RT, after which the RNAlater® was drained and samples were frozen at −80° C. In addition, samples of aqueous humor, vitreous humor, serum, and cerebrospinal fluid were collected at necropsy and stored at −80° C.

TABLE 10
List of tissues collected.
Tissue

Adrenal gland
brain (cortical slices) coronal axis
dorsal root ganglion (cervical)
dorsal root ganglion (thoracic)
dorsal root ganglion (lumbar)
gonad (testes and ovaries)
heart, basal (left atrium)
heart, apex
heart, right ventricle
kidney
liver
Lung (superior lobe)
lymph nodes, cervical
skeletal muscle, bicep brachii
skeletal muscle, diaphragm
skeletal muscle, quadriceps
spinal cord (cervical)
spinal cord (thoracic)
spinal cord (lumbar)
spleen

For biodistribution and transduction analyses, total DNA and RNA was extracted from tissue samples with Trizol/chloroform and isopropanol precipitation. RNA samples were treated with TURBO DNase (Invitrogen). Reverse transcription was done with Protoscript II Reverse Transcriptase (NEB) with primers that were specific to the vector transgene and included unique molecular identifiers (UMIs). Control reactions lacking the reverse transcriptase enzyme (−RT control) were also prepared. Quantification of biodistribution and transduction was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample and that did not contain errors in the variant sequence, as measured by a separate sequencing assay that targeted the variant regions of the input plasmid sample.

To aggregate packaging replicates, the read counts from replicate virus production samples were summed. To aggregate transduction samples, the UMI counts from samples from the same tissue were summed.

Virus packaging, biodistribution and transduction of tissue were calculated using a Bayesian model with aggregated production, biodistribution and/or transduction samples as the input. Briefly, probabilistic programming and stochastic variational inference were used to model the measurement process and sources of decoupling (e.g., cross-packaging, template switching, and errors in DNA synthesis) between the actual test virus particles and their designed sequences, and to calculate virus production, biodistribution and transduction (in various tissue samples), and error rates. The output was the log 2-transformed mean of the calculated distribution relative to the wild-type (WT) AAV2. Thus, positive values indicate better performance than WT for the measured property, and negative values indicate worse-than-WT performance. Transduction and biodistribution for Library Experiment 1 is reported in Table 1 (transduction) and Table 5 (biodistribution). Transduction and biodistribution for Library Experiment 2 is reported in Tables 6-9. Where indicated, macula transduction and biodistribution refers to measurements taken from tissues consisting of the neural retina layer of the macula. Retina or non-macula retina transduction and biodistribution refers to measurements taken from tissues consisting of the neural retina layer of the non-macular retina areas of the eye. Measurements including the choroid and/or RPE are taken from tissues consisting of the choroid layer of the whole retina. Without being bound by theory, because the complexity of the libraries for this experiment were high relative to the total overall dose (1E8 variants with total doses of approximately 7E11 vg/eye), relative transduction and biodistribution values from Library Experiment 2 are compressed and, therefore, represent an underrepresentation of the relative transduction and biodistribution rates. The results demonstrate however, that the relative rank ordering of the variants that were included in both Library Experiment 1 and Library Experiment 2 was consistent (Spearman correlation=0.72), confirming the top variants as having significant transduction (for example, better than wild-type AAV2) improvements. Variants from this experiment are included in a follow-on library experiment of similar complexity to Library Experiment 1 (e.g., 1-2E5 variants per library), and as described in Example 2, and properties are confirmed as described herein for Library Experiment 1 and in Example 2.

### Library Experiment 1 Results

To assess efficiency of retinal transduction, measurements were obtained in both eyes of two animals across retinal regions. Data quality was ascertained through the segregation of negative controls and correlation of measurements between tissue replicates and animals. Aggregated (across eyes and animals) biodistribution and transduction measurements in eye regions of interest for virus particles comprising the capsid polypeptides described herein are provided in Table 1 and Table 5. Of note, variants with 20-60x improved transduction rates in target retinal and/or trabecular meshwork tissue relative to WT controls were discovered. Interestingly, some improved retina transducers also displayed improved transduction of the trabecular meshwork following intracameral administration, but did not display enhanced transduction of the liver following IV administration, indicating a specificity for tissues of the eye. A subset of variants with improvements specifically in peripheral retina or macula transduction were also identified. In addition, the library was assessed for other relevant parameters, such as productivity of individual variants, transduction efficiency in non-ocular tissues, and biodistribution in ocular and non-ocular tissues, In conclusion, these results demonstrate the power of machine learning models in the design of AAV variants with improved clinically relevant properties.

### Library Experiment 2 Results

Production, transduction and biodistribution measurements for variants from Library Experiment 1 that were included in Library Experiment 2 are shown in FIG. 3 (1V T administration) and FIG. 4 (IC administration). All values are log(2) relative to wt_AAV2. Columns labeled “_std” provide the 95% confidence intervals for the associated biodistribution or transduction measurements reported immediately to the left of the “_std” columns. “Retina_macula” provides measurements from aggregation of the neural retina layer of all macula samples collected. “Retina_non macula” provides measurements from aggregation of the neural retina layer of all non-macular posterior eye regions collected. In FIG. 4, “choroid” and “retina” provide measurements from aggregation of the choroid layer or the neural retina layer across all posterior eye samples.

### Example 2: Medium Throughput Experiment

### In Vivo Evaluation of Medium-Throughput Library in Non-Human Primate

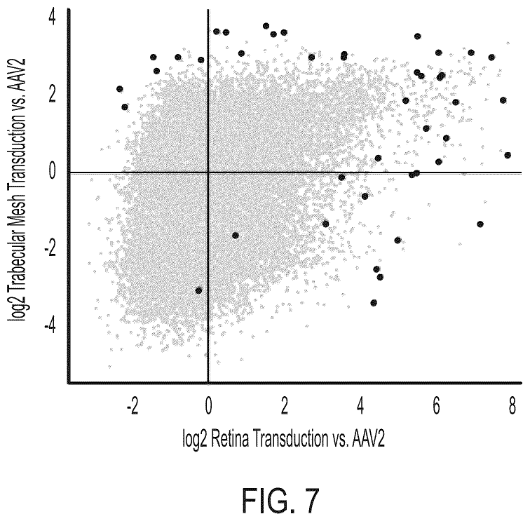

The virus particles comprising a selection of the variant capsids provided in Table 2 (sequences) are produced individually via transient triple transfection of adherent HEK293T followed by iodixanol gradient purification. Representation of individual variants within the final pooled test article were balanced to be within 10-fold range where possible. Each variant capsid was produced with a genome encoding a unique barcode and a fluorescent reporter gene under the control of a ubiquitous promoter (cbh). In all, each variant was produced with separate genomes comprising 8 unique barcodes, providing a measure of biological replicates within the study. Variants were selected from the ocular NHP dataset from Library Experiment 1 based on an algorithm that optimizes for capsid performance while balancing diversity and measurement certainty. In one arm of the experiment, variants were selected based on their transduction performance in posterior eye (including for example retina, macula, non-macular retina, neural retina and choroid/RPE) and anterior eye (including for example tissues of the trabecular meshwork and Schlemm's canal) via intravitreal (IVT) delivery. In the other arm of the experiment, variants were selected based on their transduction performance in the posterior eye via intracameral (IC) delivery. FIG. 7 shows plots all measured variants from Library Experiment 1 as a function of relative trabecular meshwork transduction by IC administration (y-axis) and relative retina (non-macula) transduction via IVT administration (x-axis). Dots represent individual capsid variants from Library Experiment 1. Dark black dots are variants chosen for this medium throughput study. As shown in FIG. 7 and as described herein, variants with high transduction across both trabecular meshwork and retina were identified, as well as variants with specificity for the retina or specificity for the trabecular meshwork tissue. Without being bound by theory, capsids with specificity for one region of the eye could have benefit for gene therapies by providing increased targeting to the tissue of interest. The study also included variants that contain stop codons in VP1 and VP2 as transduction negative controls (expected to produce virus but not transduce cells), and containing vp3 stop codons as production negative controls (not expected to produce virus).

Production efficiency is assessed as described above. Equivalent amounts (vg) of each virus particle are pooled (approximately 50-100 variants total) in equimolar amounts, and injected into AGM or other non-human primate, for example, Cynomolgus macaque at doses used in Example 1. Virus properties, including biodistribution and tissue transduction are assessed, for example, as described in Example 1.

We focused our study on variants identified in Library Experiment 1 (for example, variants described herein) datasets with improved transduction of the retina and trabecular meshwork. First, we selected variants from the ocular NHP dataset from Library Experiment 1 based on an algorithm that optimizes for capsid performance while balancing diversity and measurement certainty. Next, we synthesized 86 variants and benchmarks, and produced these separately with co-purification, balancing representation of individual variants to be within a defined range of abundance in the final vector preparations. Variants were paired with genomes bearing identifying barcode sets as well as diverse random sequence IDs for quantification of transduction events in bulk data.

All NHP experiments were conducted in accordance with institutional policies and NIH guidelines. Two young adult male cynomolgus macaques (Macaca fascicularis) weighing 2.8-3 kg, one seronegative (serum NAb titers <1:20 based on in vitro NAb assay) and one seropositive (1:128) for anti-AAV2 neutralizing antibodies were selected for the study. Prior to test article administrations samples of blood, aqueous humor (50 μL) and vitreous humor (up to 50 μL) were collected. The animals were anesthetized with ketamine and dexmedetomidine and received intravitreal (IVT; 2.63E11 vg/eye in 50 μL) and intracameral (IC; 1.11E11 vg/eye in 50 μL) injections of the vector libraries. During the in-life period the animals were monitored for signs of ocular inflammation via indirect ophthalmoscopy and slit-lamp biomicroscopy and treated with weekly IM injections of steroids (methylprednisolone, 80 mg) and topical steroids (Durezol), and atropine as needed according to the animal facility's SOPs and recommendations from the veterinarian. Confocal scanning laser ophthalmoscopy (cSLO) with green fluorescent protein (GFP) imaging using the Heidelberg Spectralis HRA/OCT system was used to take fluorescent images of each eye prior to necropsy. The animals were sacrificed 4 weeks after the injections and tissues were collected for biodistribution and transduction analyses.

Retinas and trabecular meshwork were dissected as shown in FIG. 1. All Ocular tissues were weighed and flash frozen on dry ice following dissection. A list of all Ocular tissues collected is shown in Table 11.

TABLE 11
Samples from Medium Throughput Study
Front of the Eye Samples

| Sample # | Region |
|---|---|
| 1 | Iris/ciliary body (superior) |
| 2 | Iris/ciliary body (nasal) |
| 3 | Iris/ciliary body (inferior) |
| 4 | Iris/ciliary body (temporal) |
| 5 | Region containing Trabecula/Schlemm's (superior) |
| 6 | Region containing Trabecula/Schlemm's (nasal) |
| 7 | Region containing Trabecula/Schlemm's (inferior) |
| 8 | Region containing Trabecula/Schlemm's (temporal) |
| 9 | Cornea (combined) |
| 10 | Remaining limbal tissue (combined) |

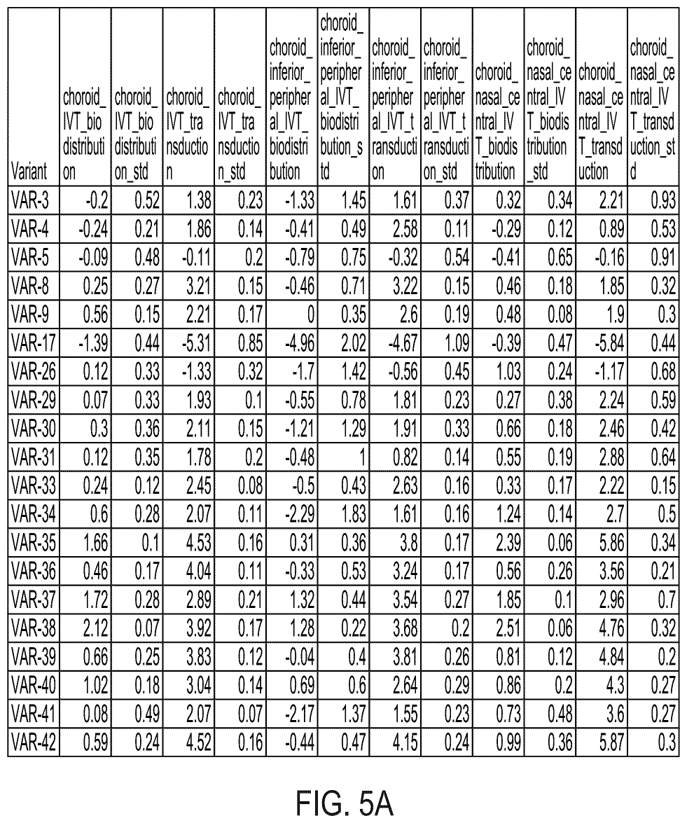

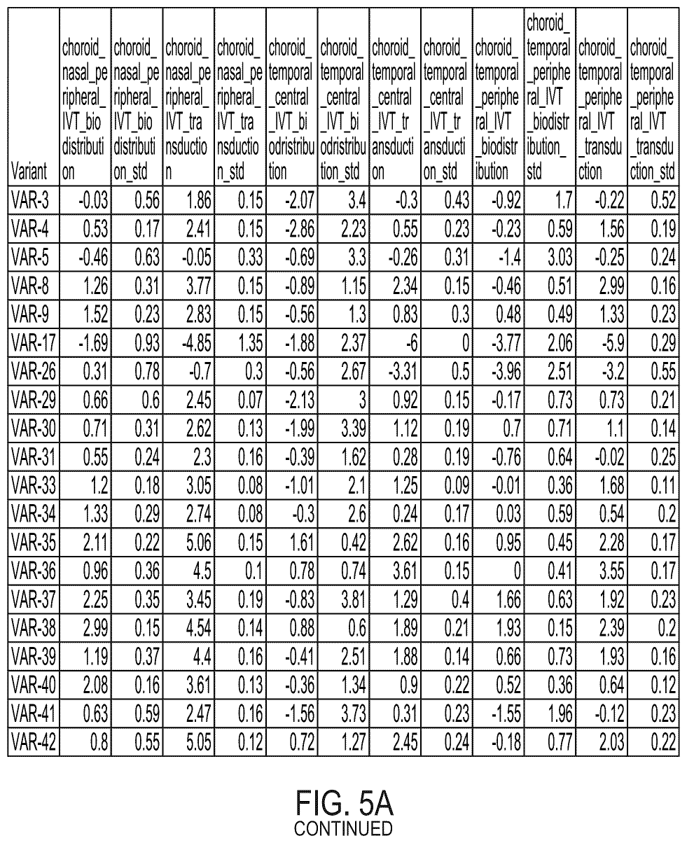

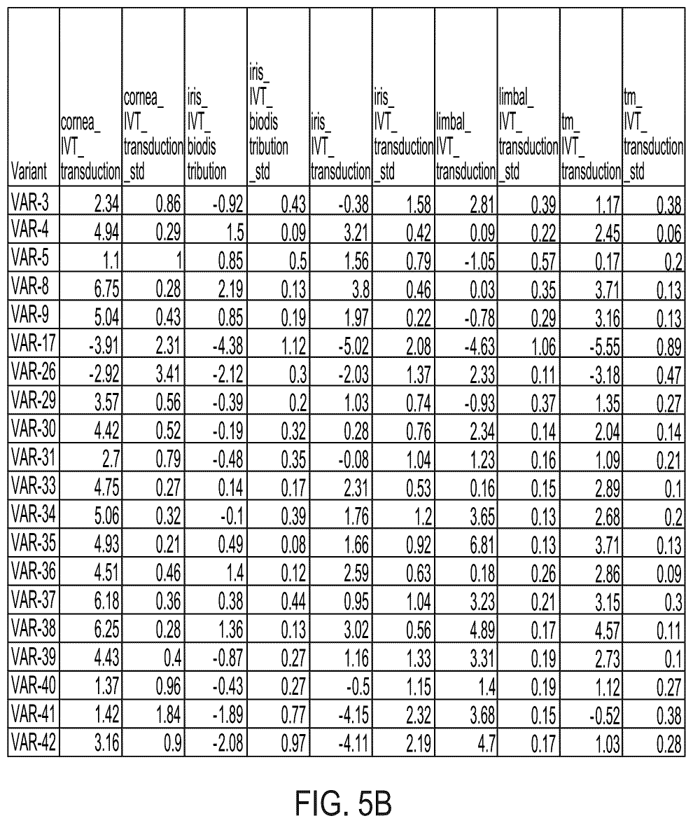

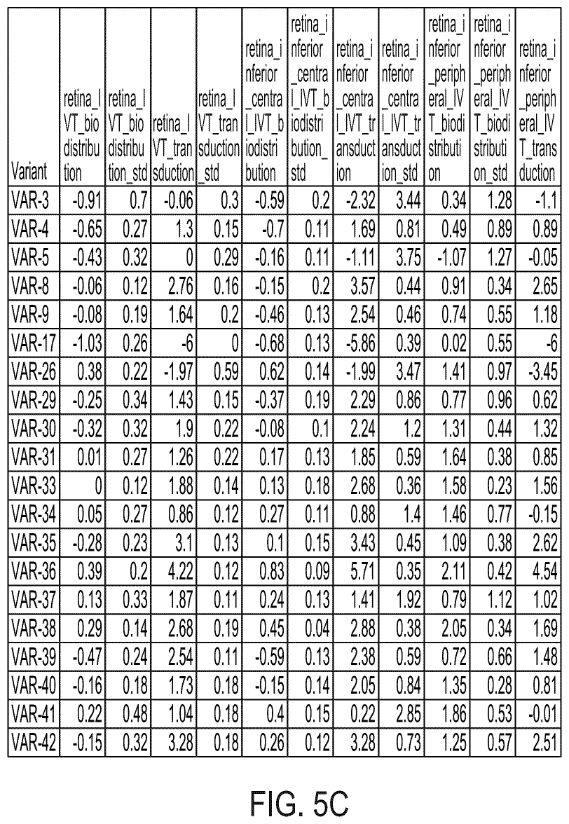

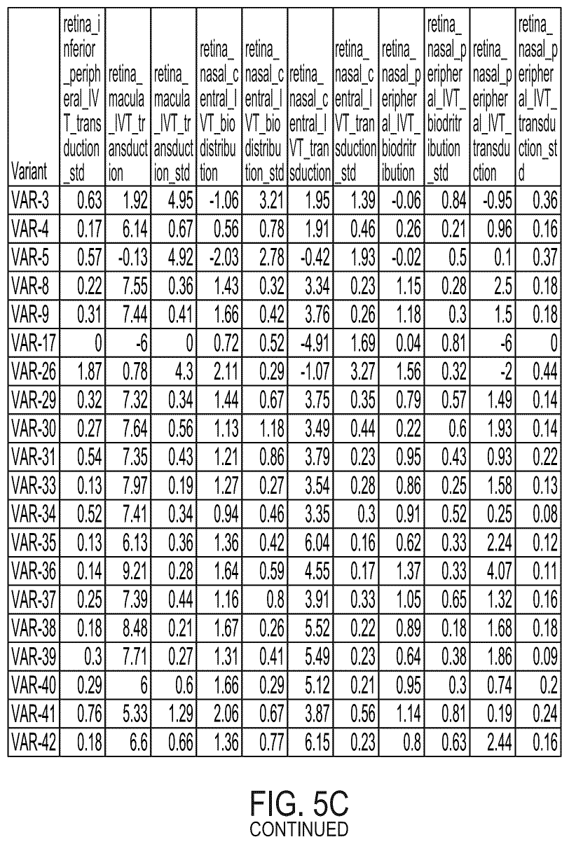

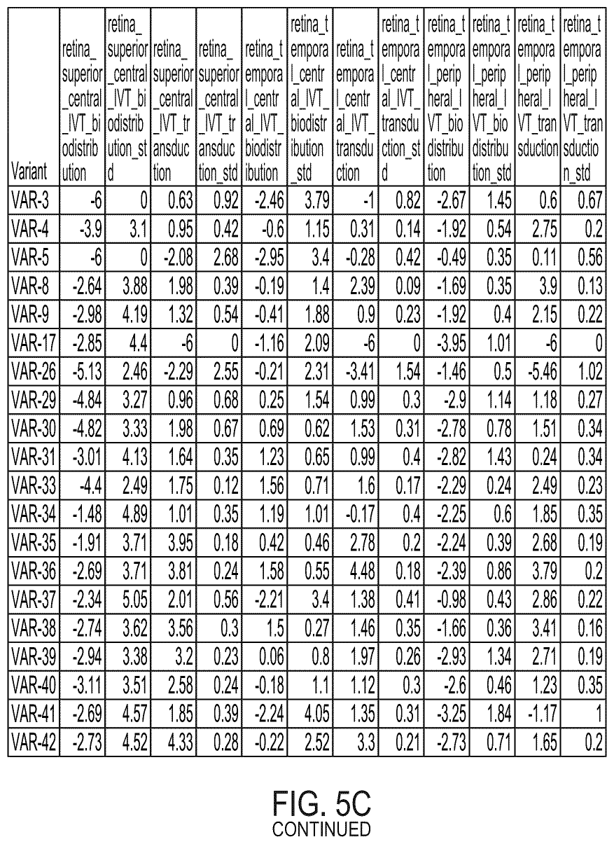

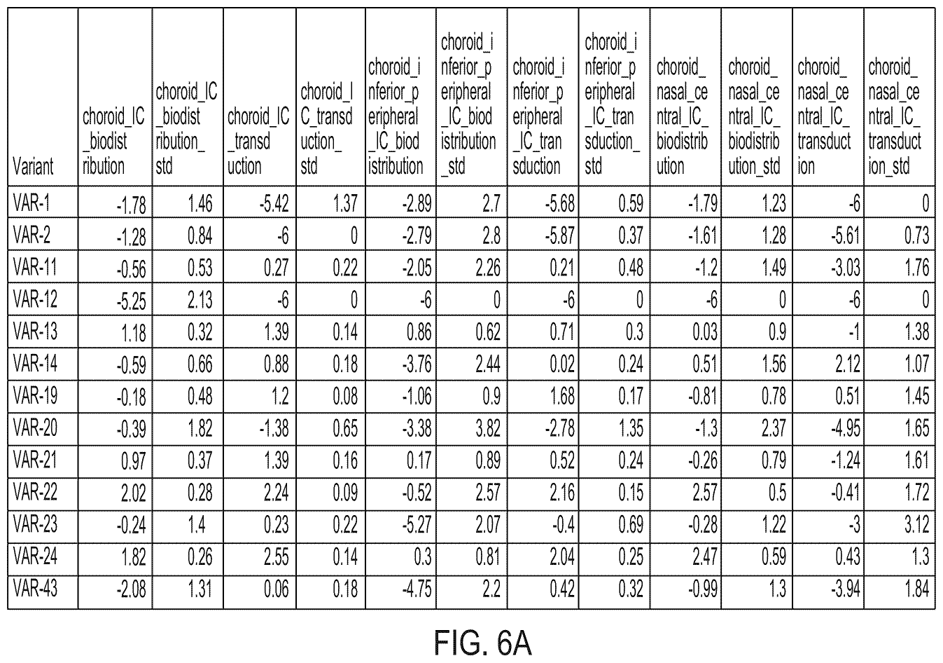

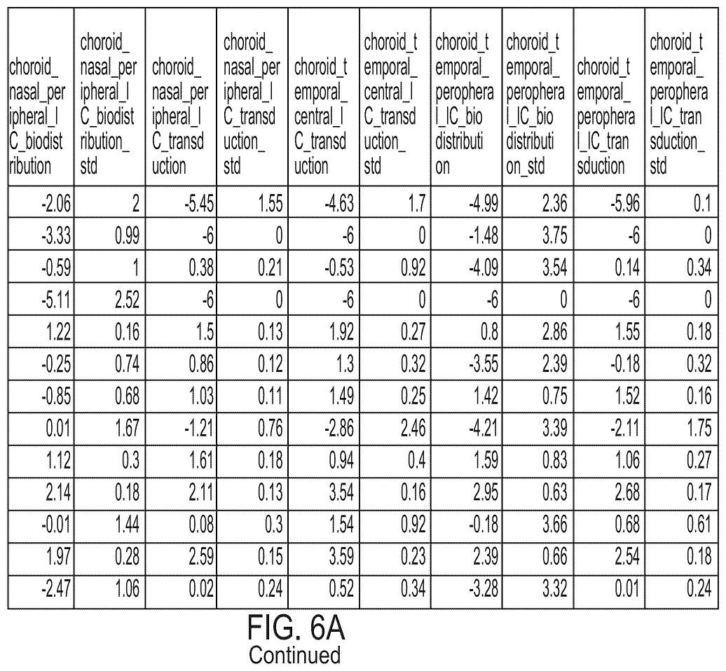

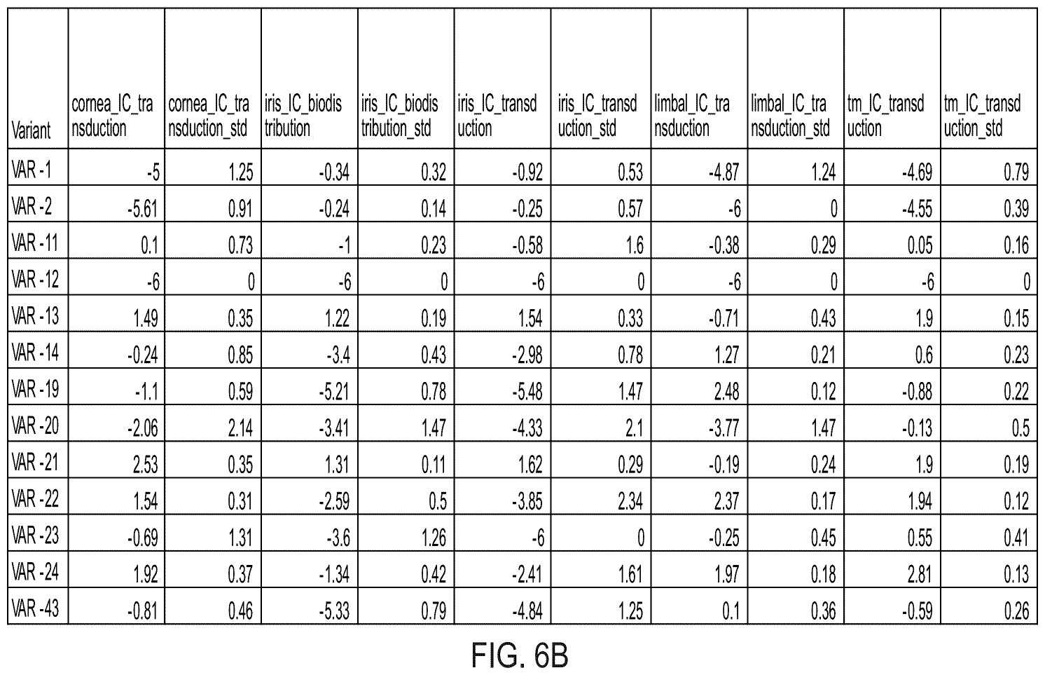

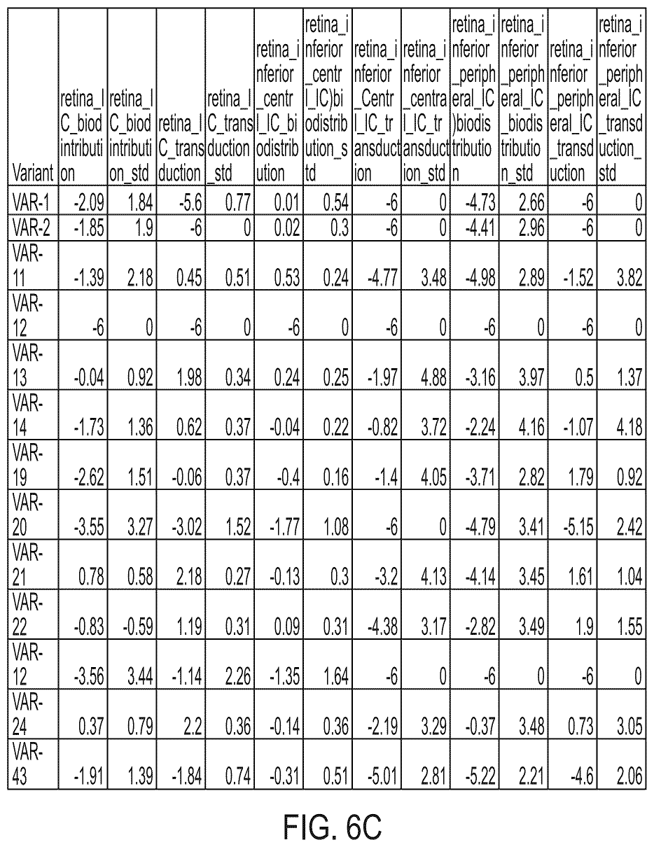

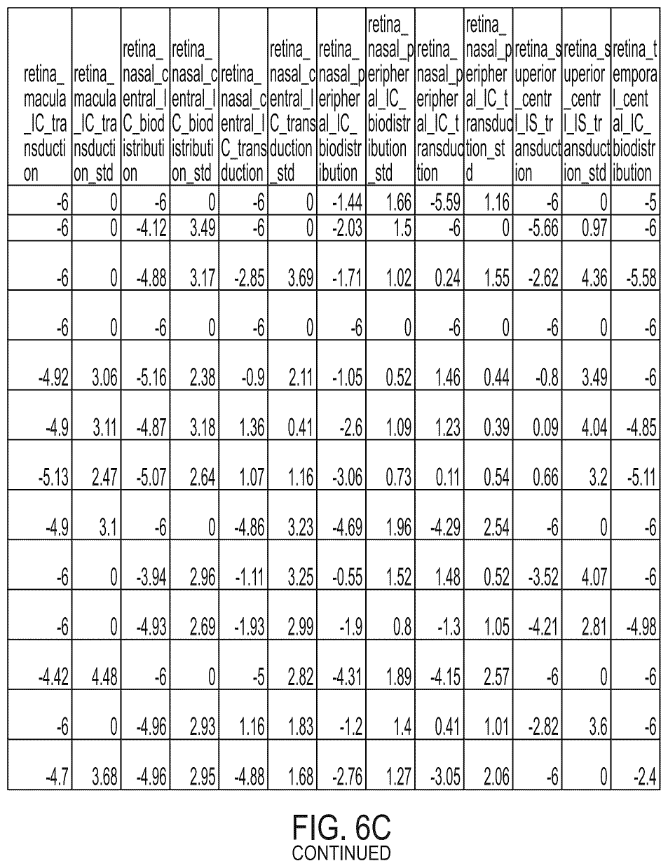

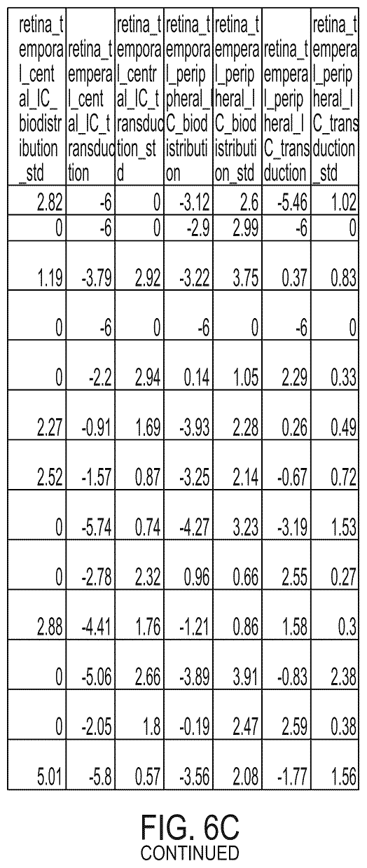

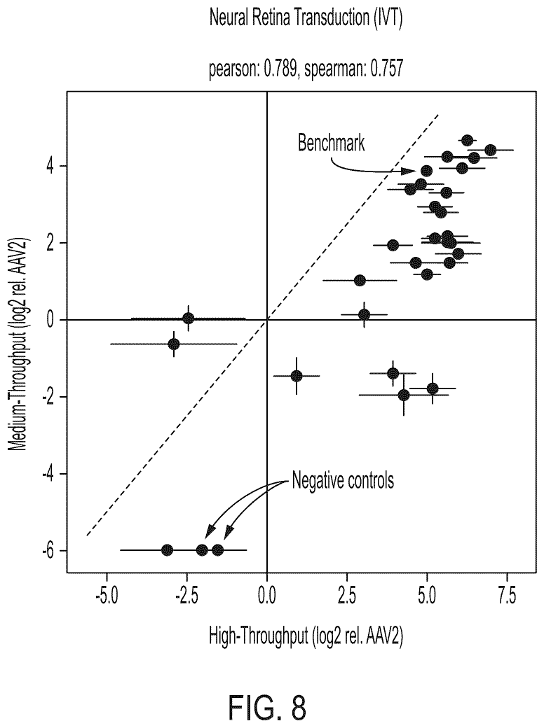

No significant deviations were reported during the in-life period of the study, and no major ocular inflammation was observed. 4 weeks following injection, transduction was measured by bulk barcode-seq as well as snRNA-seq. The data enabled us to observe correlations between Library Experiment 1 measurements and property measurements from this experiment, to compare transduction efficiency between bulk and single-cell measurements, and to determine transduction rates for high-performing variants across major cell types within the eye. In addition to identifying highly promising capsids for improved ocular gene therapy delivery, this high-resolution dataset provides validation of Library Experiment 1 measurement quality and valuable input data for future machine-guided design directed at improving cell transduction, specificity and tissue distribution. Results from bulk tissue for variants included in this medium throughput study described in this Example 2 are shown in FIGS. 5A-5C (IVT administration) and FIGS. 6A-6C (IC administration). All values are log(2) relative to wild-type AAV2. FIG. 8 plots the measured Library Experiment 1 neural retina transduction (x-axis) against the medium throughput measured retina transduction for the same variant from this experiment (y-axis). As shown, both the retinal transduction measurements, as well as the rank ordering amongst all variants tested, was highly correlated across the two studies (Pearson=0.789; Spearman=0.757).

Single-cell RNA sequencing has been previously demonstrated to allow characterization of cell-type specific tropism of barcoded rAAVs (Brown et al., Front. Immunol., 2021). However, obtaining single cell suspension from certain tissue types and/or flash frozen samples from externally-sourced non human primate (NHP) studies can be extremely challenging. We developed an approach that combines single-nuclei RNA sequencing (snRNA-Seq) with targeted amplicon sequencing to reliably detect cell-type specific transduction from up to 50-100 barcoded rAAVs with minimal sequencing depth, with an initial focus on tissues of the eye, and applied these methods to the tissues collected from the experiment described in this example. To implement this approach we have: 1) developed optimized protocols for isolation of high quality single nuclei suspensions from flash frozen NHP retina, 2) used the 10x Genomics Chromium platform to encapsulate these nuclei and generate gene expression libraries for reliable identification of cell types, and 3) leveraged the 10x feature barcode kit to selectively amplify (for sequencing) barcoded viral transcripts that were captured using the 10× CS1 feature designed into the viral genomes. Using this approach we investigated cell-type specific tropism of multiple rAAVs in the NHP (cynomolgus macaque) retina. Our snRNA-seq gene expression analysis identified all the major retina cell types including therapeutically relevant cells such as Rods, Cones and Retinal ganglion cells. Viral transduction events, as assessed from our targeted library sequencing, were detected in almost all clusters and we could successfully quantify differences in transduction rates between rAAVs and benchmarks. Overall, we demonstrate that snRNA sequencing can be used to both effectively determine cell-type specific tropism of barcoded rAAVs, and quantitate relative transduction between multiple rAAVs in a single experiment. These developments open up opportunities for further designing and validating rAAVs capable of cell-type specific targeting for gene therapy. Additionally, this approach enables a medium throughput identification of rAAVs with desired properties for further study.

Details of the single nuclear experimental workflow that was applied to the medium throughput study described in this example are below:

### Materials

EZ lysis buffer+0.2 U/μl of murine RNAse inhibitor
1×PBS+9% BSA+0.2 U/μl murine RNAse inhibitor
1×PBS+5% BSA+0.2 U/μl of murine RNAse inhibitor/Wash
1×PBS+2% BSA+0.2 U/μl of murine RNAse inhibitor
Two 15 ml tubes pre-coated with 2% BSA+lx PBS+0.2 U/μl RNAse inhibitor
Six 1.5 ml protein lobind tubes pre-coated with 2% BSA+lx PBS+0.2 U/μl RNAse inhibitor
One 2 ml dounce homogenizer+Pestle A and Pestle B
2×0.4 μm filters
2×0.7 μm filters
1 ml wide bore pipette tips
Hemocytometer
4-6 0.5 ml protein lobind tube with 15 μl of 1×PBS+5% BSA+
   Single Nuclei Dissociation from Retina and Trabecular Meshwork

Mincing: For the retina, the tissue sample was placed in a tube on ice and 100 μl of EZ lysis buffer+RNAse inhibitor was added. The tissue was minced with a pair of microscissors for about 1 min while holding the tube on ice. 50 μl of the minced sample was transferred to a 2 ml dounce homogenizer. 1-2 ml of Trizol was added to the rest of the sample for paired bulk RNA extraction and sequencing.

For the trabecular meshwork, the tissue was transferred to a new 1.5 ml tube and 50 μl of EZ lysis buffer+RNAse inhibitor was added. The tissue was minced with a pair of microscissors for about 1 min while holding the tube on ice. The minced tissue was transferred to a 2 ml dounce homogenizer.

Dounce homogenization: More EZ lysis buffer+RNAse inhibitor was added to the dounce homogenizer with sample to make up the volume to 2 ml. A loose fitting pestle (Pestle A) was used to dounce the sample with 10 steady strokes (about 1 stroke per second). The sample was allowed to stand on ice for 20 seconds and then was dounced with a tight fitting pestle (Pestle B) with 5 steady strokes. The sample was again allowed to stand on ice for 20 seconds and dounced for another 5 strokes with Pestle B.

Filtration and clean up: Post douce the sample (2 ml) was immediately transferred to a 15 ml falcon tube containing 2 ml of 9.5%+1×PBS+RNAse Inhibitor. The sample was mixed and first filtered through a 70 micron filter and then through a 40 micron filter. A small (5 μl) aliquot of the filtered sample was diluted 1:4 in 1×PBS+2% BSA+propidium iodide (PI) for counting.

The sample (about 4 ml now) was split equally into four 1.5 ml protein lobind centrifuge tubes precoated with 2% BSA+1×PBS+RNAseInhibitor. The samples were centrifuged at 200 RCF for 5 mins at 4 C. The supernatant was discarded and the pellet was resuspended in 5% BSA+1×PBS+RNAse-Inhibitor+PI.

The nuclei were then sorted on a WOLF sorter. We gated for intact nuclei that were positively stained for PI. We also discarded any doublets by gating with the area under the curse for PI as a proxy for doublets. The FACS cleaned nuclei were centrifuged at 200 RFC for 5 mins at 4 C. The pellet was resuspended in 2% BSA+1×PBS+RNAse-inhibitor and counted. Final nuclei concentration was adjusted as needed for 10× encapsulation.

10× Encapsulation and library preparation: We then used the 10× chromium platform for single cell encapsulation as per the manufacturer's standard instructions. Reverse transcription was performed as per 10× protocols. cDNA amplification was performed using the 10× feature barcode cDNA amplification kit with a Dyno viral transcript specific forward primer spiked in.

Post cDNA amplification a portion of the cDNA library was used to generate gene expression library as per 10× standard protocol. A small portion of the same cDNA library was used to generate targeted libraries by PCR amplifying the Dyno barcode region. Primers binding to either the Nextera Handle or the TruSeq Handle, in combination with a viral transcript specific primer, were used for the targeted amplification. When amplifying using the TruSeq-Handle primer, a gel extraction step was performed right after the targeted amplification to select out the product of interest from the larger linearly amplified background. Once the targeted amplification product was purified we performed pre-indexing and indexing PCRs and sequenced the libraries using an illumina Next Seq sequencer.

### Results

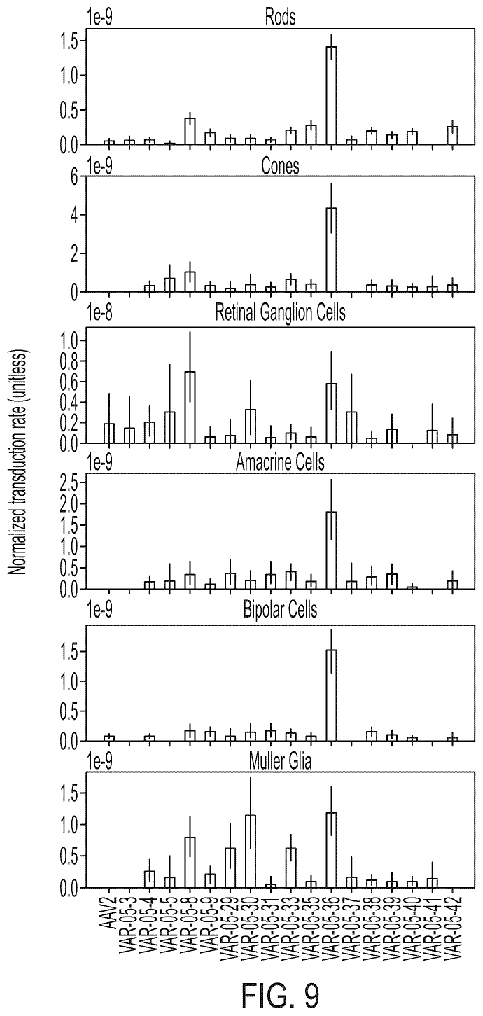

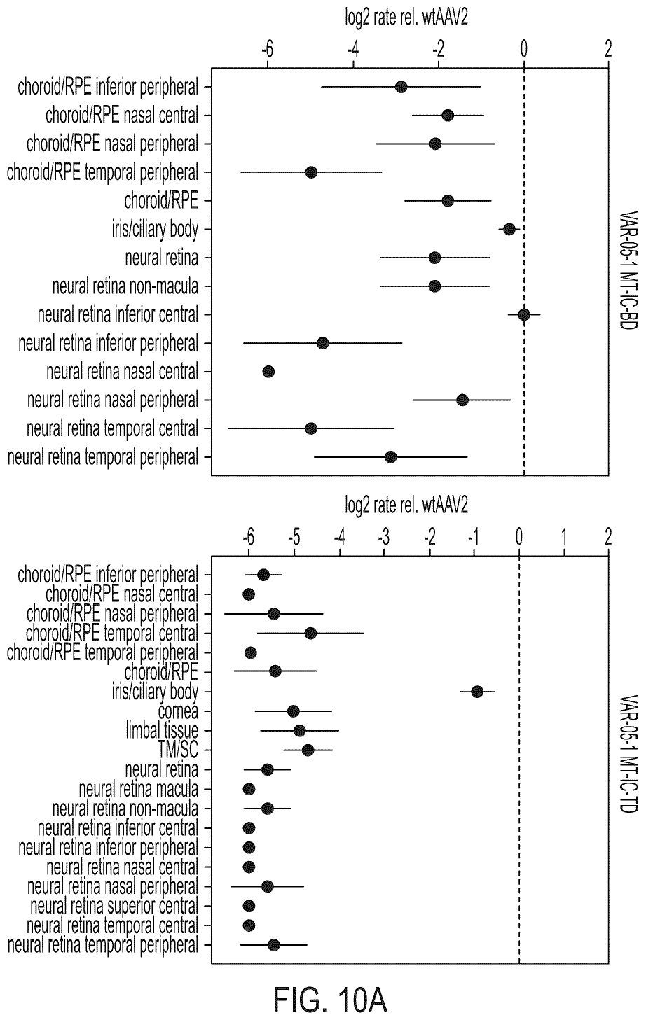

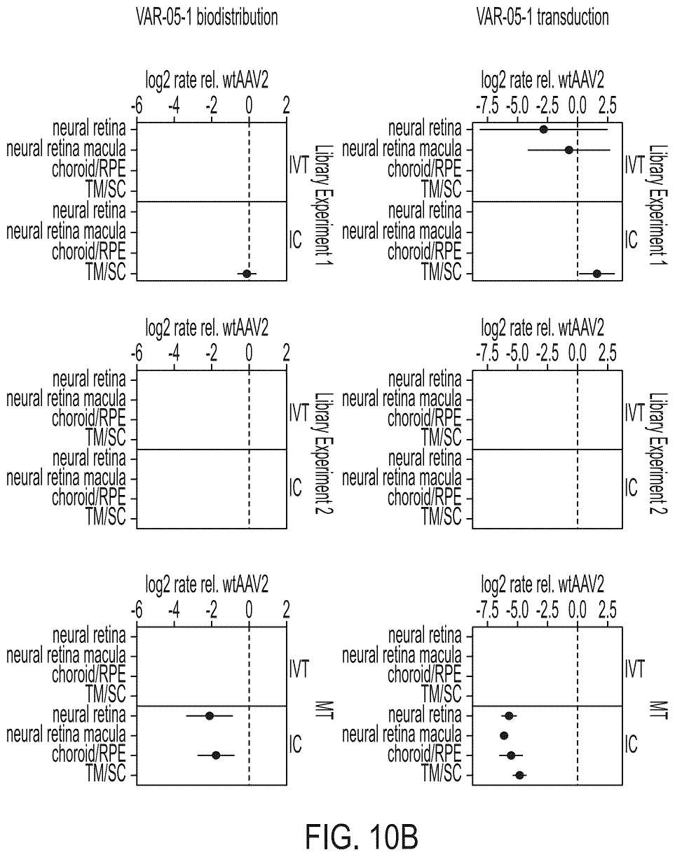

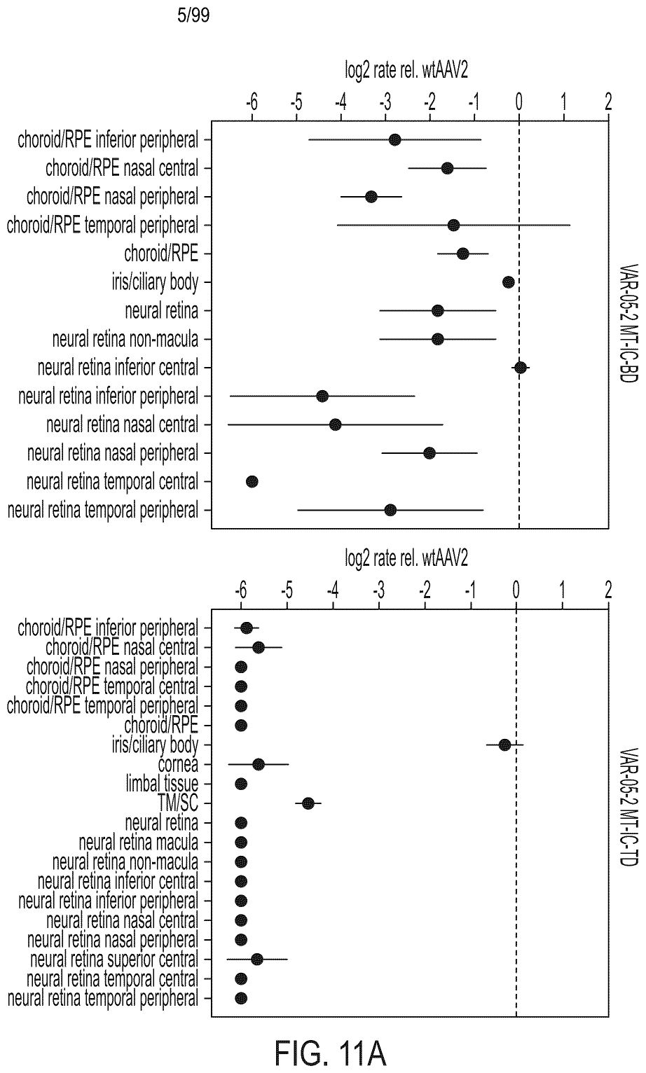

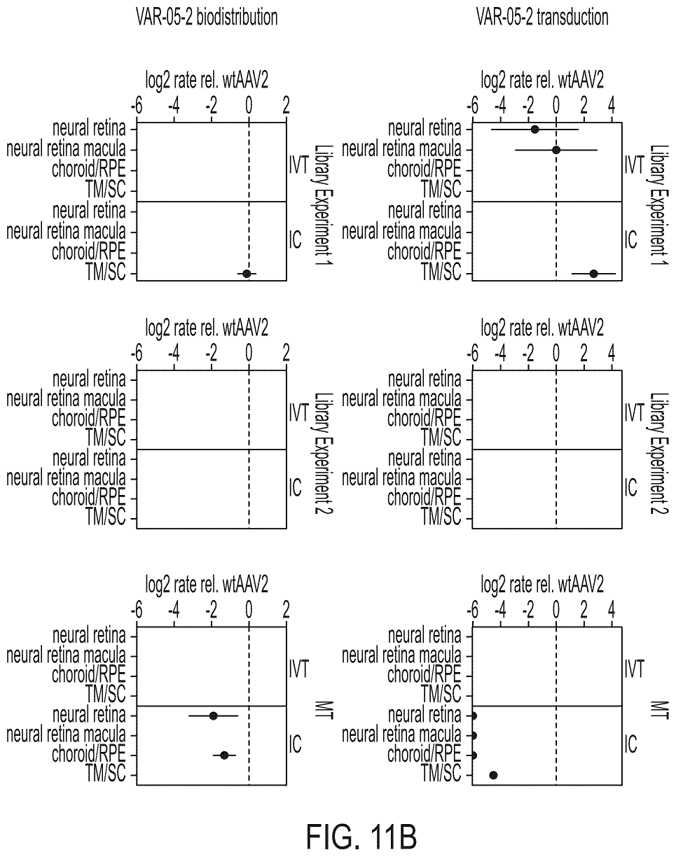

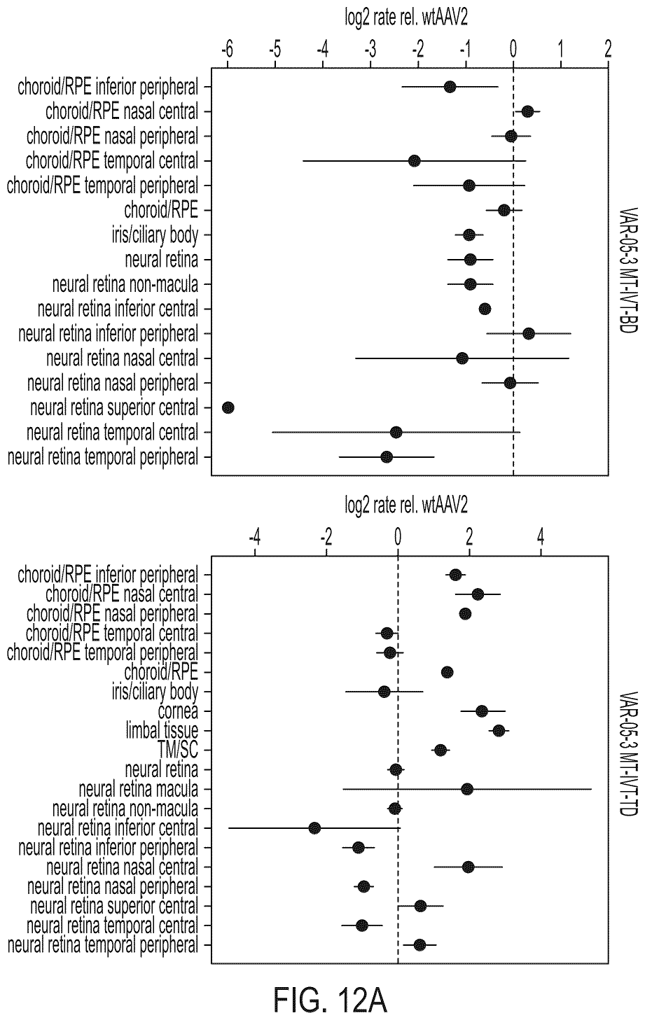

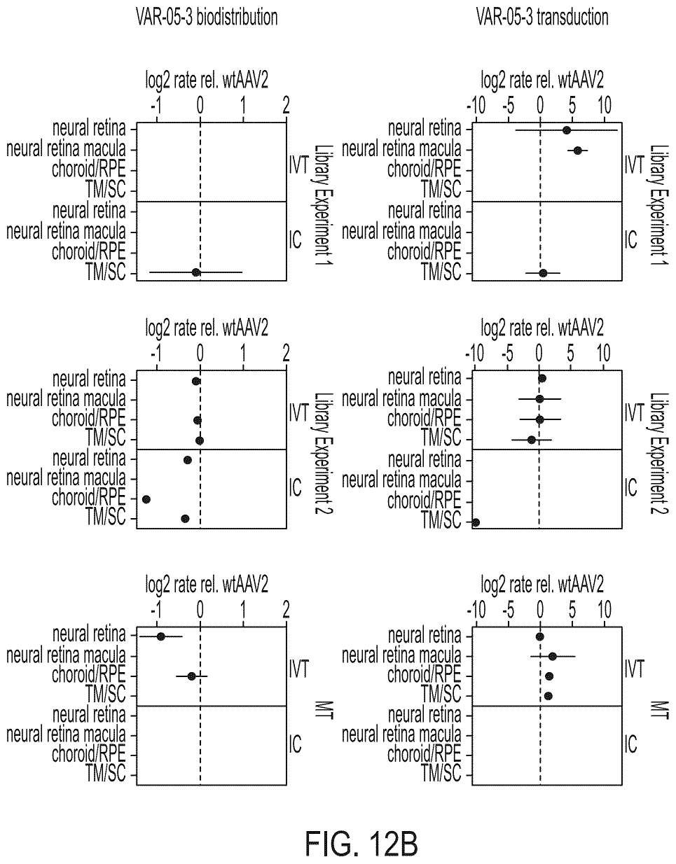

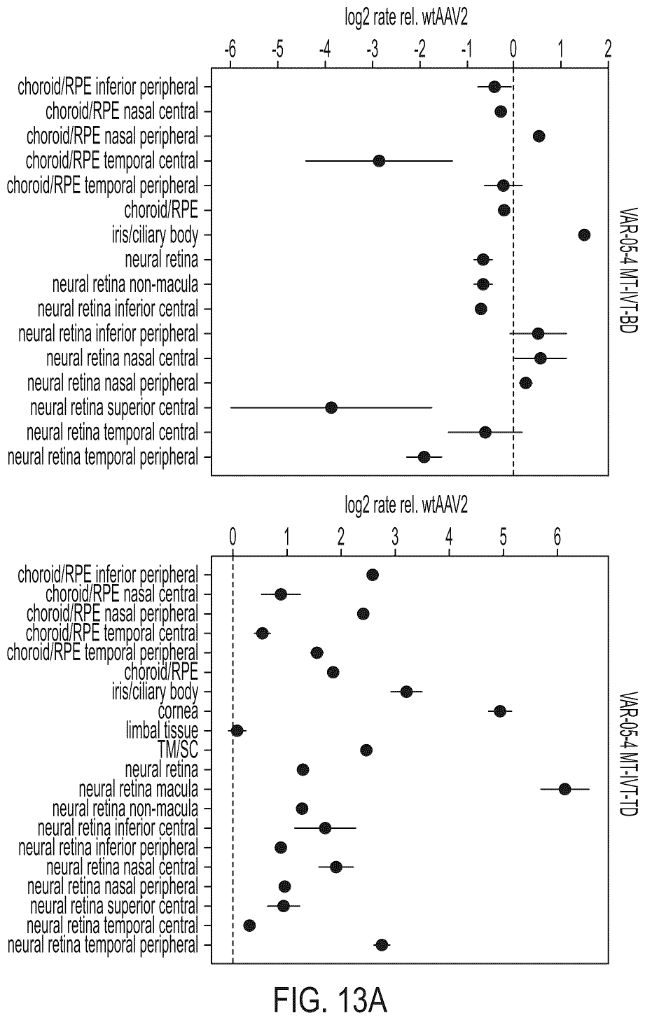

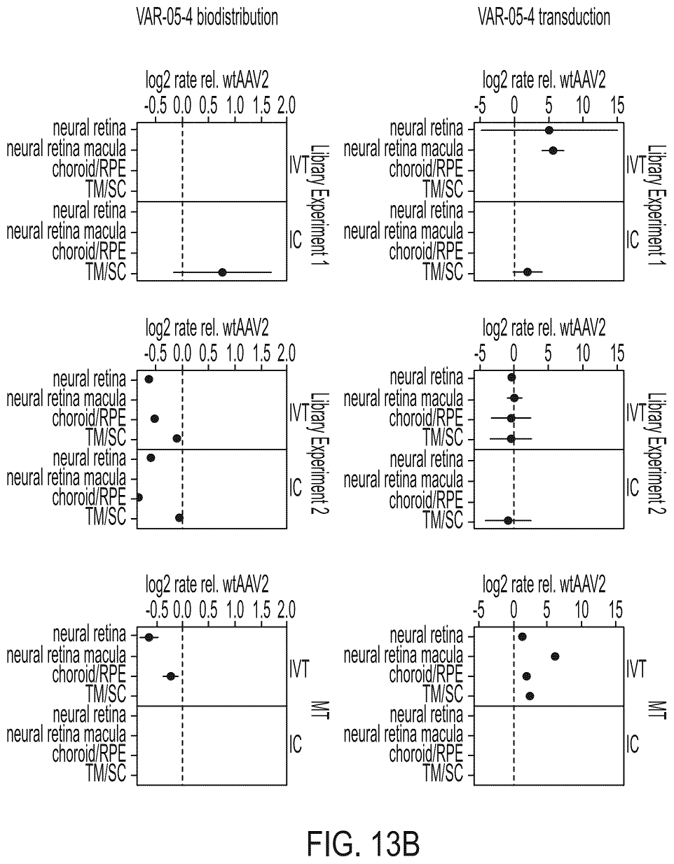

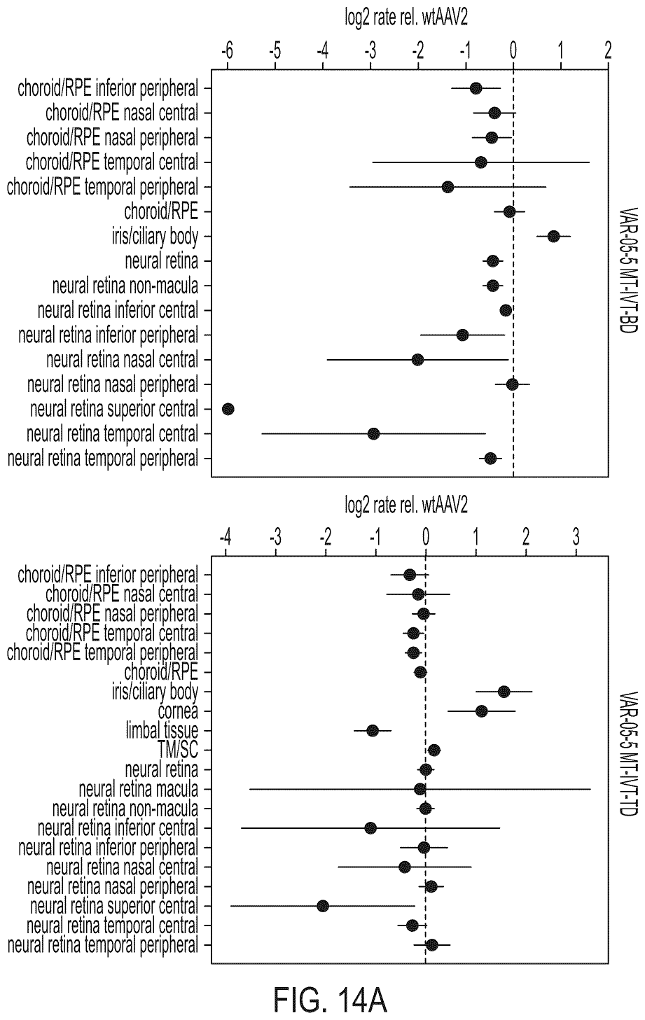

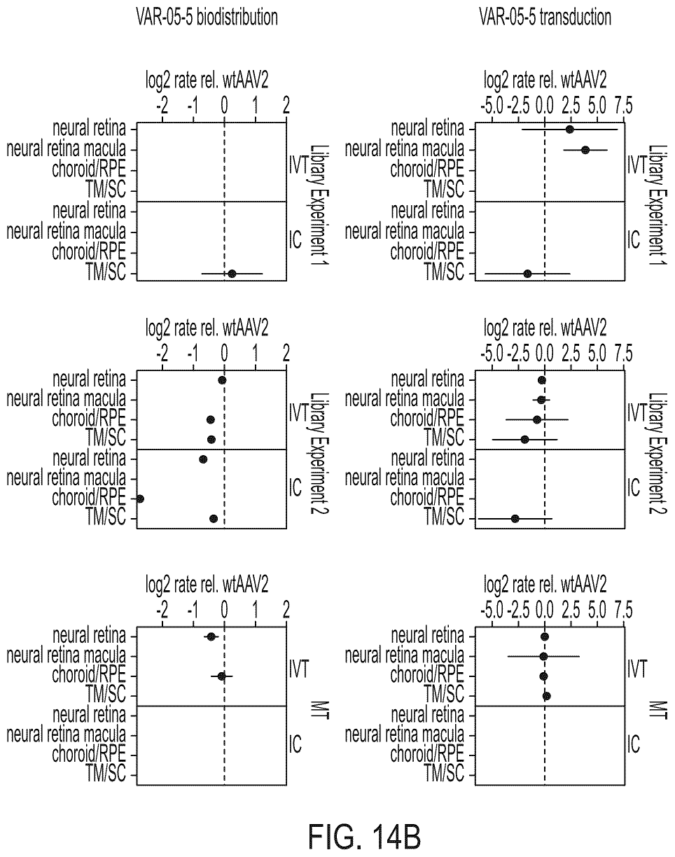

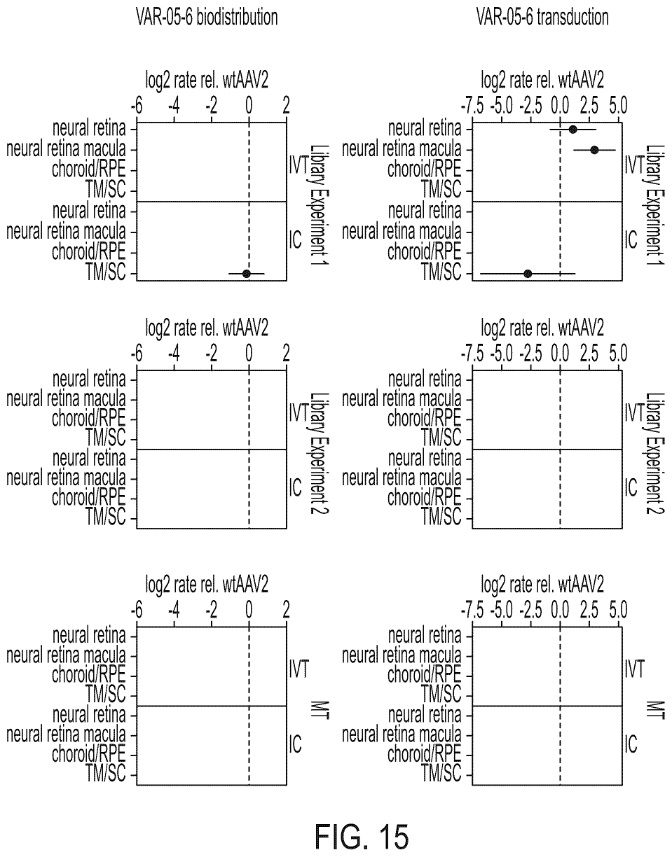

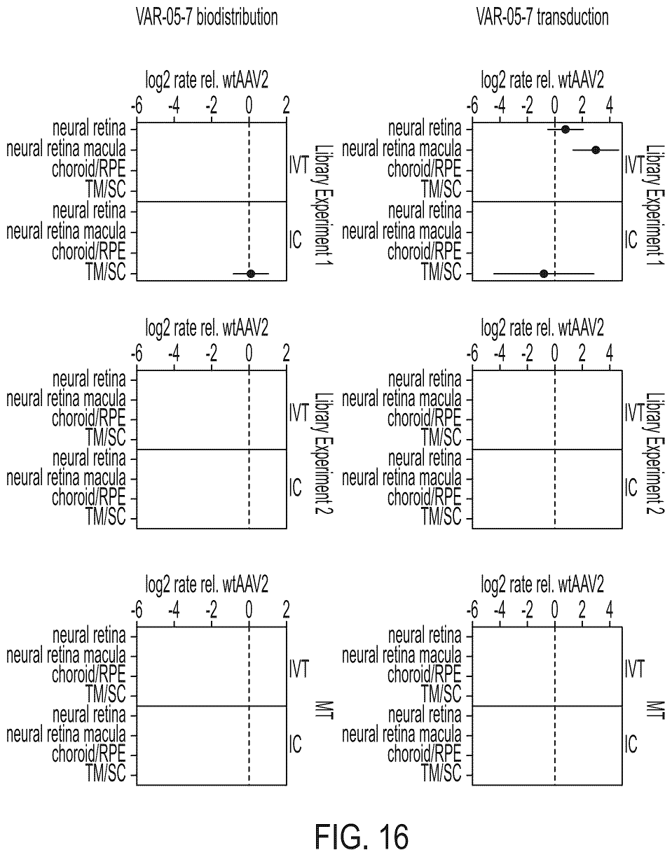

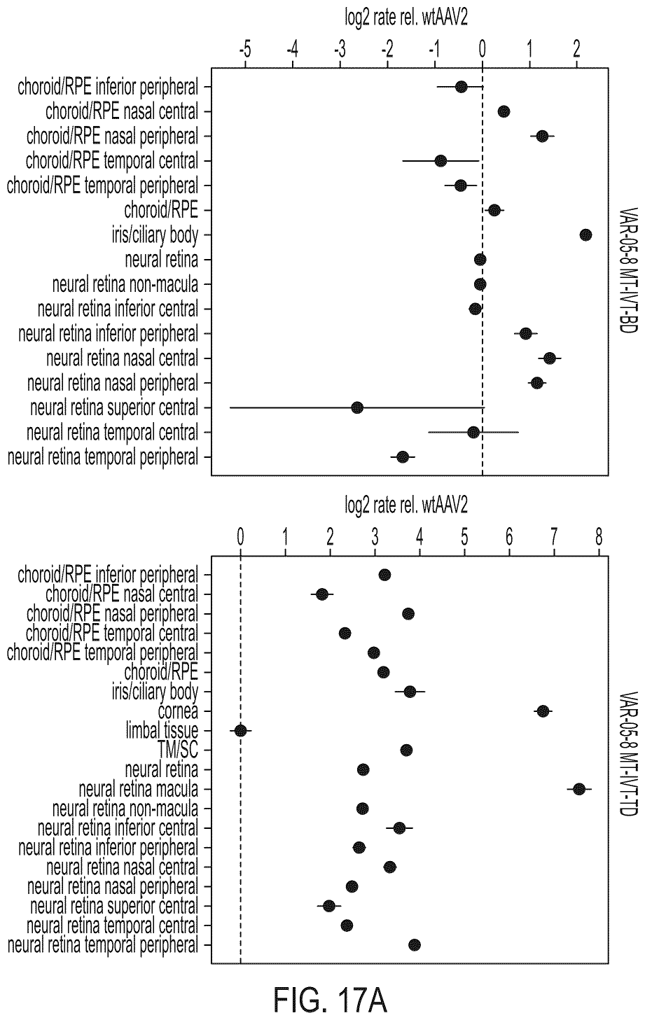

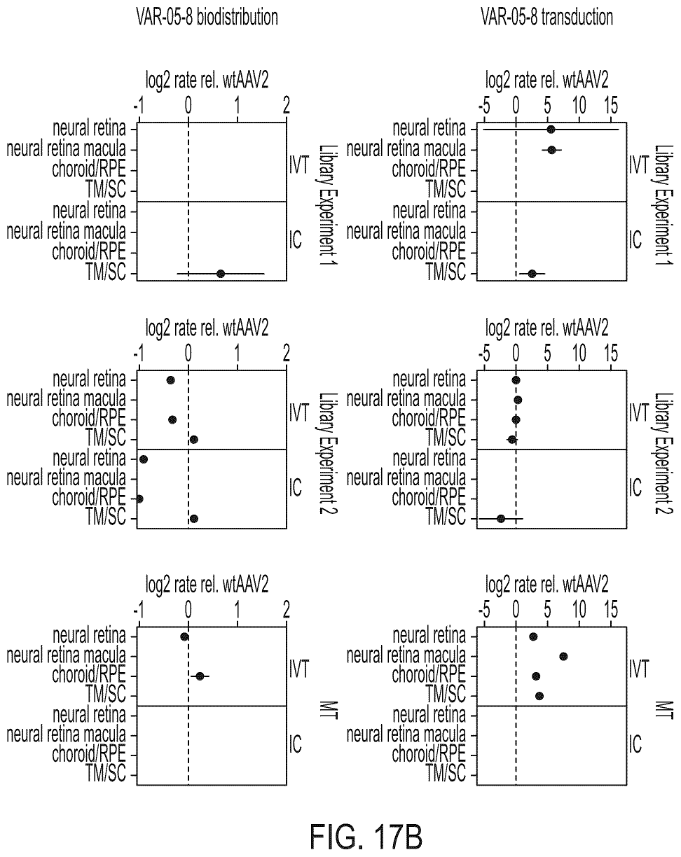

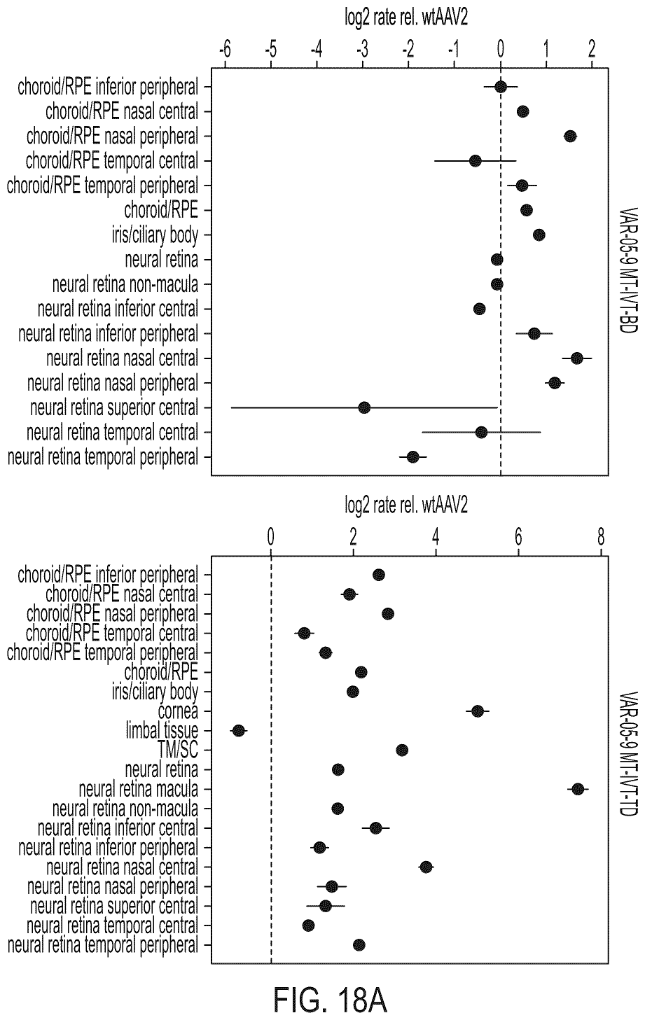

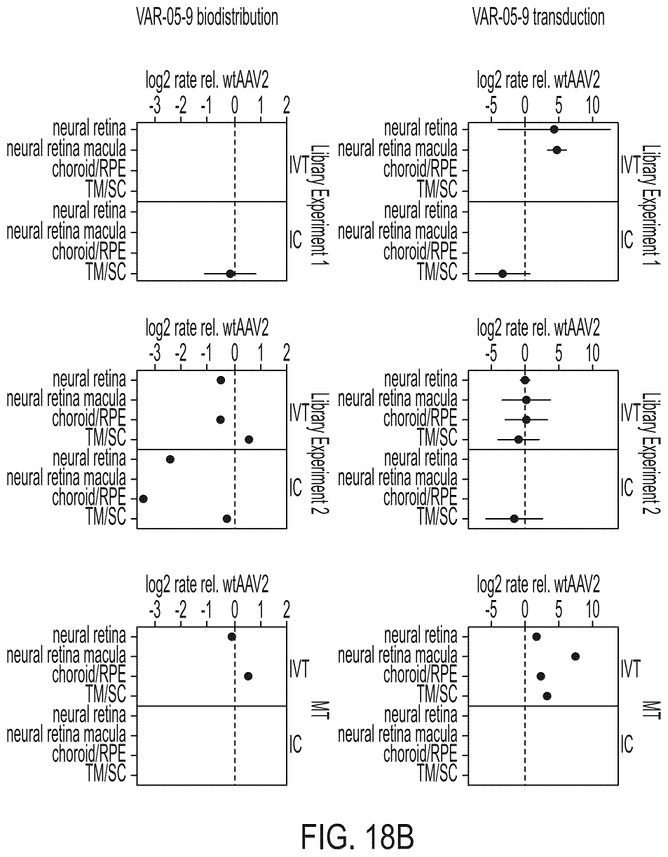

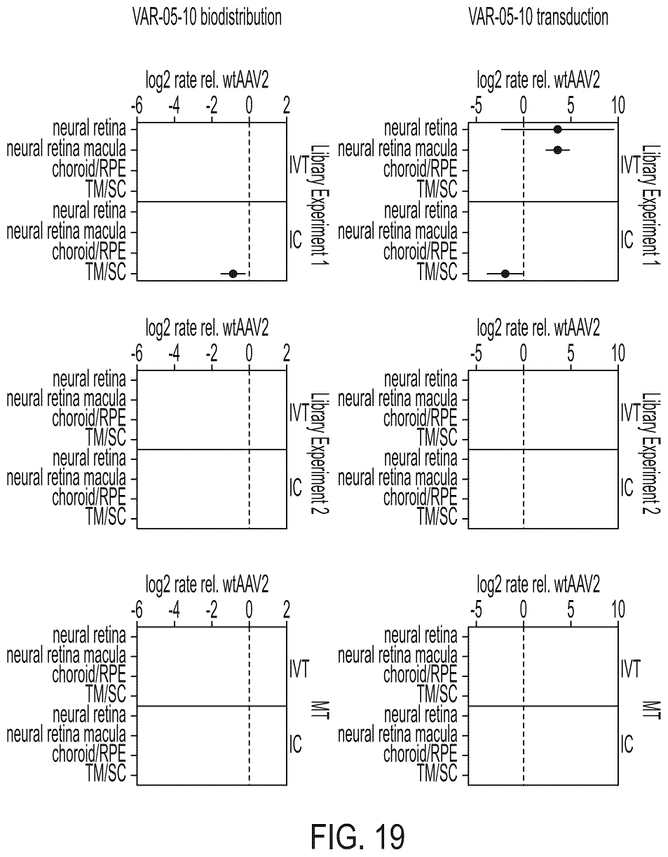

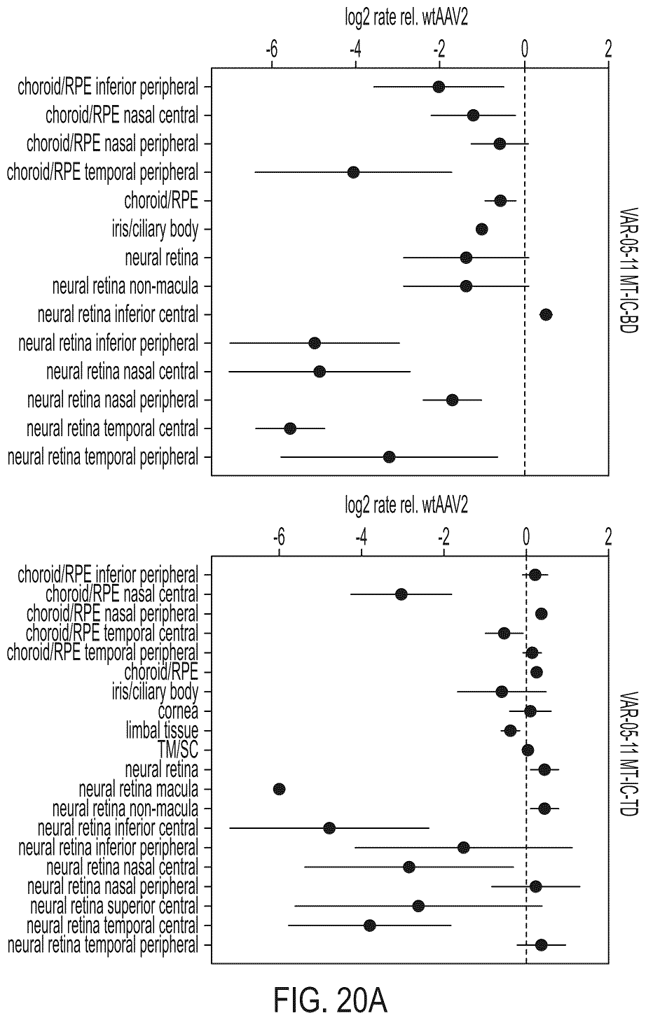

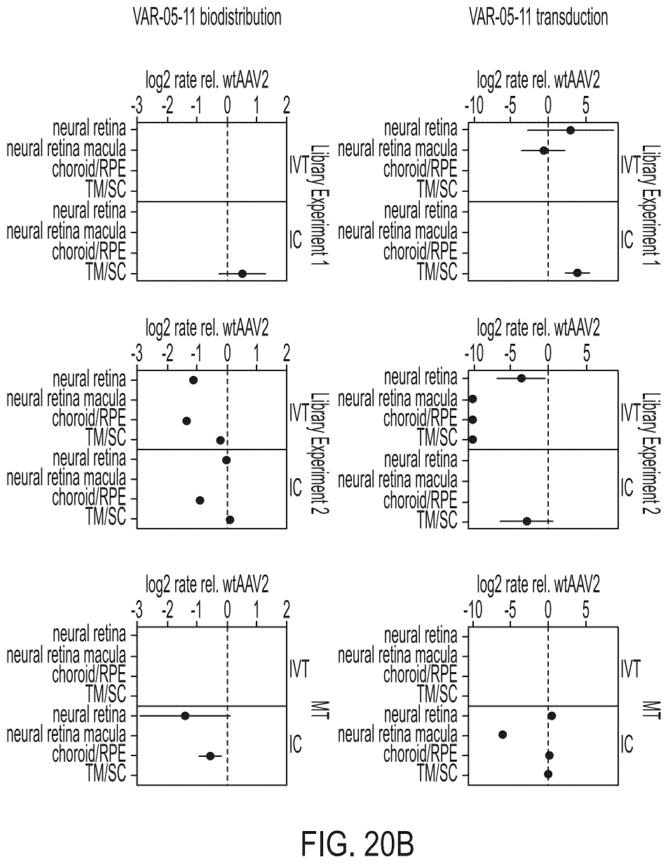

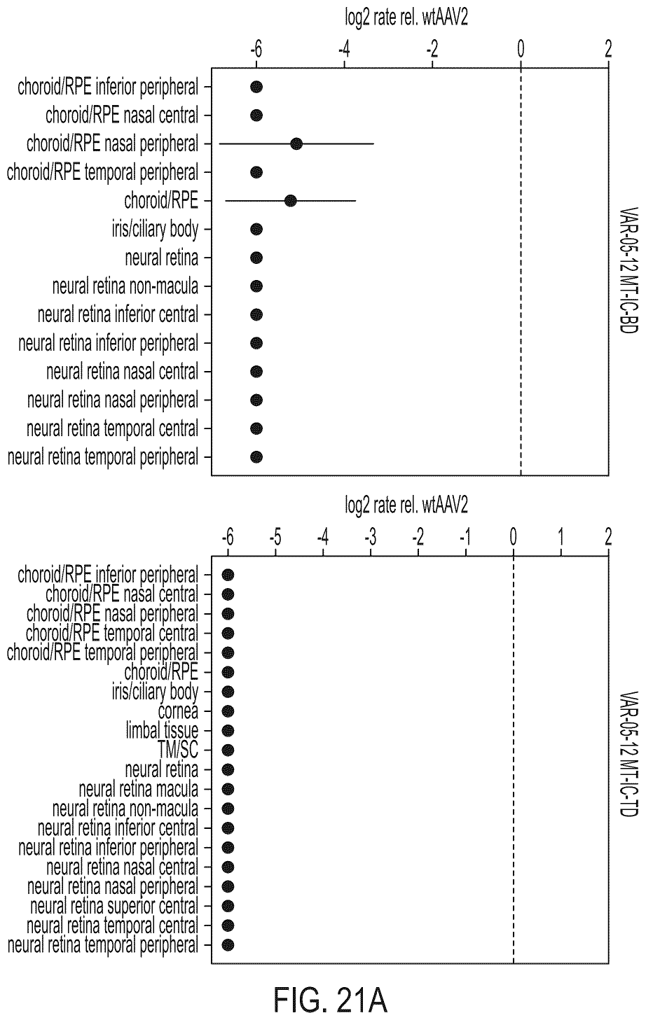

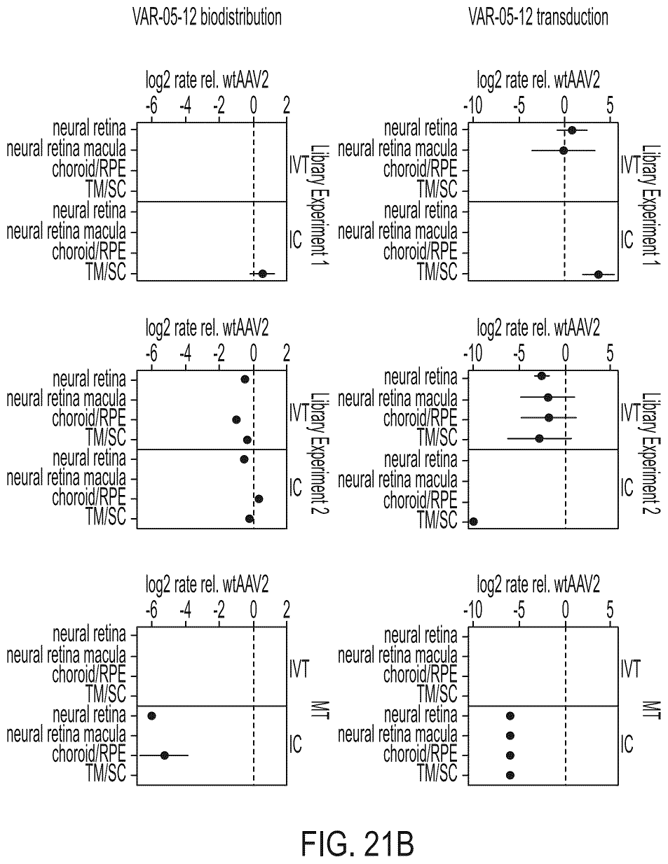

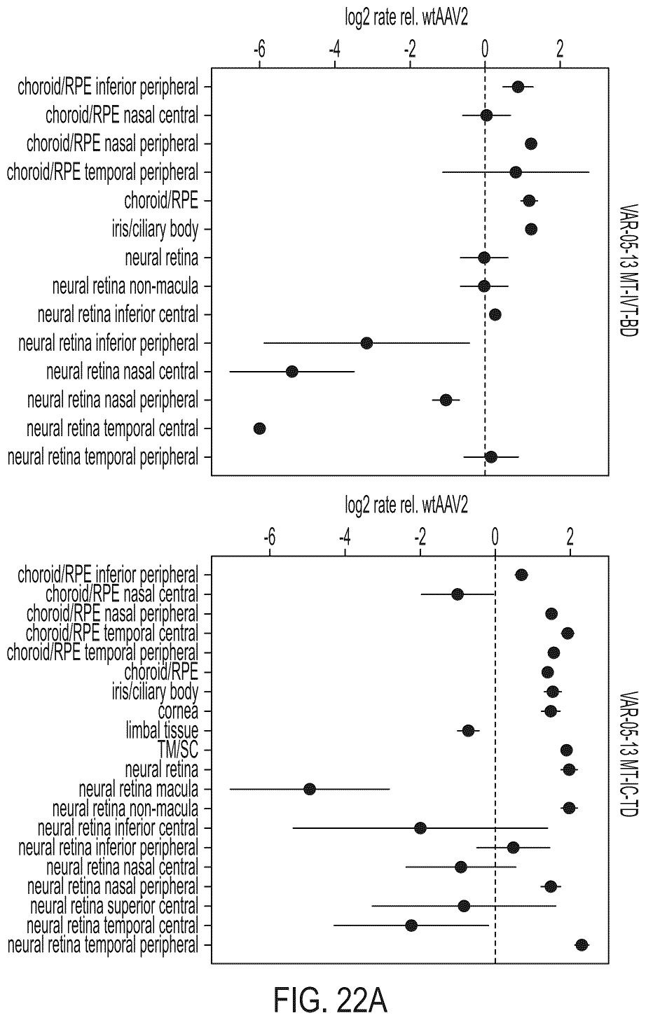

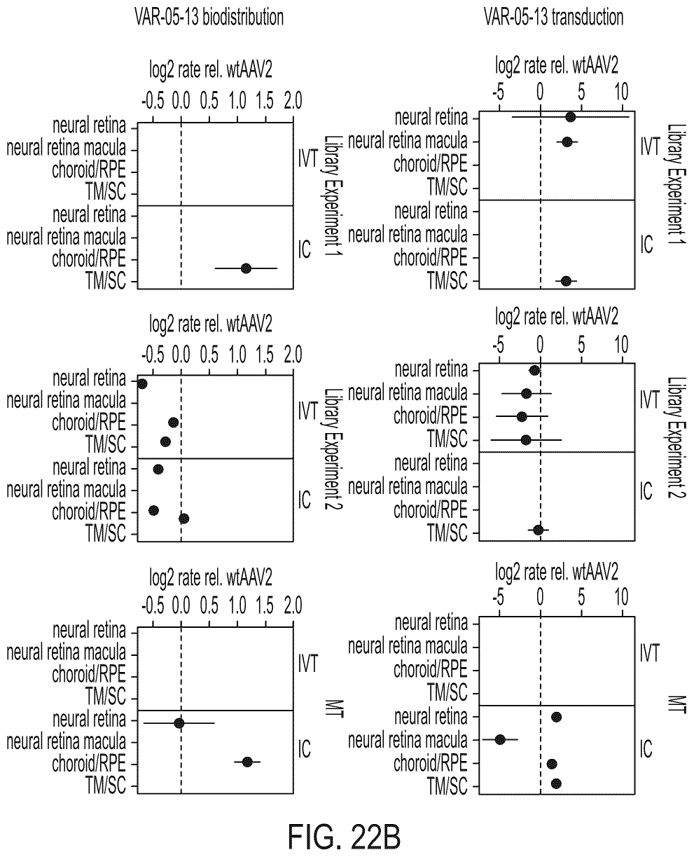

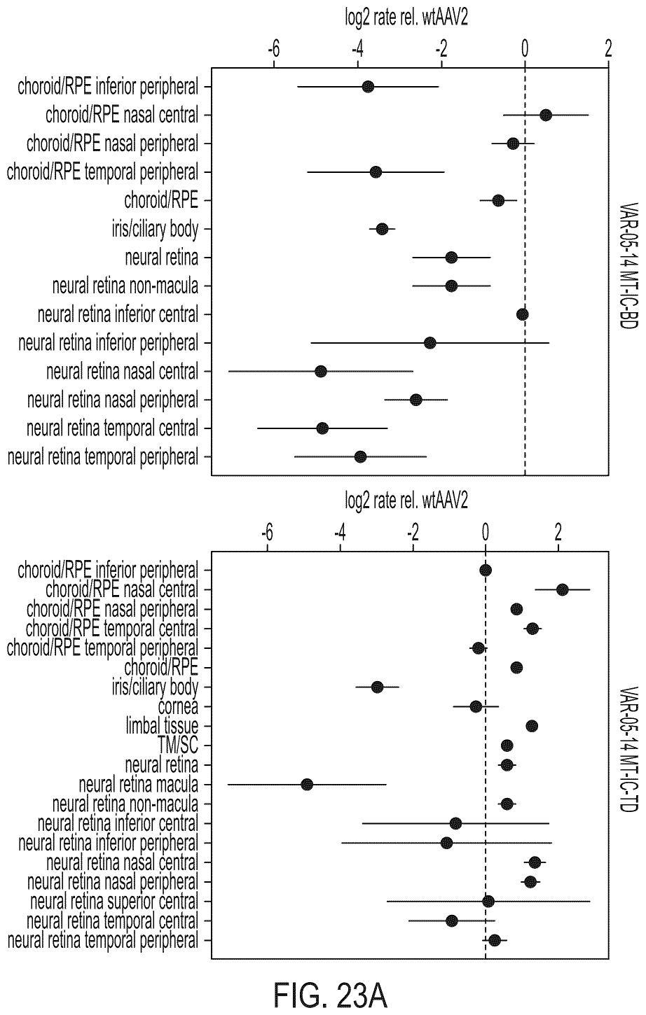

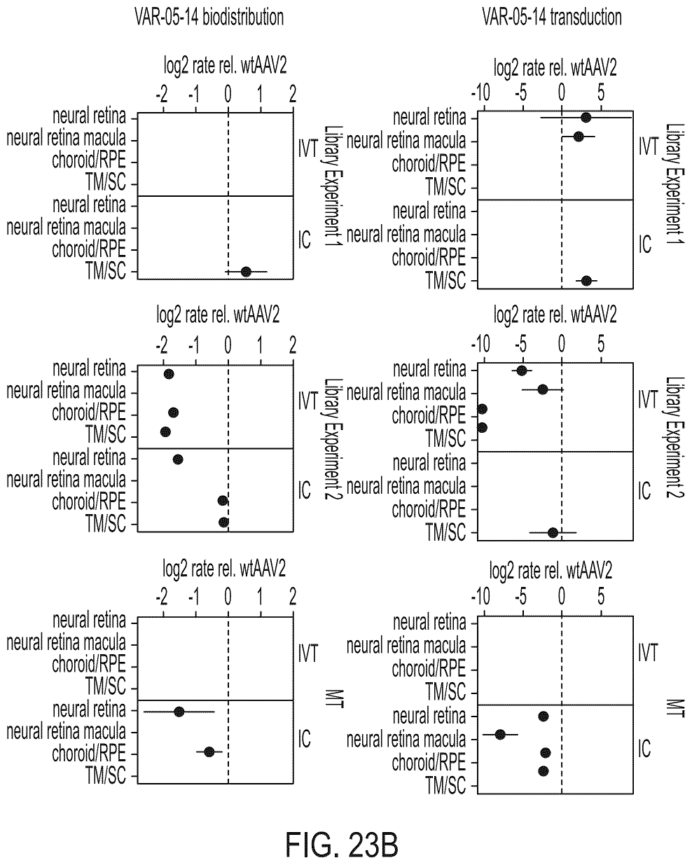

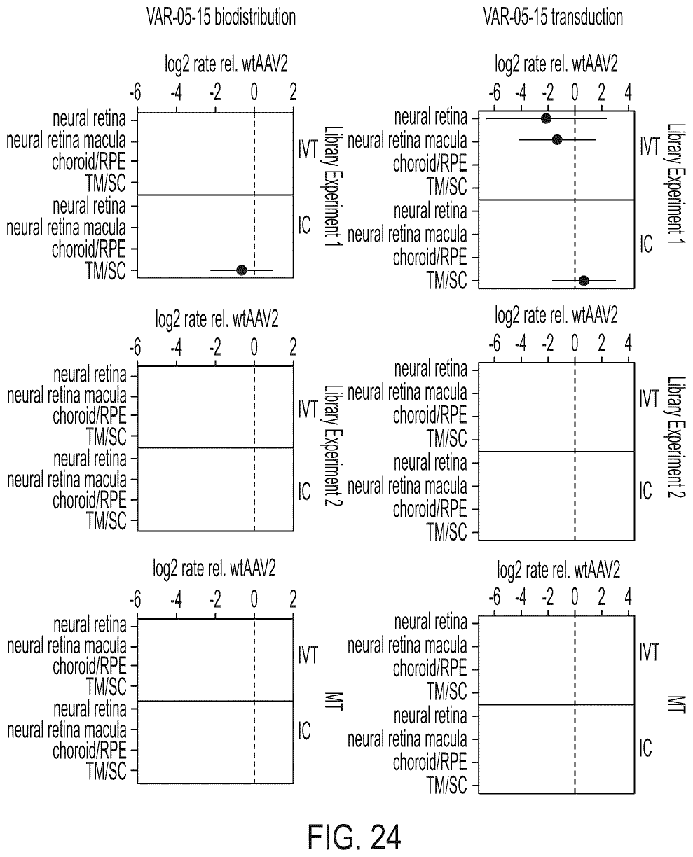

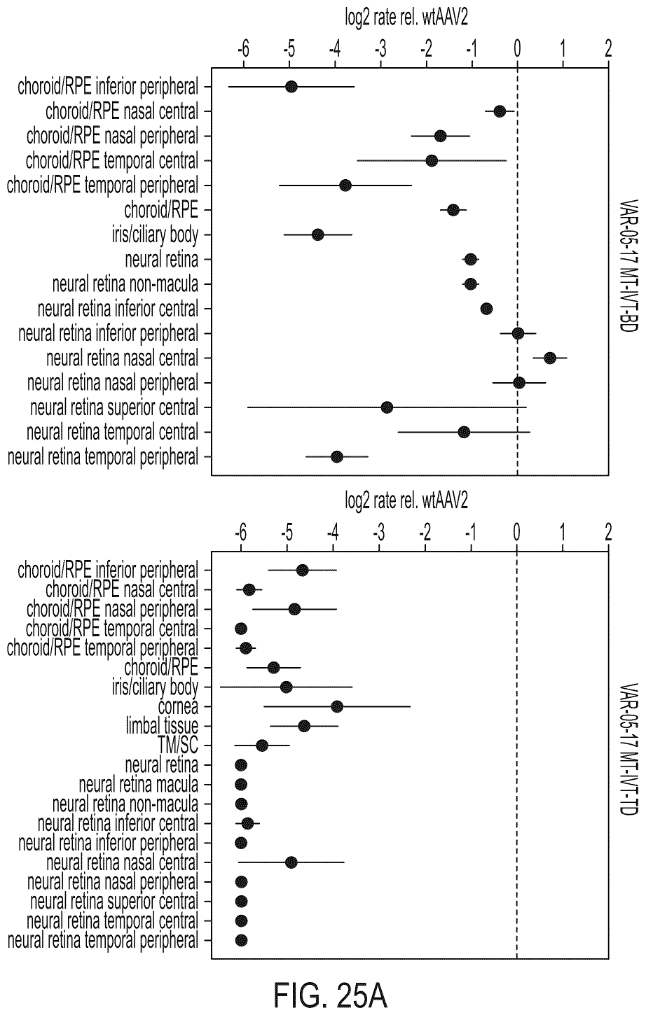

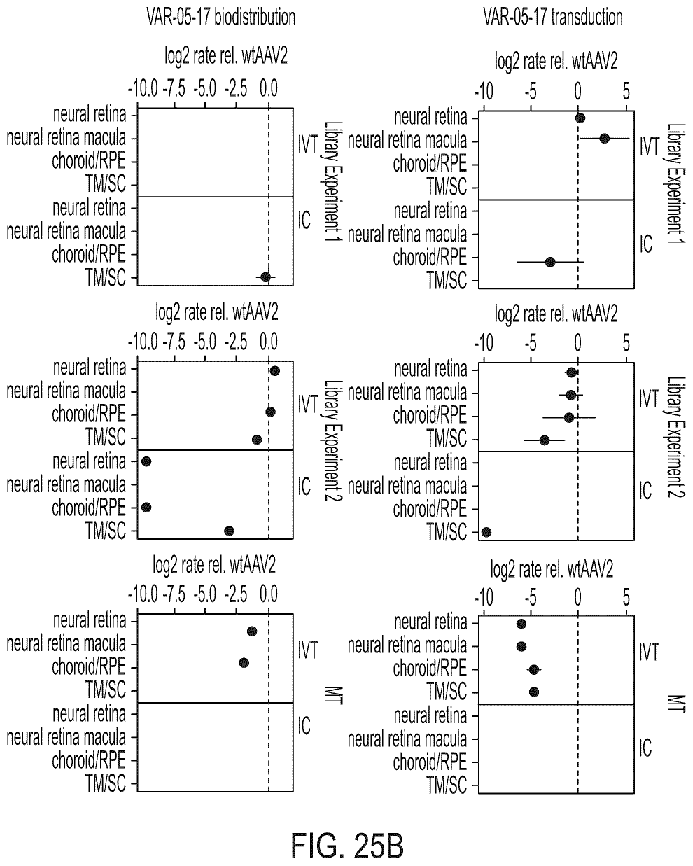

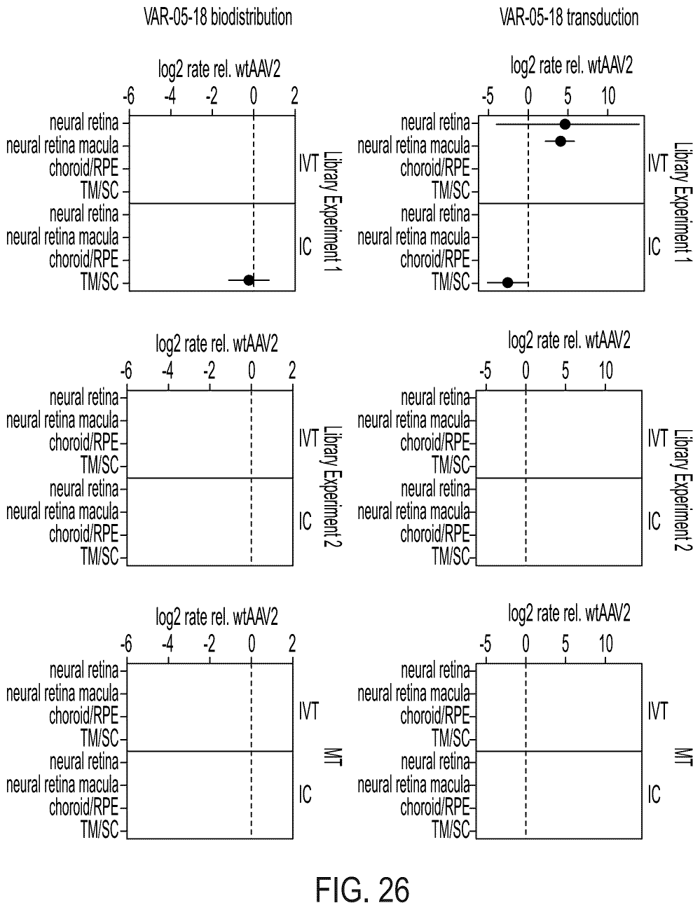

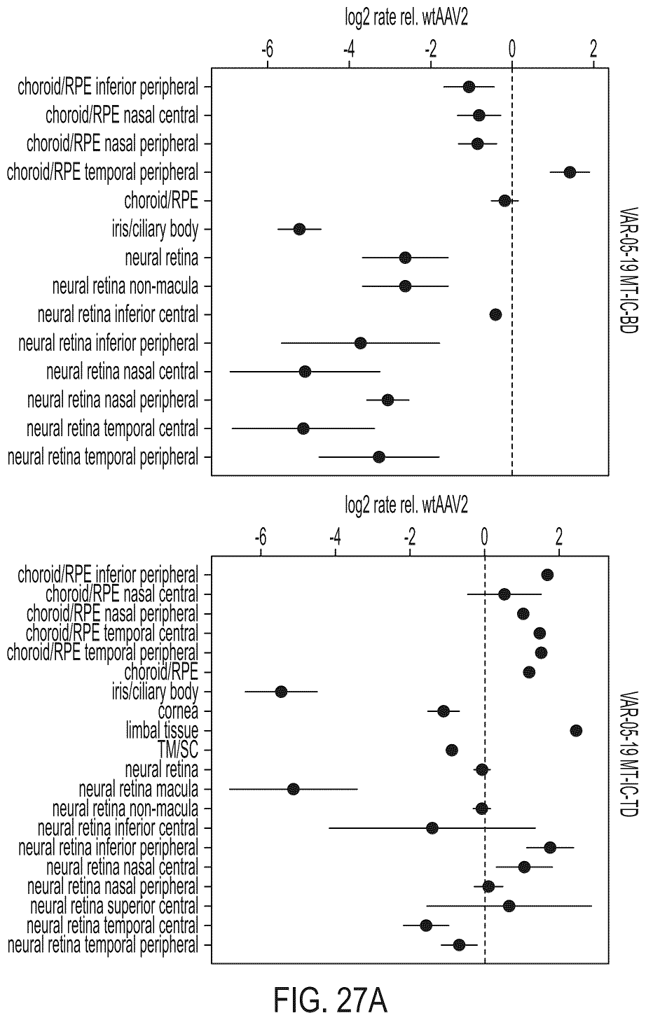

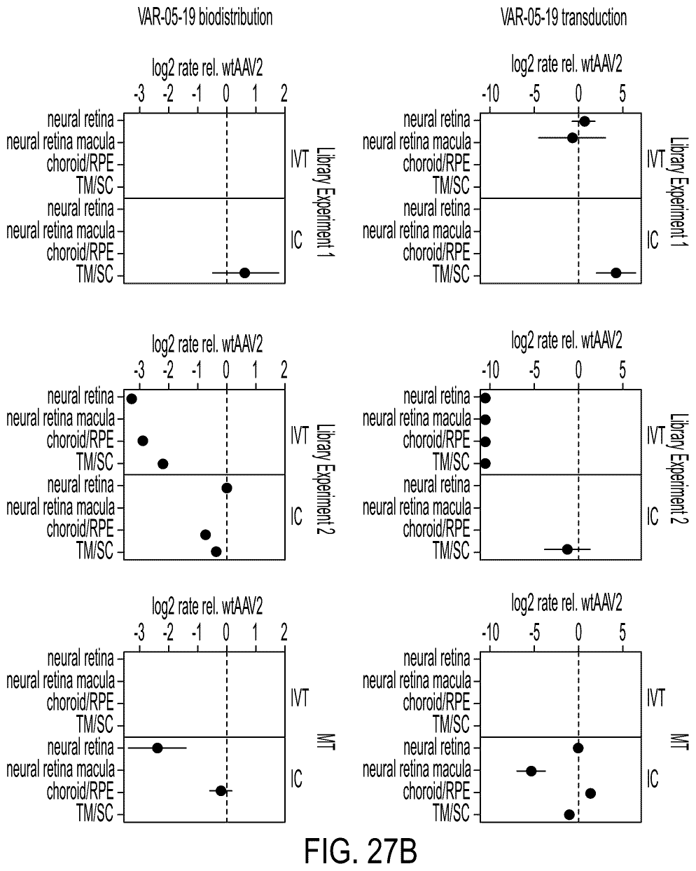

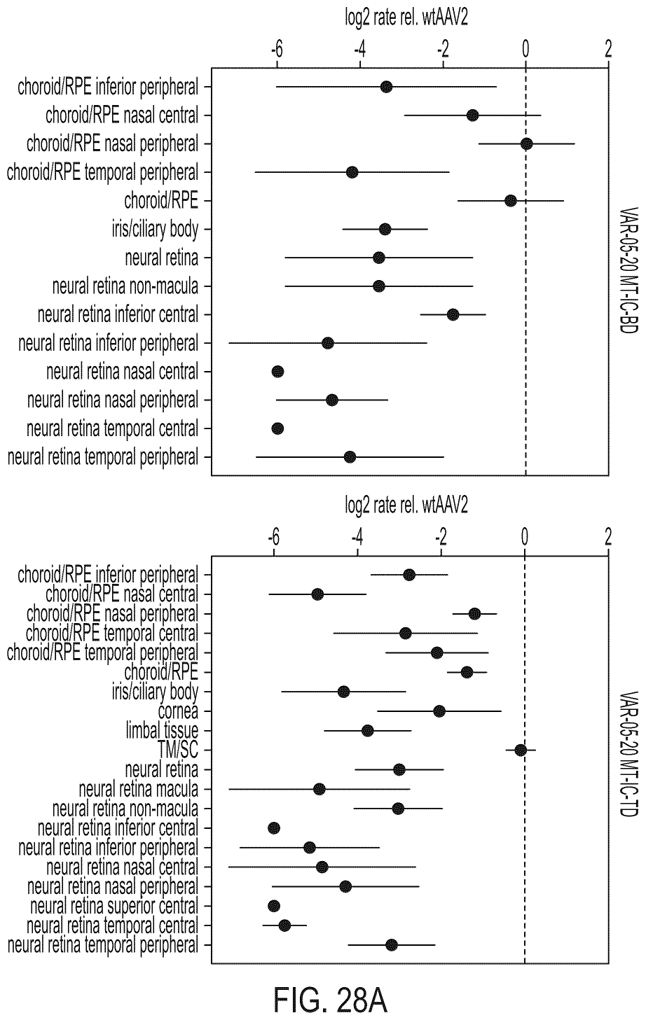

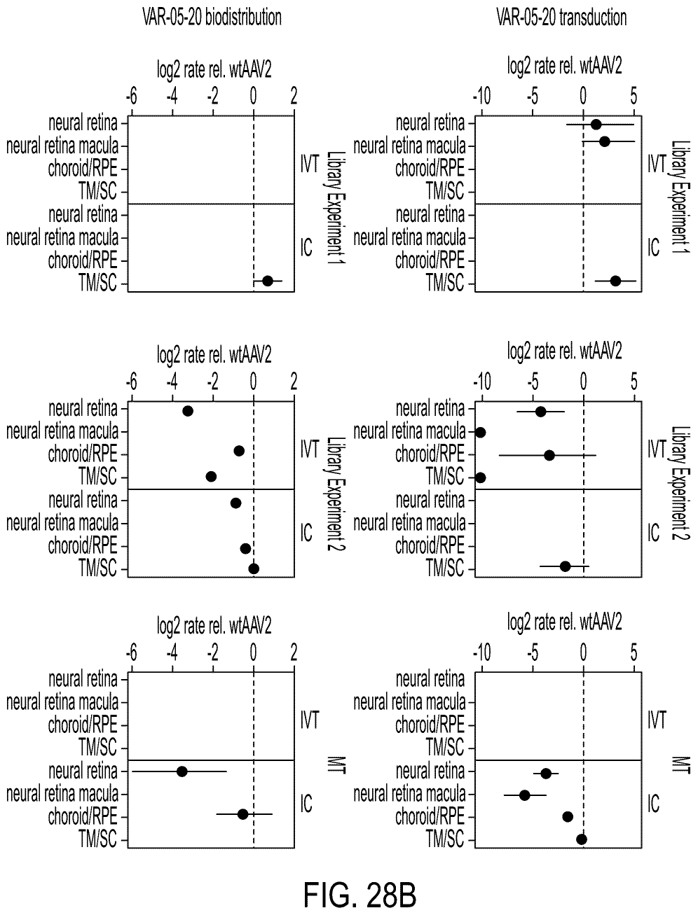

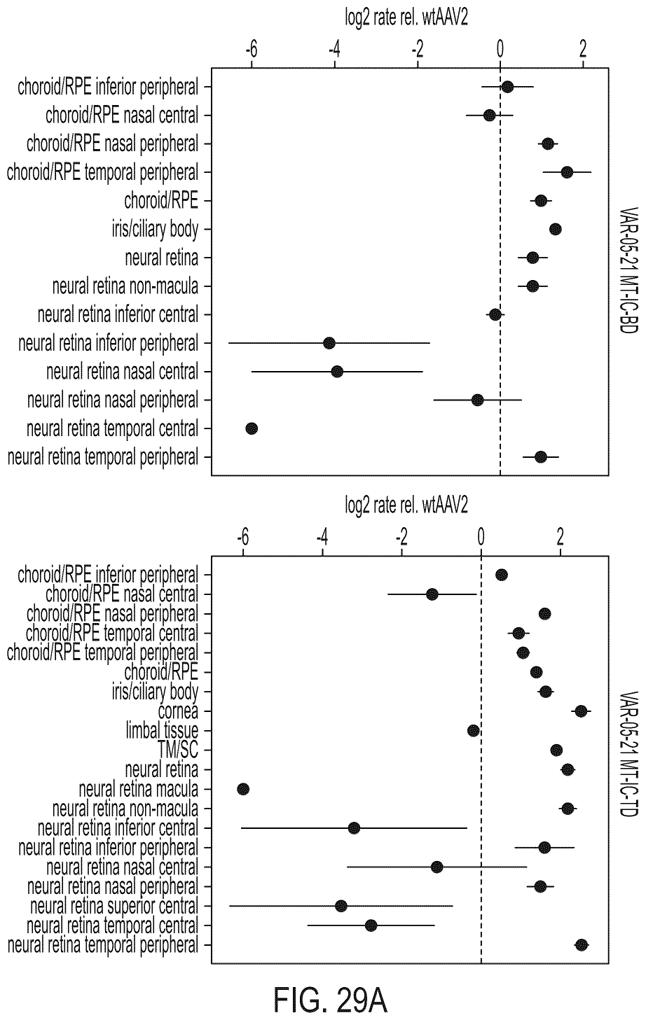

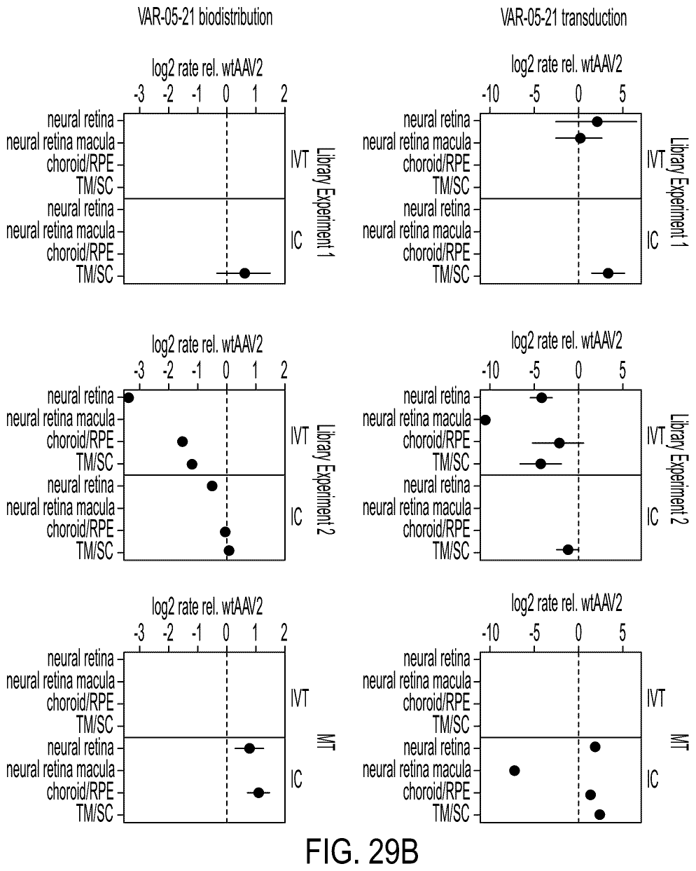

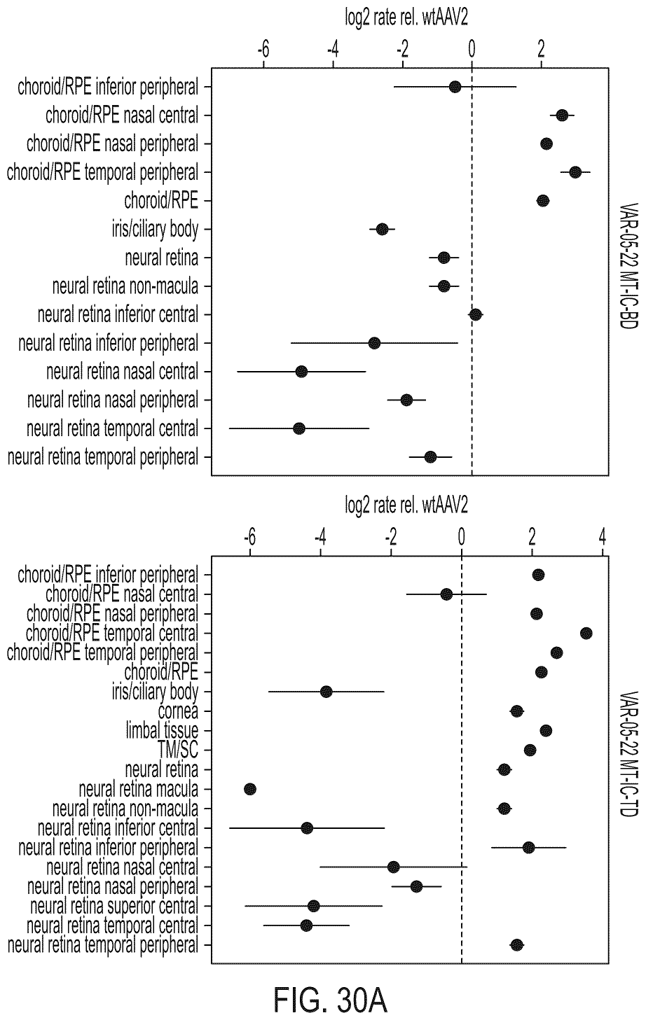

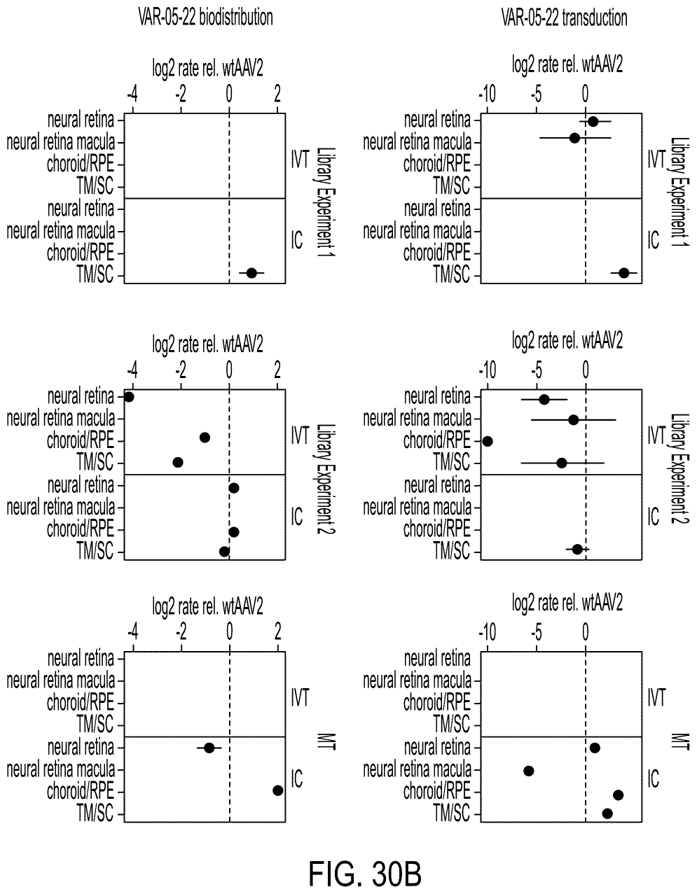

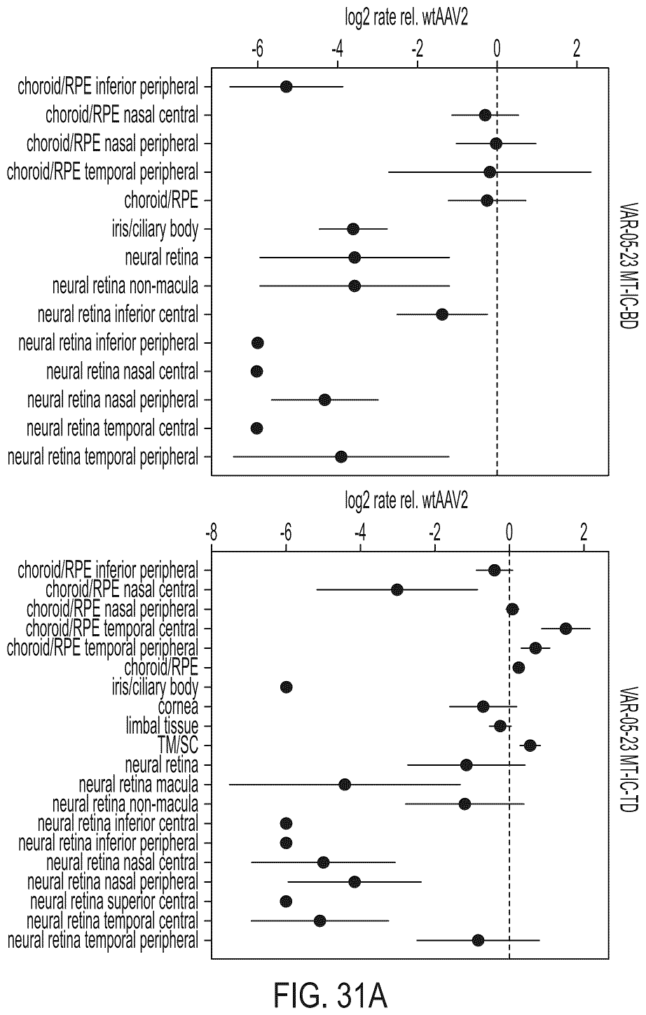

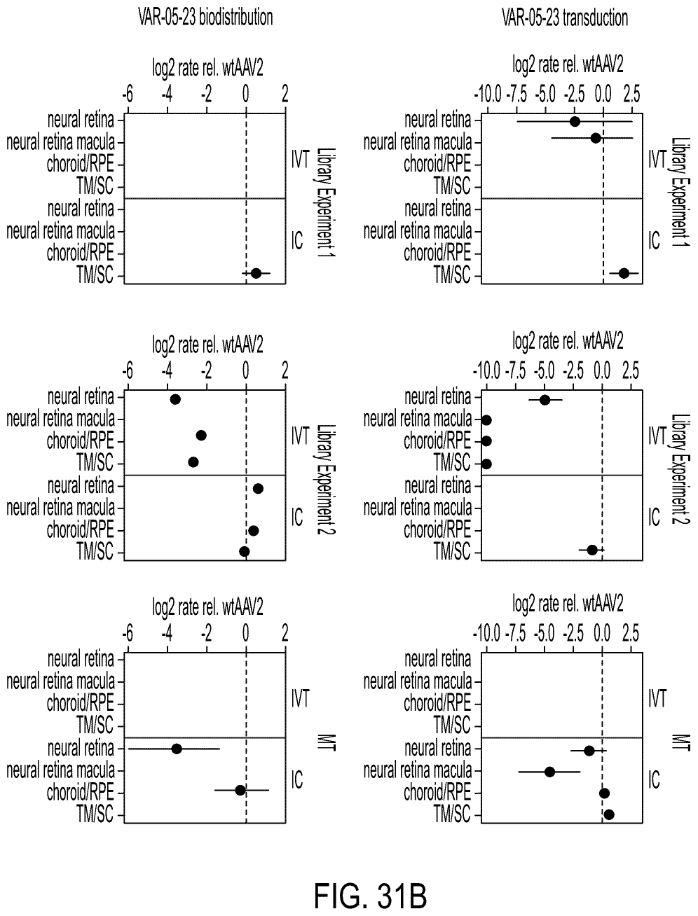

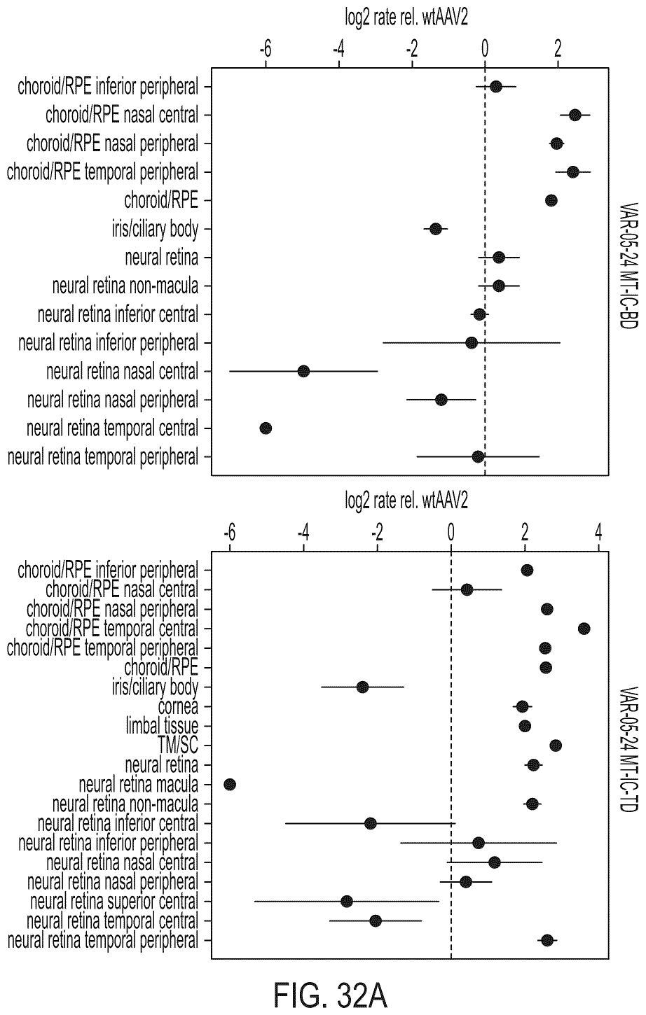

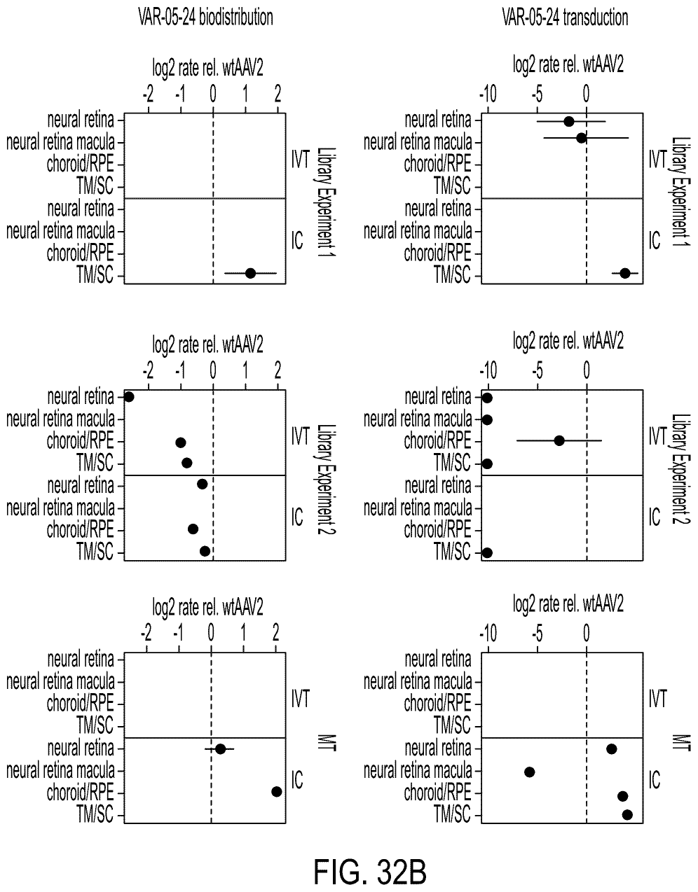

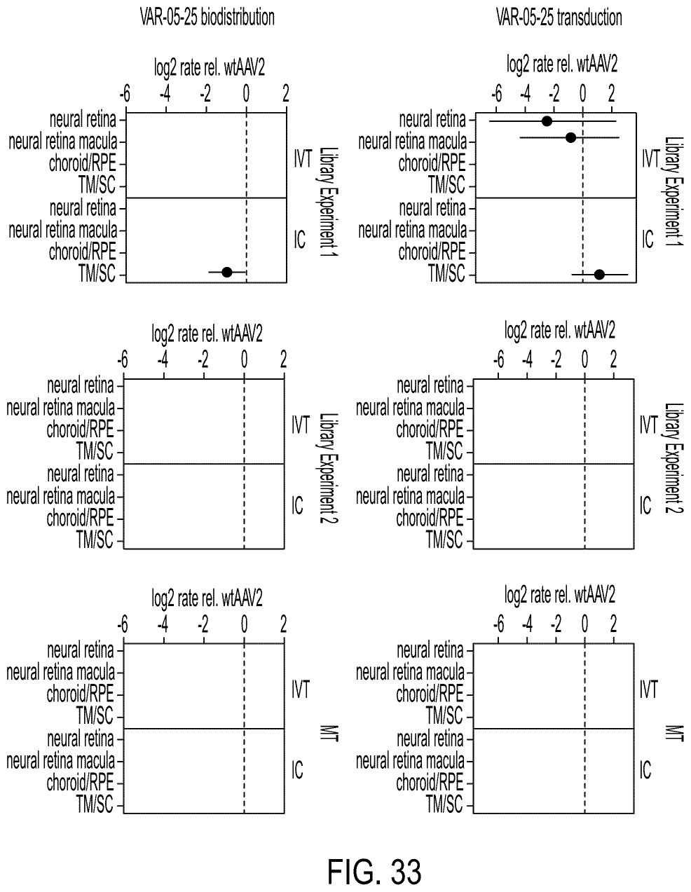

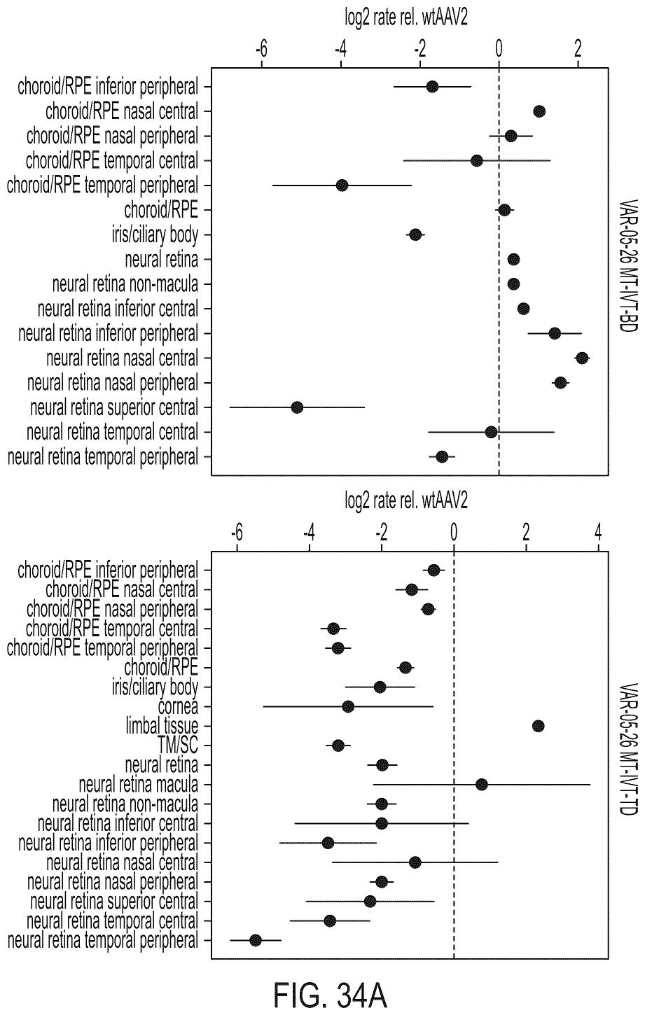

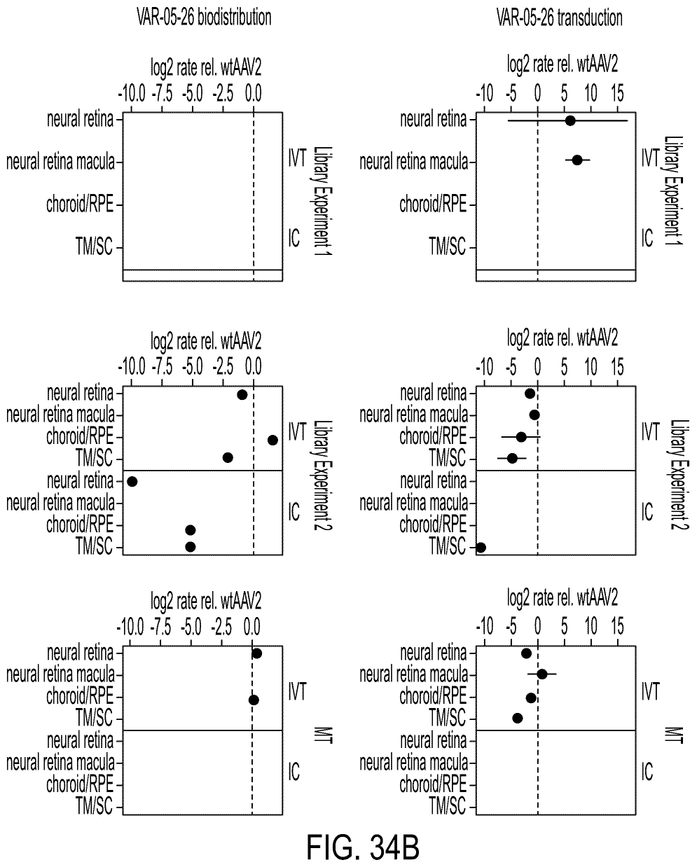

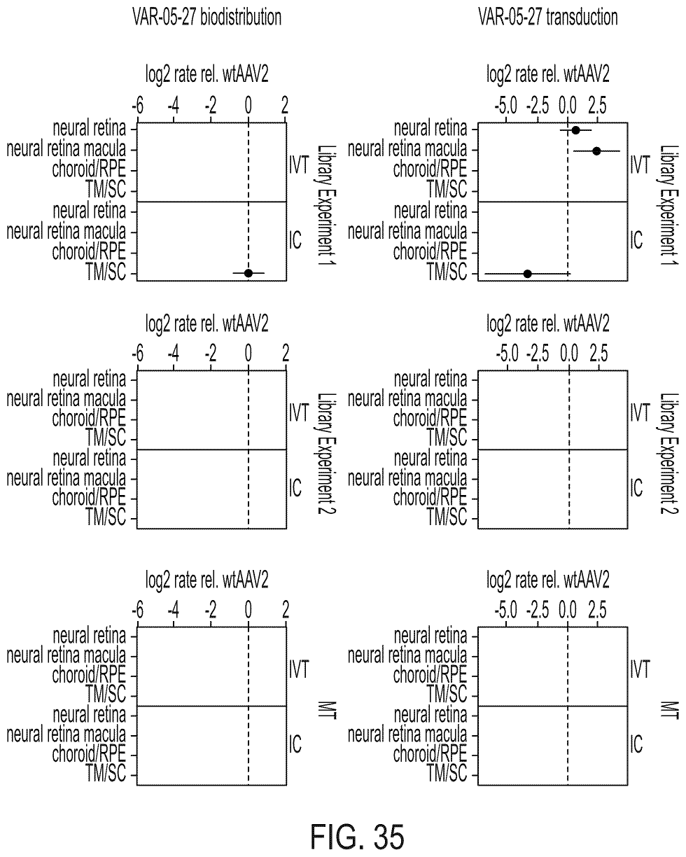

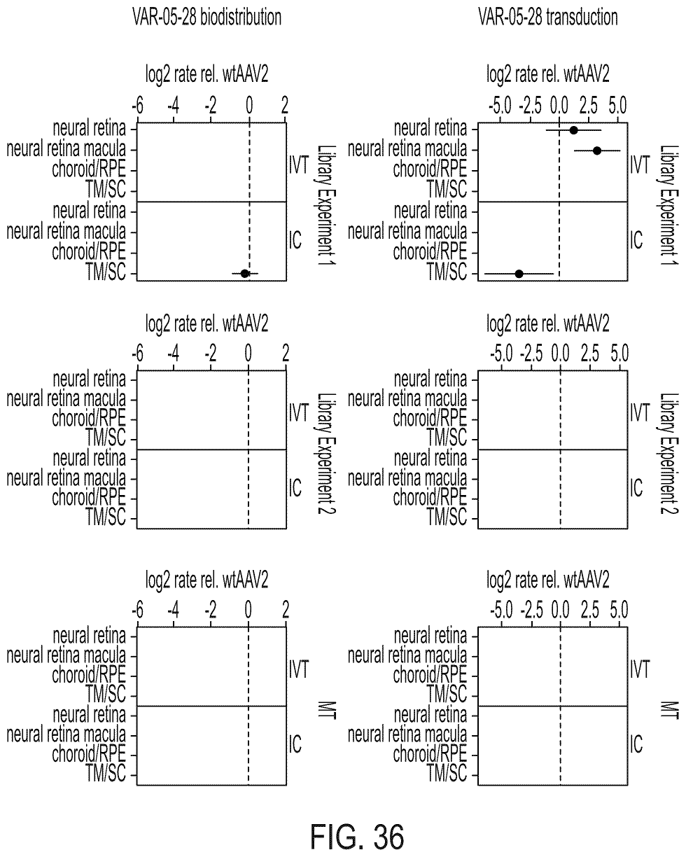

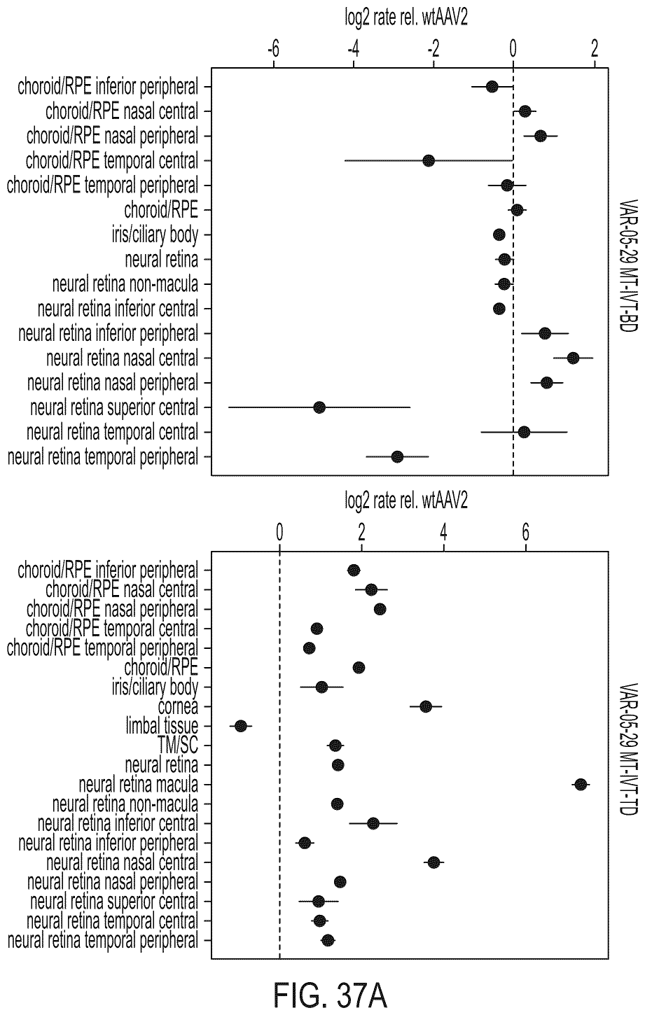

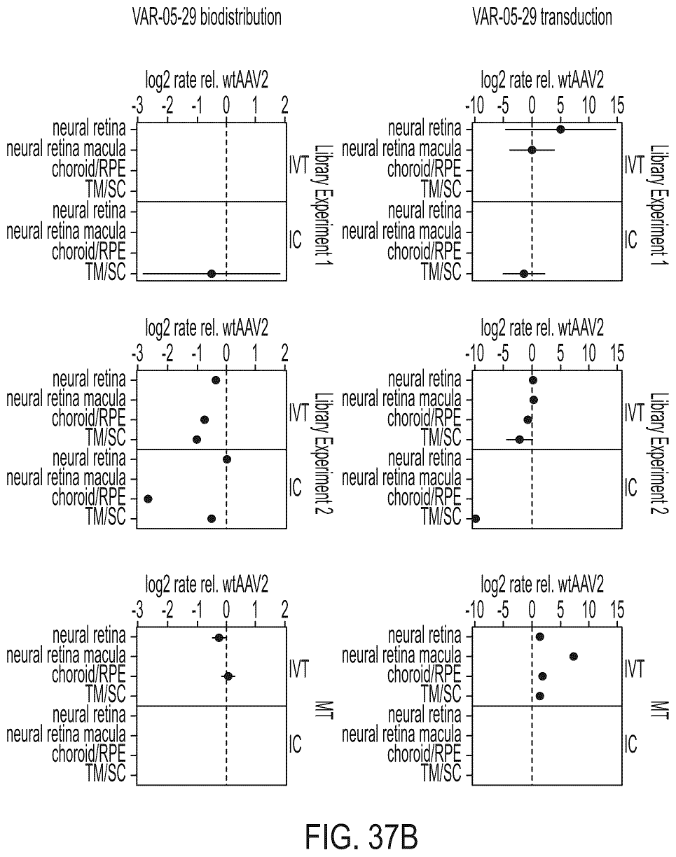

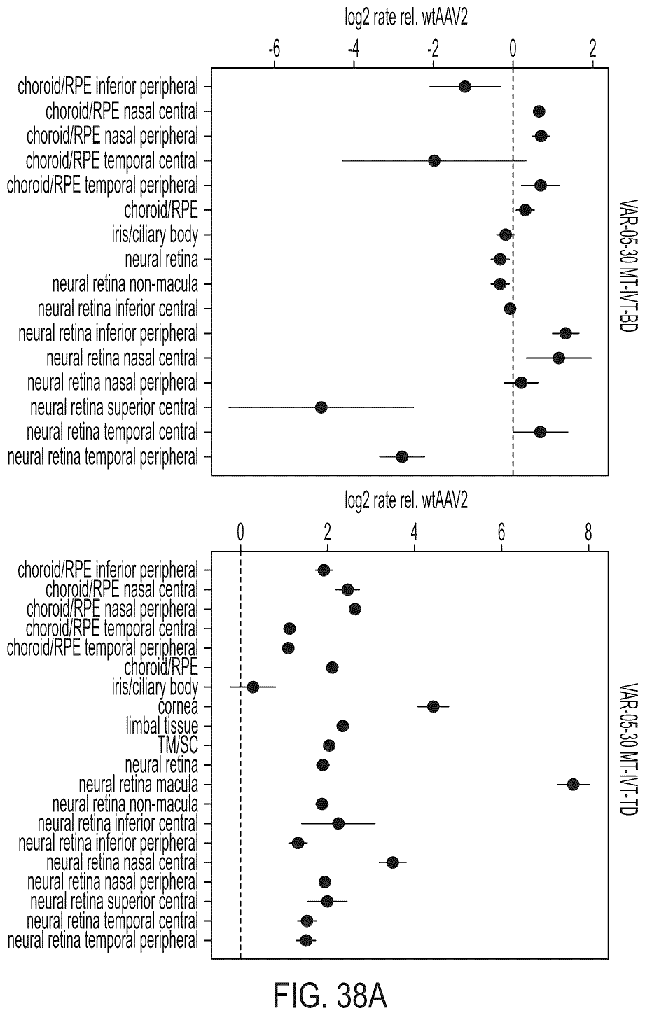

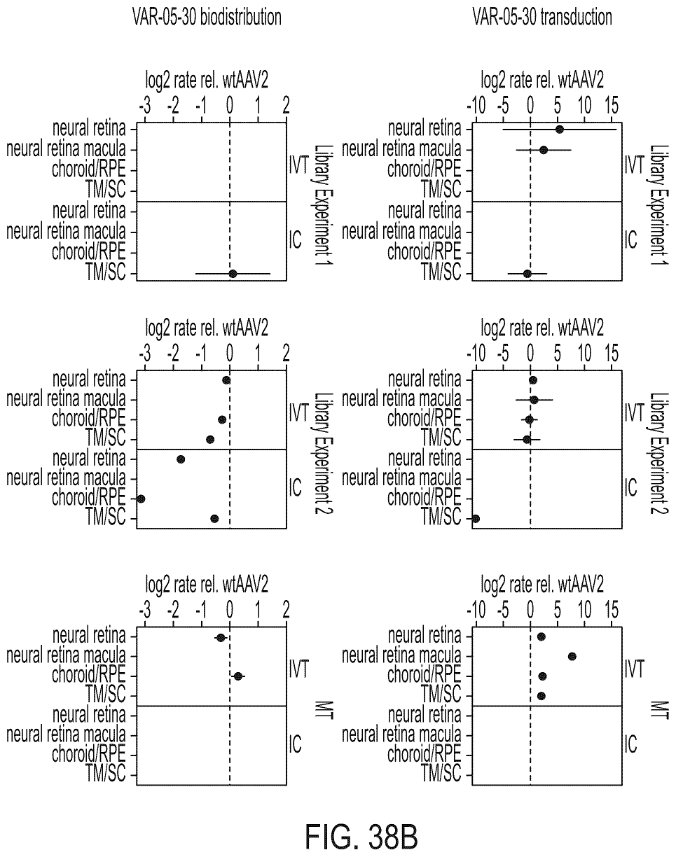

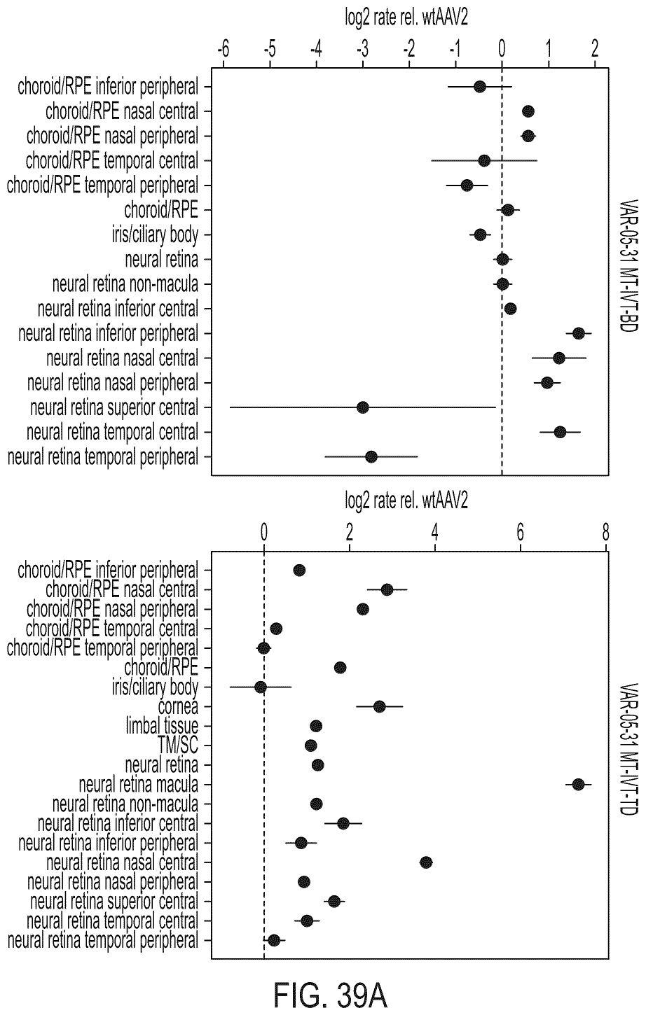

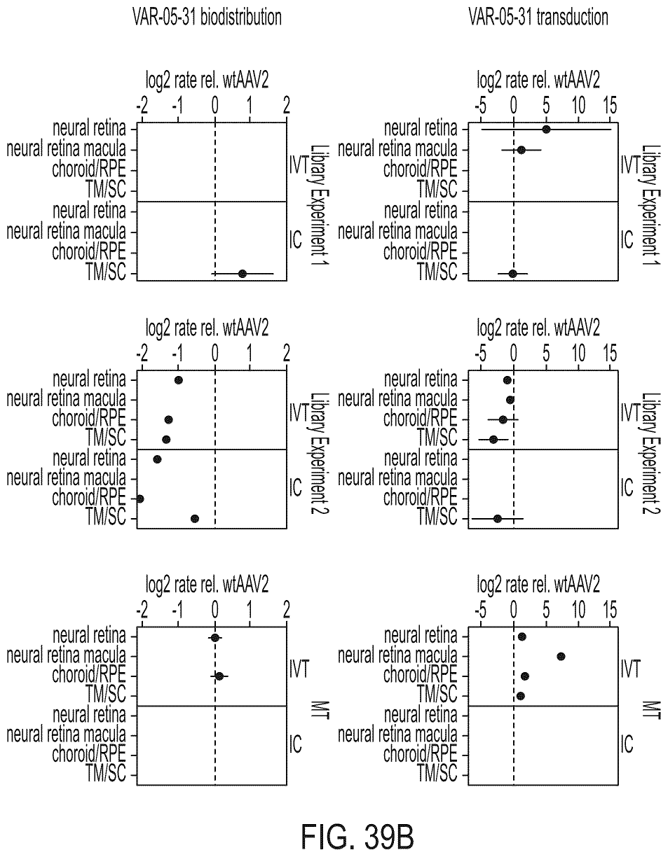

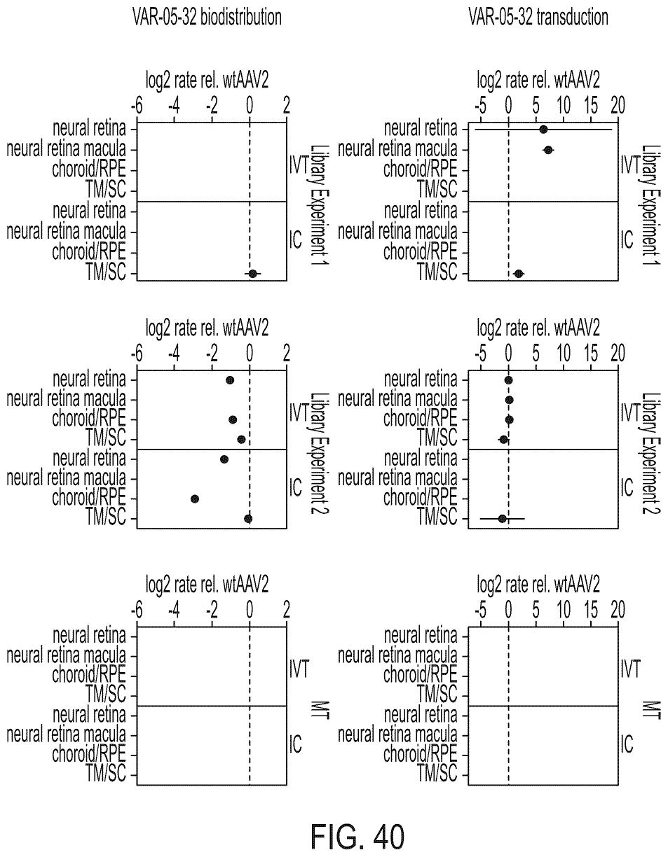

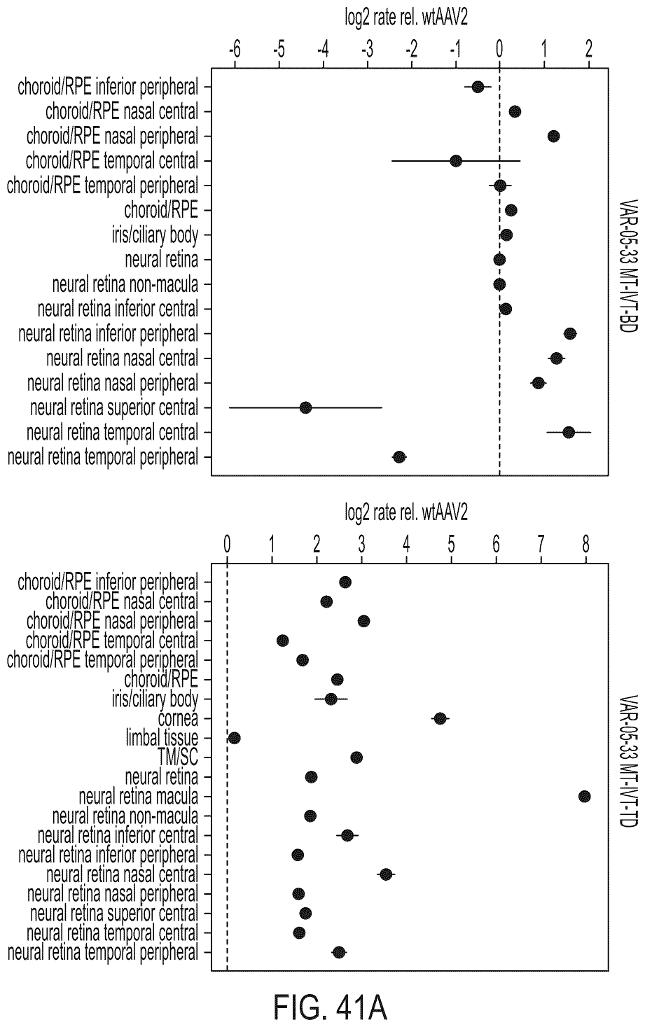

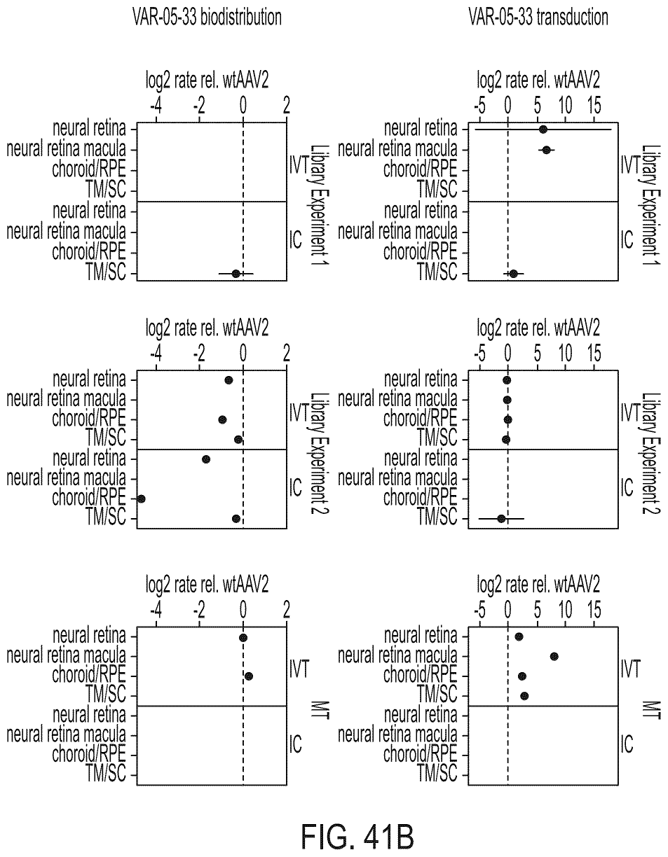

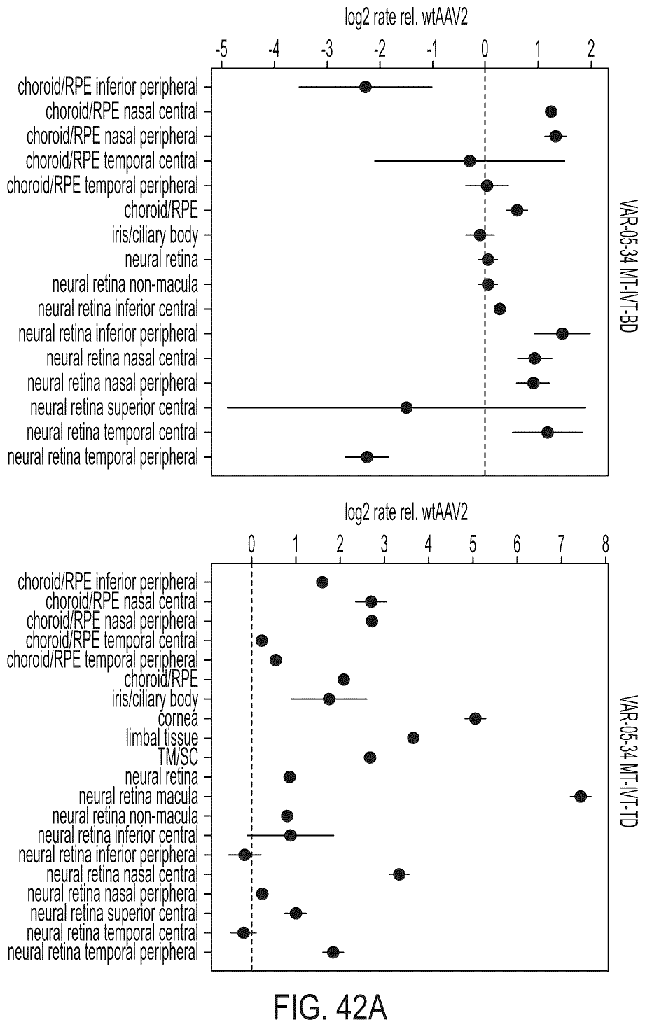

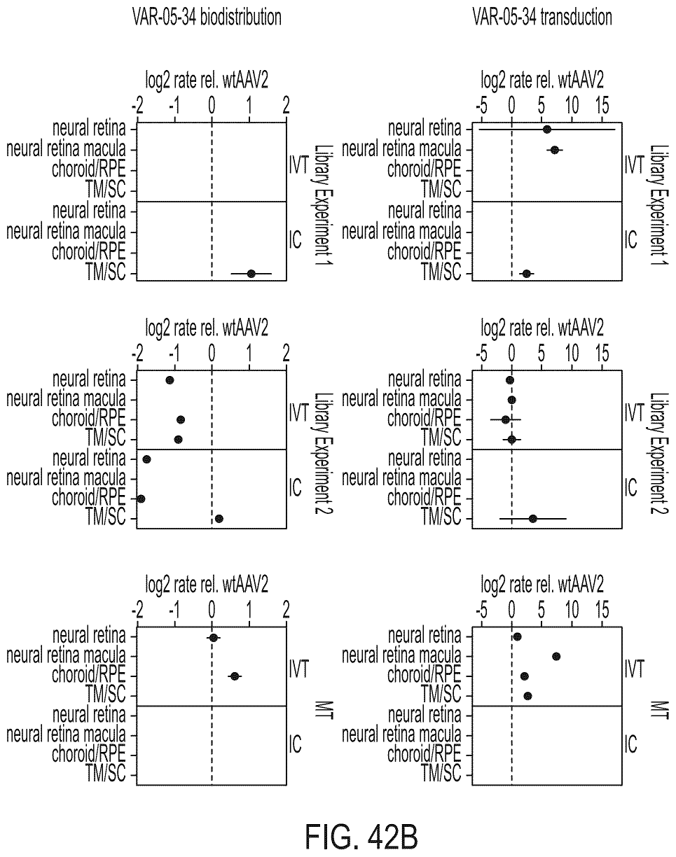

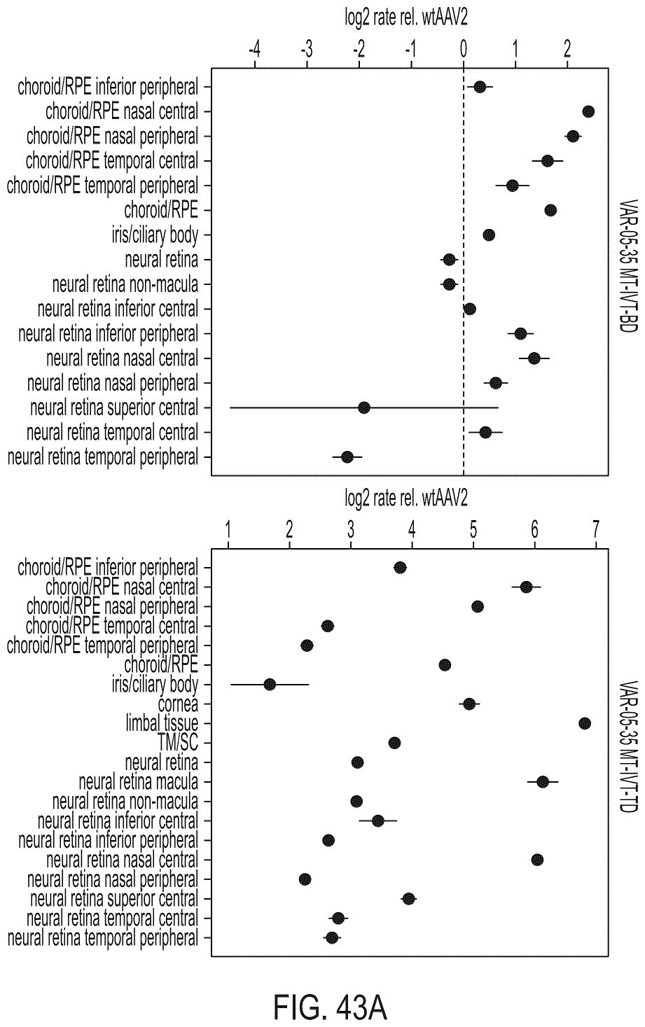

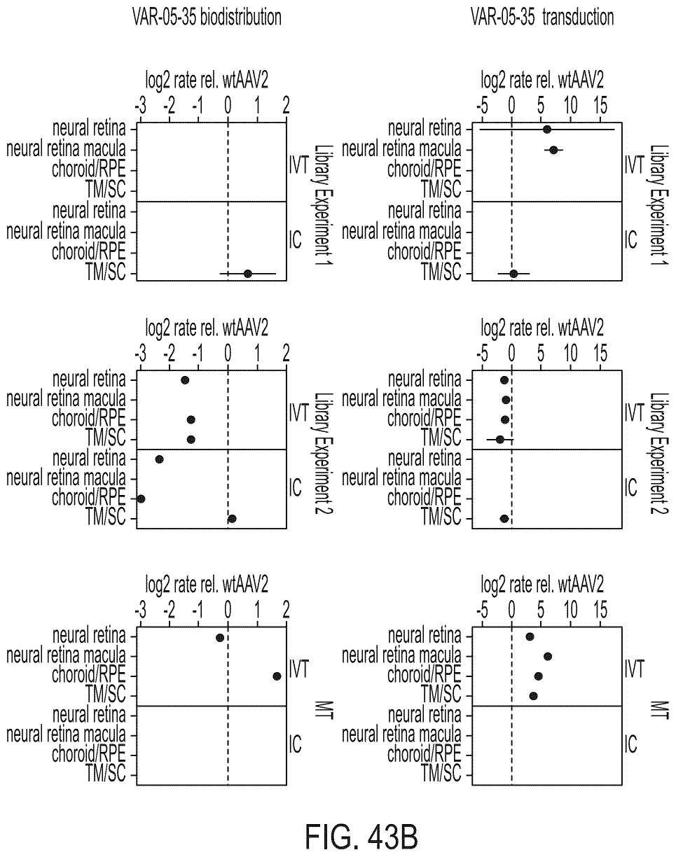

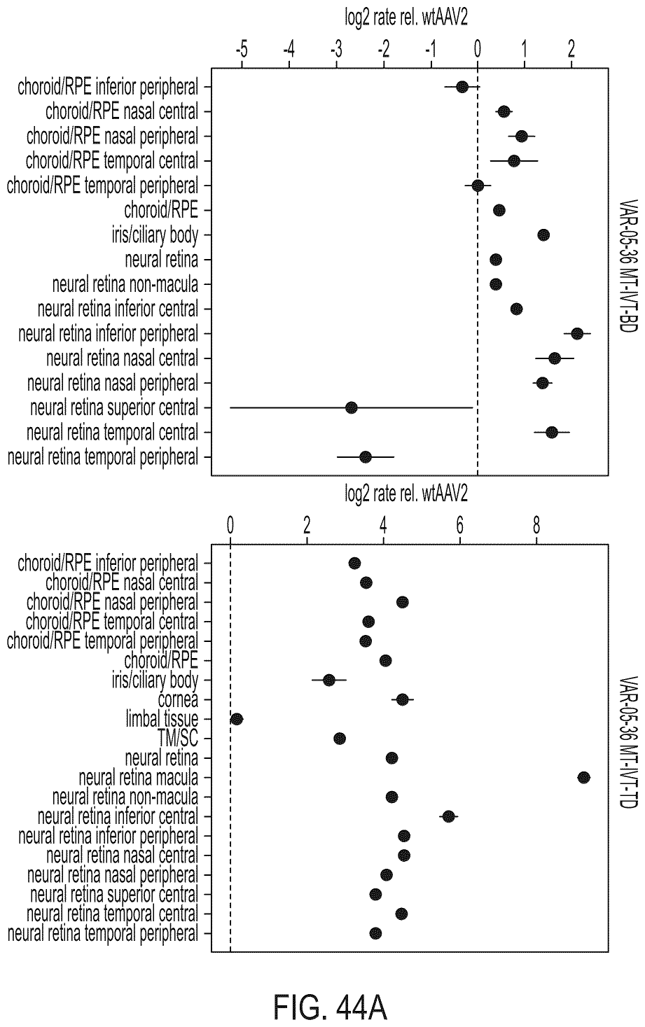

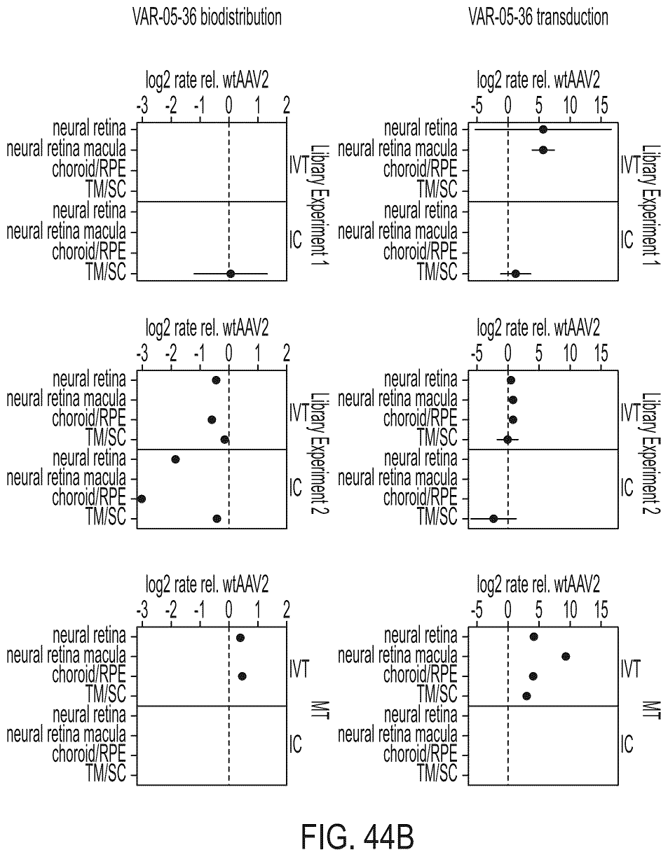

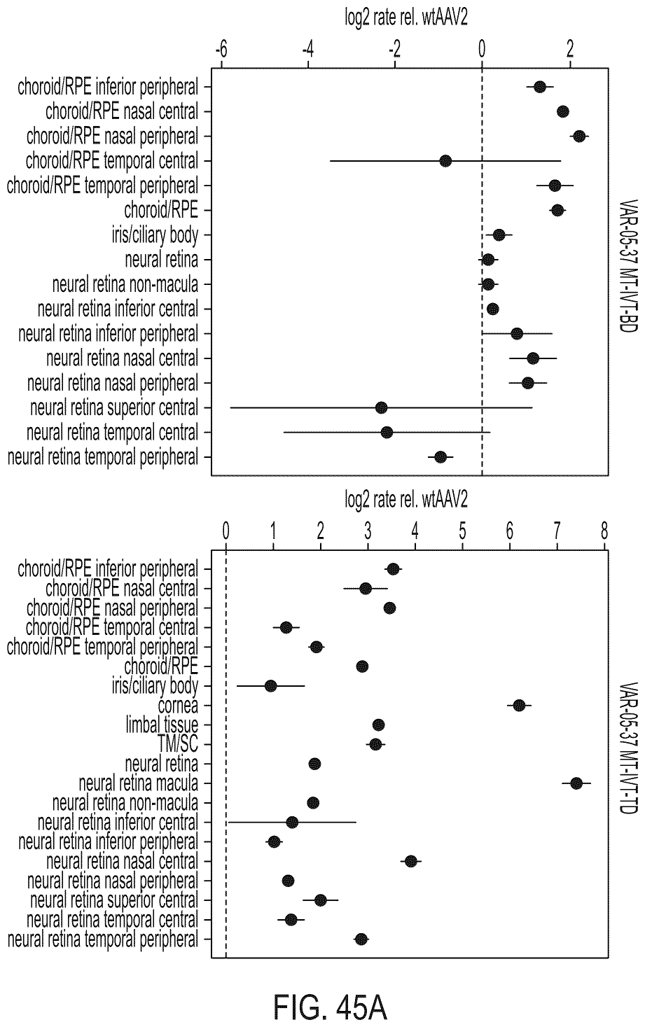

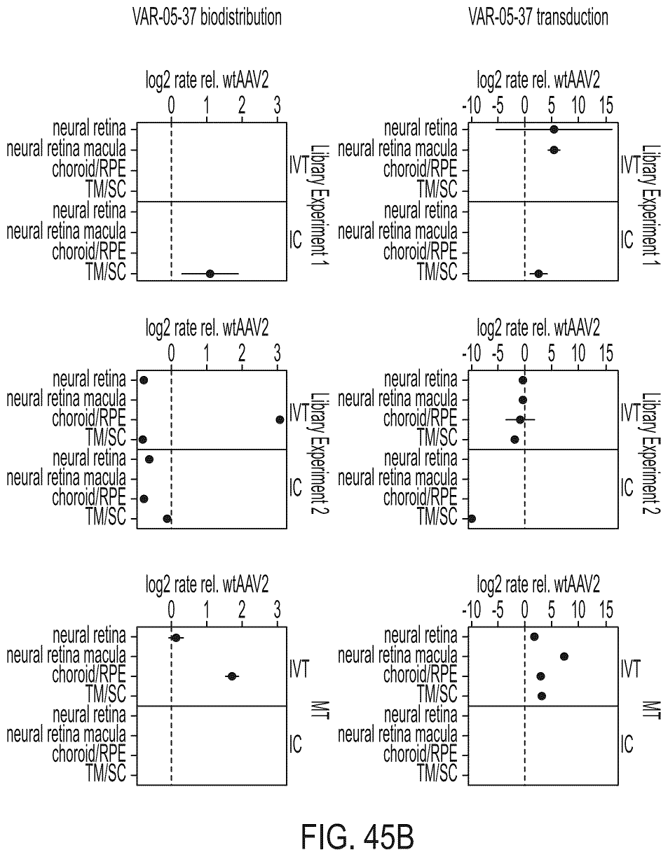

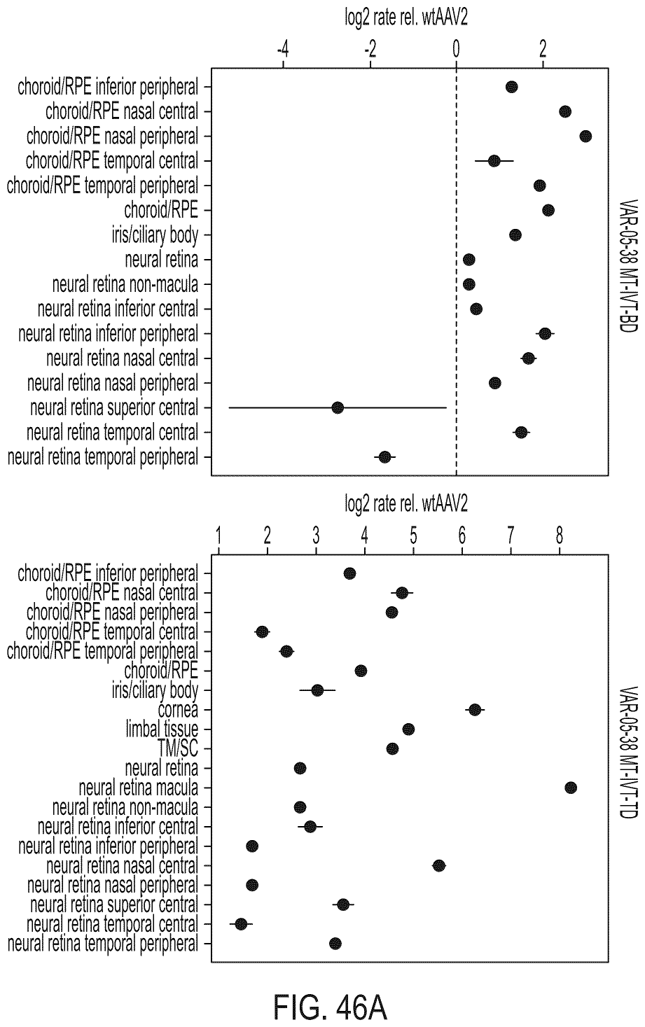

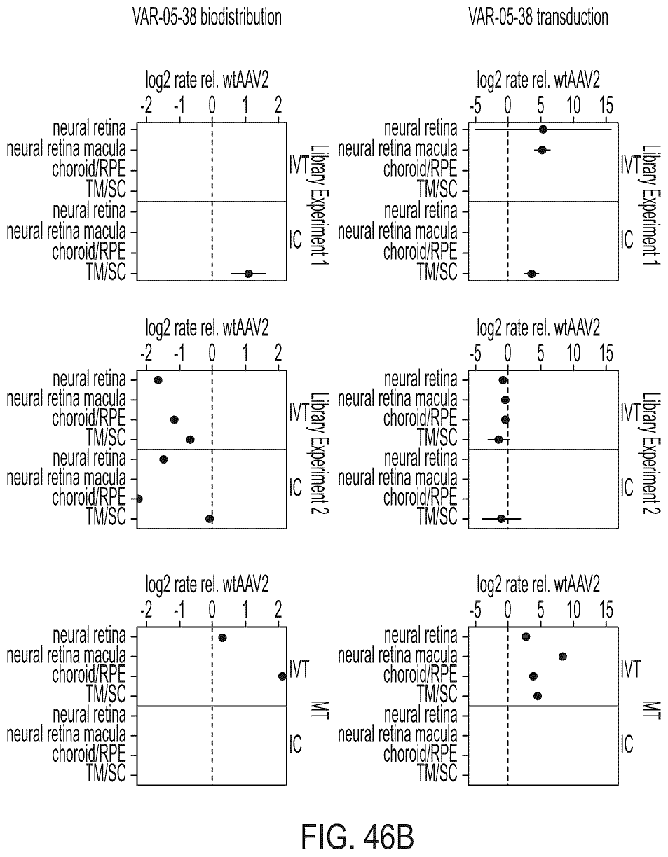

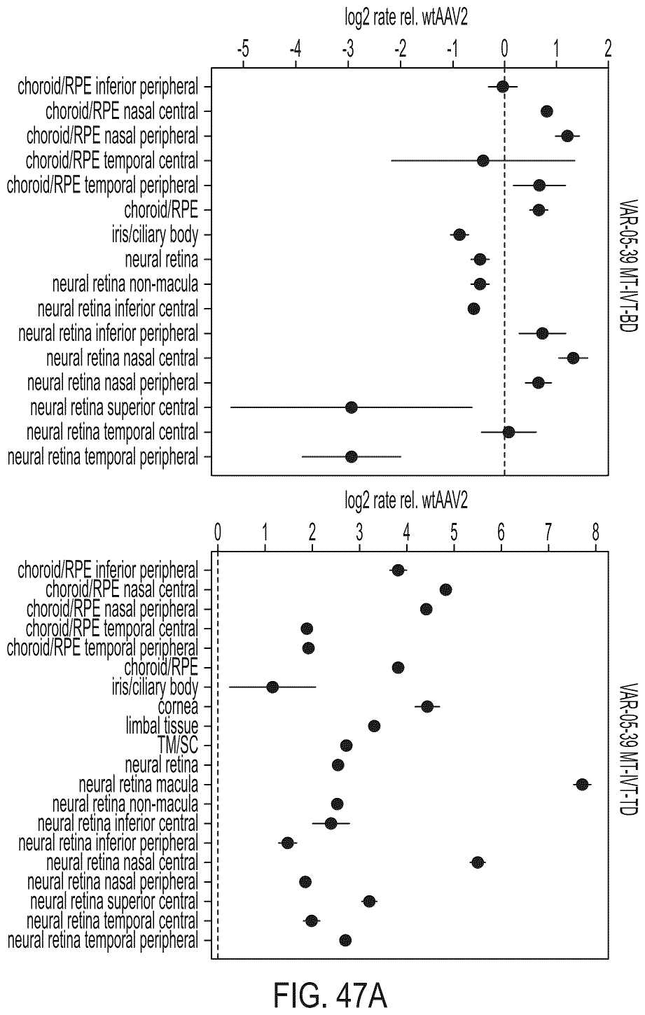

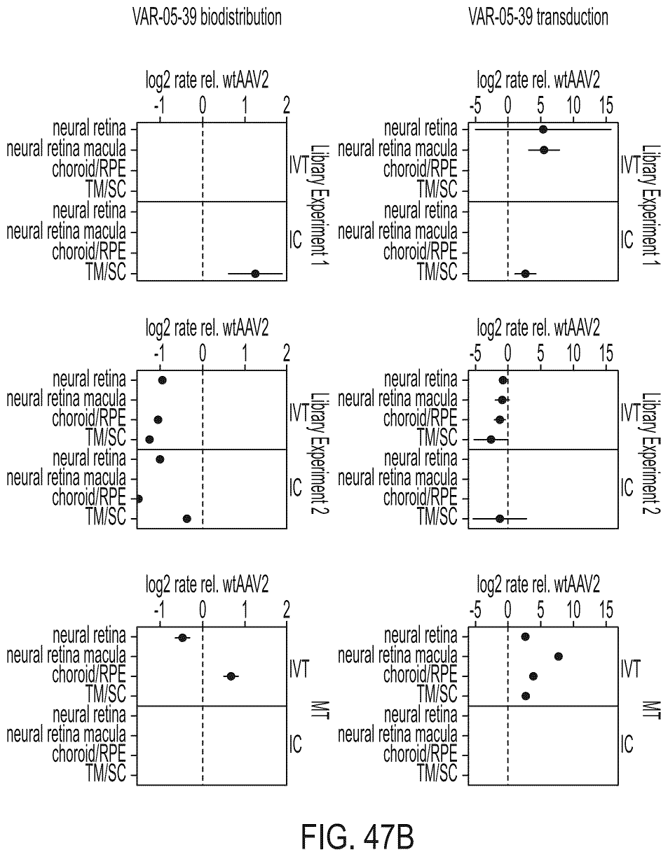

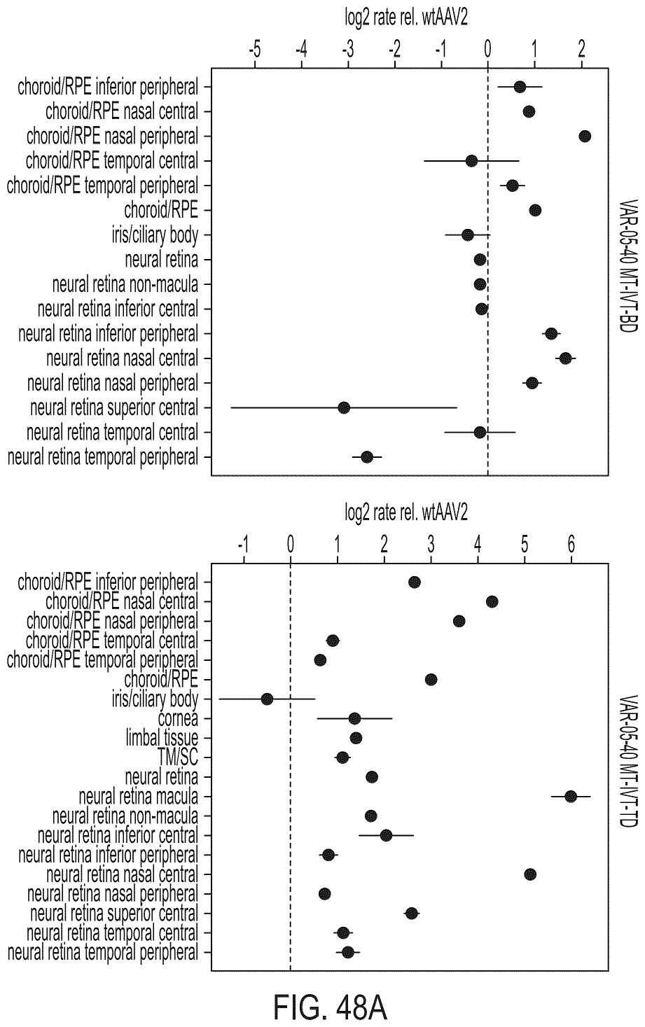

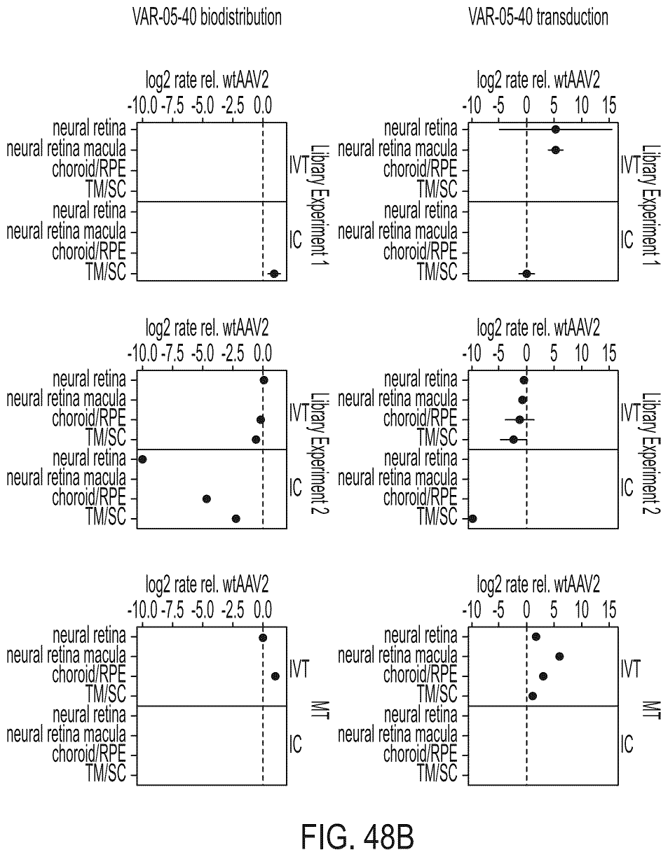

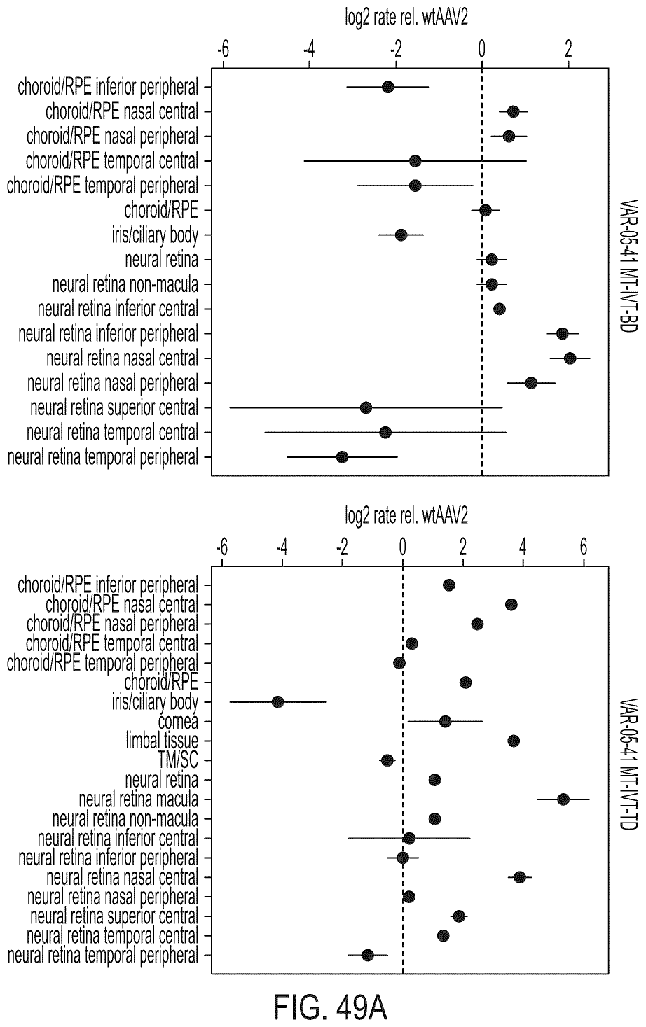

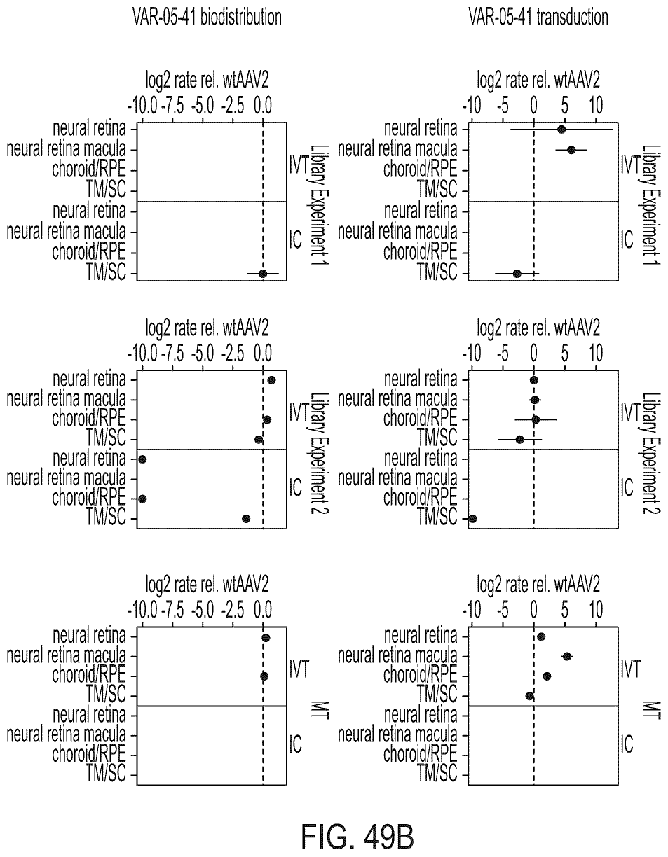

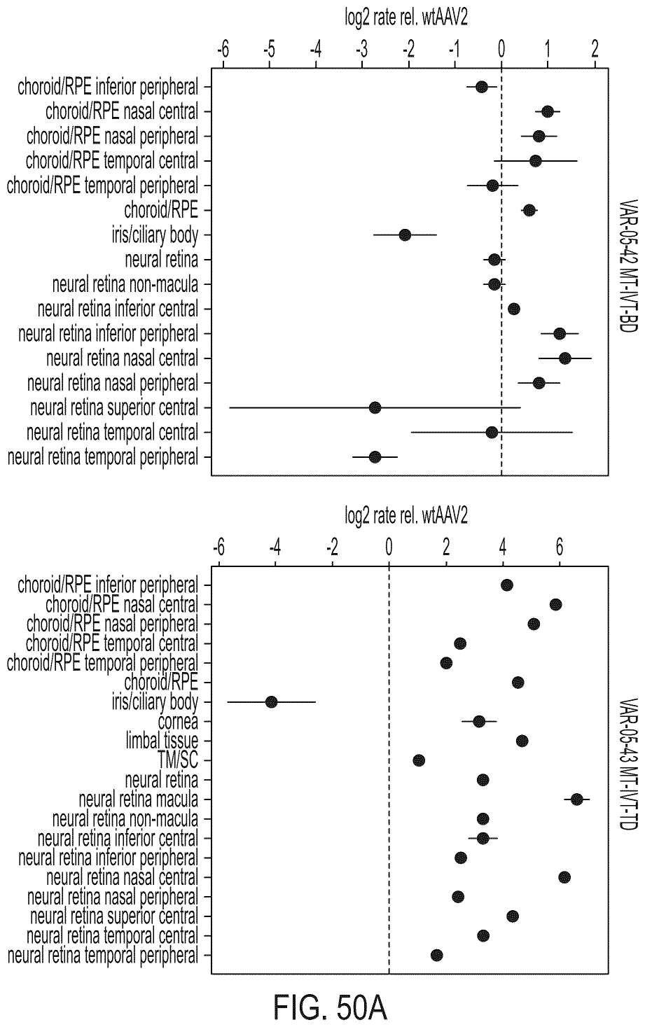

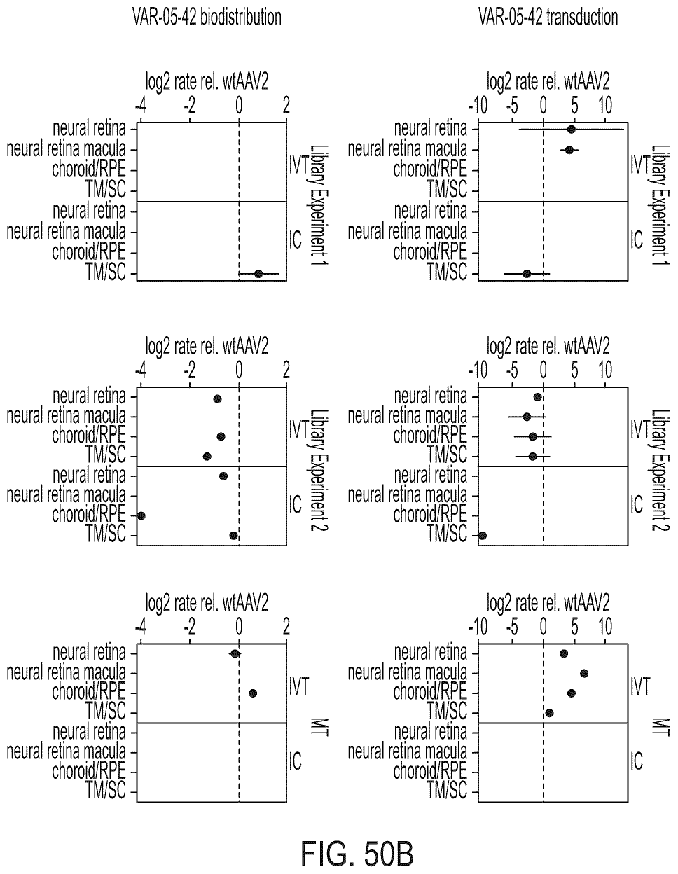

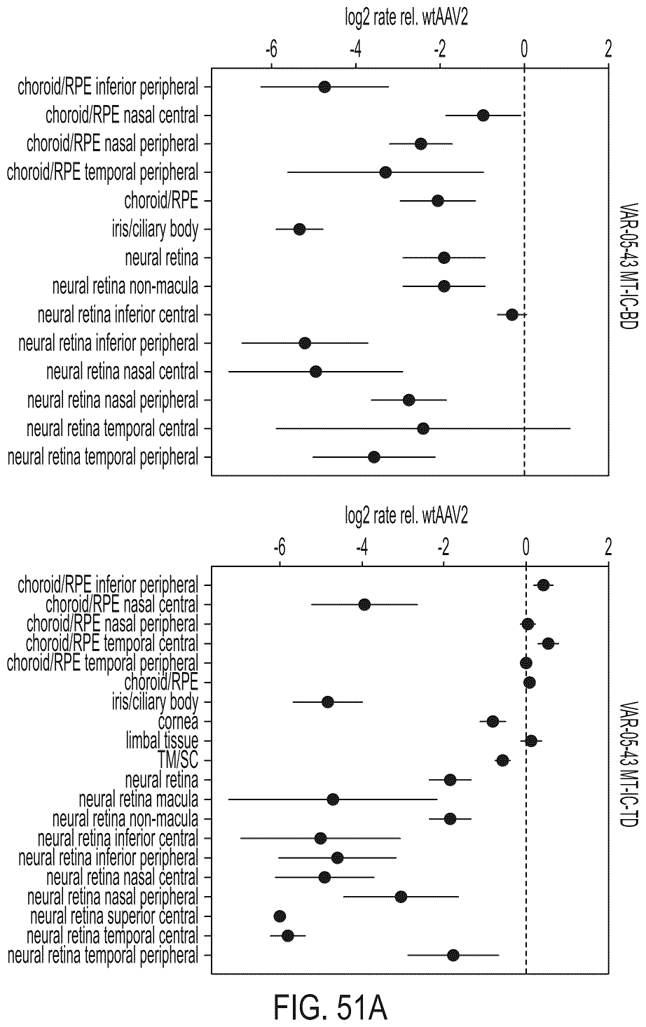

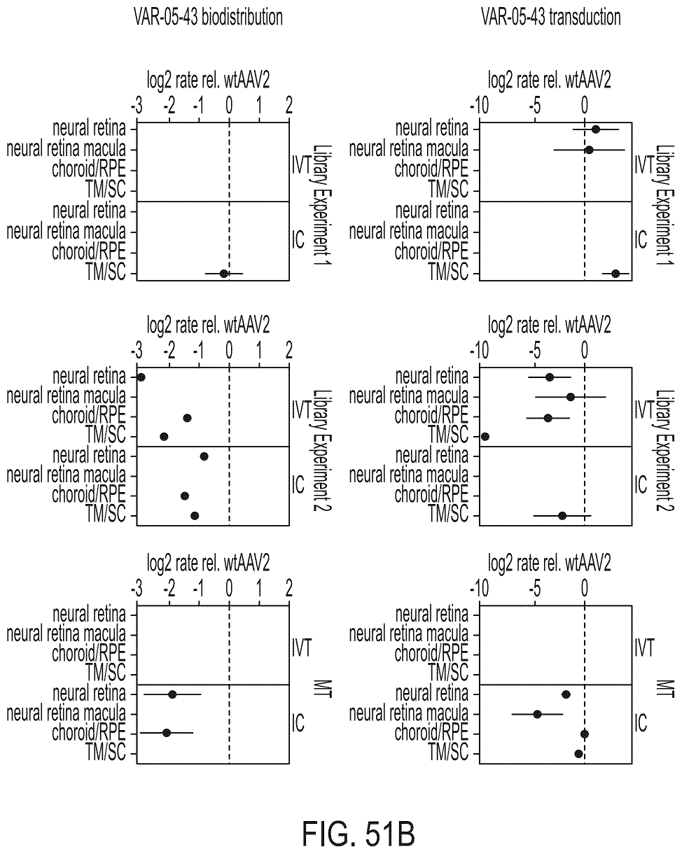

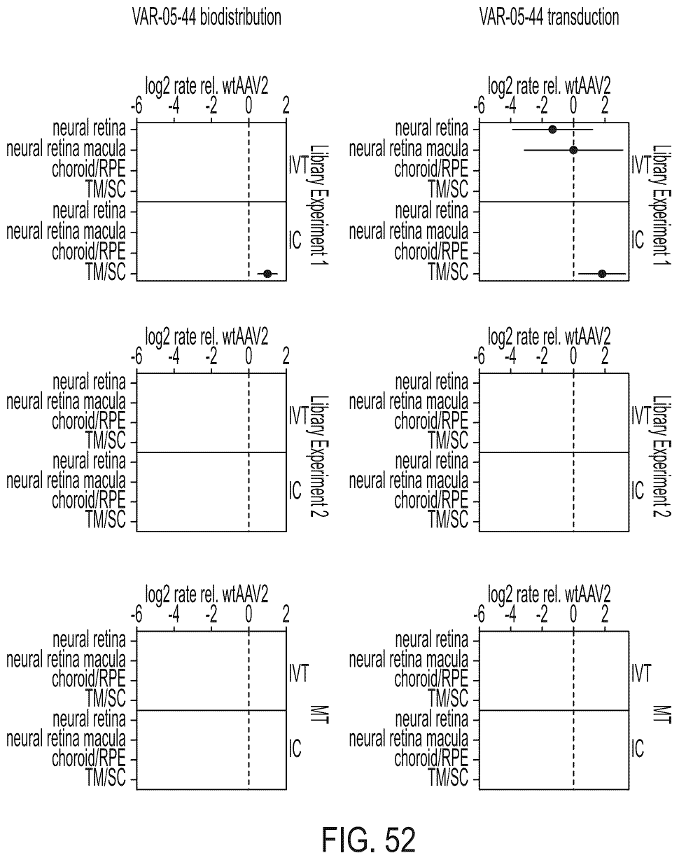

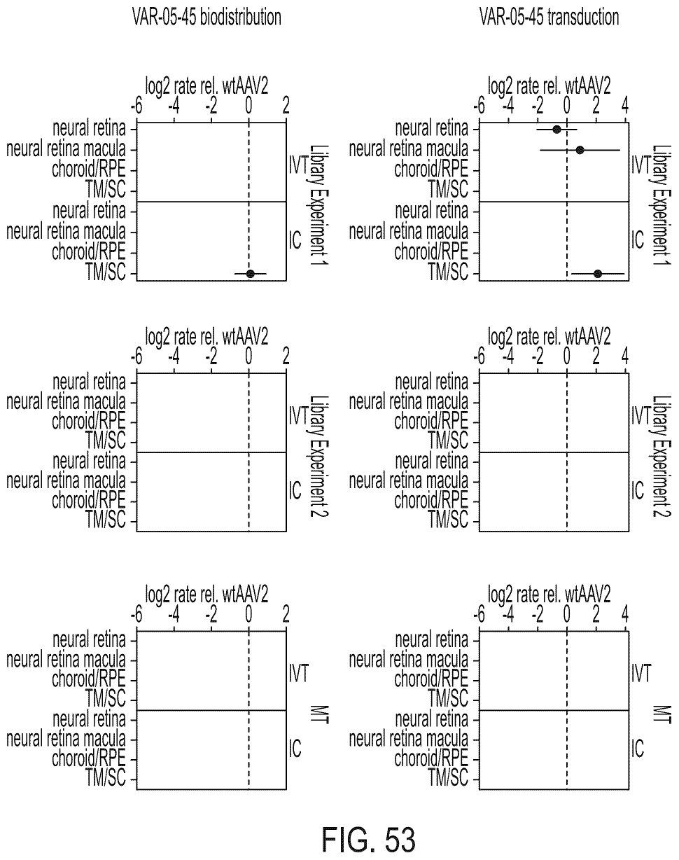

Control experiments comprising a mixture of 5% HEK293 cells transduced with the library of variants and 95% untransduced NHP liver cells were performed to determine the sensitivity of the assay. After processing and data analysis, we were able to determine that 6% of all nuclei processed were HEK-293 nuclei and detected viral transcripts from the library in approximately 20% of the HEK-293 nuclei. This sensitivity is sufficient to identify and characterize viral vectors comprising variant capsid polypeptides from the medium throughput study described herein. Next, we sequenced data from nuclei isolated from NHP posterior eye and anterior eye tissue. We plotted RNA transcript data on a UMAP plot (Leiden clustering) to show major cell types when projected onto an annotated eye reference dataset using PCA (Swamy, V S et al., Gigascience 2021, incorporated herein by reference). This allowed us to annotated major cell types of the eye, including Amacrine cells, bipolar cells, cones, horizontal cells, microglia, Muller glia, retinal ganglion cells, and rods. Using cell-type specific markers from the literature (Menon M et al., Nature Comm. 2019; Peng Y et al., Cell, 2019, both incorporated herein by reference) we confirmed expected expression patterns from these cell clusters. FIG. 9 shows the cell type transduction for variants from the medium throughput study from samples collected from the posterior eye. The error bars show 90% confidence interval, estimated by randomly resampling the cells 2000 times. As shown in FIG. 9, variants described herein have enhanced transduction of rods and cones, among other cells, relative to AAV2 wild-type. In addition, variants described herein are capable of transducing Muller glia and Amacrine cells which showed no detectable wild-type AAV2 transduction.

### Example 3

In order to identify structure-function relationships between the mutation sets (i.e., groups of two or more mutations) associated with the variant capsid polypeptides described herein and increased retina transduction, all mutations relative to wild-type AAV2 present in each of the variants in Table 2 were decomposed into their constituent mutations such that each substitution or deletion counted as an individual mutation, each insertion sequence counted as a single mutation, and all possible prefixes and suffixes of an insertion counted as individual mutations. For example, an insertion ABC at position n would be decomposed into both ABC_n to represent the full insertion and also [AB+_n, A+_n, +BC_n, +C_n] where ‘+’ represents one or more of any amino acid in that insertion position.

Next, for all possible subcombinations of mutations (referred to as “Common Mutation Sets”) generated as described above, we identified and grouped all variants tested in either Library Experiment 1 or Library Experiment 2 that contained the same mutations present in the Common Mutation Set and had an edit distance of at most 3 from a sequence containing only the Common Mutation Set present in the specific subcombination. To calculate this, we first reduced mutation sets containing redundant insertion prefixes or suffixes to the minimal descriptive set of mutations (for example, the set {AB+_n, ABC_n} is reduced to {ABC_n} and the set {+BC_n, +C_n} is reduced to {+BC_n}). For reduced mutation sets containing insertion mutations including a ‘+’ we subtracted one when calculating edit distance to allow a single wildcard. We then calculated the median neural retina transduction measurements (log 2 relative to wild-type AAV2) across all variants associated with each Common Mutation Set. We chose the median because it is a robust statistic that is more resilient to outliers than the mean.

The resulting Common Mutation Sets of minimal structural elements (i.e., subcombinations of mutations found in the variants described herein) were filtered according to one of the following two criteria: (1) at least 5 unique capsid polypeptide variants comprising the submotif present in the library experiment and median log 2 relative to wild-type AAV2 whole retina transduction greater than 0 or (2) less than 5 unique capsid polypeptide variants comprising the submotif and median log 2 relative to wild-type AAV2 whole retina transduction greater than 0.5. The resulting Common Mutation Sets are those that the data indicate are associated with increased whole retinal transduction and thus comprise unique structure-function relationships identified and described herein. Results are shown in Table 12 (submotifs identified from an analysis of Library Experiment 1 variants) and Table 13 (submotifs identified from an analysis of Library Experiment 2 variants).

### REFERENCES

Ogden P J, Kelsic E D, Sinai S, Church G M, Comprehensive AAV capsid fitness landscape reveals a viral gene and enables machine-guided design. Science. 2019 Nov. 29; 366(6469):1 139-1143. doi: 10.1126/science.aaw2900. PMID: 31780559: PMCID: PMC7197022.

The results show that variant capsid polypeptides provided for herein produce virus particles that have increased packaging, increased biodistribution, increased transduction and/or increased expression of a transgene (payload) in various regions of the eye relative to a wild-type AAV2 upon intravitreal or intracameral injection. In addition, the variant capsid polypeptides described herein provide selective biodistribution and/or expression in regions of the eye that include target cell populations for gene therapy (for example, macula-selectivity, non-macula retina selectivity, macula/retina selectivity and/or trabecular meshwork selectivity). Without limitation, the capsid polypeptides, nucleic acids and virus particles described herein are used to deliver therapeutics to the eye, e.g., to certain cell types of the eye, and are used to treat disorders of the eye as described herein, with higher efficiency.

1. A variant capsid polypeptide comprising a polypeptide that has at least 85, 90, 91, 92, 93, 94, 95, 96, 97, 98, 99%, or 100% identity to a VP1, VP2, or VP3 sequence of SEQ ID NO: 37, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46.

2. The variant capsid polypeptide of claim 1, wherein the polypeptide comprises:
a mutation selected from a mutation associated with any of VAR-1 to VAR-2, and a mutation selected from a mutation associated with any of VAR-3 to VAR-16;
a mutation selected from a mutation associated with any of VAR-1 to VAR-2 and a mutation selected from a mutation associated with any of VAR-17 to VAR-45; or
a mutation selected from a mutation associated with any of VAR-3 to VAR-16 and a mutation selected from a mutation associated with any of VAR-17 to VAR-45.

3. The variant capsid polypeptide of claim 2, wherein:
the mutation associated with any of VAR-1 to VAR-2 comprises mutations corresponding to residues 1-36 as compared to SEQ ID NO: 1;
the mutation associated with any of VAR-3 to VAR-16 comprises mutations corresponding to residues 431-466 as compared to SEQ ID NO: 1; and
the mutation associated with any of VAR-17 to VAR45 comprises mutations corresponding to residues 552-617 as compared to SEQ ID NO: 1.

4. The variant capsid polypeptide of any of the preceding claims, wherein the polypeptide comprises a variant of SEQ ID NO: 1, wherein the variant capsid polypeptide comprises a mutation selected from a mutation associated with any of VAR-1 to VAR-2 and a mutation associated with any of VAR-3 to VAR-16.

5. The variant capsid polypeptide of any of the preceding claims, wherein the polypeptide comprises a variant of SEQ ID NO: 1, wherein the variant capsid polypeptide comprises a mutation selected from a mutation associated with any of VAR-1 to VAR-2 and a mutation associated with any of VAR-17 to VAR-45.

6. The variant capsid polypeptide of any of the preceding claims, wherein the polypeptide comprises a variant of SEQ ID NO: 1, wherein the variant capsid polypeptide comprises a mutation selected from a mutation associated with any of VAR-3 to VAR-16 and a mutation associated with any of VAR-17 to VAR-45.

7. The variant capsid polypeptide of any of the preceding claims, wherein the polypeptide comprises a variant of SEQ ID NO: 1, wherein the variant capsid polypeptide comprises a mutation that corresponds to a mutation at position 3, 6, 11, 12, 14, 15, 19, 21, 23, 24, 25, 29, 31, 33, 446, 449, 450, 451, 452, 454, 455, 456, 457, 458, 459, 460, 461, 464, 552, 554, 555, 556, 557, 558, 559, 561, 566, 575, 578, 580, 581, 585, 586, 587, 588, 589, 590, 591, 593, 594, 597, or 600, an insertion between positions 446 and 447, 447 and 448, 448 and 449, 449 and 450, 451 and 452, 453 and 454, 581 and 582, 583 and 584, 584 and 585, 585 and 586, 586 and 587, 587 and 588, 588 and 589, 589 and 590, 590 and 591, 591 and 592, or 592 and 593 according to SEQ ID NO: 1, optionally wherein the mutation comprises an insertion, a deletion or a substitution.

8. The variant capsid polypeptide of any of the preceding claims, wherein the polypeptide comprises a variant of SEQ ID NO: 1, wherein the variant capsid polypeptide comprises a mutation that corresponds to a mutation at position 3, 6, 11, 12, 14, 15, 19, 21, 23, 24, 25, 29, 31, 33, or any combination thereof according to SEQ ID NO: 1, and wherein the mutation comprises a deletion or a substitution.

9. The variant capsid polypeptide of any of the preceding claims, wherein the polypeptide comprises a variant of SEQ ID NO: 1, wherein the variant capsid polypeptide comprises a mutation that corresponds to a mutation at position 446, 448, 449, 450, 451, 452, 453, 454, 455, 456, 457, 458, 459, 460, 461, 464, or any combination thereof, an insertion between positions 446 and 447, 447 and 448, 448 and 449, 449 and 450, 451 and 452, 453 and 454, or any combination thereof according to SEQ ID NO: 1, and wherein the mutation comprises an insertion, a deletion or a substitution.

10. The variant capsid polypeptide of any of the preceding claims, wherein the polypeptide comprises a variant of SEQ ID NO: 1, wherein the variant capsid polypeptide comprises a mutation that corresponds to a mutation at position 552, 554, 555, 556, 557, 558, 559, 561, 566, 575, 578, 580, 581, 585, 586, 587, 588, 589, 590, 591, 593, 594, 597, 600 or any combination thereof, an insertion between positions 583 and 584, 584 and 585, 585 and 586, 586 and 587, 587 and 588, 588 and 589, 589 and 590, 590 and 591, 591 and 592, 592 and 593, or any combination thereof according to SEQ ID NO: 1, and wherein the mutation comprises an insertion, a deletion or a substitution.

11. A variant capsid polypeptide comprising a variant of SEQ ID NO: 1 comprising a mutation selected from a mutation associated with any of VAR-3 to VAR-16, wherein the mutation is
an insertion, e.g., an insertion of 4 or more amino acids, e.g., 4-5 amino acids, e.g., 4-6 amino acids, e.g., 4-7 amino acids, e.g., 7 amino acids, that corresponds to an insertion between positions 446 and 454 as compared to SEQ ID NO: 1, and wherein the insertion comprises a polypeptide that has at least 60%, 70%, 80%, 90%, or 100% identity to SEQ ID NO: 93-122;
a substitution, e.g., a substitution of at least 2 or more residues, e.g., at least 6-10 residues, e.g., at least 7-10 residues, e.g., at least 8-10 residues, e.g., at least 9-10 residues, e.g., at least 10 residues that correspond to a substitution at positions between 445 and 465 as compared to SEQ ID NO: 1; and
any combination thereof;
and wherein the variant comprises at least 3 mutations selected from: T455G/L/Q, T456G, Q457T, S458Q, R459G/T; and further comprises at least 3 other mutations between positions 445-465 as compared to SEQ ID NO: 1, and wherein the mutations are substitutions, insertions, or deletions.

12. A variant capsid polypeptide comprising a variant of SEQ ID NO: 1 comprising a mutation selected from a mutation associated with any of VAR-3 to VAR-16, wherein the mutation is between positions 552 and 588 as compared to SEQ ID NO:1, and wherein the mutation comprises:
an insertion, e.g., an insertion of 4 or more amino acids, e.g., 4-5 amino acids, e.g., 4-6 amino acids, that corresponds to an insertion between positions 583 and 588 as compared to SEQ ID NO: 1, and wherein the insertion comprises a polypeptide that has at least 60%, 70%, 80%, 90%, or 100% identity to SEQ ID NO: 93-122 or comprises a fragment of 4 or more amino acids of SEQ ID NO: 93-122;
a substitution, e.g., a substitution of at least 2 or more residues, e.g., at least 3-9 residues, e.g., at least 4-9 residues, e.g., at least 5-9 residues, e.g., at least 6-9 residues, e.g., at least 7-9 residues, e.g., at least 8-9 residues, e.g., at least 9 residues that correspond to a substitution at positions between 552 and 588 as compared to SEQ ID NO: 1; and
any combination thereof.

13. A variant capsid polypeptide comprising a variant of SEQ ID NO: 1 comprising a mutation selected from a mutation associated with any of VAR-17 to VAR-45, wherein:
the mutation is between positions 552 and 566 as compared to SEQ ID NO: 1, and wherein the mutation comprises:
(a) a substitution comprising the following consensus formula:
A-n-L-S/A-D/R/N-L-L-L-n-S-n-n-n-n-K/A
wherein n is wild type residue as set forth in SEQ ID NO: 1;
(b) a substitution comprising
1, 2, 3, 4, 5, 6, or 7 of the non-naturally occurring amino acids of the consensus; or
(c) a substitution at any of positions 557, 558 or 559 as compared to SEQ ID NO: 1, and wherein the substitution introduces a leucine at any of positions 557, 558, 559, or any combination thereof.

14. A variant capsid polypeptide comprising a variant of SEQ ID NO: 1 further comprising a mutation selected from a mutation associated with any of VAR-17 to VAR-45, wherein the mutation is between positions 575 and 588 as compared to SEQ ID NO: 1, and wherein the mutation comprises:
(a) a substitution comprising the following consensus formula:
E-n-n-A/I/D/V-n-A-A/D-n-n-n-S/del-R/Q-S
wherein n is wild type residue as set forth in SEQ ID NO: 1 and del is a deletion;
(b) a substitution comprising
1-5 non-naturally occurring amino acids of the consensus formula E-n-n-A/I/D/V-n-A-A/D-n-n-n-S/del-R/Q-S; or
(c) a substitution at any of positions 578, 580 or 581 as compared to SEQ ID NO: 1, and wherein the substitution at position 578 is isoleucine, substitution at position 580 is alanine, and substitution at position 581 is aspartic acid.

15. A variant capsid polypeptide comprising a variant of SEQ ID NO: 1 further comprising a mutation selected from a mutation associated with any of VAR-17 to VAR-45, wherein the mutation is between positions 552 and 588 as compared to SEQ ID NO: 1, and wherein
the mutation is a substitution between positions 552 and 566 as compared to SEQ ID NO: 1 comprising:
(a) a substitution comprising the following consensus formula:
A-n-L-S/A-D/R/N-L-L-L-n-S-n-n-n-n-K/A
wherein n is wild type residue as set forth in SEQ ID NO: 1;
(b) a substitution comprising
1, 2, 3, 4, 5, 6, or 7 of the non-naturally occurring amino acids of the consensus; or
(c) a substitution at any of positions 557, 558 or 559 as compared to SEQ ID NO: 1, and wherein the substitution introduces a leucine at any of positions 557, 558, 559, or any combination thereof; and
the mutation is a substitution between positions 575 and 588 as compared to SEQ ID NO: 1 comprising:
(a) a substitution comprising the following consensus formula:
E-n-n-A/I/D/V-n-A-A/D-n-n-n-S/del-R/Q-S
wherein n is wild type residue as set forth in SEQ ID NO: 1 and del is a deletion;
(b) a substitution comprising
1-5 non-naturally occurring amino acids of the consensus formula E-n-n-A/I/D/V-n-A-A/D-n-n-n-S/del-R/Q-S; or
(c) a substitution at any of positions 578, 580 or 581 as compared to SEQ ID NO: 1, and wherein the substitution at position 578 is isoleucine, substitution at position 580 is alanine, and substitution at position 581 is aspartic acid.

16. A variant capsid polypeptide comprising a variant of SEQ ID NO: 1 comprising a mutation, wherein the mutation is between positions 584 and 617 as compared to SEQ ID NO:1, and wherein the mutation comprises:
an insertion, e.g., an insertion of 1 or more amino acids, e.g., 1-5 amino acids, e.g., 5-8 amino acids, e.g., 7-10 amino acids that corresponds to an insertion between positions 584 and 593 as compared to SEQ ID NO: 1, and wherein the insertion comprises a polypeptide that has at least 60%, 70%, 80%, 90%, or 100% identity to SEQ ID NO: 93-122, or a single residue selected from: F, I, P, G, or a fragment of at least 4 amino acids of any of SEQ ID NO: 93-122;
a substitution, e.g., a substitution of at least 1 or more residues, e.g., at least 1-2 residues, e.g., at least 2-7 residues, e.g., at least 2-8 residues that correspond to a substitution at positions between 584 and 617 as compared to SEQ ID NO: 1; and
any combination thereof.

17. The variant capsid polypeptide of claim 17, wherein the insertion comprises 7-10 amino acids and has at least 60%, 70%, 80%, 90%, or 100% identity to LALGETTRPA (SEQ ID NO: 93) or comprises a fragment of at least 4, at least 5, at least 6, or at least 7 amino acids of LALGETTRPA (SEQ ID NO: 93), e.g., comprises LGETTRP (SEQ ID NO: 94).

18. The variant capsid polypeptide of any of the preceding claims, wherein:
the capsid polypeptide comprises a mutation that corresponds to a mutation at position 14, 15, 19, and 24 as compared to SEQ ID NO: 1;
the capsid polypeptide comprises a mutation that corresponds to a mutation at position 3, 6, 11, 12, 21, 23, 25, 29, 31, and 33 as compared to SEQ ID NO: 1;
the capsid polypeptide comprises a mutation that corresponds to a mutation at position 449, 450, 451, 455, 456, 457, 458, and 459 as compared to SEQ ID NO: 1;
the capsid polypeptide comprises a mutation that corresponds to a mutation at position 449, 450, 452, 456, 457, and 459 as compared to SEQ ID NO: 1;
the capsid polypeptide comprises a mutation that corresponds to a mutation at position 457 and 458 as compared to SEQ ID NO: 1;
the capsid polypeptide comprises a mutation that corresponds to a mutation at position 446 as compared to SEQ ID NO: 1;
the capsid polypeptide comprises a mutation that corresponds to a mutation at position 449 as compared to SEQ ID NO: 1;
the capsid polypeptide comprises a mutation that corresponds to a mutation at position 451, 455, 456, 457, 458, 459, and 461 as compared to SEQ ID NO: 1;
the capsid polypeptide comprises a mutation that corresponds to a mutation at position 448 as compared to SEQ ID NO: 1;
the capsid polypeptide comprises a mutation that corresponds to a mutation at position 453 as compared to SEQ ID NO: 1;
the capsid polypeptide comprises a mutation that corresponds to a mutation at position 456, 457, 458, 459, 460, and 461, as compared to SEQ ID NO: 1;
the capsid polypeptide comprises a mutation that corresponds to a mutation at position 449, 450, 451, 454, 455, 456, 458, 459, 461, and 464 as compared to SEQ ID NO: 1;
the capsid polypeptide comprises a mutation that corresponds to a mutation at position 446, 448, 449, 451, 453, 455, 456, 457, and 459 as compared to SEQ ID NO: 1;
the capsid polypeptide comprises a mutation that corresponds to a mutation at position 447 as compared to SEQ ID NO: 1;
the capsid polypeptide comprises a mutation that corresponds to a mutation at position 581 as compared to SEQ ID NO: 1;
the capsid polypeptide comprises a mutation that corresponds to a mutation at position 584 as compared to SEQ ID NO: 1;
the capsid polypeptide comprises a mutation that corresponds to a mutation at position 555, 561, 566, 578, 580, 581, and 585 as compared to SEQ ID NO: 1;
the capsid polypeptide comprises a mutation that corresponds to a mutation at position 557 and 578 as compared to SEQ ID NO: 1;
the capsid polypeptide comprises a mutation that corresponds to a mutation at position 556, 558, 578, and 580 as compared to SEQ ID NO: 1;
the capsid polypeptide comprises a mutation that corresponds to a mutation at position 554, 556, 559, 561, 566, 581, 585, 586, and 587 as compared to SEQ ID NO: 1;
the capsid polypeptide comprises a mutation that corresponds to a mutation at position 552, 555, 556, 559, 561, 566, 578, 581, and 586 as compared to SEQ ID NO: 1;
the capsid polypeptide comprises a mutation that corresponds to a mutation at position 575 and 578 as compared to SEQ ID NO: 1;
the capsid polypeptide comprises a mutation that corresponds to a mutation at position 583 as compared to SEQ ID NO: 1;
the capsid polypeptide comprises a mutation that corresponds to a mutation at position 585, 588, 589, 590, 591, 593, 597, and 600 as compared to SEQ ID NO: 1;
the capsid polypeptide comprises a mutation that corresponds to a mutation at position 592 as compared to SEQ ID NO: 1;
the capsid polypeptide comprises a mutation that corresponds to a mutation at position 585 as compared to SEQ ID NO: 1;
the capsid polypeptide comprises a mutation that corresponds to a mutation at position 586 as compared to SEQ ID NO: 1;
the capsid polypeptide comprises a mutation that corresponds to a mutation at position 589 as compared to SEQ ID NO: 1;
the capsid polypeptide comprises a mutation that corresponds to a mutation at position 587 as compared to SEQ ID NO: 1;
the capsid polypeptide comprises a mutation that corresponds to a mutation at position 590 as compared to SEQ ID NO: 1;
the capsid polypeptide comprises a mutation that corresponds to a mutation at position 585, 586, 587, 591, 594, 597, and 600 as compared to SEQ ID NO: 1;
the capsid polypeptide comprises a mutation that corresponds to a mutation at position 591 as compared to SEQ ID NO: 1; or
the capsid polypeptide comprises a mutation that corresponds to a mutation at position 588 as compared to SEQ ID NO: 1.

19. The variant capsid polypeptide of any of the preceding claims, wherein the capsid polypeptide comprises an insertion, e.g., an insertion of 1 or more amino acids, e.g., 1-10 amino acids, e.g., 2-8 amino acids, e.g., 3-7 amino acids, e.g., 7 amino acids, that corresponds to an insertion between positions 446 and 447, 447 and 448, 448 and 449, 449 and 450, 451, and 452, 453 and 454, 581 and 582, 583 and 584, 584 and 585, 585 and 586, 586 and 587, 587 and 588, 588 and 589, 589 and 590, 590 and 591, 591 and 592, or 592 and 593, as compared to SEQ ID NO: 1.

20. The variant capsid polypeptide of any of the preceding claims, wherein:
the capsid polypeptide comprises a mutation that corresponds to a T14L, L15V, I19A, and K24H mutation as compared to SEQ ID NO: 1;
the capsid polypeptide comprises a mutation that corresponds to a A3V, Y6F, L11F, E12Q, Q21L, W23I, L25C, P29A, P31N, and K33R mutation as compared to SEQ ID NO: 1;
the capsid polypeptide comprises a mutation that corresponds to a N449Q, T450M, P451T, T455L, T456G, Q457T, S458Q, and R459M mutation as compared to SEQ ID NO: 1;
the capsid polypeptide comprises a mutation that corresponds to a N449Q, residue P451 deletion, S452T, T456G, Q457T, and R459G mutation as compared to SEQ ID NO: 1;
the capsid polypeptide comprises a mutation that corresponds to a Q457F and S458C mutation as compared to SEQ ID NO: 1;
the capsid polypeptide comprises a mutation that corresponds to an insertion between residues 446 and 447 as compared to SEQ ID NO: 1, wherein the insertion comprises a polypeptide of KCQEGMA (SEQ ID NO: 95) or a fragment of at least 4, at least 5, or at least 6 amino acids thereof;
the capsid polypeptide comprises a mutation that corresponds to an insertion between residues 449 and 450 as compared to SEQ ID NO: 1, wherein the insertion comprises a polypeptide of LMVDRLG (SEQ ID NO: 96) or a fragment of at least 4, at least 5, or at least 6 amino acids thereof;
the capsid polypeptide comprises a mutation that corresponds to a P451T, T455G, T456G, Q457T, S458Q, R459T, and Q461A mutation as compared to SEQ ID NO: 1;
the capsid polypeptide comprises a mutation that corresponds to an insertion between residues 448 and 449 as compared to SEQ ID NO: 1, wherein the insertion comprises a polypeptide of HCQECPI (SEQ ID NO: 97) or a fragment of at least 4, at least 5, or at least 6 amino acids thereof;
the capsid polypeptide comprises a mutation that corresponds to an insertion between residues 453 and 454 as compared to SEQ ID NO: 1, wherein the insertion comprises a polypeptide of FSGLEN (SEQ ID NO: 98) or a fragment of at least 5 amino acids thereof;
the capsid polypeptide comprises a mutation that corresponds to a residue deletion at positions T456, Q457, S458, R459, L460, and Q461 as compared to SEQ ID NO: 1;
the capsid polypeptide comprises a mutation that corresponds to a N449I, T450N, P451G, residue T454 deletion, T455Q, T456N, S458Q, R459T, Q461K, and Q464V mutation as compared to SEQ ID NO: 1;
the capsid polypeptide comprises a mutation that corresponds to a S446A, residue T448 and N449 deletion, P451Q, G453T, T455G, T456G, Q457T, and R459G mutation as compared to SEQ ID NO: 1;
the capsid polypeptide comprises a mutation that corresponds to a N449Q, T450S, S452G, T455A, Q457F, S458M, R459D, and an insertion between residues 451 and 452 as compared to SEQ ID NO: 1, wherein the insertion comprises an amino acid Y;
the capsid polypeptide comprises a mutation that corresponds to an insertion between residues 447 and 448 as compared to SEQ ID NO: 1, wherein the insertion comprises a polypeptide of HETEFNF (SEQ ID NO: 99) or a fragment of at least 4, at least 5, or at least 6 amino acids thereof;
the capsid polypeptide comprises a mutation that corresponds to a P451T, T455G, T456G, Q457T, S458Q, R459T, and Q461A mutation as compared to SEQ ID NO: 1;
the capsid polypeptide comprises a mutation that corresponds to an insertion between residues 581 and 582 as compared to SEQ ID NO: 1, wherein the insertion comprises a polypeptide of FALMEP (SEQ ID NO: 100) or a fragment of at least 4, or at least 5 amino acids thereof;
the capsid polypeptide comprises a mutation that corresponds to an insertion between residues 584 and 585 as compared to SEQ ID NO: 1, wherein the insertion comprises a polypeptide of RAYNPD (SEQ ID NO: 101) or a fragment of at least 4, or at least 5 amino acids thereof;
the capsid polypeptide comprises a mutation that corresponds to a residue R585 deletion and a E555A, D461S, R566K, S578V, S580A, and T581A mutation as compared to SEQ ID NO: 1;
the capsid polypeptide comprises a mutation that corresponds to a V557L and S578D mutation as compared to SEQ ID NO: 1;
the capsid polypeptide comprises a mutation that corresponds to a K556N, M558L, S578I, and S580A mutation as compared to SEQ ID NO: 1;
the capsid polypeptide comprises a mutation that corresponds to a I554L, K556R, I559L, D561S, R566A, T581D, R585S, G586R, and N587S mutation as compared to SEQ ID NO: 1;
the capsid polypeptide comprises a mutation that corresponds to a V552A, E555S, K556D, I559L, D561S, R566K, S578I, T581D, and G586Q mutation as compared to SEQ ID NO: 1;
the capsid polypeptide comprises a mutation that corresponds to a Q575E and S578A mutation as compared to SEQ ID NO: 1;
the capsid polypeptide comprises a mutation that corresponds to an insertion between residues 583 and 584 as compared to SEQ ID NO: 1, wherein the insertion comprises a polypeptide of LNWTAE (SEQ ID NO: 102) or a fragment of at least 4, or at least 5 amino acids thereof;
the capsid polypeptide comprises a mutation that corresponds to a R585S, R588T, Q589N, A590P, A591I, A593G, T597S, and V600A mutation as compared to SEQ ID NO: 1;
the capsid polypeptide comprises a mutation that corresponds to a R585N and G586A mutation, and an insertion between residues 584 and 585 as compared to SEQ ID NO: 1, wherein the insertion comprises a polypeptide of LAKEFTTR (SEQ ID NO: 103) or a fragment of at least 4, at least 5, at least 6, or at least 7 amino acids thereof (e.g., a fragment comprising or consisting of KEFTTR (SEQ ID NO: 104));
the capsid polypeptide comprises a mutation that corresponds to an insertion between residues 592 and 593 as compared to SEQ ID NO: 1, wherein the insertion comprises a polypeptide of LHPLE (SEQ ID NO: 105) or a fragment of at least 4 amino acids thereof;
the capsid polypeptide comprises a mutation that corresponds to an insertion between residues 585 and 586 as compared to SEQ ID NO: 1, wherein the insertion comprises an amino acid F;
the capsid polypeptide comprises a mutation that corresponds to an insertion between residues 586 and 587 as compared to SEQ ID NO: 1, wherein the insertion comprises a polypeptide of DQDFKNR (SEQ ID NO: 106) or a fragment of at least 4, at least 5, or at least 6 amino acids thereof;
the capsid polypeptide comprises a mutation that corresponds to an insertion between residues 589 and 590 as compared to SEQ ID NO: 1, wherein the insertion comprises an amino acid I;
the capsid polypeptide comprises a mutation that corresponds to an insertion between residues 587 and 588 as compared to SEQ ID NO: 1, wherein the insertion comprises a polypeptide of LAIEQTRPA (SEQ ID NO: 107) or a fragment of at least 4, at least 5,at least 6, at least 7, or at least 8 amino acids thereof (e.g., a fragment comprising or consisting of IEQTRPA (SEQ ID NO: 108));
the capsid polypeptide comprises a mutation that corresponds to a G586P and N587A mutation, and an insertion between residues 584 and 585 as compared to SEQ ID NO: 1, wherein the insertion comprises a polypeptide of RARLDETT (SEQ ID NO: 109) or a fragment of at least 4, at least 5, at least 6, or at least 7 amino acids thereof (e.g., a fragment comprising or consisting of RLDETT (SEQ ID NO: 110));
the capsid polypeptide comprises a mutation that corresponds to a N587A mutation, and an insertion between residues 586 and 587 as compared to SEQ ID NO: 1, wherein the insertion comprises a polypeptide of LALAEITRP (SEQ ID NO: 111) or a fragment of at least 4, at least 5, at least 6, at least 7, or at least 8 amino acids thereof (e.g., a fragment comprising or consisting of LAEITRP (SEQ ID NO: 112));
the capsid polypeptide comprises a mutation that corresponds to a N587A mutation, and an insertion between residues 586 and 587 as compared to SEQ ID NO: 1, wherein the insertion comprises a polypeptide of LANGEQTRP (SEQ ID NO: 113) or a fragment of at least 4, at least 5, at least 6, at least 7, or at least 8 amino acids thereof (e.g., a fragment comprising or consisting of NGEQTRP (SEQ ID NO: 114));
the capsid polypeptide comprises a mutation that corresponds to an insertion between residues 586 and 587 as compared to SEQ ID NO: 1, wherein the insertion comprises a polypeptide of ATDTKT (SEQ ID NO: 115) or a fragment of at least 4, or at least 5 amino acids thereof;
the capsid polypeptide comprises a mutation that corresponds to an insertion between residues 590 and 591 as compared to SEQ ID NO: 1, wherein the insertion comprises an amino acid P;
the capsid polypeptide comprises a mutation that corresponds to an insertion between residues 587 and 588 as compared to SEQ ID NO: 1, wherein the insertion comprises a polypeptide of APGETTRPA (SEQ ID NO: 116) or a fragment of at least 4, at least 5, at least 6, at least 7, or at least 8 amino acids thereof;
the capsid polypeptide comprises a mutation that corresponds to an insertion between residues 589 and 590 as compared to SEQ ID NO: 1, wherein the insertion comprises an amino acid P;
the capsid polypeptide comprises a mutation that corresponds to a R585S, G586S, N587A, A591E, D594R, T597A, and V600I mutation as compared to SEQ ID NO: 1;
the capsid polypeptide comprises a mutation that corresponds to an insertion between residues 586 and 587 as compared to SEQ ID NO: 1, wherein the insertion comprises a polypeptide of FHNEGKY (SEQ ID NO: 118) or a fragment of at least 4, at least 5, or at least 6 amino acids thereof;
the capsid polypeptide comprises a mutation that corresponds to an insertion between residues 591 and 592 as compared to SEQ ID NO: 1, wherein the insertion comprises an amino acid G;
the capsid polypeptide comprises a mutation that corresponds to an insertion between residues 588 and 589 as compared to SEQ ID NO: 1, wherein the insertion comprises a polypeptide of QPWEPDK (SEQ ID NO: 119) or a fragment of at least 4, at least 5, or at least 6 amino acids thereof;
the capsid polypeptide comprises a mutation that corresponds to an insertion between residues 592 and 593 as compared to SEQ ID NO: 1, wherein the insertion comprises a polypeptide of ALALSTTN (SEQ ID NO: 120) or a fragment of at least 4, at least 5, at least 6, or at least 7 amino acids thereof (e.g., a fragment comprising or consisting of ALSTTN (SEQ ID NO: 121)); or
the capsid polypeptide comprises a mutation that corresponds to an insertion between residues 585 and 586 as compared to SEQ ID NO: 1, wherein the insertion comprises a polypeptide of PWGTAG (SEQ ID NO: 122) or a fragment of at least 4, or at least 5 amino acids thereof.

21. A variant capsid polypeptide, comprising (a) a polypeptide of any one of SEQ ID NO: 37, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46, (b) comprising the VP2 or VP3 sequence of any one of SEQ ID NO: 37, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46; (c) a polypeptide comprising a sequence having at least 90%, at least 95%, at least 96%, at least 97%, at least 98%, or at least 99% identity thereto, wherein said sequence comprises at least one (e.g., one, two, three or more, e.g., all) of the mutation differences associated with any of SEQ ID NO: 2 through SEQ ID NO: 46, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutation differences associated with any of SEQ ID NO: 2 through SEQ ID NO: 46, relative to SEQ ID NO: 1.

22. The variant capsid polypeptide of any of the preceding claims, wherein the variant capsid polypeptide is a VP1 polypeptide, a VP2 polypeptide or a VP3 polypeptide.

23. A variant capsid polypeptide comprising a mutation that corresponds to a leucine at a position corresponding to 554 of SEQ ID NO: 1 (e.g., comprising I554L as compared to SEQ ID NO: 1).

24. The variant capsid polypeptide of claim 22 or any one of the preceding claims, wherein:
the capsid polypeptide has up to 12 additional mutations, e.g., between 8 and 12 mutations, as compared to SEQ ID NO: 1; or
the capsid polypeptide has up to 16 additional mutations, e.g., between 11 and 16 mutations, as compared to VAR-22 (SEQ ID NO: 23).

25. The variant capsid polypeptide of claim 22 or any one of the preceding claims, further comprising:
a mutation that corresponds to a serine at a position corresponding to 561 of SEQ ID NO: 1 (e.g., comprising D561S as compared to SEQ ID NO: 1); or
a mutation that corresponds to an aspartic acid at a position corresponding to 581 of SEQ ID NO: 1 (e.g., comprising T581D as compared to SEQ ID NO: 1).

26. A variant capsid polypeptide comprising a mutation that corresponds to a leucine at a position corresponding to 559 of SEQ ID NO: 1 (e.g., comprising I559L as compared to SEQ ID NO: 1).

27. The variant capsid polypeptide of claim 26 or any one of the preceding claims, wherein:
the capsid polypeptide has up to 13 additional mutations, e.g., between 4 and 13 mutations, as compared to SEQ ID NO: 1;
the capsid polypeptide has between 8 and 19 mutations as compared to VAR-22 (SEQ ID NO: 23); or
the capsid polypeptide has up to 20 additional mutations, e.g., between 5 and 20 mutations, as compared to VAR-23 (SEQ ID NO: 24).

28. The variant capsid polypeptide of claim 26 or any one of the preceding claims, further comprising a mutation that corresponds to a serine at a position corresponding to 561 of SEQ ID NO: 1 (e.g., comprising D561S as compared to SEQ ID NO: 1), optionally wherein capsid polypeptide further comprises an aspartic acid at a position corresponding to 581 of SEQ ID NO: 1 (e.g., comprising a T581D as compared to SEQ ID NO: 1).

29. A variant capsid polypeptide comprising a mutation that corresponds to asparagine at a position corresponding to 556 of SEQ ID NO: 1 (e.g., comprising K556N as compared to SEQ ID NO: 1).

30. The variant capsid polypeptide of claim 29 or any one of the preceding claims, wherein:
the capsid polypeptide has up to 12 additional mutations, e.g., between 4 and 12 mutations, as compared to SEQ ID NO: 1; or
the capsid polypeptide has up to 12 additional mutations, e.g., between 9 and 12 mutations, as compared to VAR-21 (SEQ ID NO: 22).

31. A variant capsid polypeptide comprising a mutation that corresponds to a serine at a position corresponding to 561 of SEQ ID NO: 1 (e.g., comprising D561S as compared to SEQ ID NO: 1).

32. The variant capsid polypeptide of claim 31 or any one of the preceding claims, wherein:
the capsid polypeptide has up to 12 additional mutations, e.g., between 7 and 12 mutations, as compared to SEQ ID NO: 1;
the capsid polypeptide has up to 14 additional mutations, e.g., between 11 and 14 mutations, as compared to VAR-19 (SEQ ID NO: 20);
the capsid polypeptide has up to 14 additional mutations, e.g., between 10 and 14 mutations, as compared to VAR-22 (SEQ ID NO: 23); or
the capsid polypeptide has up to 14 additional mutations, e.g., between 9 and 14 mutations, as compared to VAR-23 (SEQ ID NO: 24).

33. The variant capsid polypeptide of claim 31 or any one of the preceding claims, further comprising a mutation that corresponds to an aspartic acid at a position corresponding to 581 of SEQ ID NO:1 (e.g., comprising T581D as compared to SEQ ID NO: 1).

34. A capsid polypeptide comprising a mutation at a position corresponding to 587 of SEQ ID NO: 1, optionally wherein the mutation is to an alanine (e.g., wherein the mutation is N587A according to SEQ ID NO: 1), optionally wherein the capsid polypeptide further comprises an insertion (e.g., an insertion of 4 or more amino acids, optionally an insertion of 7 or more amino acids, optionally an insertion of 7-10 amino acids, optionally an insertion of 7, 8 or 9 amino acids) between two adjacent amino acids between 580 and 587 (optionally between 586 and 587) according to SEQ ID NO: 1.

35. A nucleic acid molecule comprising sequence encoding a variant capsid polypeptide of any one of claims 1-34.

36. The nucleic acid molecule of claim 35, comprising one or more regulatory elements operably linked to the sequence encoding the variant capsid polypeptide.

37. The nucleic acid molecule of any of claims 35-36, comprising SEQ ID NO: 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, or a fragment thereof, or a variant thereof having at least 85, 90, 91, 92, 93, 94, 95, 96, 97, 98, 99, or 100% sequence identity thereto.

38. A virus particle (e.g., adeno-associated virus (“AAV”) particle) comprising the variant capsid polypeptide of any one of claims 1-34 or comprising a variant capsid polypeptide encoded by the nucleic acid molecule of any one of claims 35-37.

39. The virus particle of claim 38, comprising a nucleic acid comprising a heterologous transgene and one or more regulatory elements.

40. A virus particle of any of claims 38-39 comprising the variant capsid polypeptide of any one of claims 1-34, wherein said virus particle, or a virus particle comprising said variant capsid polypeptide or a virus particle comprising a variant capsid polypeptide encoded by a nucleic acid molecule of any one of claims 35-37 exhibits increased ocular transduction, e.g., as measured in a mouse or in NHP, e.g., as described herein, relative to wild-type AAV2 (e.g., a virus particle comprising capsid polypeptides of SEQ ID NO: 1 or encoded by SEQ ID NO: 92).

41. The nucleic acid molecule of any one of claims 35-37, wherein the nucleic acid molecule is double-stranded or single-stranded, optionally wherein the nucleic acid molecule is linear or circular, e.g., wherein the nucleic acid molecule is a plasmid.

42. A method of producing a virus particle comprising a variant AAV2 capsid polypeptide, said method comprising introducing a nucleic acid molecule of any one of claims 35-37 or 41 into a cell (e.g., a HEK293 cell), and harvesting said virus particle therefrom.

43. A method of delivering a payload (e.g., a nucleic acid) to a cell comprising contacting the cell with a dependoparvovirus particle comprising a variant capsid polypeptide of any one of claims 1-34 or the virus particle of any of claims 38-40 and a payload.

44. The method of claim 43, wherein the cell is an ocular cell, and wherein the ocular cell is in the retina, the macula, or the trabecular meshwork.

45. A method of delivering a payload (e.g., a nucleic acid) to a subject comprising administering to the subject a dependoparvovirus particle comprising a variant capsid polypeptide of any one of claims 1-34 and the payload, or administering to the subject the virus particle of any one of claims 38-40.

46. The method of claim 45, wherein the particle delivers the payload to the eye, and wherein the particle delivers the payload to the retina, the macular, or the trabecular meshwork.

47. The method of any one of claims 45-46, wherein the particle delivers the payload to the eye with increased transduction in one or more regions of the eye as compared to a virus particle comprising capsid polypeptides of SEQ ID NO: 1, and wherein the one or more regions of the eye is selected from the retina, the macula, the trabecular meshwork, or any combination thereof.

48. The variant capsid polypeptide of any of claims 1-34, the virus particle of any of claims 38-40 or the method of any one of claims 42-47, wherein the particle (e.g., particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, or 150-times as compared to a virus particle comprising capsid polypeptides of SEQ ID NO: 1.

49. The variant capsid polypeptide of any of claims 1-34, the virus particle of any of claims 38-40 or the method of any one of claims 42-47, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is:
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to macular tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to non-macular retina tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue relative to trabecular meshwork tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to trabecular meshwork tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to macular tissue and non-macular retina tissue relative to trabecular meshwork tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue and non-macular retina tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to macular tissue;
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue relative to non-macular retina tissue; or
at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to trabecular meshwork tissue, macular tissue, and non-macular retina tissue.

50. The variant capsid polypeptide of any of claims 1-34, the virus particle of any of claims 38-40 or the method of any one of claims 42-47, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1 without increased biodistribution in one or more regions of the eye as compared to a virus particle comprising capsid polypeptides of SEQ ID NO: 1.

51. The method of any one of claims 43-50, wherein the administration to the subject is via an intravitreal injection, or an intracameral injection.

52. A method of treating a disease or condition in a subject, comprising administering to the subject a dependoparvovirus particle in an amount effective to treat the disease or condition, wherein the dependoparvovirus particle is a particle comprising a capsid polypeptide of any one of claims 1-34 and 48-50, or encoded by the nucleic acid of any one of claims 35-37 or 41, or is a virus particle of any one of claims 38-40.

53. A cell, cell-free system, or other translation system, comprising the capsid polypeptide, nucleic acid molecule, or virus particle of any one of claims 1-41 or 48-50.

54. A method of making a dependoparvovirus (e.g., an adeno-associated dependoparvovirus (AAV) particle, comprising:
providing a cell, cell-free system, or other translation system, comprising a nucleic acid of any of claims 35-37 or 41; and
cultivating the cell, cell-free system, or other translation system, under conditions suitable for the production of the dependoparvovirus particle,
thereby making the dependoparvovirus particle.

55. The method of claim 54, wherein the cell, cell-free system, or other translation system comprises a second nucleic acid molecule and said second nucleic acid molecule is packaged in the dependoparvovirus particle, and wherein the second nucleic acid comprises a payload, e.g., a heterologous nucleic acid sequence encoding a therapeutic product.

56. The method of any one of claims 54-55, wherein the nucleic acid of any of claims 35-37 or 41 mediates the production of a dependoparvovirus particle which does not include said nucleic acid of any of claims 35-37 or 41.

57. The method of any one of claims 54-56, wherein the nucleic acid of any of claims 35-37 or 41 mediates the production of a dependoparvovirus particle at a level at least 10%, at least 20%, at least 50%, at least 100%, at least 200% or greater than the production level mediated by the nucleic acid of SEQ ID NO: 92.

58. A composition, e.g., a pharmaceutical composition, comprising a virus particle of any one of claims 38-40 or a virus particle produced by the method of any one of claims 126 or 150-154, and a pharmaceutically acceptable carrier.

59. The variant capsid polypeptide of any of claims 1-34 and 48-50, the nucleic acid molecule of any of claims 35-37 or 41, or the virus particle of any of claims 38-40 for use in treating a disease or condition in a subject.

60. The variant capsid polypeptide of any of claims 1-34 and 48-50, the nucleic acid molecule of any of claims 35-37 or 41, or the virus particle of any of claims 38-40 for use in the manufacture of a medicament for use in treating a disease or condition in a subject.


SEQUENCE LISTING (163 sequences)

SEQ ID NO: 1 - protein, 735 aa, Adeno-associated virus 2 [sequence omitted]

SEQ ID NO: 2 - protein, 735 aa, Artificial Sequence
  Description of Artificial Sequence Synthetic polypeptide
MAADGYLPDWLEDLVSEGARQWWHLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, Artificial Sequence
  Description of Artificial Sequence Synthetic polypeptide
MAVDGFLPDWFQDTLSEGIRLWIKCKPGAPNPRPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQMTSGTLGTQMLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQTTGTTGTSGLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTFCRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKCQEGMARTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNLMVDRLGTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTTSGTGGTQTLAFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTHCQECPINTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGFSGLENTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 729 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTINGSGQNQQTLKFSVAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 733 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLARTQSTTGGTSGLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTQSPYGGTATFMDLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRHETEFNFTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 735 aa, Artificial Sequence; identical to SEQ ID NO: 9
  Description of Artificial Sequence Synthetic polypeptide

SEQ ID NO: 18 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTFALMEPNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRAYNPDRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 734 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIAKVMITSEEEIKTTNPVATEQYGVVAANLQGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKLMITDEEEIRTTNPVATEQYGDVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIENVLITDEEEIRTTNPVATEQYGIVATNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDLERVMLTSEEEIATTNPVATEQYGSVSDNLQSRSRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNADISDVMLTSEEEIKTTNPVATEQYGIVSDNLQRQNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEEYGAVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLLNWTAEQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTNPITGDVNSQGALPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - protein, 743 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQLAKEFTTRNANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 740 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATLHPLEADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRFGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 31 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGDQDFKNRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQIAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 33 - protein, 744 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAIEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 34 - protein, 743 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRARLDETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 744 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALAEITRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 36 - protein, 744 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLANGEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 37 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGATDTKTNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAPATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 39 - protein, 744 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAPGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 40 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQPAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 41 - protein, 735 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSSARQAETARVNAQGILPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 42 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGFHNEGKYNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 43 - protein, 736 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAAGTADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 44 - protein, 742 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQPWEPDKQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 45 - protein, 743 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATALALSTTNADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 46 - protein, 741 aa, Artificial Sequence
  Description of Artificial Sequence Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRPWGTAGGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - DNA, 2208 bp, Artificial Sequence
  Description of Artificial Sequence Synthetic polynucleotide
atggctgccgatggttatcttccagattggctcgaggacctggtatctgaaggagcgagacagtggtggcatctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 48 - DNA, 2208 bp, Artificial Sequence
  Description of Artificial Sequence Synthetic polynucleotide
atggctgttgatggttttcttccagattggtttcaagacactctctctgaaggaataagattatggatcaagtgcaaacctggcgcgccaaacccacgccccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 49 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccggctcatgaatcctctcatcgaccagtacctgtattacttgagcagaacacagatgaccagtggaaccttgggtacacagatgcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 50 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggacagactcatgaatcctctcatcgaccagtacctgtattacttgagcagaacacaaactactggaaccaccggtacctcaggacttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 51 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtcttatgaatcctctcatcgaccagtacctgtattacttgagcagaacaaacactccaagtggaaccaccacgttttgtaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 52 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctgatgaatcctctcatcgaccagtacctgtattacttgagcaagtgccaggaaggaatggctagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 53 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctaatgaatcctctcatcgaccagtacctgtattacttgagcagaacaaacctcatggttgaccgacttgggactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 54 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgactcatgaatcctctcatcgaccagtacctgtattacttgagcagaacaaacactacaagtggaaccggaggaacacagacgcttgctttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 55 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctgatgaatcctctcatcgaccagtacctgtattacttgagcagaacacattgtcaggagtgtccaattaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 56 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctaatgaatcctctcatcgaccagtacctgtattacttgagcagaacaaacactccaagtggattctctggcttggagaacaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 57 - DNA, 2190 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggacagactcatgaatcctctcatcgaccagtacctgtattacttgagcagaacaaacactccaagtggaaccaccttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 58 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctgatgaatcctctcatcgaccagtacctgtattacttgagcagaacaatcaacgggagtggacagaaccagcaaactcttaagttttctgtggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 59 - DNA, 2202 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtcttatgaatcctctcatcgaccagtacctgtattacttggctagaactcaaagtacaaccgggggtacctcagggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 60 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgcctcatgaatcctctcatcgaccagtacctgtattacttgagcagaacacaatcgccatacgggggaaccgccacgttcatggaccttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 61 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtttgatgaatcctctcatcgaccagtacctgtattacttgagcagacacgaaaccgaattcaattttacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 62 - DNA, 2208 bp, Artificial Sequence; identical to SEQ ID NO: 54
  Description of Artificial Sequence Synthetic polynucleotide

SEQ ID NO: 63 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctacctttgccctcatggagccgaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 64 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcgtgcgtacaatcctgatagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 65 - DNA, 2205 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgcaaaggtcatgattacatccgaagaggaaatcaagacaaccaatcccgtggctacggagcagtatggtgtggtagctgctaacctccagggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 66 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaagctcatgattacagacgaagaggaaatcaggacaaccaatcccgtggctacggagcagtatggtgatgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 67 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaacgtcctcattacagacgaagaggaaatcaggacaaccaatcccgtggctacggagcagtatggtattgtagctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 68 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacctcgaacgtgtcatgcttacatcggaagaggaaatcgccacaaccaatcccgtggctacggagcagtatggttctgtatctgacaacctccagtcgagatcaagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 69 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgctgacatttctgacgtcatgcttacaagcgaagaggaaatcaaaacaaccaatcccgtggctacggagcagtatggtattgtatctgacaacctccagagacagaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 70 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggaggagtatggtgctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 71 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctcctcaattggactgccgaacagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 72 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagtctggcaacacaaatcctattaccggagatgtcaactcgcaaggcgcgcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 73 - DNA, 2232 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagttggcgaaagaattcactacgcggaatgcgaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 74 - DNA, 2223 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccttacatccgctggaggcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 75 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagatttggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 76 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcgaccaagatttcaaaaaccgaaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 77 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaaatcgcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 78 - DNA, 2235 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaaccttgctatcgaacaaacaagaccagcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 79 - DNA, 2232 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagagctcgattggatgagacaacacgccctgctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 80 - DNA, 2235 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcttggctctcgctgagatcaccagaccggcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 81 - DNA, 2235 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcctggcgaacggcgaacaaaccaggcctgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 82 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcgctacggatactaagacgaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 83 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcaccagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 84 - DNA, 2235 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacgcacccggagagactactaggccagcaagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 85 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaaccggcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 86 - DNA, 2208 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagtcttctgctagacaagcagagacagcacgggtcaacgctcaaggcattcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 87 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcttccataatgaaggaaaatacaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 88 - DNA, 2211 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctgggaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 89 - DNA, 2229 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaaccgtgggaaccggataagcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 90 - DNA, 2232 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcactggcgcttagtacaactaatgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 91 - DNA, 2226 bp, Artificial Sequence
  Description of Artificial Sequence Synthetic polynucleotide
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcggccttggggcacagctggaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 92 - DNA, 2208 bp, Adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 93 - protein, 10 aa, Artificial Sequence
  Description of Artificial Sequence Synthetic peptide
LALGETTRPA

SEQ ID NO: 94 - protein, 7 aa, Artificial Sequence
  Description of Artificial Sequence Synthetic peptide
LGETTRP

SEQ ID NO: 95 - protein, 7 aa, Artificial Sequence
  Description of Artificial Sequence Synthetic peptide
KCQEGMA

SEQ ID NO: 96 - protein, 7 aa, Artificial Sequence
  Description of Artificial Sequence Synthetic peptide
LMVDRLG

SEQ ID NO: 97 - protein, 7 aa, Artificial Sequence
  Description of Artificial Sequence Synthetic peptide
HCQECPI

SEQ ID NO: 98 - protein, 6 aa, Artificial Sequence
  Description of Artificial Sequence Synthetic peptide
FSGLEN

SEQ ID NO: 99 - protein, 7 aa, Artificial Sequence
  Description of Artificial Sequence Synthetic peptide
HETEFNF

SEQ ID NO: 100 - protein, 6 aa, Artificial Sequence
  Description of Artificial Sequence Synthetic peptide
FALMEP

SEQ ID NO: 101 - protein, 6 aa, Artificial Sequence
  Description of Artificial Sequence Synthetic peptide
RAYNPD

SEQ ID NO: 102 - protein, 6 aa, Artificial Sequence
  Description of Artificial Sequence Synthetic peptide
LNWTAE

SEQ ID NO: 103 - protein, 8 aa, Artificial Sequence
  Description of Artificial Sequence Synthetic peptide
LAKEFTTR

SEQ ID NO: 104 - protein, 6 aa, Artificial Sequence
  Description of Artificial Sequence Synthetic peptide
KEFTTR

SEQ ID NO: 105 - protein, 5 aa, Artificial Sequence
  Description of Artificial Sequence Synthetic peptide
LHPLE

SEQ ID NO: 106 - protein, 7 aa, Artificial Sequence
  Description of Artificial Sequence Synthetic peptide
DQDFKNR

SEQ ID NO: 107 - protein, 9 aa, Artificial Sequence
  Description of Artificial Sequence Synthetic peptide
LAIEQTRPA

SEQ ID NO: 108 - protein, 7 aa, Artificial Sequence
  Description of Artificial Sequence Synthetic peptide
IEQTRPA

SEQ ID NO: 109 - protein, 8 aa, Artificial Sequence
  Description of Artificial Sequence Synthetic peptide
RARLDETT

SEQ ID NO: 110 - protein, 6 aa, Artificial Sequence
  Description of Artificial Sequence Synthetic peptide
RLDETT

SEQ ID NO: 111 - protein, 9 aa, Artificial Sequence
  Description of Artificial Sequence Synthetic peptide
LALAEITRP

SEQ ID NO: 112 - protein, 7 aa, Artificial Sequence
  Description of Artificial Sequence Synthetic peptide
LAEITRP

SEQ ID NO: 113 - protein, 9 aa, Artificial Sequence
  Description of Artificial Sequence Synthetic peptide
LANGEQTRP

SEQ ID NO: 114 - protein, 7 aa, Artificial Sequence
  Description of Artificial Sequence Synthetic peptide
NGEQTRP

SEQ ID NO: 115 - protein, 6 aa, Artificial Sequence
  Description of Artificial Sequence Synthetic peptide
ATDTKT

SEQ ID NO: 116 - protein, 9 aa, Artificial Sequence
  Description of Artificial Sequence Synthetic peptide
APGETTRPA

SEQ ID NO: 117 - protein, 6 aa, Artificial Sequence
  Description of Artificial Sequence Synthetic peptide
GETTRP

SEQ ID NO: 118 - protein, 7 aa, Artificial Sequence
  Description of Artificial Sequence Synthetic peptide
FHNEGKY

SEQ ID NO: 119 - protein, 7 aa, Artificial Sequence
  Description of Artificial Sequence Synthetic peptide
QPWEPDK

SEQ ID NO: 120 - protein, 8 aa, Artificial Sequence
  Description of Artificial Sequence Synthetic peptide
ALALSTTN

SEQ ID NO: 121 - protein, 6 aa, Artificial Sequence
  Description of Artificial Sequence Synthetic peptide
ALSTTN

SEQ ID NO: 122 - protein, 6 aa, Artificial Sequence
  Description of Artificial Sequence Synthetic peptide
PWGTAG

SEQ ID NO: 123 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 124 - DNA, 2175 bp, Adeno-associated virus 5
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 125 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 126 - DNA, 2217 bp, Adeno-associated virus 8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 127 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 128 - DNA, 2211 bp, Adeno-associated virus 9
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 129 - protein, 738 aa, Unknown
  Description of Unknown AAVrh74 sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 130 - protein, 738 aa, Unknown
  Description of Unknown AAVrh74 sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 131 - DNA, 2217 bp, Unknown
  Description of Unknown AAVrh74 sequence
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 132 - protein, 15 aa, Artificial Sequence
  Description of Artificial Sequence Synthetic peptide
  MOD_RES (2)..(2): Any amino acid, preferably D
  MOD_RES (4)..(4): S or A
  MOD_RES (5)..(5): D, R or N
  MOD_RES (9)..(9): Any amino acid, preferably T
  MOD_RES (11)..(11): Any amino acid, preferably E
  MOD_RES (12)..(12): Any amino acid, preferably E
  MOD_RES (13)..(13): Any amino acid, preferably E
  MOD_RES (14)..(14): Any amino acid, preferably I
  MOD_RES (15)..(15): K or A
AXLXXLLLXSXXXXX

SEQ ID NO: 133 - protein, 13 aa, Artificial Sequence
  Description of Artificial Sequence Synthetic peptide
  MOD_RES (2)..(2): Any amino acid, preferably Y
  MOD_RES (3)..(3): Any amino acid, preferably G
  MOD_RES (4)..(4): A, I, D, or V
  MOD_RES (5)..(5): Any amino acid, preferably V
  MOD_RES (7)..(7): A or D
  MOD_RES (8)..(8): Any amino acid, preferably N
  MOD_RES (9)..(9): Any amino acid, preferably L
  MOD_RES (10)..(10): Any amino acid, preferably Q
  MOD_RES (11)..(11): S or absent
  MOD_RES (12)..(12): R or Q
EXXXXAXXXXXXS

SEQ ID NO: 134 - protein, 8 aa, Artificial Sequence
  Description of Artificial Sequence Synthetic peptide
  MOD_RES (1)..(2): Any amino acid or absent
XXEQTRPA

SEQ ID NO: 135 - protein, 6 aa, Artificial Sequence
  Description of Artificial Sequence Synthetic peptide
  MOD_RES (5)..(6): Any amino acid or absent
RAYNXX

SEQ ID NO: 136 - protein, 7 aa, Artificial Sequence
  Description of Artificial Sequence Synthetic peptide
  MOD_RES (6)..(7): Any amino acid or absent
DQDFKXX

SEQ ID NO: 137 - protein, 7 aa, Artificial Sequence
  Description of Artificial Sequence Synthetic peptide
  MOD_RES (6)..(7): Any amino acid or absent
IEQTRXX

SEQ ID NO: 138 - protein, 9 aa, Artificial Sequence
  Description of Artificial Sequence Synthetic peptide
  MOD_RES (8)..(9): Any amino acid or absent
LAIEQTRXX

SEQ ID NO: 139 - protein, 7 aa, Artificial Sequence
  Description of Artificial Sequence Synthetic peptide
  MOD_RES (1)..(2): Any amino acid or absent
XXQTRPA

SEQ ID NO: 140 - protein, 7 aa, Artificial Sequence
  Description of Artificial Sequence Synthetic peptide
  MOD_RES (6)..(7): Any amino acid or absent
RARLDXX

SEQ ID NO: 141 - protein, 8 aa, Artificial Sequence
  Description of Artificial Sequence Synthetic peptide
  MOD_RES (7)..(8): Any amino acid or absent
RARLDEXX

SEQ ID NO: 142 - protein, 9 aa, Artificial Sequence
  Description of Artificial Sequence Synthetic peptide
  MOD_RES (8)..(9): Any amino acid or absent
RARLDETXX

SEQ ID NO: 143 - protein, 8 aa, Artificial Sequence
  Description of Artificial Sequence Synthetic peptide
  MOD_RES (1)..(2): Any amino acid or absent
XXGEQTRP

SEQ ID NO: 144 - protein, 7 aa, Artificial Sequence
  Description of Artificial Sequence Synthetic peptide
  MOD_RES (1)..(2): Any amino acid or absent
XXEQTRP

SEQ ID NO: 145 - protein, 6 aa, Artificial Sequence
  Description of Artificial Sequence Synthetic peptide
  MOD_RES (5)..(6): Any amino acid or absent
ATDTXX

SEQ ID NO: 146 - protein, 7 aa, Artificial Sequence
  Description of Artificial Sequence Synthetic peptide
  MOD_RES (6)..(7): Any amino acid or absent
ATDTKXX

SEQ ID NO: 147 - protein, 7 aa, Artificial Sequence
  Description of Artificial Sequence Synthetic peptide
  MOD_RES (1)..(2): Any amino acid or absent
XXTDTKT

SEQ ID NO: 148 - protein, 8 aa, Artificial Sequence
  Description of Artificial Sequence Synthetic peptide
  MOD_RES (1)..(2): Any amino acid or absent
XXEQTRPA

SEQ ID NO: 149 - protein, 5 aa, Artificial Sequence
  Description of Artificial Sequence Synthetic peptide
  MOD_RES (5)..(5): Any amino acid
  See specification as filed for detailed description of substitutions and preferred embodiments
RAYNX

SEQ ID NO: 150 - protein, 6 aa, Artificial Sequence
  Description of Artificial Sequence Synthetic peptide
  MOD_RES (6)..(6): Any amino acid
  See specification as filed for detailed description of substitutions and preferred embodiments
DQDFKX

SEQ ID NO: 151 - protein, 8 aa, Artificial Sequence
  Description of Artificial Sequence Synthetic peptide
  MOD_RES (8)..(8): Any amino acid
  See specification as filed for detailed description of substitutions and preferred embodiments
LAIEQTRX

SEQ ID NO: 152 - protein, 7 aa, Artificial Sequence
  Description of Artificial Sequence Synthetic peptide
  MOD_RES (1)..(1): Any amino acid
  See specification as filed for detailed description of substitutions and preferred embodiments
XEQTRPA

SEQ ID NO: 153 - protein, 6 aa, Artificial Sequence
  Description of Artificial Sequence Synthetic peptide
  MOD_RES (1)..(1): Any amino acid
  See specification as filed for detailed description of substitutions and preferred embodiments
XQTRPA

SEQ ID NO: 154 - protein, 8 aa, Artificial Sequence
  Description of Artificial Sequence Synthetic peptide
  MOD_RES (8)..(8): Any amino acid
  See specification as filed for detailed description of substitutions and preferred embodiments
RARLDETX

SEQ ID NO: 155 - protein, 7 aa, Artificial Sequence
  Description of Artificial Sequence Synthetic peptide
  MOD_RES (7)..(7): Any amino acid
  See specification as filed for detailed description of substitutions and preferred embodiments
RARLDEX

SEQ ID NO: 156 - protein, 6 aa, Artificial Sequence
  Description of Artificial Sequence Synthetic peptide
  MOD_RES (6)..(6): Any amino acid
  See specification as filed for detailed description of substitutions and preferred embodiments
RARLDX

SEQ ID NO: 157 - protein, 7 aa, Artificial Sequence
  Description of Artificial Sequence Synthetic peptide
  MOD_RES (1)..(1): Any amino acid
  See specification as filed for detailed description of substitutions and preferred embodiments
XGEQTRP

SEQ ID NO: 158 - protein, 6 aa, Artificial Sequence
  Description of Artificial Sequence Synthetic peptide
  MOD_RES (1)..(1): Any amino acid
  See specification as filed for detailed description of substitutions and preferred embodiments
XEQTRP

SEQ ID NO: 159 - protein, 5 aa, Artificial Sequence
  Description of Artificial Sequence Synthetic peptide
  MOD_RES (5)..(5): Any amino acid
  See specification as filed for detailed description of substitutions and preferred embodiments
ATDTX

SEQ ID NO: 160 - protein, 6 aa, Artificial Sequence
  Description of Artificial Sequence Synthetic peptide
  MOD_RES (6)..(6): Any amino acid
  See specification as filed for detailed description of substitutions and preferred embodiments
ATDTKX

SEQ ID NO: 161 - protein, 6 aa, Artificial Sequence
  Description of Artificial Sequence Synthetic peptide
  MOD_RES (1)..(1): Any amino acid
  See specification as filed for detailed description of substitutions and preferred embodiments
XTDTKT

SEQ ID NO: 162 - protein, 735 aa, Adenoassociated virus 2
MAADGYLPDWLEDTLSEGIRQWWKLRPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 163 - protein, 4 aa, Unknown
  Description of Unknown Box polypeptide 38 sequence
DEAH